(19) United States
(12) Patent Application Publication
(10) Pub. No.: US 2011/0136115 A1
(43) Pub. Date: Jun. 9, 2011

(54) METHODS AND NUCLEIC ACIDS FOR THE ANALYSIS OF GENE EXPRESSION ASSOCIATED WITH THE DEVELOPMENT OF PROSTATE CELL PROLIFERATIVE DISORDERS

(75) Inventors: Andrew Z. Sledziewski, Shoreline, WA (US); Shannon Payne, Seattle, WA (US); Matthias Schuster, Parc Palais (SG); Joern Lewin, Berlin (DE); Thomas Schlegel, Berlin (DE); Andrew M. Morotti, Seattle, WA (US)

(73) Assignee: EPIGENOMICS AG, BERLIN (DE)

(21) Appl. No.: 12/744,318

(22) PCT Filed: Nov. 11, 2008
(86) PCT No.: PCT/EP08/09510

§ 371 Date: Feb. 2, 2011

(30) Foreign Application Priority Data
Nov. 23, 2007 (EP) 07121408.4
Oct. 13, 2008 (EP) 08166448.4

(51) Int. Cl. C12Q 1/68; C07H 21/04

(52) U.S. Cl. 435/6.11; 536/23.1; 435/6.1

(57) ABSTRACT

The invention provides methods, nucleic acids and kits for detecting prostate cell proliferative disorders. The invention discloses genomic sequences the methylation patterns of which have utility for the improved detection of said disorder, thereby enabling the improved diagnosis and treatment of patients.

### FIELD OF THE INVENTION

The present invention relates to genomic DNA sequences that exhibit altered expression patterns in disease states relative to normal. Particular embodiments provide methods, nucleic acids, nucleic acid arrays and kits useful for detecting, or for diagnosing prostate carcinoma.

### PRIOR ART

Prostate cancer is the most common cancer and the third leading cause of death in American men (Jemal et al., Cancer statistics, 2006. CA Cancer J. Clin. 2006; 56(2):106-30). Incidence and mortality rates for this disease increase greatly with age, with more than 65% of all prostate cancer cases diagnosed in men older than 65 (Jemal et al., 2006; supra). Stage of disease at diagnosis also affects overall survival rates. Due to widespread use of the prostate-specific antigen (PSA) screening test, nearly 90% of new patients are diagnosed with local or regional disease (Jemal et al., 2006; supra). Patients with local or regional disease when diagnosed have a five-year relative survival rate approaching 100% (Jemal et al., 2006; supra).

The current guidelines for prostate cancer screening, according to the American Cancer Society, advises testing for elevated PSA and digital rectal examination annually beginning at age 50. For men at high risk of developing prostate cancer (African American men and men with one or more first degree relatives diagnosed at an early age), screening should begin at age 45. Positive findings on either of these exams is confirmed by prostate biopsy. The advent of PSA screening has changed the landscape of prostate cancer diagnosis. Incidence rates in prostate cancer have increased dramatically in the last 20 years, while diagnosis in males older than 65 has leveled off. PSA testing suffers from two disadvantages. The first is its low specificity as PSA is elevated in a number of benign conditions in addition to prostate cancer. This results in a large number of prostate biopsies being conducted in men who do not have prostate cancer. The second disadvantage is that despite the relatively high sensitivity of PSA, there are men who harbor prostate cancer in the absence of elevated PSA (>4 ng/ml) (Thompson et al., Prevalence of prostate cancer among men with a prostate-specific antigen level <or=4.0 ng per milliliter. N Engl J. Med. 2004 May 27; 350(22):2239-46. Erratum in: N Engl J. Med. 2004 30; 351(14):1470). It has also been estimated that up to 10% of prostate biopsies under currrent guidelines are falsely negative, resulting in decreased sensitivity even with biopsy (Djavan et al., Optimal predictors of prostate cancer on repeat prostate biopsy: a prospective study of 1,051 men. J. Urol. 2000; 163(4):1144-8; discussion 1148-9; Mian et al., Predictors of cancer in repeat extended multisite prostate biopsy in men with previous negative extended multisite biopsy. Urology. 2002; 60(5):836-40; Gupta et al., Transrectal ultrasound guided biopsy for detecting early prostate cancer: An Indian experience, Indian J. Cancer. 2005; 42(3):151-4; Hanley et al., DNA integrity assay: a plasma-based screening tool for the detection of prostate cancer. Clin Cancer Res. 2006 1; 12(15):4569-74). Improved tests with increased specificity and sensitivity are clearly needed.

Multifactorial approach. Cancer diagnostics has traditionally relied upon the detection of single molecular markers (e.g., gene mutations, elevated PSA levels). Unfortunately, cancer is a disease state in which single markers have typically failed to detect or differentiate many forms of the disease. Thus, assays that recognize only a single marker have been shown to be of limited predictive value. A fundamental aspect of this invention is that methylation-based cancer diagnostics and the screening, diagnosis, and therapeutic monitoring of such diseases will provide significant improvements over the state-of-the-art that uses single marker analyses by the use of a selection of multiple markers. The multiplexed analytical approach is particularly well suited for cancer diagnostics since cancer is not a simple disease, this multi-factorial “panel” approach is consistent with the heterogeneous nature of cancer, both cytologically and clinically.

Key to the successful implementation of a panel approach to methylation based diagnostic tests is the design and development of optimized panels of markers that can characterize and distinguish disease states. The present invention describes a plurality of particularly efficient and unique panels of genes, the methylation analysis of one or a combination of the members of the panel enabling the detection of prostate cell proliferative disorders with a particularly high sensitivity, specificity and/or predictive value.

Development of medical tests. Two key evaluative measures of any medical screening or diagnostic test are its sensitivity and specificity, which measure how well the test performs to accurately detect all affected individuals without exception, and without falsely including individuals who do not have the target disease (predicitive value). Historically, many diagnostic tests have been criticized due to poor sensitivity and specificity.

A true positive (TP) result is where the test is positive and the condition is present. A false positive (FP) result is where the test is positive but the condition is not present. A true negative (TN) result is where the test is negative and the condition is not present. A false negative (FN) result is where the test is negative but the condition is not present. In this context: Sensitivity=TP/(TP+FN); Specificity=TN/(FP+TN); and Predictive value=TP/(TP+FP).

Sensitivity is a measure of a test's ability to correctly detect the target disease in an individual being tested. A test having poor sensitivity produces a high rate of false negatives, i.e., individuals who have the disease but are falsely identified as being free of that particular disease. The potential danger of a false negative is that the diseased individual will remain undiagnosed and untreated for some period of time, during which the disease may progress to a later stage wherein treatments, if any, may be less effective. An example of a test that has low sensitivity is a protein-based blood test for HIV. This type of test exhibits poor sensitivity because it fails to detect the presence of the virus until the disease is well established and the virus has invaded the bloodstream in substantial numbers. In contrast, an example of a test that has high sensitivity is viral-load detection using the polymerase chain reaction (PCR). High sensitivity is achieved because this type of test can detect very small quantities of the virus. High sensitivity is particularly important when the consequences of missing a diagnosis are high.

Specificity, on the other hand, is a measure of a test's ability to identify accurately patients who are free of the disease state. A test having poor specificity produces a high rate of false positives, i.e., individuals who are falsely identified as having the disease. A drawback of false positives is that they force patients to undergo unnecessary medical procedures treatments with their attendant risks, emotional and financial stresses, and which could have adverse effects on the patient's health. A feature of diseases which makes it difficult to develop diagnostic tests with high specificity is that disease mechanisms, particularly in cancer, often involve a plurality of genes and proteins. Additionally, certain proteins may be elevated for reasons unrelated to a disease state. An example of a test that has high specificity is a gene-based test that can detect a p53 mutation. Specificity is important when the cost or risk associated with further diagnostic procedures or further medical intervention are very high.

### BRIEF DESCRIPTION OF THE DRAWINGS

FIG. 1 provides an overview of the log mean methylation measured by means of the HM assay according to Example 1. For each analysed gene (as labeled to the right of the figures), two plots are provided, the left hand side plots provide the multiclass analysis, sensitivity is shown on the Y-axis, DNA methylation measured in (log 10 ng/ml) is shown on the X-axis. The right hand plot provides an ROC wherein sensitivity is shown on the Y-axis and 1-specificity is shown on the X-axis. (A—Normal blood, B—Normal prostate, C—NAT prostate, D—Tumor prostate).

FIG. 2 provides an overview of the log mean methylation measured by means of HM real-time PCR assays of post-prostatic massage urine of PCa and negative class I (young asymptomatic individuals) according to Example 1. For each analysed gene (as labeled above each plot), sensitivity is shown on the Y-axis, DNA methylation measured in (log 10 ng/ml) is shown on the X-axis.

FIG. 3 provides an overview of the log mean methylation measured by means of HM real-time PCR assays of post-prostatic massage urine of PCa and negative class II (young asymptomatic plus biopsy negative individuals) according to Example 1. For each analysed gene (as labeled above each plot), sensitivity is shown on the Y-axis, DNA methylation measured in (log 10 ng/ml) is shown on the X-axis.

FIG. 4 shows the log mean methylation measured by means of HM real-time PCR assays of plasma of PCa and negative class I (young asymptomatic individuals). For each analysed gene (as labeled above each plot), sensitivity is shown on the Y-axis, DNA methylation measured in (log 10 ng/ml) is shown on the X-axis.

FIG. 5 shows the log mean methylation measured by means of HM real-time PCR assays of plasma of PCa and negative class II (young asymptomatic plus biopsy negative individuals). For each analysed gene (as labeled above each plot), sensitivity is shown on the Y-axis, DNA methylation measured in (log 10 ng/ml) is shown on the X-axis.

FIG. 6 shows biomarker performance in discrimination of PCa patients from asymptomatic males (Example 1: Performance of biomarkers for Pca vs. negative class I (asymptomatic males)). A, Table compares the AUC, sensitivity and Wilcoxon p values of the four biomarkers for PCa versus asymptomatic males in urine DNA. B, Table compares the AUC, sensitivity and Wilcoxon p values of the four biomarkers for PCa versus asymptomatic males in plasma DNA. C, ROC curves for individual PCa biomarkers in urine DNA.

FIG. 7 shows biomarker performance in discrimination of PCa from biopsy negative patients (Example 1: Performance of biomarkers for PCa vs. biopsy negative males). A, Table compares the AUC and Wilcoxon p values of the four biomarkers for PCa versus biopsy negative patients in urine DNA. Sensitivities are given for 50%, 80%, 90% and 95% specificities. B, Table compares the AUC and Wilcoxon p values of the four biomarkers for PCa versus biopsy negative patients in plasma DNA. Sensitivities are given for 50%, 80%, 90% and 95% specificities. C, Table compares the AUC and Wilcoxon p values of PSA-related biomarkers for PCa versus biopsy negative patients. Sensitivities are given for 50%, 80%, 90% and 95% specificities. D, ROC curves for individual PCa biomarkers in urine DNA.

FIG. 8 shows CARTPT biomarker performance. FIG. 8A shows CARTPT biomarker performance in discrimination of PCa from benign tissues (log mean methylation measured by means of real-time quantitative PCR (qPCR); Example 2) and FIG. 8 B shows CARTPT biomarker performance in discrimination of PCa from biopsy negative patient urine (left ROC curve, right log mean methylation measured by means of real-time qPCR; Example 3).

FIG. 9 shows CX43/GJA1 biomarker performance. FIG. 9A shows CX43/GJA1 biomarker performance in discrimination of PCa from benign tissues (log mean methylation measured by means of real-time qPCR; Example 2) and FIG. 9B shows CX43/GJA1 biomarker performance in discrimination of PCa from biopsy negative patient urine (left ROC curve, right log mean methylation measured by means of real-time qPCR; Example 3).

FIG. 10 shows GPR68 biomarker performance in discrimination of high Gleason sum PCa from low Gleason sum PCa tissues (log mean methylation measured by means of real-time qPCR; Example 2). 15/26 (58%) Gleason 8/9; 3/12 (25%) Gleason 5/6; AUC=0.66; Wilcoxon p=0.05 Gleason 9-10 vs. 5-8 (The numbers 5-9 within the diagram indicate the Gleason scores 5, 6, 7, 8, and 9).

FIG. 11 shows FGF13 biomarker performance. FIG. 11A shows FGF13 biomarker performance in discrimination of PCa from benign tissues (log mean methylation measured by means of real-time qPCR; Example 2) and FIG. 11B shows FGF13 biomarker performance in discrimination of PCa from biopsy negative patient urine (left ROC curve, right log mean methylation measured by means of real-time qPCR; Example 3).

FIG. 12 shows SPG20 biomarker performance. FIG. 12A shows SPG20 biomarker performance in discrimination of PCa from benign tissues (log mean methylation measured by means of real-time qPCR, Example 2) and FIG. 12B shows SPG20 biomarker performance of PCa from biopsy negative patient urine (left ROC curve, right log mean methylation measured by means of real-time qPCR; Example 3).

FIG. 13 shows NKX2-6 biomarker performance in discrimination of PCa from benign tissues (log mean methylation measured by means of real-time qPCR, Example 2).

FIG. 14 shows RARB biomarker performance in discrimination of PCa from benign tissues (log mean methylation measured by means of real-time qPCR, Example 2).

FIG. 15 shows PDE4D biomarker performance in discrimination of PCa from benign tissues (log mean methylation measured by means of real-time qPCR, Example 2).

FIG. 16 shows MN1 biomarker performance. FIG. 16A shows MN1 biomarker performance in discrimination of PCa from benign tissues (log mean methylation measured by means of real-time qPCR, Example 2) and FIG. 16B shows MN1 biomarker performance in discrimination of PCa from biopsy negative patient urine (left ROC curve, right log mean methylation measured by means of real-time qPCR; Example 3).

FIG. 17 shows KLF8 biomarker performance. FIG. 17A shows KLF8 biomarker performance in discrimination of PCa from benign tissues (log mean methylation measured by means of real-time qPCR, Example 2) and FIG. 17B shows KLF8 biomarker performance in discrimination of PCa from biopsy negative patient urine (left ROC curve, right log mean methylation measured by means of real-time qPCR; Example 3).

FIG. 18 shows MOBKL2B biomarker performance. FIG. 18A shows MOBKL2B biomarker performance in discrimination of PCa from benign tissues (log mean methylation measured by means of real-time qPCR, Example 2) and FIG. 18B shows MOBKL2B biomarker performance of PCa from biopsy negative patient urine (left ROC curve, right log mean methylation measured by means of real-time qPCR; Example 3).

FIG. 19 shows HIST1H2BD biomarker performance in discrimination of PCa from benign tissues (log mean methylation measured by means of real-time qPCR, Example 2).

FIG. 20 shows NFATC3 biomarker performance. FIG. 20A shows NFATC3 biomarker performance in discrimination of PCa from benign tissues (log mean methylation measured by means of real-time qPCR, Example 2) and FIG. 20B shows NFATC3 biomarker performance in discrimination of PCa from biopsy negative patient urine (left ROC curve, right log mean methylation measured by means of real-time qPCR; Example 3).

### SUMMARY OF THE INVENTION

The present invention provides a method for detecting prostate cell proliferative disorders, most preferably, prostate carcinoma in a subject comprising determining the expression levels of at least one gene selected from the group consisting of RASSF2A; TFAP2E; HIST1H4K; GSTPi; MME; RARB; NKX3-1; NPAL3; ACVR2A; ARL4C; PDE4D; CARTPT; HIST1H2BD; CUL1; MOBKL2B; IFLTD1 (KRAS); ANXA2; NFATC3; MN1; KLF8; FGF13; NKX2-6; SPG20; DSE/SART2; GJA1 (CX43); SPATA6; MCC; GPR68 (INTRON 1 AND/OR EXON 2 REGION) in a biological sample isolated from said subject wherein underexpression and/or CpG methylation is indicative of the presence of said disorder.

Preferably a plurality of genes (herein also referred to as a “gene panel”) are analysed. Preferably 2, 3 or 4 genes are analysed. In one embodiment of the method said panel comprises at least one gene selected from the group consisting of RASSF2A; TFAP2E; HIST1H4K; GSTPi; MME; RARB; NKX3-1; NPAL3; ACVR2A; ARL4C; PDE4D; CARTPT; HIST1H2BD; CUL1; MOBKL2B; IFLTD1 (KRAS); ANXA2; NFATC3; MN1; KLF8; FGF13; NKX2-6; SPG20; DSE/SART2; GJA1 (CX43); SPATA6; MCC; GPR68 (INTRON 1 AND/OR EXON 2 REGION); and/or their promoter or regulatory regions. Preferably said group comprises the gene HIST1H4K.

Particularly preferred are the following combinations of genes:

### −HIST1H4K +RASSF2A or HIST1H4K +GSTPi

Various aspects of the present invention provide an efficient and unique genetic marker, whereby expression analysis of said marker enables the detection of prostate cell proliferative disorders, most preferably, prostate carcinoma with a particularly high sensitivity, specificity and/or predictive value.

In one embodiment the invention provides a method for detecting prostate cell proliferative disorders, most preferably, prostate carcinoma in a subject comprising determining the expression levels of at least one gene selected from the group consisting of RASSF2A; TFAP2E; HIST1H4K; GSTPi; MME; RARB; NKX3-1; NPAL3; ACVR2A; ARL4C; PDE4D; CARTPT; HIST1H2BD; CUL1; MOBKL2B; IFLTD1 (KRAS); ANXA2; NFATC3; MN1; KLF8; FGF13; NKX2-6; SPG20; DSE/SART2; GJA1 (CX43); SPATA6; MCC; GPR68 (INTRON 1 AND/OR EXON 2 REGION); in a biological sample isolated from said subject wherein under-expression and/or CpG methylation is indicative of the presence of said disorder. In one embodiment said expression level is determined by detecting the presence, absence or level of mRNA transcribed from said gene. In a further embodiment said expression level is determined by detecting the presence, absence or level of a polypeptide encoded by said gene or sequence thereof.

In a further preferred embodiment said expression is determined by detecting the presence or absence of CpG methylation within said gene, wherein the presence of methylation indicates the presence of prostate cell proliferative disorders, more specifically, prostate carcinoma.

Said method comprises the following steps: i) contacting genomic DNA isolated from a biological sample (preferably selected from the group consisting of ejaculate, blood plasma, blood serum, whole blood, isolated blood cells, cells isolated from the blood) obtained from the subject with at least one reagent, or series of reagents that distinguishes between methylated and non-methylated CpG dinucleotides within at least one and more preferably a plurality of target regions of the genomic DNA, wherein the nucleotide sequence of said target region comprises at least one CpG dinucleotide sequence of at least one gene selected from the group consisting of RASSF2A; TFAP2E; HIST1H4K; GSTPi; MME; RARB; NKX3-1; NPAL3; ACVR2A; ARL4C; PDE4D; CARTPT; HIST1H2BD; CUL1; MOBKL2B; IFLTD1 (KRAS); ANXA2; NFATC3; MN1; KLF8; FGF13; NKX2-6; SPG20; DSE/SART2; GJA1 (CX43); SPATA6; MCC; GPR68 (INTRON 1 AND/OR EXON 2 REGION); and ii) detecting carcinoma, at least in part.

Preferably a plurality of genes (herein also referred to as a “gene panel”) are analysed. Preferably 2, 3 or 4 genes are analysed. In one embodiment of the method said panel comprises at least one gene selected from the group consisting of RASSF2A; TFAP2E; HIST1H4K; GSTPi; MME; RARB; NKX3-1; NPAL3; ACVR2A; ARL4C; PDE4D; CARTPT; HIST1H2BD; CUL1; MOBKL2B; IFLTD1 (KRAS); ANXA2; NFATC3; MN1; KLF8; FGF13; NKX2-6; SPG20; DSE/SART2; GJA1 (CX43); SPATA6; MCC; GPR68 (INTRON 1 AND/OR EXON 2 REGION); and/or their promoter or regulatory regions. Preferably said group comprises the gene HIST1H4K.

Particularly preferred are the following combinations of genes:

### HIST1H4K +RASSF2A or HIST1H4K +GSTPi

Preferably the target region comprises, or hybridizes under stringent conditions to a sequence of at least 16, 50, 100 or 500 contiguous nucleotides of at least one sequence selected from the group consisting of SEQ ID NO: 1-4; 37-65.

Said use of the gene may be enabled by means of any analysis of the expression of the gene, by means of mRNA expression analysis or protein expression analysis. However, in the most preferred embodiment of the invention the detection of detecting prostate cell proliferative disorders, most preferably, prostate carcinomas is enabled by means of analysis of the methylation status of at least one gene selected from the group consisting of RASSF2A; TFAP2E; HIST1H4K; GSTPi; MME; RARB; NKX3-1; NPAL3; ACVR2A; ARL4C; PDE4D; CARTPT; HIST1H2BD; CUL1; MOBKL2B; IFLTD1 (KRAS); ANXA2; NFATC3; MN1; KLF8; FGF13; NKX2-6; SPG20; DSE/SART2; GJA1 (CX43); SPATA6; MCC; GPR68 (INTRON 1 AND/OR EXON 2 REGION); and/or its promoter or regulatory elements.

Preferably a plurality of genes (herein also referred to as a “gene panel”) are analysed. Preferably 2, 3 or 4 genes are analysed. In one embodiment of the method said panel comprises at least one gene selected from the group consisting of RASSF2A; TFAP2E; HIST1H4K; GSTPi; MME; RARB; NKX3-1; NPAL3; ACVR2A; ARL4C; PDE4D; CARTPT; HIST1H2BD; CUL1; MOBKL2B; IFLTD1 (KRAS); ANXA2; NFATC3; MN1; KLF8; FGF13; NKX2-6; SPG20; DSE/SART2; GJA1 (CX43); SPATA6; MCC; GPR68 (INTRON 1 AND/OR EXON 2 REGION); and/or their promoter or regulatory regions. Preferably said group comprises the gene HIST1H4K.

Particularly preferred are the following combinations of genes:

### HIST1H4K +RASSF2A or HIST1H4K +GSTPi

The invention provides a method for the analysis of biological samples for features associated with the development of cancer, the method characterized in that the nucleic acid, or a fragment thereof of at least one sequence selected from the group consisting of SEQ ID NO: 1-4; 37-65 is contacted with a reagent or series of reagents capable of distinguishing between methylated and non methylated CpG dinucleotides within the genomic sequence.

The present invention provides a method for ascertaining epigenetic parameters of genomic DNA associated with the development of prostate cancer. The method has utility for the improved detection and diagnosis of said prostate cell proliferative disorders, in particular prostate cancer.

Preferably, the source of the test sample is selected from the group consisting of cells or cell lines, histological slides, biopsies, paraffin-embedded tissue, body fluids, ejaculate, urine, blood, and combinations thereof. More preferably, the source is selected from the group consisting of ejaculate, urine, blood plasma, blood serum, whole blood, isolated blood cells, cells isolated from the blood obtained from the subject.

Specifically, the present invention provides a method for detecting prostate cancer suitable for use in a diagnostic tool, comprising: obtaining a biological sample comprising genomic nucleic acid(s); contacting the nucleic acid(s), or a fragment thereof, with a reagent or a plurality of reagents sufficient for distinguishing between methylated and non methylated CpG dinucleotide sequences within at least one and more preferably a plurality of target sequence(s) of the subject nucleic acid, wherein each of said target sequences comprises, or hybridises under stringent conditions to, a sequence comprising at least 16, 50, 100 or 500 contiguous nucleotides of a sequence selected from the group consisting of SEQ ID NO: 1-4; 37-65, said contiguous nucleotides comprising at least one CpG dinucleotide sequence; and determining, based at least in part on said distinguishing, the methylation state of at least one and more preferably a plurality of target CpG dinucleotide sequence, or an average, or a value reflecting an average methylation state of a plurality of target CpG dinucleotide sequences.

Preferably, distinguishing between methylated and non methylated CpG dinucleotide sequences within the target sequence comprises methylation state-dependent conversion or non-conversion of at least one such CpG dinucleotide sequence to the corresponding converted or non-converted dinucleotide sequence within a sequence selected from the group consisting of SEQ ID NO: 5-20; 66-181, and contiguous regions thereof corresponding to the target sequence.

Additional embodiments provide a method for the detection of detecting prostate cell proliferative disorders, most preferably, prostate cancer comprising: obtaining a biological sample having subject genomic DNA; extracting the genomic DNA; treating the genomic DNA, or a fragment thereof, with one or more reagents to convert 5-position unmethylated cytosine bases to uracil or to another base that is detectably dissimilar to cytosine in terms of hybridization properties; contacting the treated genomic DNA, or the treated fragment thereof, with an amplification enzyme and at least two primers comprising, in each case a contiguous sequence at least 9 nucleotides in length that is complementary to, or hybridizes under moderately stringent or stringent conditions to a sequence selected from the group consisting SEQ ID NO: 5-20; 66-181, and complements thereof, wherein the treated DNA or the fragment thereof is either amplified to produce an amplificate, or is not amplified; and determining, based on a presence or absence of, or on a property of said amplificate, the methylation state or an average, or a value reflecting an average of the methylation level of at least one, but more preferably a plurality of CpG dinucleotides of a sequence selected from the group consisting of SEQ ID NO: 1-4; 37-65.

Preferably, determining comprises use of at least one method selected from the group consisting of I) hybridizing at least one nucleic acid molecule comprising a contiguous sequence at least 9 nucleotides in length that is complementary to, or hybridizes under moderately stringent or stringent conditions to a sequence selected from the group consisting of SEQ ID NO: 5-20; 66-181, and complements thereof; ii) hybridizing at least one nucleic acid molecule, bound to a solid phase, comprising a contiguous sequence at least 9 nucleotides in length that is complementary to, or hybridizes under moderately stringent or stringent conditions to a sequence selected from the group consisting of SEQ ID NO: 5-20; 66-181, and complements thereof; iii) hybridizing at least one nucleic acid molecule comprising a contiguous sequence at least 9 nucleotides in length that is complementary to, or hybridizes under moderately stringent or stringent conditions to a sequence selected from the group consisting of SEQ ID NO: 5-20; 66-181, and complements thereof, and extending at least one such hybridized nucleic acid molecule by at least one nucleotide base; and iv) sequencing of the amplificate.

Further embodiments provide a method for the analysis (i.e. detection of classification) of carcinoma, comprising: obtaining a biological sample having subject genomic DNA; extracting the genomic DNA; contacting the genomic DNA, or a fragment thereof, comprising one or more sequences selected from the group consisting of SEQ ID NO: 1-4; 37-65 or a sequence that hybridizes under stringent conditions thereto, with one or more methylation-sensitive restriction enzymes, wherein the genomic DNA is either digested thereby to produce digestion fragments, or is not digested thereby; and determining, based on a presence or absence of, or on property of at least one such fragment, the methylation state of at least one CpG dinucleotide sequence at least one sequence selected from the group consisting of SEQ ID NO: 1-4; 37-65, or an average, or a value reflecting an average methylation state of a plurality of CpG dinucleotide sequences thereof. Preferably, the digested or undigested genomic DNA is amplified prior to said determining.

Additional embodiments provide novel genomic and chemically modified nucleic acid sequences, as well as oligonucleotides and/or PNA-oligomers for analysis of cytosine methylation patterns within sequences from the group consisting of SEQ ID NO: 1-4; 37-65.

### DETAILED DESCRIPTION OF THE INVENTION

### Definitions

The term “Observed/Expected Ratio” (“O/E Ratio”) refers to the frequency of CpG dinucleotides within a particular DNA sequence, and corresponds to the [number of CpG sites/(number of C bases×number of G bases)]/band length for each fragment.

The term “CpG island” refers to a contiguous region of genomic DNA that satisfies the criteria of (1) having a frequency of CpG dinucleotides corresponding to an “Observed/Expected Ratio”>0.6, and (2) having a “GC Content”>0.5. CpG islands are typically, but not always, between about 0.2 to about 1 kb, or to about 2 kb in length.

The term “methylation state” or “methylation status” refers to the presence or absence of 5-methylcytosine (“5-mCyt”) at one or a plurality of CpG dinucleotides within a DNA sequence. Methylation states at one or more particular CpG methylation sites (each having two CpG dinucleotide sequences) within a DNA sequence include “unmethylated,” “fully-methylated” and “hemi-methylated.”

The term “hemi-methylation” or “hemimethylation” refers to the methylation state of a double stranded DNA wherein only one strand thereof is methylated.

The term ‘AUC’ as used herein is an abbreviation for the area under a curve. In particular it refers to the area under a Receiver Operating Characteristic (ROC) curve. The ROC curve is a plot of the true positive rate against the false positive rate for the different possible cut points of a diagnostic test. It shows the trade-off between sensitivity and specificity depending on the selected cut point (any increase in sensitivity will be accompanied by a decrease in specificity). The area under an ROC curve (AUC) is a measure for the accuracy of a diagnostic test (the larger the area the better, optimum is 1, a random test would have a ROC curve lying on the diagonal with an area of 0.5; for reference: J.P. Egan. Signal Detection Theory and ROC Analysis, Academic Press, New York, 1975).

The term “hypermethylation” refers to the average methylation state corresponding to an increased presence of 5-mCyt at one or a plurality of CpG dinucleotides within a DNA sequence of a test DNA sample, relative to the amount of 5-mCyt found at corresponding CpG dinucleotides within a normal control DNA sample.

The term “hypomethylation” refers to the average methylation state corresponding to a decreased presence of 5-mCyt at one or a plurality of CpG dinucleotides within a DNA sequence of a test DNA sample, relative to the amount of 5-mCyt found at corresponding CpG dinucleotides within a normal control DNA sample.

The term “microarray” refers broadly to both “DNA microarrays,” and ‘DNA chip(s),’ as recognized in the art, encompasses all art-recognized solid supports, and encompasses all methods for affixing nucleic acid molecules thereto or synthesis of nucleic acids thereon.

“Genetic parameters” are mutations and polymorphisms of genes and sequences further required for their regulation. To be designated as mutations are, in particular, insertions, deletions, point mutations, inversions and polymorphisms and, particularly preferred, SNPs (single nucleotide polymorphisms).

“Epigenetic parameters” are, in particular, cytosine methylation. Further epigenetic parameters include, for example, the acetylation of histones which, however, cannot be directly analysed using the described method but which, in turn, correlate with the DNA methylation.

The term “bisulfite reagent” refers to a reagent comprising bisulfite, disulfite, hydrogen sulfite or combinations thereof, useful as disclosed herein to distinguish between methylated and unmethylated CpG dinucleotide sequences.

The term “Methylation assay” refers to any assay for determining the methylation state of one or more CpG dinucleotide sequences within a sequence of DNA.

The term “MS.AP-PCR” (Methylation-Sensitive Arbitrarily-Primed Polymerase Chain Reaction) refers to the art-recognized technology that allows for a global scan of the genome using CG-rich primers to focus on the regions most likely to contain CpG dinucleotides, and described by Gonzalgo et al., Cancer Research 57:594-599, 1997.

The term “MethyLight™” refers to the art-recognized fluorescence-based real-time PCR technique described by Eads et al., Cancer Res. 59:2302-2306, 1999.

The term “HeavyMethyl™” assay, in the embodiment thereof implemented herein, refers to an assay, wherein methylation specific blocking probes (also referred to herein as blockers) covering CpG positions between, or covered by the amplification primers enable methylation-specific selective amplification of a nucleic acid sample.

The term “HeavyMethyl™ MethyLight™” assay, in the embodiment thereof implemented herein, refers to a HeavyMethyl™ MethyLight™ assay, which is a variation of the MethyLight™ assay, wherein the MethyLight™ assay is combined with methylation specific blocking probes covering CpG positions between the amplification primers.

The term “Ms-SNuPE” (Methylation-sensitive Single Nucleotide Primer Extension) refers to the art-recognized assay described by Gonzalgo & Jones, Nucleic Acids Res. 25:2529-2531, 1997.

The term “MSP” (Methylation-specific PCR) refers to the art-recognized methylation assay described by Herman et al. Proc. Natl. Acad. Sci. USA 93:9821-9826, 1996, and by U.S. Pat. No. 5,786,146.

The term “COBRA” (Combined Bisulfite Restriction Analysis) refers to the art-recognized methylation assay described by Xiong & Laird, Nucleic Acids Res. 25:2532-2534, 1997.

The term “MCA” (Methylated CpG Island Amplification) refers to the methylation assay described by Toyota et al., Cancer Res. 59:2307-12, 1999, and in WO 00/26401A1.

The term “hybridisation” is to be understood as a bond of an oligonucleotide to a complementary sequence along the lines of the Watson-Crick base pairings in the sample DNA, forming a duplex structure.

“Stringent hybridisation conditions,” as defined herein, involve hybridising at 68° C. in 5×SSC/5×Denhardt's solution/1.0% SDS, and washing in 0.2×SSC/0.1% SDS at room temperature, or involve the art-recognized equivalent thereof (e.g., conditions in which a hybridisation is carried out at 60° C. in 2.5×SSC buffer, followed by several washing steps at 37° C. in a low buffer concentration, and remains stable). Moderately stringent conditions, as defined herein, involve including washing in 3×SSC at 42° C., or the art-recognized equivalent thereof. The parameters of salt concentration and temperature can be varied to achieve the optimal level of identity between the probe and the target nucleic acid. Guidance regarding such conditions is available in the art, for example, by Sambrook et al., 1989, Molecular Cloning, A Laboratory Manual, Cold Spring Harbor Press, N.Y.; and Ausubel et al. (eds.), 1995, Current Protocols in Molecular Biology, (John Wiley & Sons, N.Y.) at Unit 2.10.

The terms “Methylation-specific restriction enzymes” or “methylation-sensitive restriction enzymes” shall be taken to mean an enzyme that selectively digests a nucleic acid dependant on the methylation state of its recognition site. In the case of such restriction enzymes which specifically cut if the recognition site is not methylated or hemimethylated, the cut will not take place, or with a significantly reduced efficiency, if the recognition site is methylated. In the case of such restriction enzymes which specifically cut if the recognition site is methylated, the cut will not take place, or with a significantly reduced efficiency if the recognition site is not methylated. Preferred are methylation-specific restriction enzymes, the recognition sequence of which contains a CG dinucleotide (for instance cgcg or cccggg). Further preferred for some embodiments are restriction enzymes that do not cut if the cytosine in this dinucleotide is methylated at the carbon atom C5.

“Non-methylation-specific restriction enzymes” or “non-methylation-sensitive restriction enzymes” are restriction enzymes that cut a nucleic acid sequence irrespective of the methylation state with nearly identical efficiency. They are also called “methylation-unspecific restriction enzymes.”

In reference to composite array sequences, the phrase “contiguous nucleotides” refers to a contiguous sequence region of any individual contiguous sequence of the composite array, but does not include a region of the composite array sequence that includes a “node,” as defined herein above.

The terms “RASSF2A; TFAP2E; HIST1H4K; GSTPi; MME; RARB; NKX3-1; NPAL3; ACVR2A; ARL4C; PDE4D; CARTPT; HIST1H2BD; CUL1; MOBKL2B; IFLTD1 (KRAS); ANXA2; NFATC3; MN1; KLF8; FGF13; NKX2-6; SPG20; DSE/SART2; GJA1 (CX43); SPATA6; MCC; GPR68 (INTRON 1 AND/OR EXON 2 REGION);” shall be taken to include all transcript variants thereof and all promoter and regulatory elements thereof. Furthermore as a plurality of SNPs are known within said genes the term shall be taken to include all sequence variants thereof.

The present invention provides a method for detecting carcinoma in a subject comprising determining the expression levels of at least one gene selected from the group consisting of RASSF2A; TFAP2E; HIST1H4K; GSTPi; MME; RARB; NKX3-1; NPAL3; ACVR2A; ARL4C; PDE4D; CARTPT; HIST1H2BD; CUL1; MOBKL2B; IFLTD1 (KRAS); ANXA2; NFATC3; MN1; KLF8; FGF13; NKX2-6; SPG20; DSE/SART2; GJA1 (CX43); SPATA6; MCC; GPR68 (INTRON 1 AND/OR EXON 2 REGION); in a biological sample isolated from said subject wherein underexpression and/or CpG methylation is indicative of the presence or class of said disorder. Said markers may be used for the diagnosis of prostate cancer including early detection during the pre-cancerous stages of the disease. The markers of the present invention are particularly efficient in detecting malignant prostate cell proliferative disorders such as prostate carcinoma, thereby providing improved means for the early detection, classification and treatment of said disorders.

In addition to the embodiments above wherein the methylation analysis of at least one gene selected from the group consisting of RASSF2A; TFAP2E; HIST1H4K; GSTPi; MME; RARB; NKX3-1; NPAL3; ACVR2A; ARL4C; PDE4D; CARTPT; HIST1H2BD; CUL1; MOBKL2B; IFLTD1 (KRAS); ANXA2; NFATC3; MN1; KLF8; FGF13; NKX2-6; SPG20; DSE/SART2; GJA1 (CX43); SPATA6; MCC; GPR68 (INTRON 1 AND/OR EXON 2 REGION); is analysed, the invention presents further panels of genes comprising at least one gene selected from the group consisting of RASSF2A; TFAP2E; HIST1H4K; GSTPi; MME; RARB; NKX3-1; NPAL3; ACVR2A; ARL4C; PDE4D; CARTPT; HIST1H2BD; CUL1; MOBKL2B; IFLTD1 (KRAS); ANXA2; NFATC3; MN1; KLF8; FGF13; NKX2-6; SPG20; DSE/SART2; GJA1 (CX43); SPATA6; MCC; GPR68 (INTRON 1 AND/OR EXON 2 REGION); with novel utility for the detection of cell proliferative disorders, in particular prostate cancer. Preferably a plurality of genes (herein also referred to as a “gene panel”) are analysed. Preferably 2, 3 or 4 genes are analysed. In one embodiment of the method said panel comprises at least one gene selected from the group consisting of RASSF2A; TFAP2E; HIST1H4K; GSTPi; MME; RARB; NKX3-1; NPAL3; ACVR2A; ARL4C; PDE4D; CARTPT; HIST1H2BD; CUL1; MOBKL2B; IFLTD1 (KRAS); ANXA2; NFATC3; MN1; KLF8; FGF13; NKX2-6; SPG20; DSE/SART2; GJA1 (CX43); SPATA6; MCC; GPR68 (INTRON 1 AND/OR EXON 2 REGION); and/or their promoter or regulatory regions. Preferably said group comprises the gene HIST1H4K.

Particularly preferred are the following combinations of genes:

### HIST1H4K +RASSF2A or HIST1H4K +GSTPi

Bisulfite modification of DNA is an art-recognized tool used to assess CpG methylation status. 5-methylcytosine is the most frequent covalent base modification in the DNA of eukaryotic cells. It plays a role, for example, in the regulation of the transcription, in genetic imprinting, and in tumorigenesis. Therefore, the identification of 5-methylcytosine as a component of genetic information is of considerable interest. However, 5-methylcytosine positions cannot be identified by sequencing, because 5-methylcytosine has the same base pairing behavior as cytosine. Moreover, the epigenetic information carried by 5-methylcytosine is completely lost during, e.g., PCR amplification.

The most frequently used method for analyzing DNA for the presence of 5-methylcytosine is based upon the specific reaction of bisulfite with cytosine whereby, upon subsequent alkaline hydrolysis, cytosine is converted to uracil which corresponds to thymine in its base pairing behavior. Significantly, however, 5-methylcytosine remains unmodified under these conditions. Consequently, the original DNA is converted in such a manner that methylcytosine, which originally could not be distinguished from cytosine by its hybridization behavior, can now be detected as the only remaining cytosine using standard, art-recognized molecular biological techniques, for example, by amplification and hybridization, or by sequencing. All of these techniques are based on differential base pairing properties, which can now be fully exploited.

The prior art, in terms of sensitivity, is defined by a method comprising enclosing the DNA to be analysed in an agarose matrix, thereby preventing the diffusion and renaturation of the DNA (bisulfite only reacts with single-stranded DNA), and replacing all precipitation and purification steps with fast dialysis (Olek A, et al., A modified and improved method for bisulfite based cytosine methylation analysis, Nucleic Acids Res. 24:5064-6, 1996). It is thus possible to analyse individual cells for methylation status, illustrating the utility and sensitivity of the method. An overview of art-recognized methods for detecting 5-methylcytosine is provided by Rein, T., et al., Nucleic Acids Res., 26:2255, 1998.

The bisulfite technique, barring few exceptions (e.g., Zeschnigk M, et al., Eur J Hum Genet. 5:94-98, 1997), is currently only used in research. In all instances, short, specific fragments of a known gene are amplified subsequent to a bisulfite treatment, and either completely sequenced (Olek & Walter, Nat. Genet. 1997 17:275-6, 1997), subjected to one or more primer extension reactions (Gonzalgo & Jones, Nucleic Acids Res., 25:2529-31, 1997; WO 95/00669; U.S. Pat. No. 6,251,594) to analyse individual cytosine positions, or treated by enzymatic digestion (Xiong & Laird, Nucleic Acids Res., 25:2532-4, 1997). Detection by hybridisation has also been described in the art (Olek et al., WO 99/28498). Additionally, use of the bisulfite technique for methylation detection with respect to individual genes has been described (Grigg & Clark, Bioessays, 16:431-6, 1994; Zeschnigk M, et al., Hum Mol. Genet., 6:387-95, 1997; Feil R, et al., Nucleic Acids Res., 22:695-1994; Martin V, et al., Gene, 157:261-4, 1995; WO 97/46705 and WO 95/15373).

The present invention provides for the use of the bisulfite technique, in combination with one or more methylation assays, for determination of the methylation status of CpG dinucleotide sequences within SEQ ID NO: 1-4; 37-65. Genomic CpG dinucleotides can be methylated or unmethylated (alternatively known as up- and down-methylated respectively). However the methods of the present invention are suitable for the analysis of biological samples of a heterogeneous nature e.g. a low concentration of tumor cells within a background of blood or ejaculate. Accordingly, when analyzing the methylation status of a CpG position within such a sample the person skilled in the art may use a quantitative assay for determining the level (e.g. percent, fraction, ratio, proportion or degree) of methylation at a particular CpG position as opposed to a methylation state. Accordingly the term methylation status or methylation state should also be taken to mean a value reflecting the degree of methylation at a CpG position. Unless specifically stated the terms “hypermethylated” or “upmethylated” shall be taken to mean a methylation level above that of a specified cut-off point, wherein said cut-off may be a value representing the average or median methylation level for a given population, or is preferably an optimized cut-off level. The “cut-off” is also referred herein as a “threshold”. In the context of the present invention the terms “methylated”, “hypermethylated” or “upmethylated” shall be taken to include a methylation level above the cut-off be zero (0) % (or equivalents thereof) methylation for all CpG positions within and associated with (e.g. in promoter or regulatory regions) the genes RASSF2A; TFAP2E; HIST1H4K; GSTPi; MME; RARB; NKX3-1; NPAL3; ACVR2A; ARL4C; PDE4D; CARTPT; HIST1H2BD; CULL MOBKL2B; IFLTD1 (KRAS); ANXA2; NFATC3; MN1; KLF8; FGF13; NKX2-6; SPG20; DSE/SART2; GJA1 (CX43); SPATA6; MCC; GPR68 (INTRON 1 AND/OR EXON 2 REGION).

Preferably a plurality of genes (herein also referred to as a “gene panel”) are analysed. Preferably 2, 3 or 4 genes are analysed. In one embodiment of the method said panel comprises at least one gene selected from the group consisting of RASSF2A; TFAP2E; HIST1H4K; GSTPi; MME; RARB; NKX3-1; NPAL3; ACVR2A; ARL4C; PDE4D; CARTPT; HIST1H2BD; CUL1; MOBKL2B; IFLTD1 (KRAS); ANXA2; NFATC3; MN1; KLF8; FGF13; NKX2-6; SPG20; DSE/SART2; GJA1 (CX43); SPATA6; MCC; GPR68 (INTRON 1 AND/OR EXON 2 REGION); and/or their promoter or regulatory regions. Preferably said group comprises the gene HIST1H4K.

Particularly preferred are the following combinations of genes:

### HIST1H4K +RASSF2A or HIST1H4K +GSTPi

According to the present invention, determination of the methylation status of CpG dinucleotide sequences within SEQ ID NO: 1-4; 37-65 has utility in the diagnosis of prostate cancer.

Methylation Assay Procedures. Various methylation assay procedures are known in the art, and can be used in conjunction with the present invention. These assays allow for determination of the methylation state of one or a plurality of CpG dinucleotides (e.g., CpG islands) within a DNA sequence. Such assays involve, among other techniques, DNA sequencing of bisulfite-treated DNA, PCR (for sequence-specific amplification), Southern blot analysis, and use of methylation-sensitive restriction enzymes.

For example, genomic sequencing has been simplified for analysis of DNA methylation patterns and 5-methylcytosine distribution by using bisulfite treatment (Frommer et al., Proc. Natl. Acad. Sci. USA 89:1827-1831, 1992). Additionally, restriction enzyme digestion of PCR products amplified from bisulfite-converted DNA is used, e.g., the method described by Sadri & Hornsby (Nucl. Acids Res. 24:5058-5059, 1996), or COBRA (Combined Bisulfite Restriction Analysis) (Xiong & Laird, Nucleic Acids Res. 25:2532-2534, 1997).

COBRA. COBRA™ analysis is a quantitative methylation assay useful for determining DNA methylation levels at specific gene loci in small amounts of genomic DNA (Xiong & Laird, Nucleic Acids Res. 25:2532-2534, 1997). Briefly, restriction enzyme digestion is used to reveal methylation-dependent sequence differences in PCR products of sodium bisulfite-treated DNA. Methylation-dependent sequence differences are first introduced into the genomic DNA by standard bisulfite treatment according to the procedure described by Frommer et al. (Proc. Natl. Acad. Sci. USA 89:1827-1831, 1992). PCR amplification of the bisulfite converted DNA is then performed using primers specific for the CpG islands of interest, followed by restriction endonuclease digestion, gel electrophoresis, and detection using specific, labeled hybridization probes. Methylation levels in the original DNA sample are represented by the relative amounts of digested and undigested PCR product in a linearly quantitative fashion across a wide spectrum of DNA methylation levels. In addition, this technique can be reliably applied to DNA obtained from microdissected paraffin-embedded tissue samples.

Typical reagents (e.g., as might be found in a typical COBRA™-based kit) for COBRA™ analysis may include, but are not limited to: PCR primers for specific gene (or bisulfite treated DNA sequence or CpG island); restriction enzyme and appropriate buffer; gene-hybridization oligonucleotide; control hybridization oligonucleotide; kinase labeling kit for oligonucleotide probe; and labeled nucleotides. Additionally, bisulfite conversion reagents may include: DNA denaturation buffer; sulfonation buffer; DNA recovery reagents or kits (e.g., precipitation, ultrafiltration, affinity column); desulfonation buffer; and DNA recovery components.

Preferably, assays such as “MethyLight™” (a fluorescence-based real-time PCR technique) (Eads et al., Cancer Res. 59:2302-2306, 1999), Ms-SNuPE™ (Methylation-sensitive Single Nucleotide Primer Extension) reactions (Gonzalgo & Jones, Nucleic Acids Res. 25:2529-2531, 1997), methylation-specific PCR (“MSP”; Herman et al., Proc. Natl. Acad. Sci. USA 93:9821-9826, 1996; U.S. Pat. No. 5,786,146), and methylated CpG island amplification (“MCA”; Toyota et al., Cancer Res. 59:2307-12, 1999) are used alone or in combination with other of these methods.

The “HeavyMethyl™” assay, technique is a quantitative method for assessing methylation differences based on methylation specific amplification of bisulfite treated DNA. Methylation specific blocking probes (also referred to herein as blockers) covering CpG positions between, or covered by the amplification primers enable methylation-specific selective amplification of a nucleic acid sample.

The term “HeavyMethyl™ MethyLight™” assay, in the embodiment thereof implemented herein, refers to a HeavyMethyl™ MethyLight™ assay, which is a variation of the MethyLight™ assay, wherein the MethyLight™ assay is combined with methylation specific blocking probes covering CpG positions between the amplification primers. The HeavyMethyl™ assay may also be used in combination with methylation specific amplification primers.

Typical reagents (e.g., as might be found in a typical MethyLight□-based kit) for HeavyMethyl™ analysis may include, but are not limited to: PCR primers for specific genes (or bisulfite treated DNA sequence or CpG island); blocking oligonucleotides; optimized PCR buffers and deoxynucleotides; and Taq polymerase.

MSP. MSP (methylation-specific PCR) allows for assessing the methylation status of virtually any group of CpG sites within a CpG island, independent of the use of methylation-sensitive restriction enzymes (Herman et al. Proc. Natl. Acad. Sci. USA 93:9821-9826, 1996; U.S. Pat. No. 5,786,146). Briefly, DNA is modified by sodium bisulfite converting all unmethylated, but not methylated cytosines to uracil, and subsequently amplified with primers specific for methylated versus unmethylated DNA. MSP requires only small quantities of DNA, is sensitive to 0.1% methylated alleles of a given CpG island locus, and can be performed on DNA extracted from paraffin-embedded samples. Typical reagents (e.g., as might be found in a typical MSP-based kit) for MSP analysis may include, but are not limited to: methylated and unmethylated PCR primers for specific gene (or bisulfite treated DNA sequence or CpG island), optimized PCR buffers and deoxynucleotides, and specific probes.

MethyLight™. The MethyLight™ assay is a high-throughput quantitative methylation assay that utilizes fluorescence-based real-time PCR (TaqMan□) technology that requires no further manipulations after the PCR step (Eads et al., Cancer Res. 59:2302-2306, 1999). Briefly, the MethyLight™ process begins with a mixed sample of genomic DNA that is converted, in a sodium bisulfite reaction, to a mixed pool of methylation-dependent sequence differences according to standard procedures (the bisulfite process converts unmethylated cytosine residues to uracil). Fluorescence-based PCR is then performed in a “biased” (with PCR primers that overlap known CpG dinucleotides) reaction. Sequence discrimination can occur both at the level of the amplification process and at the level of the fluorescence detection process.

The MethyLight™ assay may be used as a quantitative test for methylation patterns in the genomic DNA sample, wherein sequence discrimination occurs at the level of probe hybridization. In this quantitative version, the PCR reaction provides for a methylation specific amplification in the presence of a fluorescent probe that overlaps a particular putative methylation site. An unbiased control for the amount of input DNA is provided by a reaction in which neither the primers, nor the probe overlie any CpG dinucleotides. Alternatively, a qualitative test for genomic methylation is achieved by probing of the biased PCR pool with either control oligonucleotides that do not “cover” known methylation sites (a fluorescence-based version of the HeavyMethyl™ and MSP techniques), or with oligonucleotides covering potential methylation sites.

The MethyLight™ process can by used with any suitable probes e.g. “TaqMan®”, Lightcycler® etc. For example, double-stranded genomic DNA is treated with sodium bisulfite and subjected to one of two sets of PCR reactions using TaqMan® probes; e.g., with MSP primers and/or HeavyMethyl blocker oligonucleotides and TaqMan® probe. The TaqMan® probe is dual-labeled with fluorescent “reporter” and “quencher” molecules, and is designed to be specific for a relatively high GC content region so that it melts out at about 10° C. higher temperature in the PCR cycle than the forward or reverse primers. This allows the TaqMan® probe to remain fully hybridized during the PCR annealing/extension step. As the Taq polymerase enzymatically synthesizes a new strand during PCR, it will eventually reach the annealed TaqMan® probe. The Taq polymerase 5′ to 3′ endonuclease activity will then displace the TaqMan® probe by digesting it to release the fluorescent reporter molecule for quantitative detection of its now unquenched signal using a real-time fluorescent detection system.

Typical reagents (e.g., as might be found in a typical MethyLight□-based kit) for MethyLight™ analysis may include, but are not limited to: PCR primers for specific gene (or bisulfite treated DNA sequence or CpG island); TaqMan® or Lightcycler® probes; optimized PCR buffers and deoxynucleotides; and Taq polymerase.

The QM™ (quantitative methylation) assay is an alternative quantitative test for methylation patterns in genomic DNA samples, wherein sequence discrimination occurs at the level of probe hybridization. In this quantitative version, the PCR reaction provides for unbiased amplification in the presence of a fluorescent probe that overlaps a particular putative methylation site. An unbiased control for the amount of input DNA is provided by a reaction in which neither the primers, nor the probe overlie any CpG dinucleotides. Alternatively, a qualitative test for genomic methylation is achieved by probing of the biased PCR pool with either control oligonucleotides that do not “cover” known methylation sites (a fluorescence-based version of the HeavyMethyl™ and MSP techniques), or with oligonucleotides covering potential methylation sites.

The QM™ process can by used with any suitable probes e.g. “TaqMan®”, Lightcycler® etc. . . . in the amplification process. For example, double-stranded genomic DNA is treated with sodium bisulfite and subjected to unbiased primers and the TaqMan® probe. The TaqMan® probe is dual-labeled with fluorescent “reporter” and “quencher” molecules, and is designed to be specific for a relatively high GC content region so that it melts out at about 10° C. higher temperature in the PCR cycle than the forward or reverse primers. This allows the TaqMan® probe to remain fully hybridized during the PCR annealing/extension step. As the Taq polymerase enzymatically synthesizes a new strand during PCR, it will eventually reach the annealed TaqMan® probe. The Taq polymerase 5′ to 3′ endonuclease activity will then displace the TaqMan® probe by digesting it to release the fluorescent reporter molecule for quantitative detection of its now unquenched signal using a real-time fluorescent detection system.

Typical reagents (e.g., as might be found in a typical QM™-based kit) for QM™ analysis may include, but are not limited to: PCR primers for specific gene (or bisulfite treated DNA sequence or CpG island); TaqMan® or Lightcycler® probes; optimized PCR buffers and deoxynucleotides; and Taq polymerase.

Ms-SNuPE. The Ms-SNuPE™ technique is a quantitative method for assessing methylation differences at specific CpG sites based on bisulfite treatment of DNA, followed by single-nucleotide primer extension (Gonzalgo & Jones, Nucleic Acids Res. 25:2529-2531, 1997). Briefly, genomic DNA is reacted with sodium bisulfite to convert unmethylated cytosine to uracil while leaving 5-methylcytosine unchanged. Amplification of the desired target sequence is then performed using PCR primers specific for bisulfite-converted DNA, and the resulting product is isolated and used as a template for methylation analysis at the CpG site(s) of interest. Small amounts of DNA can be analyzed (e.g., microdissected pathology sections), and it avoids utilization of restriction enzymes for determining the methylation status at CpG sites.

Typical reagents (e.g., as might be found in a typical Ms-SNuPE™-based kit) for Ms-SNuPE™ analysis may include, but are not limited to: PCR primers for specific gene (or bisulfite treated DNA sequence or CpG island); optimized PCR buffers and deoxynucleotides; gel extraction kit; positive control primers; Ms-SNuPE™primers for specific gene; reaction buffer (for the Ms-SNuPE reaction); and labelled nucleotides. Additionally, bisulfite conversion reagents may include: DNA denaturation buffer; sulfonation buffer; DNA recovery regents or kit (e.g., precipitation, ultrafiltration, affinity column); desulfonation buffer; and DNA recovery components.

The genomic sequences according to SEQ ID NO: 1-4; 37-65, and non-naturally occurring treated variants thereof according to SEQ ID NO: 5-20; 66-181, were determined to have novel utility for the early detection, classification and/or treatment of cell proliferative disorders, in particular prostate carcinoma

In one embodiment the invention of the method comprises the following steps: i) contacting genomic DNA (preferably isolated from body fluids) obtained from the subject with at least one reagent, or series of reagents that distinguishes between methylated and non-methylated CpG dinucleotides within at least one gene selected from the group consisting of RASSF2A; TFAP2E; HIST1H4K; GSTPi; MME; RARB; NKX3-1; NPAL3; ACVR2A; ARL4C; PDE4D; CARTPT; HIST1H2BD; CUL1; MOBKL2B; IFLTD1 (KRAS); ANXA2; NFATC3; MN1; KLF8; FGF13; NKX2-6; SPG20; DSE/SART2; GJA1 (CX43); SPATA6; MCC; GPR68 (INTRON 1 AND/OR EXON 2 REGION); (including promoter and regulatory regions); and ii) detecting prostate cell proliferative disorders, most preferably, prostate carcinoma.

Preferably a plurality of genes (herein also referred to as a “gene panel”) are analysed. Preferably 2, 3 or 4 genes are analysed. In one embodiment of the method said panel comprises at least one gene selected from the group consisting of RASSF2A; TFAP2E; HIST1H4K; GSTPi; MME; RARB; NKX3-1; NPAL3; ACVR2A; ARL4C; PDE4D; CARTPT; HIST1H2BD; CUL1; MOBKL2B; IFLTD1 (KRAS); ANXA2; NFATC3; MN1; KLF8; FGF13; NKX2-6; SPG20; DSE/SART2; GJA1 (CX43); SPATA6; MCC; GPR68 (INTRON 1 AND/OR EXON 2 REGION); and/or their promoter or regulatory regions. Preferably said group comprises the gene HIST1H4K.

Particularly preferred are the following combinations of genes:

### HIST1H4K +RASSF2A or HIST1H4K +GSTPi

Genomic DNA may be isolated by any means standard in the art, including the use of commercially available kits. Briefly, wherein the DNA of interest is encapsulated in by a cellular membrane the biological sample must be disrupted and lysed by enzymatic, chemical or mechanical means. The DNA solution may then be cleared of proteins and other contaminants, e.g., by digestion with proteinase K. The genomic DNA is then recovered from the solution. This may be carried out by means of a variety of methods including salting out, organic extraction or binding of the DNA to a solid phase support. The choice of method will be affected by several factors including time, expense and required quantity of DNA. All clinical sample types comprising neoplastic matter or pre-neoplastic matter are suitable for use in the present method, preferred are cell lines, histological slides, biopsies, paraffin-embedded tissue, body fluids, ejaculate, urine, blood plasma, blood serum, whole blood, isolated blood cells, cells isolated from the blood and combinations thereof. Body fluids are the preferred source of the DNA; particularly preferred are ejaculate, urine, blood plasma, blood serum, whole blood, isolated blood cells and cells isolated from the blood.

The genomic DNA sample is then treated with at least one reagent, or series of reagents that distinguishes between methylated and non-methylated CpG dinucleotides within at least one and more preferably a plurality of target region(s) of the genomic DNA, wherein each target region comprises, or hybridizes under stringent conditions to a sequence of at least 16, 50, 100 or 500 contiguous nucleotides of at least one sequence selected from the group consisting of SEQ ID NO: 1-4; 37-65 respectively, wherein said contiguous nucleotides comprise at least one CpG dinucleotide sequence.

It is particularly preferred that said reagent converts cytosine bases which are unmethylated at the 5′-position to uracil, thymine, or another base which is dissimilar to cytosine in terms of hybridisation behaviour. However in an alternative embodiment said reagent may be a methylation sensitive restriction enzyme.

Wherein the genomic DNA sample is treated in such a manner that cytosine bases which are unmethylated at the 5′-position are converted to uracil, thymine, or another base which is dissimilar to cytosine in terms of hybridization behavior It is preferred that this treatment is carried out with bisulfite (hydrogen sulfite, disulfite) and subsequent alkaline hydrolysis. Such a treatment results in the conversion of SEQ ID NO: 1-4; 37-65 to SEQ ID NO: 5-12; 66-123 (see Table 1) wherein said CpG dinucleotides are methylated or SEQ ID NO: 13-20; 124-181 wherein said CpG dinucleotides are unmethylated (see Table 1).

The treated DNA is then analysed in order to determine the methylation state of at least one gene selected from the group consisting of RASSF2A; TFAP2E; HIST1H4K; GSTPi; MME; RARB; NKX3-1; NPAL3; ACVR2A; ARL4C; PDE4D; CARTPT; HIST1H2BD; CUL1; MOBKL2B; IFLTD1 (KRAS); ANXA2; NFATC3; MN1; KLF8; FGF13; NKX2-6; SPG20; DSE/SART2; GJA1 (CX43); SPATA6; MCC; GPR68 (INTRON 1 AND/OR EXON 2 REGION); prior to the treatment.

Preferably a plurality of genes (herein also referred to as a “gene panel”) are analysed. Preferably 2, 3 or 4 genes are analysed. In one embodiment of the method said panel comprises at least one gene selected from the group consisting of RASSF2A; TFAP2E; HIST1H4K; GSTPi; MME; RARB; NKX3-1; NPAL3; ACVR2A; ARL4C; PDE4D; CARTPT; HIST1H2BD; CUL1; MOBKL2B; IFLTD1 (KRAS); ANXA2; NFATC3; MN1; KLF8; FGF13; NKX2-6; SPG20; DSE/SART2; GJA1 (CX43); SPATA6; MCC; GPR68 (INTRON 1 AND/OR EXON 2 REGION); and/or their promoter or regulatory regions. Preferably said group comprises the gene HIST1H4K.

Particularly preferred are the following combinations of genes:

### HIST1H4K +RASSF2A or HIST1H4K +GSTPi

It is particularly preferred that each target region comprises, or hybridizes under stringent conditions to at least 16, 50, 100 or 500 contiguous nucleotides of a gene selected from the group consisting of RASSF2A; TFAP2E; HIST1H4K; GSTPi; MME; RARB; NKX3-1; NPAL3; ACVR2A; ARL4C; PDE4D; CARTPT; HIST1H2BD; CUL1; MOBKL2B; IFLTD1 (KRAS); ANXA2; NFATC3; MN1; KLF8; FGF13; NKX2-6; SPG20; DSE/SART2; GJA1 (CX43); SPATA6; MCC; GPR68 (INTRON 1 AND/OR EXON 2 REGION). It is preferred that the sequence of said genes according to SEQ ID NO: 1-4; 37-65 is analysed as provided in Table 1 and the accompanying sequence listing. The method of analysis may be selected from those known in the art, including those listed herein. Particularly preferred are MethyLight™, MSP and the use of blocking oligonucleotides (HeavyMethyl™) as described herein. It is further preferred that any oligonucleotides used in such analysis (including primers, blocking oligonucleotides and detection probes) should be reverse complementary, identical, or hybridise under stringent or highly stringent conditions to an at least 16-base-pair long segment of the base sequences of one or more of SEQ ID NO: 5-20; 66-181 and sequences complementary thereto.

Aberrant methylation, more specifically hypermethylation of RASSF2A; TFAP2E; HIST1H4K; GSTPi; MME; RARB; NKX3-1; NPAL3; ACVR2A; ARL4C; PDE4D; CARTPT; HIST1H2BD; CUL1; MOBKL2B; IFLTD1 (KRAS); ANXA2; NFATC3; MN1; KLF8; FGF13; NKX2-6; SPG20; DSE/SART2; GJA1 (CX43); SPATA6; MCC; GPR68 (INTRON 1 AND/OR EXON 2 REGION); (as well as promoter and/or regulatory regions thereof) is associated with the presence of prostate cell proliferative disorders, in particular, prokate cancer. Accordingly wherein a biological sample presents within any degree of methylation, said sample should be determined as being of a cell proliferative disorder, in particular cancer.

Said method may be enabled by means of any analysis of the expression of an RNA transcribed therefrom or polypeptide or protein translated from said RNA, preferably by means of mRNA expression analysis or polypeptide expression analysis. Accordingly the present invention also provides diagnostic assays and methods, both quantitative and qualitative for detecting the expression of at least one gene selected from the group consisting of RASSF2A; TFAP2E; HIST1H4K; GSTPi; MME; RARB; NKX3-1; NPAL3; ACVR2A; ARL4C; PDE4D; CARTPT; HIST1H2BD; CULL MOBKL2B; IFLTD1 (KRAS); ANXA2; NFATC3; MN1; KLF8; FGF13; NKX2-6; SPG20; DSE/SART2; GJA1 (CX43); SPATA6; MCC; GPR68 (INTRON 1 AND/OR EXON 2 REGION); in a subject and determining therefrom upon the presence or absence of prostate cell proliferative disorders, most preferably, prostate cancer in said subject.

Aberrant expression of mRNA transcribed from at least one gene selected from the group consisting of RASSF2A; TFAP2E; HIST1H4K; GSTPi; MME; RARB; NKX3-1; NPAL3; ACVR2A; ARL4C; PDE4D; CARTPT; HIST1H2BD; CUL1; MOBKL2B; IFLTD1 (KRAS); ANXA2; NFATC3; MN1; KLF8; FGF13; NKX2-6; SPG20; DSE/SART2; GJA1 (CX43); SPATA6; MCC; GPR68 (INTRON 1 AND/OR EXON 2 REGION); is associated with the presence of prostate cell proliferative disorders, in particular, prostate cancer in a subject. According to the present invention, under expression (and/or presence methylation) is associated with the presence of cancer, and vice versa over-expression (and/or absence of methylation) is associated with the absence of cancer.

Preferably the mRNA expression of plurality of genes (herein also referred to as a “gene panel”) are analysed. Preferably 2, 3 or 4 genes are analysed. In one embodiment of the method said panel comprises at least one gene selected from the group consisting of RASSF2A; TFAP2E; HIST1H4K; GSTPi; MME; RARB; NKX3-1; NPAL3; ACVR2A; ARL4C; PDE4D; CARTPT; HIST1H2BD; CUL1; MOBKL2B; IFLTD1 (KRAS); ANXA2; NFATC3; MN1; KLF8; FGF13; NKX2-6; SPG20; DSE/SART2; GJA1 (CX43); SPATA6; MCC; GPR68 (INTRON 1 AND/OR EXON 2 REGION); and/or their promoter or regulatory regions. Preferably said group comprises the gene HIST1H4K.

Particularly preferred are the following combinations of genes:

### HIST1H4K +RASSF2A or HIST1H4K +GSTPi

To detect the presence of mRNA encoding a gene or genomic sequence, a sample is obtained from a patient. The sample may be any suitable sample comprising cellular matter of the tumor. Suitable sample types include cell lines, histological slides, biopsies, paraffin-embedded tissue, body fluids, ejaculate, urine, blood plasma, blood serum, whole blood, isolated blood cells, cells isolated from the blood and all possible combinations thereof. It is preferred that said sample types are ejaculate or body fluids selected from the group consisting ejaculate, urine, blood plasma, blood serum, whole blood, isolated blood cells, cells isolated from the blood.

The sample may be treated to extract the RNA contained therein. The resulting nucleic acid from the sample is then analysed. Many techniques are known in the state of the art for determining absolute and relative levels of gene expression, commonly used techniques suitable for use in the present invention include in situ hybridisation (e.g. FISH), Northern analysis, RNase protection assays (RPA), microarrays and PCR-based techniques, such as quantitative PCR and differential display PCR or any other nucleic acid detection method.

Particularly preferred is the use of the reverse transcription/polymerisation chain reaction technique (RT-PCR). The method of RT-PCR is well known in the art (for example, see Watson and Fleming, supra).

The RT-PCR method can be performed as follows. Total cellular RNA is isolated by, for example, the standard guanidium isothiocyanate method and the total RNA is reverse transcribed. The reverse transcription method involves synthesis of DNA on a template of RNA using a reverse transcriptase enzyme and a 3′ end oligonucleotide dT primer and/or random hexamer primers. The cDNA thus produced is then amplified by means of PCR. (Belyaysky et al, Nucl Acid Res 17:2919-2932, 1989; Krug and Berger, Methods in Enzymology, Academic Press, N.Y., Vol. 152, pp. 316-325, 1987 which are incorporated by reference). Further preferred is the “Real-time” variant of RT-PCR, wherein the PCR product is detected by means of hybridisation probes (e.g. TaqMan™, Lightcycler™, Molecular Beacons™ & Scorpion™ primer) or SYBR green. The detected signal from the probes or SYBR green is then quantitated either by reference to a standard curve or by comparing the Ct values to that of a calibration standard. Analysis of housekeeping genes is often used to normalize the results.

In Northern blot analysis total or poly(A)— mRNA is run on a denaturing agarose gel and detected by hybridisation to a labelled probe in the dried gel itself or on a membrane. The resulting signal is proportional to the amount of target RNA in the RNA population.

Comparing the signals from two or more cell populations or tissues reveals relative differences in gene expression levels. Absolute quantitation can be performed by comparing the signal to a standard curve generated using known amounts of an in vitro transcript corresponding to the target RNA. Analysis of housekeeping genes, genes whose expression levels are expected to remain relatively constant regardless of conditions, is often used to normalize the results, eliminating any apparent differences caused by unequal transfer of RNA to the membrane or unequal loading of RNA on the gel.

The first step in Northern analysis is isolating pure, intact RNA from the cells or tissue of interest. Because Northern blots distinguish RNAs by size, sample integrity influences the degree to which a signal is localized in a single band. Partially degraded RNA samples will result in the signal being smeared or distributed over several bands with an overall loss in sensitivity and possibly an erroneous interpretation of the data. In Northern blot analysis, DNA, RNA and oligonucleotide probes can be used and these probes are preferably labelled (e.g. radioactive labels, mass labels, chemiluminescent labels or fluorescent labels). The size of the target RNA, not the probe, will determine the size of the detected band, so methods such as random-primed labelling, which generates probes of variable lengths, are suitable for probe synthesis. The specific activity of the probe will determine the level of sensitivity, so it is preferred that probes with high specific activities, are used.

In an RNase protection assay (RPA), the RNA target and an RNA probe of a defined length are hybridised in solution. Following hybridisation, the RNA is digested with RNases specific for single-stranded nucleic acids to remove any unhybridized, single-stranded target RNA and probe. The RNases are inactivated, and the RNA is separated e.g. by denaturing polyacrylamide gel electrophoresis. The amount of intact RNA probe is proportional to the amount of target RNA in the RNA population. RPA can be used for relative and absolute quantitation of gene expression and also for mapping RNA structure, such as intron/exon boundaries and transcription start sites. The RNase protection assay is preferable to Northern blot analysis as it generally has a lower limit of detection.

The antisense RNA probes used in RPA are generated by in vitro transcription of a DNA template with a defined endpoint and are typically in the range of 50-600 nucleotides. The use of RNA probes that include additional sequences not homologous to the target RNA allows the protected fragment to be distinguished from the full-length probe. RNA probes are typically used instead of DNA probes due to the ease of generating single-stranded RNA probes and the reproducibility and reliability of RNA:RNA duplex digestion with RNases (Ausubel et al. 2003), particularly preferred are probes with high specific activities.

Particularly preferred is the use of microarrays. The microarray analysis process can be divided into two main parts. First is the immobilization of known gene sequences onto glass slides or other solid support followed by hybridisation of the fluorescently labelled cDNA (comprising the sequences to be interrogated) to the known genes immobilized on the glass slide (or other solid phase). After hybridisation, arrays are scanned using a fluorescent microarray scanner. Analysing the relative fluorescent intensity of different genes provides a measure of the differences in gene expression.

DNA arrays can be generated by immobilizing presynthesized oligonucleotides onto prepared glass slides or other solid surfaces. In this case, representative gene sequences are manufactured and prepared using standard oligonucleotide synthesis and purification methods. These synthesized gene sequences are complementary to the RNA transcript(s) of the genes RASSF2A; TFAP2E; HIST1H4K; GSTPi; MME; RARB; NKX3-1; NPAL3; ACVR2A; ARL4C; PDE4D; CARTPT; HIST1H2BD; CUL1; MOBKL2B; IFLTD1 (KRAS); ANXA2; NFATC3; MN1; KLF8; FGF13; NKX2-6; SPG20; DSE/SART2; GJA1 (CX43); SPATA6; MCC; GPR68 (INTRON 1 AND/OR EXON 2 REGION); and tend to be shorter sequences in the range of 25-70 nucleotides. Alternatively, immobilized oligos can be chemically synthesized in situ on the surface of the slide. In situ oligonucleotide synthesis involves the consecutive addition of the appropriate nucleotides to the spots on the microarray; spots not receiving a nucleotide are protected during each stage of the process using physical or virtual masks. Preferably said synthesized nucleic acids are locked nucleic acids.

In expression profiling microarray experiments, the RNA templates used are representative of the transcription profile of the cells or tissues under study. RNA is first isolated from the cell populations or tissues to be compared. Each RNA sample is then used as a template to generate fluorescently labelled cDNA via a reverse transcription reaction. Fluorescent labelling of the cDNA can be accomplished by either direct labelling or indirect labelling methods. During direct labelling, fluorescently modified nucleotides (e.g., Cy®3- or Cy®5-dCTP) are incorporated directly into the cDNA during the reverse transcription. Alternatively, indirect labelling can be achieved by incorporating aminoallyl-modified nucleotides during cDNA synthesis and then conjugating an N-hydroxysuccinimide (NHS)-ester dye to the aminoallyl-modified cDNA after the reverse transcription reaction is complete. Alternatively, the probe may be unlabelled, but may be detectable by specific binding with a ligand which is labelled, either directly or indirectly. Suitable labels and methods for labelling ligands (and probes) are known in the art, and include, for example, radioactive labels which may be incorporated by known methods (e.g., nick translation or kinasing). Other suitable labels include but are not limited to biotin, fluorescent groups, chemiluminescent groups (e.g., dioxetanes, particularly triggered dioxetanes), enzymes, antibodies, and the like.

To perform differential gene expression analysis, cDNA generated from different RNA samples are labelled with Cy®3. The resulting labelled cDNA is purified to remove unincorporated nucleotides, free dye and residual RNA. Following purification, the labelled cDNA samples are hybridised to the microarray. The stringency of hybridisation is determined by a number of factors during hybridisation and during the washing procedure, including temperature, ionic strength, length of time and concentration of formamide. These factors are outlined in, for example, Sambrook et al. (Molecular Cloning: A Laboratory Manual, 2nd ed., 1989). The microarray is scanned post-hybridisation using a fluorescent microarray scanner. The fluorescent intensity of each spot indicates the level of expression of the analysed gene; bright spots correspond to strongly expressed genes, while dim spots indicate weak expression.

Once the images are obtained, the raw data must be analysed. First, the background fluorescence must be subtracted from the fluorescence of each spot. The data is then normalized to a control sequence, such as exogenously added nucleic acids (preferably RNA or DNA), or a housekeeping gene panel to account for any non-specific hybridisation, array imperfections or variability in the array set-up, cDNA labelling, hybridisation or washing. Data normalization allows the results of multiple arrays to be compared.

Another aspect of the invention relates to a kit for use in diagnosis of prostate cell proliferative disorders, most preferably, prostate cancer in a subject according to the methods of the present invention, said kit comprising: a means for measuring the level of transcription of at least one gene selected from the group consisting of RASSF2A; TFAP2E; HIST1H4K; GSTPi; MME; RARB; NKX3-1; NPAL3; ACVR2A; ARL4C; PDE4D; CARTPT; HIST1H2BD; CUL1; MOBKL2B; IFLTD1 (KRAS); ANXA2; NFATC3; MN1; KLF8; FGF13; NKX2-6; SPG20; DSE/SART2; GJA1 (CX43); SPATA6; MCC; GPR68 (INTRON 1 AND/OR EXON 2 REGION). In a preferred embodiment the means for measuring the level of transcription comprise oligonucleotides or polynucleotides able to hybridise under stringent or moderately stringent conditions to the transcription products of a gene selected from the group consisting of RASSF2A; TFAP2E; HIST1H4K; GSTPi; MME; RARB; NKX3-1; NPAL3; ACVR2A; ARL4C; PDE4D; CARTPT; HIST1H2BD; CUL1; MOBKL2B; IFLTD1 (KRAS); ANXA2; NFATC3; MN1; KLF8; FGF13; NKX2-6; SPG20; DSE/SART2; GJA1 (CX43); SPATA6; MCC; GPR68 (INTRON 1 AND/OR EXON 2 REGION). In a most preferred embodiment the level of transcription is determined by techniques selected from the group of Northern Blot analysis, reverse transcriptase PCR, real-time PCR, RNAse protection, and microarray. In another embodiment of the invention the kit further comprises means for obtaining a biological sample of the patient. Preferred is a kit, which further comprises a container which is most preferably suitable for containing the means for measuring the level of transcription and the biological sample of the patient, and most preferably further comprises instructions for use and interpretation of the kit results.

In a preferred embodiment the kit comprises (a) a plurality of oligonucleotides or polynucleotides able to hybridise under stringent or moderately stringent conditions to the transcription products of at least one gene selected from the group consisting of RASSF2A; TFAP2E; HIST1H4K; GSTPi; MME; RARB; NKX3-1; NPAL3; ACVR2A; ARL4C; PDE4D; CARTPT; HIST1H2BD; CUL1; MOBKL2B; IFLTD1 (KRAS); ANXA2; NFATC3; MN1; KLF8; FGF13; NKX2-6; SPG20; DSE/SART2; GJA1 (CX43); SPATA6; MCC; GPR68 (INTRON 1 AND/OR EXON 2 REGION); (b) a container, preferably suitable for containing the oligonucleotides or polynucleotides and a biological sample of the patient comprising the transcription products wherein the oligonucleotides or polynucleotides can hybridise under stringent or moderately stringent conditions to the transcription products, (c) means to detect the hybridisation of (b); and optionally, (d) instructions for use and interpretation of the kit results

The kit may also contain other components such as hybridisation buffer (where the oligonucleotides are to be used as a probe) packaged in a separate container. Alternatively, where the oligonucleotides are to be used to amplify a target region, the kit may contain, packaged in separate containers, a polymerase and a reaction buffer optimised for primer extension mediated by the polymerase, such as PCR. Preferably said polymerase is a reverse transcriptase. It is further preferred that said kit further contains an Rnase reagent.

The present invention further provides for methods for the detection of the presence of the polypeptide encoded by said gene sequences in a sample obtained from a patient.

Aberrant levels of polypeptide expression of the polypeptides encoded by at least one gene selected from the group consisting of RASSF2A; TFAP2E; HIST1H4K; GSTPi; MME; RARB; NKX3-1; NPAL3; ACVR2A; ARL4C; PDE4D; CARTPT; HIST1H2BD; CUL1; MOBKL2B; IFLTD1 (KRAS); ANXA2; NFATC3; MN1; KLF8; FGF13; NKX2-6; SPG20; DSE/SART2; GJA1 (CX43); SPATA6; MCC; GPR68 (INTRON 1 AND/OR EXON 2 REGION); are associated with the presence of cancer.

According to the present invention, under expression of said polypeptides is associated with the presence of prostate cell proliferative disorders, in particular, prostate cancer.

Any method known in the art for detecting polypeptides can be used. Such methods include, but are not limited to masss-spectrometry, immunodiffusion, immunoelectrophoresis, immunochemical methods, binder-ligand assays, immunohistochemical techniques, agglutination and complement assays (e.g., see Basic and Clinical Immunology, Sites and Terr, eds., Appleton & Lange, Norwalk, Conn. pp 217-262, 1991 which is incorporated by reference). Preferred are binder-ligand immunoassay methods including reacting antibodies with an epitope or epitopes and competitively displacing a labelled polypeptide or derivative thereof.

Certain embodiments of the present invention comprise the use of antibodies specific to the polypeptide(s) encoded by the RASSF2A; TFAP2E; HIST1H4K; GSTPi; MME; RARB; NKX3-1; NPAL3; ACVR2A; ARL4C; PDE4D; CARTPT; HIST1H2BD; CUL1; MOBKL2B; IFLTD1 (KRAS); ANXA2; NFATC3; MN1; KLF8; FGF13; NKX2-6; SPG20; DSE/SART2; GJA1 (CX43); SPATA6; MCC; GPR68 (INTRON 1 AND/OR EXON 2 REGION) genes.

Such antibodies are useful for cancer diagnosis. In certain embodiments production of monoclonal or polyclonal antibodies can be induced by the use of an epitope encoded by a polypeptide of a gene selected from the group consisting of RASSF2A; TFAP2E; HIST1H4K; GSTPi; MME; RARB; NKX3-1; NPAL3; ACVR2A; ARL4C; PDE4D; CARTPT; HIST1H2BD; CUL1; MOBKL2B; IFLTD1 (KRAS); ANXA2; NFATC3; MN1; KLF8; FGF13; NKX2-6; SPG20; DSE/SART2; GJA1 (CX43); SPATA6; MCC; GPR68 (INTRON 1 AND/OR EXON 2 REGION) as an antigene. Such antibodies may in turn be used to detect expressed polypeptides as markers for cancer diagnosis. The levels of such polypeptides present may be quantified by conventional methods. Antibody-polypeptide binding may be detected and quantified by a variety of means known in the art, such as labelling with fluorescent or radioactive ligands. The invention further comprises kits for performing the above-mentioned procedures, wherein such kits contain antibodies specific for the investigated polypeptides.

Numerous competitive and non-competitive polypeptide binding immunoassays are well known in the art. Antibodies employed in such assays may be unlabelled, for example as used in agglutination tests, or labelled for use a wide variety of assay methods. Labels that can be used include radionuclides, enzyme's, fluorescers, chemiluminescers, enzyme substrates or co-factors, enzyme inhibitors, particles, dyes and the like. Preferred assays include but are not limited to radioimmunoassay (RIA), enzyme immunoassays, e.g., enzyme-linked immunosorbent assay (ELISA), fluorescent immunoassays and the like. Polyclonal or monoclonal antibodies or epitopes thereof can be made for use in immunoassays by any of a number of methods known in the art.

In an alternative embodiment of the method the proteins may be detected by means of western blot analysis. Said analysis is standard in the art, briefly proteins are separated by means of electrophoresis e.g. SDS-PAGE. The separated proteins are then transferred to a suitable membrane (or paper) e.g. nitrocellulose, retaining the spacial separation achieved by electrophoresis. The membrane is then incubated with a blocking agent to bind remaining sticky places on the membrane, commonly used agents include generic protein (e.g. milk protein). An antibody specific to the protein of interest is then added, said antibody being detectably labelled for example by dyes or enzymatic means (e.g. alkaline phosphatase or horseradish peroxidase). The location of the antibody on the membrane is then detected.

In an alternative embodiment of the method the proteins may be detected by means of immunochemistry (the use of antibodies to probe specific antigens in a sample). Said analysis is standard in the art, wherein detection of antigens in tissues is known as immunohistochemistry, while detection in cultured cells is generally termed immunocytochemistry. Briefly the primary antibody to be detected by binding to its specific antigen. The antibody-antigen complex is then bound by a secondary enzyme conjugated antibody. In the presence of the necessary substrate and chromogen the bound enzyme is detected according to coloured deposits at the antibody-antigen binding sites. There is a wide range of suitable sample types, antigen-antibody affinity, antibody types, and detection enhancement methods. Thus optimal conditions for immunohistochemical or immunocytochemical detection must be determined by the person skilled in the art for each individual case.

One approach for preparing antibodies to a polypeptide is the selection and preparation of an amino acid sequence of all or part of the polypeptide, chemically synthesising the amino acid sequence and injecting it into an appropriate animal, usually a rabbit or a mouse (Milstein and Kohler Nature 256:495-497, 1975; Gulfre and Milstein, Methods in Enzymology: Immunochemical Techniques 73:1-46, Langone and Banatis eds., Academic Press, 1981 which are incorporated by reference in its entirety). Methods for preparation of the polypeptides or epitopes thereof include, but are not limited to chemical synthesis, recombinant DNA techniques or isolation from biological samples.

In the final step of the method the diagnosis of the patient is determined, whereby under-expression (of RASSF2A; TFAP2E; I-IIST1H4K; GSTPi; MME; RARB; NKX3-1; NPAL3; ACVR2A; ARL4C; PDE4D; CARTPT; HIST1H2BD; CUL1; MOBKL2B; IFLTD1 (KRAS); ANXA2; NFATC3; MN1; KLF8; FGF13; NKX2-6; SPG20; DSE/SART2; GJA1 (CX43); SPATA6; MCC; GPR68 (INTRON 1 AND/OR EXON 2 REGION); mRNA or polypeptides) is indicative of the presence of cancer. The term under-expression shall be taken to mean expression at a detected level less than a pre-determined cut off which may be selected from the group consisting of the mean, median or an optimised threshold value.

Another aspect of the invention provides a kit for use in diagnosis of cancer in a subject according to the methods of the present invention, comprising: a means for detecting polypeptides of at least one gene selected from the group consisting of RASSF2A; TFAP2E; HIST1H4K; GSTPi; MME; RARB; NKX3-1; NPAL3; ACVR2A; ARL4C; PDE4D; CARTPT; HIST1H2BD; CUL1; MOBKL2B; IFLTD1 (KRAS); ANXA2; NFATC3; MN1; KLF8; FGF13; NKX2-6; SPG20; DSE/SART2; GJA1 (CX43); SPATA6; MCC; GPR68 (INTRON 1 AND/OR EXON 2 REGION). The means for detecting the polypeptides comprise preferably antibodies, antibody derivatives, or antibody fragments. The polypeptides are most preferably detected by means of Western Blotting utilizing a labelled antibody. In another embodiment of the invention the kit further comprising means for obtaining a biological sample of the patient. Preferred is a kit, which further comprises a container suitable for containing the means for detecting the polypeptides in the biological sample of the patient, and most preferably further comprises instructions for use and interpretation of the kit results. In a preferred embodiment the kit comprises: (a) a means for detecting polypeptides of at least one gene selected from the group consisting of RASSF2A; TFAP2E; HIST1H4K; GSTPi; MME; RARB; NKX3-1; NPAL3; ACVR2A; ARL4C; PDE4D; CARTPT; HIST1H2BD; CUL1; MOBKL2B; IFLTD1 (KRAS); ANXA2; NFATC3; MN1; KLF8; FGF13; NKX2-6; SPG20; DSE/SART2; GJA1 (CX43); SPATA6; MCC; GPR68 (INTRON 1 AND/OR EXON 2 REGION); (b) a container suitable for containing the said means and the biological sample of the patient comprising the polypeptides wherein the means can form complexes with the polypeptides; (c) a means to detect the complexes of (b); and optionally (d) instructions for use and interpretation of the kit results.

The kit may also contain other components such as buffers or solutions suitable for blocking, washing or coating, packaged in a separate container.

Particular embodiments of the present invention provide a novel application of the analysis of methylation levels and/or patterns within said sequences that enables a precise detection, characterisation and/or treatment of prostate carcinoma. Early detection of cancer is directly linked with disease prognosis, and the disclosed method thereby enables the physician and patient to make better and more informed treatment decisions.

The present invention provides novel uses for the genomic sequences SEQ ID NO: 1-4; 37-65. Additional embodiments provide modified variants of SEQ ID NO: 1-4; 37-65, as well as oligonucleotides and/or PNA-oligomers for analysis of cytosine methylation patterns within SEQ ID NO: 1-4; 37-65 or within the modified variants of SEQ ID NO: 1-4; 37-65.

An objective of the invention comprises analysis of the methylation state of one or more CpG dinucleotides within SEQ ID NO: 1-4; 37-65 and sequences complementary thereto.

The disclosed invention provides treated nucleic acids, derived from genomic SEQ ID NO: 1-4; 37-65, wherein the treatment is suitable to convert at least one unmethylated cytosine base of the genomic DNA sequence to uracil or another base that is detectably dissimilar to cytosine in terms of hybridization. The genomic sequences in question may comprise one, or more consecutive methylated CpG positions. Said treatment preferably comprises use of a reagent selected from the group consisting of bisulfate, hydrogen sulfite, disulfite, and combinations thereof. In a preferred embodiment of the invention, the invention provides a non-naturally occurring modified nucleic acid comprising a sequence of at least 16 contiguous nucleotide bases in length of a sequence selected from the group consisting of SEQ ID NO: 5-20; 66-181. In further preferred embodiments of the invention said nucleic acid is at least 50, 100, 150, 200, 250 or 500 base pairs in length of a segment of the nucleic acid sequence disclosed in SEQ ID NO: 5-20; 66-181. Particularly preferred is a nucleic acid molecule that is identical or complementary to all or a portion of the sequences SEQ ID NO: 5-20; 66-181 but not SEQ ID NO: 1-4; 37-65 or other naturally occurring DNA.

It is preferred that said sequence comprises at least one CpG, TpA or CpA dinucleotide and sequences complementary thereto. The sequences of SEQ ID NO: 5-20; 66-181 provide non-naturally occurring modified versions of the nucleic acid according to SEQ ID NO: 1-4; 37-65, wherein the modification of each genomic sequence results in the synthesis of a nucleic acid having a sequence that is unique and distinct from said genomic sequence as follows. For each sense strand genomic DNA, e.g., SEQ ID NO: 1, four converted versions are disclosed. A first version wherein “C” is converted to “T,” but “CpG” remains “CpG” (i.e., corresponds to case where, for the sense strand genomic sequence, all “C” residues of CpG dinucleotide sequences are methylated and are thus not converted); a second version discloses the complement of the disclosed genomic DNA sequence (i.e. antisense strand), wherein “C” is converted to “T,” but “CpG” remains “CpG” (i.e., corresponds to case where, for all “C” residues of CpG dinucleotide sequences are methylated and are thus not converted). The ‘upmethylated’ converted sequences of SEQ ID NO: 1-4; 37-65 correspond to SEQ ID NO: 5-12; 66-123. A third chemically converted version of each genomic sequences is provided, wherein “C” is converted to “T” for all “C” residues, including those of “CpG” dinucleotide sequences (i.e., corresponds to case where, for the sense strand genomic sequences, all “C” residues of CpG dinucleotide sequences are unmethylated); a fourth chemically converted version of each sequence, discloses the complement of the disclosed genomic DNA sequence (i.e. antisense strand), wherein “C” is converted to “T” for all “C” residues, including those of “CpG” dinucleotide sequences (i.e., corresponds to case where, for the complement (antisense strand) of each genomic sequence, all “C” residues of CpG dinucleotide sequences are unmethylated). The ‘downmethylated’ converted sequences of SEQ ID NO: 1-4; 37-65 corresponds to SEQ ID NO: 13-20; 124-181. See Table 1 for further details.

Significantly, heretofore, the nucleic acid sequences and molecules according SEQ ID NO: 5-20; 66-181 were not implicated in or connected with the detection, classification or treatment of cancer.

In an alternative preferred embodiment, the invention further provides oligonucleotides or oligomers suitable for use in the methods of the invention for detecting the cytosine methylation state within genomic or treated (chemically modified) DNA, according to SEQ ID NO: 1-4; 37-65 or SEQ ID NO: 5-20; 66-181. Said oligonucleotide or oligomer nucleic acids provide novel diagnostic means. Said oligonucleotide or oligomer comprising a nucleic acid sequence having a length of at least nine (9) nucleotides which is identical to, hybridizes, under moderately stringent or stringent conditions (as defined herein above), to a treated nucleic acid sequence according to SEQ ID NO: 5-20; 66-181 and/or sequences complementary thereto, or to a genomic sequence according to SEQ ID NO: 1-4; 37-65 and/or sequences complementary thereto.

Thus, the present invention includes nucleic acid molecules (e.g., oligonucleotides and peptide nucleic acid (PNA) molecules (PNA-oligomers)) that hybridize under moderately stringent and/or stringent hybridization conditions to all or a portion of the sequences SEQ ID NO: 1-4; 37-65 or to the complements thereof. Particularly preferred is a nucleic acid molecule that hybridizes under moderately stringent and/or stringent hybridization conditions to all or a portion of the sequences SEQ ID NO: 5-20; 66-181 but not SEQ ID NO: 1-4; 37-65 or other human genomic DNA.

The identical or hybridizing portion of the hybridizing nucleic acids is typically at least 9, 16, 20, 25, or 35 nucleotides in length. However, longer molecules have inventive utility, and are thus within the scope of the present invention.

Preferably, the hybridizing portion of the inventive hybridizing nucleic acids is at least 95%, or at least 98%, or 100% identical to the sequence, or to a portion thereof of SEQ ID NO: 5-20; 66-181, or to the complements thereof.

Hybridizing nucleic acids of the type described herein can be used, for example, as a primer (e.g., a PCR primer), or a diagnostic and/or prognostic probe or primer. Preferably, hybridization of the oligonucleotide probe to a nucleic acid sample is performed under stringent conditions and the probe is 100% identical to the target sequence. Nucleic acid duplex or hybrid stability is expressed as the melting temperature or Tm, which is the temperature at which a probe dissociates from a target DNA. This melting temperature is used to define the required stringency conditions.

For target sequences that are related and substantially identical to the corresponding sequence of SEQ ID NO: 1-4; 37-65 (such as allelic variants and SNPs), rather than identical, it is useful to first establish the lowest temperature at which only homologous hybridization occurs with a particular concentration of salt (e.g., SSC or SSPE). Then, assuming that 1% mismatching results in a 1° C. decrease in the Tm, the temperature of the final wash in the hybridization reaction is reduced accordingly (for example, if sequences having >95% identity with the probe are sought, the final wash temperature is decreased by 5° C.). In practice, the change in Tm can be between 0.5° C. and 1.5° C. per 1% mismatch.

Examples of inventive oligonucleotides of length X (in nucleotides), as indicated by polynucleotide positions with reference to, e.g., SEQ ID NO: 2, include those corresponding to sets (sense and antisense sets) of consecutively overlapping oligonucleotides of length X, where the oligonucleotides within each consecutively overlapping set (corresponding to a given X value) are defined as the finite set of Z oligonucleotides from nucleotide positions:
n to (n+(X−1));
where n=1, 2, 3, . . . (Y—(X−1));
where Y equals the length (nucleotides or base pairs) of SEQ ID NO: 2 (6096);
where X equals the common length (in nucleotides) of each oligonucleotide in the set (e.g., X=20 for a set of consecutively overlapping 20-mers); and
   where the number (Z) of consecutively overlapping oligomers of length X for a given SEQ ID NO 1 of length Y is equal to Y−(X−1). For example Z=6096−19=6077 for either sense or antisense sets of SEQ ID NO: 2, where X=20.

Preferably, the set is limited to those oligomers that comprise at least one CpG, TpG or CpA dinucleotide.

Examples of inventive 20-mer oligonucleotides include the following set of 2,261 oligomers (and the antisense set complementary thereto), indicated by polynucleotide positions with reference to SEQ ID NO: 1-4; 37-65:

1-20, 2-21, 3-22, 4-23, 5-24, . . . and 6077-6096.

Preferably, the set is limited to those oligomers that comprise at least one CpG, TpG or CpA dinucleotide.

Likewise, examples of inventive 25-mer oligonucleotides include the following set of 2,256 oligomers (and the antisense set complementary thereto), indicated by polynucleotide positions with reference to SEQ ID NO: 2:

1-25, 2-26, 3-27, 4-28, 5-29, . . . and 6072-6096.

Preferably, the set is limited to those oligomers that comprise at least one CpG, TpG or CpA dinucleotide.

The present invention encompasses, for each of SEQ ID NO: 1-4; 37-65 (sense and antisense), multiple consecutively overlapping sets of oligonucleotides or modified oligonucleotides of length X, where, e.g., X=9, 10, 17, 20, 22, 23, 25, 27, 30 or 35 nucleotides.

The oligonucleotides or oligomers according to the present invention constitute effective tools useful to ascertain genetic and epigenetic parameters of the genomic sequences corresponding to SEQ ID NO: 1-4; 37-65. Preferred sets of such oligonucleotides or modified oligonucleotides of length X are those consecutively overlapping sets of oligomers corresponding to SEQ ID NO: 1-4; 37-65 (and to the complements thereof). Preferably, said oligomers comprise at least one CpG, TpG or CpA dinucleotide.

Particularly preferred oligonucleotides or oligomers according to the present invention are those in which the cytosine of the CpG dinucleotide (or of the corresponding converted TpG or CpA dinculeotide) sequences is within the middle third of the oligonucleotide; that is, where the oligonucleotide is, for example, 13 bases in length, the CpG, TpG or CpA dinucleotide is positioned within the fifth to ninth nucleotide from the 5′-end.

The oligonucleotides of the invention can also be modified by chemically linking the oligonucleotide to one or more moieties or conjugates to enhance the activity, stability or detection of the oligonucleotide. Such moieties or conjugates include chromophores, fluorophors, lipids such as cholesterol, cholic acid, thioether, aliphatic chains, phospholipids, polyamines, polyethylene glycol (PEG), palmityl moieties, and others as disclosed in, for example, U.S. Pat. Nos. 5,514,758, 5,565,552, 5,567,810, 5,574,142, 5,585,481, 5,587,371, 5,597,696 and 5,958,773. The probes may also exist in the form of a PNA (peptide nucleic acid) which has particularly preferred pairing properties. Thus, the oligonucleotide may include other appended groups such as peptides, and may include hybridization-triggered cleavage agents (Krol et al., BioTechniques 6:958-976, 1988) or intercalating agents (Zon, Pharm. Res. 5:539-549, 1988). To this end, the oligonucleotide may be conjugated to another molecule, e.g., a chromophore, fluorophor, peptide, hybridization-triggered cross-linking agent, transport agent, hybridization-triggered cleavage agent, etc.

The oligonucleotide may also comprise at least one art-recognized modified sugar and/or base moiety, or may comprise a modified backbone or non-natural internucleoside linkage.

The oligonucleotides or oligomers according to particular embodiments of the present invention are typically used in ‘sets,’ which contain at least one oligomer for analysis of each of the CpG dinucleotides of a genomic sequence selected from the group consisting SEQ ID NO: 1-4; 37-65 and sequences complementary thereto, or to the corresponding CpG, TpG or CpA dinucleotide within a sequence of the treated nucleic acids according to SEQ ID NO: 5-20; 66-181 and sequences complementary thereto. However, it is anticipated that for economic or other factors it may be preferable to analyse a limited selection of the CpG dinucleotides within said sequences, and the content of the set of oligonucleotides is altered accordingly.

Therefore, in particular embodiments, the present invention provides a set of at least two (2) oligomers (oligonucleotides and/or PNA-oligomers) that are useful for detecting the cytosine methylation state in treated genomic DNA (SEQ ID NO: 5-20; 66-181), or in genomic DNA (SEQ ID NO: 1-4; 37-65 and sequences complementary thereto). These oligomers may also be named probes and enable diagnosis and detection of prostate cell proliferative disorders, most preferably, prostate carcinoma. The set of oligomers may also be used for detecting single nucleotide polymorphisms (SNPs) in treated genomic DNA (SEQ ID NO: 5-20; 66-181 and sequences complementary thereto), or in genomic DNA (SEQ ID NO: 1-4; 37-65 and sequences complementary thereto).

In preferred embodiments, at least one, and more preferably all members of a set of oligonucleotides is bound to a solid phase.

In further embodiments, the present invention provides a set of at least two (2) oligonucleotides that are used as ‘primer’ oligonucleotides for amplifying DNA sequences of one of SEQ ID NO: 1-4, 37-65; SEQ ID NO: 5-20, 66-181; sequences complementary thereto; or segments thereof.

It is anticipated that the oligonucleotides may constitute all or part of an “array” or “DNA chip” (i.e., an arrangement of different oligonucleotides and/or PNA-oligomers bound to a solid phase). Such an array of different oligonucleotide- and/or PNA-oligomer sequences can be characterized, for example, in that it is arranged on the solid phase in the form of a rectangular or hexagonal lattice. The solid-phase surface may be composed of silicon, glass, polystyrene, aluminium, steel, iron, copper, nickel, silver, or gold. Nitrocellulose as well as plastics such as nylon, which can exist in the form of pellets or also as resin matrices, may also be used. An overview of the Prior Art in oligomer array manufacturing can be gathered from a special edition of Nature Genetics (Nature Genetics Supplement, Volume 21, January 1999, and from the literature cited therein). Fluorescently labelled probes are often used for the scanning of immobilized DNA arrays. The simple attachment of Cy3 and Cy5 dyes to the 5′-OH of the specific probe are particularly suitable for fluorescence labels. The detection of the fluorescence of the hybridised probes may be carried out, for example, via a confocal microscope. Cy3 and Cy5 dyes, besides many others, are commercially available.

It is also anticipated that the oligonucleotides, or particular sequences thereof, may constitute all or part of an “virtual array” wherein the oligonucleotides, or particular sequences thereof, are used, for example, as ‘specifiers’ as part of, or in combination with a diverse population of unique labeled probes to analyze a complex mixture of analytes. Such a method, for example is described in US 2003/0013091 (U.S. Ser. No. 09/898,743, published 16 Jan. 2003). In such methods, enough labels are generated so that each nucleic acid in the complex mixture (i.e., each analyte) can be uniquely bound by a unique label and thus detected (each label is directly counted, resulting in a digital read-out of each molecular species in the mixture).

It is particularly preferred that the oligomers according to the invention are utilised for detecting, or for diagnosing prostate cell proliferative disorders, most preferably, prostate carcinoma.

In the most preferred embodiment of the method, the presence or absence of prostate cell proliferative disorders, most preferably, prostate cancer is determined. This is achieved by analysis of the methylation status of at least one and more preferably a plurality of, target sequence(s) comprising at least one CpG position said sequence comprising, or hybridizing under stringent conditions to at least 16, 50, 100 or 500 contiguous nucleotides of a sequence selected from the group consisting SEQ ID NO: 1-4; 37-65 and complements thereof.

Preferably a plurality of target regions (herein also referred to as a “gene panel”) are analysed. Preferably target regions of 2, 3 or 4 genes are analysed. In one embodiment of the method said panel comprises the target regions of at least one gene selected from the group consisting of RASSF2A; TFAP2E; HIST1H4K; GSTPi; MME; RARB; NKX3-1; NPAL3; ACVR2A; ARL4C; PDE4D; CARTPT; HIST1H2BD; CUL1; MOBKL2B; IFLTD1 (KRAS); ANXA2; NFATC3; MN1; KLF8; FGF13; NKX2-6; SPG20; DSE/SART2; GJA1 (CX43); SPATA6; MCC; GPR68 (INTRON 1 AND/OR EXON 2 REGION); and/or their promoter or regulatory regions. Preferably said panel comprises a target region of the gene HIST1H4K.

Particularly preferred are the following combinations of target regions of genes:

### HIST +RASSF2A or HIST1H4K +GSTPi

The present invention further provides a method for ascertaining genetic and/or epigenetic parameters of the genomic sequences according to SEQ ID NO: 1-4; 37-65 within a subject by analysing cytosine methylation and single nucleotide polymorphisms. Said method comprising contacting a nucleic acid comprising at least one genomic sequence selected from the group consisting SEQ ID NO: 1-4; 37-65 in a biological sample obtained from said subject with at least one reagent or a series of reagents, wherein said reagent or series of reagents, distinguishes between methylated and non-methylated CpG dinucleotides within the target nucleic acid(s).

In a preferred embodiment, said method comprises the following steps: In the first step, a sample of the tissue to be analysed is obtained. The source may be any suitable source, such as cell lines, histological slides, biopsies, paraffin-embedded tissue, body fluids, ejaculate, urine, blood plasma, blood serum, whole blood, isolated blood cells, cells isolated from the blood and all possible combinations thereof. It is preferred that said sources of DNA are ejaculate or body fluids selected from the group consisting ejaculate, urine, blood plasma, blood serum, whole blood, isolated blood cells, cells isolated from the blood.

The genomic DNA is then isolated from the sample. Genomic DNA may be isolated by any means standard in the art, including the use of commercially available kits. Briefly, wherein the DNA of interest is encapsulated in by a cellular membrane the biological sample must be disrupted and lysed by enzymatic, chemical or mechanical means. The DNA solution may then be cleared of proteins and other contaminants e.g. by digestion with proteinase K. The genomic DNA is then recovered from the solution. This may be carried out by means of a variety of methods including salting out, organic extraction or binding of the DNA to a solid phase support. The choice of method will be affected by several factors including time, expense and required quantity of DNA.

Wherein the sample DNA is not enclosed in a membrane (e.g. circulating DNA from a blood sample), methods standard in the art for the isolation and/or purification of DNA may be employed. Such methods include the use of a protein degenerating reagent e.g. chaotropic salt e.g. guanidine hydrochloride or urea; or a detergent e.g. sodium dodecyl sulphate (SDS), cyanogen bromide. Alternative methods include but are not limited to ethanol precipitation or propanol precipitation, vacuum concentration amongst others by means of a centrifuge. The person skilled in the art may also make use of devices such as filter devices e.g. ultrafiltration, silica surfaces or membranes, magnetic particles, polystyrol particles, polystyrol surfaces, positively charged surfaces, and positively charged membranes, charged membranes, charged surfaces, charged switch membranes, charged switched surfaces.

Once the nucleic acids have been extracted, the genomic double stranded DNA is used in the analysis.

In the second step of the method, the genomic DNA sample is treated in such a manner that cytosine bases which are unmethylated at the 5′-position are converted to uracil, thymine, or another base which is dissimilar to cytosine in terms of hybridisation behaviour. This will be understood as ‘pre-treatment’ or ‘treatment’ herein.

This is preferably achieved by means of treatment with a bisulfite reagent. The term “bisulfite reagent” refers to a reagent comprising bisulfite, disulfite, hydrogen sulfite or combinations thereof, useful as disclosed herein to distinguish between methylated and unmethylated CpG dinucleotide sequences. Methods of said treatment are known in the art (e.g. PCT/EP2004/011715, which is incorporated by reference in its entirety): It is preferred that the bisulfite treatment is conducted in the presence of denaturing solvents such as but not limited to n-alkylenglycol, particularly diethylene glycol dimethyl ether (DME), or in the presence of dioxane or dioxane derivatives. In a preferred embodiment the denaturing solvents are used in concentrations between 1% and 35% (v/v). It is also preferred that the bisulfite reaction is carried out in the presence of scavengers such as but not limited to chromane derivatives, e.g., 6-hydroxy-2,5,7,8-tetramethylchromane 2-carboxylic acid or trihydroxybenzoe acid and derivates thereof, e.g. gallic acid (see: PCT/EP2004/011715 which is incorporated by reference in its entirety). The bisulfite conversion is preferably carried out at a reaction temperature between 30° C. and 70° C., whereby the temperature is increased to over 85° C. for short periods of times during the reaction (see: PCT/EP2004/011715 which is incorporated by reference in its entirety). The bisulfite treated DNA is preferably purified priori to the quantification. This may be conducted by any means known in the art, such as but not limited to ultrafiltration, preferably carried out by means of Microcon™ columns (manufactured by Millipore™). The purification is carried out according to a modified manufacturer's protocol (see: PCT/EP2004/011715 which is incorporated by reference in its entirety).

In the third step of the method, fragments of the treated DNA are amplified, using sets of primer oligonucleotides according to the present invention, and an amplification enzyme. The amplification of several DNA segments can be carried out simultaneously in one and the same reaction vessel. Typically, the amplification is carried out using a polymerase chain reaction (PCR). Preferably said amplificates are 100 to 2,000 base pairs in length. The set of primer oligonucleotides includes at least two oligonucleotides whose sequences are each reverse complementary, identical, or hybridise under stringent or highly stringent conditions to an at least 16-base-pair long segment of the base sequences of one of SEQ ID NO: 5-20; 66-181 and sequences complementary thereto.

In an alternate embodiment of the method, the methylation status of pre-selected CpG positions within at least one genomic sequence selected from the group consisting SEQ ID NO: 1-4; 37-65, may be detected by use of methylation-specific primer oligonucleotides. This technique (MSP) has been described in U.S. Pat. No. 6,265,171 to Herman. The use of methylation status specific primers for the amplification of bisulfite treated DNA allows the differentiation between methylated and unmethylated nucleic acids. MSP primers pairs contain at least one primer which hybridises to a bisulfite treated CpG dinucleotide. Therefore, the sequence of said primers comprises at least one CpG dinucleotide. MSP primers specific for non-methylated DNA contain a “T” at the position of the C position in the CpG. Preferably, therefore, the base sequence of said primers is required to comprise a sequence having a length of at least 9 nucleotides which hybridises to a treated nucleic acid sequence according to one of SEQ ID NO: 5-20; 66-181 and sequences complementary thereto, wherein the base sequence of said oligomers comprises at least one CpG dinucleotide. A further preferred embodiment of the method comprises the use of blocker oligonucleotides (the HeavyMethyl™ assay). The use of such blocker oligonucleotides has been described by Yu et al., BioTechniques 23:714-720, 1997. Blocking probe oligonucleotides are hybridised to the bisulfite treated nucleic acid concurrently with the PCR primers. PCR amplification of the nucleic acid is terminated at the 5′ position of the blocking probe, such that amplification of a nucleic acid is suppressed where the complementary sequence to the blocking probe is present. The probes may be designed to hybridize to the bisulfite treated nucleic acid in a methylation status specific manner. For example, for detection of methylated nucleic acids within a population of unmethylated nucleic acids, suppression of the amplification of nucleic acids which are unmethylated at the position in question would be carried out by the use of blocking probes comprising a ‘CpA’ or ‘TpA’ at the position in question, as opposed to a ‘CpG’ if the suppression of amplification of methylated nucleic acids is desired.

For PCR methods using blocker oligonucleotides, efficient disruption of polymerase-mediated amplification requires that blocker oligonucleotides not be elongated by the polymerase. Preferably, this is achieved through the use of blockers that are 3′-deoxyoligonucleotides, or oligonucleotides derivitized at the 3′ position with other than a “free” hydroxyl group. For example, 3′-O-acetyl oligonucleotides are representative of a preferred class of blocker molecule.

Additionally, polymerase-mediated decomposition of the blocker oligonucleotides should be precluded. Preferably, such preclusion comprises either use of a polymerase lacking 5′-3′ exonuclease activity, or use of modified blocker oligonucleotides having, for example, thioate bridges at the 5′-terminii thereof that render the blocker molecule nuclease-resistant. Particular applications may not require such 5′ modifications of the blocker. For example, if the blocker- and primer-binding sites overlap, thereby precluding binding of the primer (e.g., with excess blocker), degradation of the blocker oligonucleotide will be substantially precluded. This is because the polymerase will not extend the primer toward, and through (in the 5′-3′ direction) the blocker—a process that normally results in degradation of the hybridized blocker oligonucleotide.

A particularly preferred blocker/PCR embodiment, for purposes of the present invention and as implemented herein, comprises the use of peptide nucleic acid (PNA) oligomers as blocking oligonucleotides. Such PNA blocker oligomers are ideally suited, because they are neither decomposed nor extended by the polymerase.

Preferably, therefore, the base sequence of said blocking oligonucleotides is required to comprise a sequence having a length of at least 9 nucleotides which hybridises to a treated nucleic acid sequence according to one of SEQ ID NO: 5-20; 66-181 and sequences complementary thereto, wherein the base sequence of said oligonucleotides comprises at least one CpG, TpG or CpA dinucleotide.

The fragments obtained by means of the amplification can carry a directly or indirectly detectable label. Preferred are labels in the form of fluorescence labels, radionuclides, or detachable molecule fragments having a typical mass which can be detected in a mass spectrometer. Where said labels are mass labels, it is preferred that the labelled amplificates have a single positive or negative net charge, allowing for better delectability in the mass spectrometer. The detection may be carried out and visualized by means of, e.g., matrix assisted laser desorption/ionization mass spectrometry (MALDI) or using electron spray mass spectrometry (ESI).

Matrix Assisted Laser Desorption/Ionization Mass Spectrometry (MALDI-TOF) is a very efficient development for the analysis of biomolecules (Karas & Hillenkamp, Anal Chem., 60:2299-301, 1988). An analyte is embedded in a light-absorbing matrix. The matrix is evaporated by a short laser pulse thus transporting the analyte molecule into the vapor phase in an unfragmented manner. The analyte is ionized by collisions with matrix molecules. An applied voltage accelerates the ions into a field-free flight tube. Due to their different masses, the ions are accelerated at different rates. Smaller ions reach the detector sooner than bigger ones. MALDI-TOF spectrometry is well suited to the analysis of peptides and proteins. The analysis of nucleic acids is somewhat more difficult (Gut & Beck, Current Innovations and Future Trends, 1:147-57, 1995). The sensitivity with respect to nucleic acid analysis is approximately 100-times less than for peptides, and decreases disproportionally with increasing fragment size. Moreover, for nucleic acids having a multiply negatively charged backbone, the ionization process via the matrix is considerably less efficient. In MALDI-TOF spectrometry, the selection of the matrix plays an eminently important role. For desorption of peptides, several very efficient matrixes have been found which produce a very fine crystallisation. There are now several responsive matrixes for DNA, however, the difference in sensitivity between peptides and nucleic acids has not been reduced. This difference in sensitivity can be reduced, however, by chemically modifying the DNA in such a manner that it becomes more similar to a peptide. For example, phosphorothioate nucleic acids, in which the usual phosphates of the backbone are substituted with thiophosphates, can be converted into a charge-neutral DNA using simple alkylation chemistry (Gut & Beck, Nucleic Acids Res. 23: 1367-73, 1995). The coupling of a charge tag to this modified DNA results in an increase in MALDI-TOF sensitivity to the same level as that found for peptides. A further advantage of charge tagging is the increased stability of the analysis against impurities, which makes the detection of unmodified substrates considerably more difficult.

In the fourth step of the method, the amplificates obtained during the third step of the method are analysed in order to ascertain the methylation status of the CpG dinucleotides prior to the treatment.

In embodiments where the amplificates were obtained by means of MSP amplification, the presence or absence of an amplificate is in itself indicative of the methylation state of the CpG positions covered by the primer, according to the base sequences of said primer.

Amplificates obtained by means of both standard and methylation specific PCR may be further analysed by means of oligomer-based methods such as, but not limited to, array technology and probe-based technologies as well as by means of techniques such as sequencing and template directed extension.

In one embodiment of the method, the amplificates synthesised in step three are subsequently hybridized to an array or a set of oligonucleotides and/or PNA probes. In this context, the hybridization takes place in the following manner: the set of probes used during the hybridization is preferably composed of at least 2 oligonucleotides or PNA-oligomers; in the process, the amplificates serve as probes which hybridize to oligonucleotides previously bonded to a solid phase; the non-hybridized fragments are subsequently removed; said oligonucleotides contain at least one base sequence having a length of at least 9 nucleotides which is reverse complementary or identical to a segment of the base sequences specified in the present Sequence Listing; and the segment comprises at least one CpG, TpG or CpA dinucleotide. The hybridizing portion of the hybridizing nucleic acids is typically at least 9, 15, 20, 25, 30 or 35 nucleotides in length. However, longer molecules have inventive utility, and are thus within the scope of the present invention.

In a preferred embodiment, said dinucleotide is present in the central third of the oligomer. For example, wherein the oligomer comprises one CpG dinucleotide, said dinucleotide is preferably the fifth to ninth nucleotide from the 5′-end of a 13-mer. One oligonucleotide exists for the analysis of each CpG dinucleotide within a sequence selected from the group consisting SEQ ID NO: 1-4; 37-65, and the equivalent positions within SEQ ID NO: 5-20; 66-181. Said oligonucleotides may also be present in the form of peptide nucleic acids. The non-hybridised amplificates are then removed. The hybridised amplificates are then detected. In this context, it is preferred that labels attached to the amplificates are identifiable at each position of the solid phase at which an oligonucleotide sequence is located.

In yet a further embodiment of the method, the genomic methylation status of the CpG positions may be ascertained by means of oligonucleotide probes (as detailed above) that are hybridised to the bisulfite treated DNA concurrently with the PCR amplification primers (wherein said primers may either be methylation specific or standard).

A particularly preferred embodiment of this method is the use of fluorescence-based Real Time Quantitative PCR (Heid et al., Genome Res. 6:986-994, 1996; also see U.S. Pat. No. 6,331,393) employing a dual-labelled fluorescent oligonucleotide probe (TaqMan™ PCR, using an ABI Prism 7700 Sequence Detection System, Perkin Elmer Applied Biosystems, Foster City, Calif.). The TaqMan™ PCR reaction employs the use of a non-extendible interrogating oligonucleotide, called a TaqMan™ probe, which, in preferred embodiments, is designed to hybridise to a CpG-rich sequence located between the forward and reverse amplification primers. The TaqMan™ probe further comprises a fluorescent “reporter moiety” and a “quencher moiety” covalently bound to linker moieties (e.g., phosphoramidites) attached to the nucleotides of the TaqMan™ oligonucleotide. For analysis of methylation within nucleic acids subsequent to bisulfite treatment, it is required that the probe be methylation specific, as described in U.S. Pat. No. 6,331,393, (hereby incorporated by reference in its entirety) also known as the MethyLight™ assay. Variations on the TaqMan™ detection methodology that are also suitable for use with the described invention include the use of dual-probe technology (Lightcycler™) or fluorescent amplification primers (Sunrise™ technology). Both these techniques may be adapted in a manner suitable for use with bisulfite treated DNA, and moreover for methylation analysis within CpG dinucleotides.

In a further preferred embodiment of the method, the fourth step of the method comprises the use of template-directed oligonucleotide extension, such as MS-SNuPE as described by Gonzalgo & Jones, Nucleic Acids Res. 25:2529-2531, 1997.

In yet a further embodiment of the method, the fourth step of the method comprises sequencing and subsequent sequence analysis of the amplificate generated in the third step of the method (Sanger F., et al., Proc Natl Acad Sci USA 74:5463-5467, 1977).

### BEST MODE

In the most preferred embodiment of the method the genomic nucleic acids are isolated and treated according to the first three steps of the method outlined above, namely:

a) obtaining, from a subject, a biological sample having subject genomic DNA;
b) extracting or otherwise isolating the genomic DNA;
c) treating the genomic DNA of b), or a fragment thereof, with one or more reagents to convert cytosine bases that are unmethylated in the 5-position thereof to uracil or to another base that is detectably dissimilar to cytosine in terms of hybridization properties; and wherein
d) amplifying subsequent to treatment in c) is carried out in a methylation specific manner, namely by use of methylation specific primers or blocking oligonucleotides, and further wherein
e) detecting of the amplificates is carried out by means of a real-time detection probe, as described above.

Preferably, where the subsequent amplification of d) is carried out by means of methylation specific primers, as described above, said methylation specific primers comprise a sequence having a length of at least 9 nucleotides which hybridises to a treated nucleic acid sequence according to one of SEQ ID NO: 5-20; 66-181 and sequences complementary thereto, wherein the base sequence of said oligomers comprise at least one CpG dinucleotide.

Step e) of the method, namely the detection of the specific amplificates indicative of the methylation status of one or more CpG positions of at least one genomic sequence selected from the group consisting SEQ ID NO: 1-4; 37-65 is carried out by means of real-time detection methods as described above.

Additional embodiments of the invention provide a method for the analysis of the methylation status of genomic DNA according to the invention (SEQ ID NO: 1-4; 37-65, and complements thereof) without the need for bisulfate conversion. Methods are known in the art wherein a methylation sensitive restriction enzyme reagent, or a series of restriction enzyme reagents comprising methylation sensitive restriction enzyme reagents that distinguishes between methylated and non-methylated CpG dinucleotides within a target region are utilized in determining methylation, for example but not limited to DMH (see for example but not limited to U.S. Pat. No. 6,605,432 or WO 2006/088978).

In the first step of such additional embodiments, the genomic DNA sample is isolated from tissue or cellular sources. Genomic DNA may be isolated by any means standard in the art, including the use of commercially available kits. Briefly, wherein the DNA of interest is encapsulated in by a cellular membrane the biological sample must be disrupted and lysed by enzymatic, chemical or mechanical means. The DNA solution may then be cleared of proteins and other contaminants, e.g., by digestion with proteinase K. The genomic DNA is then recovered from the solution. This may be carried out by means of a variety of methods including salting out, organic extraction or binding of the DNA to a solid phase support. The choice of method will be affected by several factors including time, expense and required quantity of DNA. All clinical sample types comprising neoplastic or potentially neoplastic matter are suitable for use in the present method, preferred are cell lines, histological slides, biopsies, paraffin-embedded tissue, body fluids, ejaculate, urine, blood plasma, blood serum, whole blood, isolated blood cells, cells isolated from the blood and combinations thereof. Body fluids are the preferred source of the DNA; particularly preferred are urine, blood plasma, blood serum, whole blood, isolated blood cells and cells isolated from the blood.

Once the nucleic acids have been extracted, the genomic double-stranded DNA is used in the analysis.

In a preferred embodiment, the DNA may be cleaved prior to treatment with methylation sensitive restriction enzymes. Such methods are known in the art and may include both physical and enzymatic means. Particularly preferred is the use of one or a plurality of restriction enzymes which are not methylation sensitive, and whose recognition sites are AT rich and do not comprise CG dinucleotides. The use of such enzymes enables the conservation of CpG islands and CpG rich regions in the fragmented DNA. The non-methylation-specific restriction enzymes are preferably selected from the group consisting of MseI, BfaI, Csp6I, Tru1I, Tvu1I, Tru9I, Tvu9I, MaeI and XspI. Particularly preferred is the use of two or three such enzymes. Particularly preferred is the use of a combination of MseI, BfaI and Csp6I.

The fragmented DNA may then be ligated to adaptor oligonucleotides in order to facilitate subsequent enzymatic amplification. The ligation of oligonucleotides to blunt and sticky ended DNA fragments is known in the art, and is carried out by means of dephosphorylation of the ends (e.g. using calf or shrimp alkaline phosphatase) and subsequent ligation using ligase enzymes (e.g. T4 DNA ligase) in the presence of dATPs. The adaptor oligonucleotides are typically at least 18 base pairs in length.

In the third step, the DNA (or fragments thereof) is then digested with one or more methylation sensitive restriction enzymes. The digestion is carried out such that hydrolysis of the DNA at the restriction site is informative of the methylation status of a specific CpG dinucleotide of at least one gene selected from the group consisting of RASSF2A; TFAP2E; HIST1H4K; GSTPi; MME; RARB; NKX3-1; NPAL3; ACVR2A; ARL4C; PDE4D; CARTPT; HIST1H2BD; CUL1; MOBKL2B; IFLTD1 (KRAS); ANXA2; NFATC3; MN1; KLF8; FGF13; NKX2-6; SPG20; DSE/SART2; GJA1 (CX43); SPATA6; MCC; GPR68 (INTRON 1 AND/OR EXON 2 REGION).

Preferably a plurality of genes (herein also referred to as a “gene panel”) are analysed. Preferably 2, 3 or 4 genes are analysed. In one embodiment of the method said panel comprises at least one gene selected from the group consisting of RASSF2A; TFAP2E; HIST1H4K; GSTPi; MME; RARB; NKX3-1; NPAL3; ACVR2A; ARL4C; PDE4D; CARTPT; HIST1H2BD; CUL1; MOBKL2B; IFLTD1 (KRAS); ANXA2; NFATC3; MN1; KLF8; FGF13; NKX2-6; SPG20; DSE/SART2; GJA1 (CX43); SPATA6; MCC; GPR68 (INTRON 1 AND/OR EXON 2 REGION); and/or their promoter or regulatory regions. Preferably said group comprises the gene HIST1H4K.

Particularly preferred are the following combinations of genes:

### HIST +RASSF2A or HIST +GSTPi

Preferably, the methylation-specific restriction enzyme is selected from the group consisting of Bsi E1, Hga I, HinPI, Hpy99I, Ava I, Bce AI, Bsa HI, BisI, BstUI, BshI236I, AccII, BstFNI, McrBC, GlaI, MvnI, HpaII (HapII), HhaI, AciI, SmaI, HinP1I, HpyCH4IV, EagI and mixtures of two or more of the above enzymes. Preferred is a mixture containing the restriction enzymes BstUI, HpaII, HpyCH4IV and HinP1I.

In the fourth step, which is optional but a preferred embodiment, the restriction fragments are amplified. This is preferably carried out using a polymerase chain reaction, and said amplificates may carry suitable detectable labels as discussed above, namely fluorophore labels, radionuclides and mass labels. Particularly preferred is amplification by means of an amplification enzyme and at least two primers comprising, in each case a contiguous sequence at least 16 nucleotides in length that is complementary to, or hybridizes under moderately stringent or stringent conditions to a sequence selected from the group consisting SEQ ID NO: 1-4; 37-65, and complements thereof. Preferably said contiguous sequence is at least 16, 20 or 25 nucleotides in length. In an alternative embodiment said primers may be complementary to any adaptors linked to the fragments.

In the fifth step the amplificates are detected. The detection may be by any means standard in the art, for example, but not limited to, gel electrophoresis analysis, hybridisation analysis, incorporation of detectable tags within the PCR products, DNA array analysis, MALDI or ESI analysis. Preferably said detection is carried out by hybridisation to at least one nucleic acid or peptide nucleic acid comprising in each case a contiguous sequence at least 16 nucleotides in length that is complementary to, or hybridizes under moderately stringent or stringent conditions to a sequence selected from the group consisting SEQ ID NO: 1-4; 37-65, and complements thereof. Preferably said contiguous sequence is at least 16, 20 or 25 nucleotides in length.

Subsequent to the determination of the methylation state or level of the genomic nucleic acids, the presence or absence of prostate cell proliferative disorders, most preferably, prostate carcinoma is deduced based upon the methylation state or level of at least one CpG dinucleotide sequence of SEQ ID NO: 1-4; 37-65, or an average, or a value reflecting an average methylation state of a plurality of CpG dinucleotide sequences of SEQ ID NO: 1-4; 37-65 wherein methylation is associated with the presence of prostate cell proliferative disorders, most preferably, prostate cancer. Wherein said methylation is determined by quantitative means the cut-off point for determining said the presence of methylation is preferably zero (i.e. wherein a sample displays any degree of methylation it is determined as having a methylated status at the analysed CpG position). Nonetheless, it is foreseen that the person skilled in the art may wish to adjust said cut-off value in order to provide an assay of a particularly preferred sensitivity or specificity. Accordingly said cut-off value may be increased (thus increasing the specificity), said cut off value may be within a range selected form the group consisting of 0%-5%, 5%-10%, 10%-15%, 15%-20%, 20%-30% and 30%-50%. Particularly preferred are the cut-offs 10%, 15%, 25%, and 30%.

### Kits

Moreover, an additional aspect of the present invention is a kit comprising: a means for determining methylation of at least one gene selected from the group consisting of RASSF2A; TFAP2E; HIST1H4K; GSTPi; MME; RARB; NKX3-1; NPAL3; ACVR2A; ARL4C; PDE4D; CARTPT; HIST1H2BD; CUL1; MOBKL2B; IFLTD1 (KRAS); ANXA2; NFATC3; MN1; KLF8; FGF13; NKX2-6; SPG20; DSE/SART2; GJA1 (CX43); SPATA6; MCC; GPR68 (INTRON 1 AND/OR EXON 2 REGION). The means for determining said methylation comprise preferably a bisulfite-containing reagent; one or a plurality of oligonucleotides consisting whose sequences in each case are identical, are complementary, or hybridise under stringent or highly stringent conditions to a 9 or more preferably 18 base long segment of a sequence selected from SEQ ID NO: 5-20; 66-181; and optionally instructions for carrying out and evaluating the described method of methylation analysis. In one embodiment the base sequence of said oligonucleotides comprises at least one CpG, CpA or TpG dinucleotide.

In a further embodiment, said kit may further comprise standard reagents for performing a CpG position-specific methylation analysis, wherein said analysis comprises one or more of the following techniques: MS-SNuPE, MSP, MethyLight™, HeavyMethyl, COBRA, and nucleic acid sequencing. However, a kit along the lines of the present invention can also contain only part of the aforementioned components.

In a preferred embodiment the kit may comprise additional bisulfite conversion reagents selected from the group consisting: DNA denaturation buffer; sulfonation buffer; DNA recovery reagents or kits (e.g., precipitation, ultrafiltration, affinity column); desulfonation buffer; and DNA recovery components.

In a further alternative embodiment, the kit may contain, packaged in separate containers, a polymerase and a reaction buffer optimised for primer extension mediated by the polymerase, such as PCR. In another embodiment of the invention the kit further comprising means for obtaining a biological sample of the patient. Preferred is a kit, which further comprises a container suitable for containing the means for determining methylation of at least one gene selected from the group consisting of RASSF2A; TFAP2E; HIST1H4K; GSTPi; MME; RARB; NKX3-1; NPAL3; ACVR2A; ARL4C; PDE4D; CARTPT; HIST1H2BD; CUL1; MOBKL2B; IFLTD1 (KRAS); ANXA2; NFATC3; MN1; KLF8; FGF13; NKX2-6; SPG20; DSE/SART2; GJA1 (CX43); SPATA6; MCC; GPR68 (INTRON 1 AND/OR EXON 2 REGION) in the biological sample of the patient, and most preferably further comprises instructions for use and interpretation of the kit results. In a preferred embodiment the kit comprises: (a) a bisulfite reagent; (b) a container suitable for containing the said bisulfite reagent and the biological sample of the patient; (c) at least one set of primer oligonucleotides containing two oligonucleotides whose sequences in each case are identical, are complementary, or hybridise under stringent or highly stringent conditions to a 9 or more preferably 18 base long segment of a sequence selected from SEQ ID NO: 5-20; 66-181; and optionally (d) instructions for use and interpretation of the kit results. In an alternative preferred embodiment the kit comprises: (a) a bisulfite reagent; (b) a container suitable for containing the said bisulfite reagent and the biological sample of the patient; (c) at least one oligonucleotides and/or PNA-oligomer having a length of at least 9 or 16 nucleotides which is identical to or hybridises to a pre-treated nucleic acid sequence according to one of SEQ ID NO: 5-20; 66-181 and sequences complementary thereto; and optionally (d) instructions for use and interpretation of the kit results.

In an alternative embodiment the kit comprises: (a) a bisulfite reagent; (b) a container suitable for containing the said bisulfite reagent and the biological sample of the patient; (c) at least one set of primer oligonucleotides containing two oligonucleotides whose sequences in each case are identical, are complementary, or hybridise under stringent or highly stringent conditions to a 9 or more preferably 18 base long segment of a sequence selected from SEQ ID NO: 5-20; 66-181; (d) at least one oligonucleotides and/or PNA-oligomer having a length of at least 9 or 16 nucleotides which is identical to or hybridises to a pre-treated nucleic acid sequence according to one of SEQ ID NO: 5-20; 66-181 and sequences complementary thereto; and optionally (e) instructions for use and interpretation of the kit results.

The kit may also contain other components such as buffers or solutions suitable for blocking, washing or coating, packaged in a separate container.

Another aspect of the invention relates to a kit for use in determining the presence of and/or diagnosing prostate cell proliferative disorders, most preferably, prostate carcinoma, said kit comprising: a means for measuring the level of transcription of at least one gene selected from the group consisting of RASSF2A; TFAP2E; HIST1H4K; GSTPi; MME; RARB; NKX3-1; NPAL3; ACVR2A; ARL4C; PDE4D; CARTPT; HIST1H2BD; CUL1; MOBKL2B; IFLTD1 (KRAS); ANXA2; NFATC3; MN1; KLF8; FGF13; NKX2-6; SPG20; DSE/SART2; GJA1 (CX43); SPATA6; MCC; GPR68 (INTRON 1 AND/OR EXON 2 REGION); and/or a means for determining methylation of at least one gene selected from the group consisting of RASSF2A; TFAP2E; HIST1H4K; GSTPi; MME; RARB; NKX3-1; NPAL3; ACVR2A; ARL4C; PDE4D; CARTPT; HIST1H2BD; CUL1; MOBKL2B; IFLTD1 (KRAS); ANXA2; NFATC3; MN1; KLF8; FGF13; NKX2-6; SPG20; DSE/SART2; GJA1 (CX43); SPATA6; MCC; GPR68 (INTRON 1 AND/OR EXON 2 REGION).

Typical reagents (e.g., as might be found in a typical COBRA™-based kit) for COBRA™ analysis may include, but are not limited to: PCR primers for at least one gene selected from the group consisting of RASSF2A; TFAP2E; HIST1H4K; GSTPi; MME; RARB; NKX3-1; NPAL3; ACVR2A; ARL4C; PDE4D; CARTPT; HIST1H2BD; CUL1; MOBKL2B; IFLTD1 (KRAS); ANXA2; NFATC3; MN1; KLF8; FGF13; NKX2-6; SPG20; DSE/SART2; GJA1 (CX43); SPATA6; MCC; GPR68 (INTRON 1 AND/OR EXON 2 REGION); restriction enzyme and appropriate buffer; gene-hybridization oligo; control hybridization oligo; kinase labeling kit for oligo probe; and labeled nucleotides. Typical reagents (e.g., as might be found in a typical MethyLight™-based kit) for MethyLight™ analysis may include, but are not limited to: PCR primers for the bisulfite converted sequence of the at least one gene selected from the group consisting of RASSF2A; TFAP2E; HIST1H4K; GSTPi; MME; RARB; NKX3-1; NPAL3; ACVR2A; ARL4C; PDE4D; CARTPT; HIST1H2BD; CUL1; MOBKL2B; IFLTD1 (KRAS); ANXA2; NFATC3; MN1; KLF8; FGF13; NKX2-6; SPG20; DSE/SART2; GJA1 (CX43); SPATA6; MCC; GPR68 (INTRON 1 AND/OR EXON 2 REGION); bisulfite specific probes (e.g. TaqMan™ or Lightcycler™); optimized PCR buffers and deoxynucleotides; and Taq polymerase.

Typical reagents (e.g., as might be found in a typical Ms-SNuPE™-based kit) for Ms-SNuPE™ analysis may include, but are not limited to: PCR primers for specific gene (or bisulfite treated DNA sequence or CpG island); optimized PCR buffers and deoxynucleotides; gel extraction kit; positive control primers; Ms-SNuPE™ primers for the bisulfite converted sequence of at least one gene selected from the group consisting of RASSF2A; TFAP2E; HIST1H4K; GSTPi; MME; RARB; NKX3-1; NPAL3; ACVR2A; ARL4C; PDE4D; CARTPT; HIST1H2BD; CUL1; MOBKL2B; IFLTD1 (KRAS); ANXA2; NFATC3; MN1; KLF8; FGF13; NKX2-6; SPG20; DSE/SART2; GJA1 (CX43); SPATA6; MCC; GPR68 (INTRON 1 AND/OR EXON 2 REGION); reaction buffer (for the Ms-SNuPE reaction); and labelled nucleotides.

Typical reagents (e.g., as might be found in a typical MSP-based kit) for MSP analysis may include, but are not limited to: methylated and unmethylated PCR primers for the bisulfite converted sequence of at least one gene selected from the group consisting of RASSF2A; TFAP2E; HIST1H4K; GSTPi; MME; RARB; NKX3-1; NPAL3; ACVR2A; ARL4C; PDE4D; CARTPT; HIST1H2BD; CUL1; MOBKL2B; IFLTD1 (KRAS); ANXA2; NFATC3; MN1; KLF8; FGF13; NKX2-6; SPG20; DSE/SART2; GJA1 (CX43); SPATA6; MCC; GPR68 (INTRON 1 AND/OR EXON 2 REGION), optimized PCR buffers and deoxynucleotides, and specific probes.

Moreover, an additional aspect of the present invention is an alternative kit comprising a means for determining methylation of at least one gene selected from the group consisting of RASSF2A; TFAP2E; HIST1H4K; GSTPi; MME; RARB; NKX3-1; NPAL3; ACVR2A; ARL4C; PDE4D; CARTPT; HIST1H2BD; CUL1; MOBKL2B; IFLTD1 (KRAS); ANXA2; NFATC3; MN1; KLF8; FGF13; NKX2-6; SPG20; DSE/SART2; GJA1 (CX43); SPATA6; MCC; GPR68 (INTRON 1 AND/OR EXON 2 REGION), wherein said means comprise preferably at least one methylation specific restriction enzyme; one or a plurality of primer oligonucleotides (preferably one or a plurality of primer pairs) suitable for the amplification of a sequence comprising at least one CpG dinucleotide of a sequence selected from the group consisting of SEQ ID NO: 1-4; 37-65; and optionally instructions for carrying out and evaluating the described method of methylation analysis. In one embodiment the base sequence of said oligonucleotides are identical, are complementary, or hybridise under stringent or highly stringent conditions to an at least 18 base long segment of a sequence selected from the group consisting of SEQ ID NO: 1-4; 37-65.

In a further embodiment said kit may comprise one or a plurality of oligonucleotide probes for the analysis of the digest fragments, preferably said oligonucleotides are identical, are complementary, or hybridise under stringent or highly stringent conditions to an at least 16 base long segment of a sequence selected from the group consisting of SEQ ID NO: 1-4; 37-65.

In a preferred embodiment the kit may comprise additional reagents selected from the group consisting: buffer (e.g. restriction enzyme, PCR, storage or washing buffers); DNA recovery reagents or kits (e.g., precipitation, ultrafiltration, affinity column) and DNA recovery components.

In a further alternative embodiment, the kit may contain, packaged in separate containers, a polymerase and a reaction buffer optimised for primer extension mediated by the polymerase, such as PCR. In another embodiment of the invention the kit further comprising means for obtaining a biological sample of the patient. In a preferred embodiment the kit comprises: (a) a methylation sensitive restriction enzyme reagent; (b) a container suitable for containing the said reagent and the biological sample of the patient; (c) at least one set of oligonucleotides one or a plurality of nucleic acids or peptide nucleic acids which are identical, are complementary, or hybridise under stringent or highly stringent conditions to an at least 9 base long segment of at least one sequence selected from the group consisting of SEQ ID NO: 1-4; 37-65; and optionally (d) instructions for use and interpretation of the kit results.

In an alternative preferred embodiment the kit comprises: (a) a methylation sensitive restriction enzyme reagent; (b) a container suitable for containing the said reagent and the biological sample of the patient; (c) at least one set of primer oligonucleotides suitable for the amplification of a sequence comprising at least one CpG dinucleotide of a sequence selected from the group consisting of SEQ ID NO: 1-4; 37-65; and optionally (d) instructions for use and interpretation of the kit results.

In an alternative embodiment the kit comprises: (a) a methylation sensitive restriction enzyme reagent; (b) a container suitable for containing the said reagent and the biological sample of the patient; (c) at least one set of primer oligonucleotides suitable for the amplification of a sequence comprising at least one CpG dinucleotide of a sequence selected from the group consisting of SEQ ID NO: 1-4; 37-65; (d) at least one set of oligonucleotides one or a plurality of nucleic acids or peptide nucleic acids which are identical, are complementary, or hybridise under stringent or highly stringent conditions to an at least 9 base long segment of a sequence selected from the group consisting of SEQ ID NO: 1-4; 37-65 and optionally (e) instructions for use and interpretation of the kit results.

The kit may also contain other components such as buffers or solutions suitable for blocking, washing or coating, packaged in a separate container.

The invention further relates to a kit for use in providing a diagnosis of the presence of a prostate cell proliferative disorders, most preferably, prostate carcinoma in a subject by means of methylation-sensitive restriction enzyme analysis. Said kit comprises a container and a DNA microarray component. Said DNA microarray component being a surface upon which a plurality of oligonucleotides are immobilized at designated positions and wherein the oligonucleotide comprises at least one CpG methylation site. At least one of said oligonucleotides is specific for at least one gene selected from the group consisting of RASSF2A; TFAP2E; HIST1H4K; GSTPi; MME; RARB; NKX3-1; NPAL3; ACVR2A; ARL4C; PDE4D; CARTPT; HIST1H2BD; CUL1; MOBKL2B; IFLTD1 (KRAS); ANXA2; NFATC3; MN1; KLF8; FGF13; NKX2-6; SPG20; DSE/SART2; GJA1 (CX43); SPATA6; MCC; GPR68 (INTRON 1 AND/OR EXON 2 REGION) and comprises a sequence of at least 15 base pairs in length but no more than 200 by of a sequence selected from the group consisting of SEQ ID NO: 1-4; 37-65. Preferably said sequence is at least 15 base pairs in length but no more than 80 by of a sequence selected from the group consisting of SEQ ID NO: 1-4; 37-65. It is further preferred that said sequence is at least 20 base pairs in length but no more than 30 by of a sequence selected from the group consisting of SEQ ID NO: 1-4; 37-65.

Said test kit preferably further comprises a restriction enzyme component comprising one or a plurality of methylation-sensitive restriction enzymes.

In a further embodiment said test kit is further characterized in that it comprises at least one methylation-specific restriction enzyme, and wherein the oligonucleotides comprise a restriction site of said at least one methylation specific restriction enzymes.

The kit may further comprise one or several of the following components, which are known in the art for DNA enrichment: a protein component, said protein binding selectively to methylated DNA; a triplex-forming nucleic acid component, one or a plurality of linkers, optionally in a suitable solution; substances or solutions for performing a ligation e.g. ligases, buffers; substances or solutions for performing a column chromatography; substances or solutions for performing an immunology based enrichment (e.g. immunoprecipitation); substances or solutions for performing a nucleic acid amplification e.g. PCR; a dye or several dyes, if applicable with a coupling reagent, if applicable in a solution; substances or solutions for performing a hybridization; and/or substances or solutions for performing a washing step.

The described invention further provides a composition of matter useful for detecting, or for diagnosing prostate carcinoma. Said composition comprising at least one nucleic acid 18 base pairs in length of a segment of the nucleic acid sequence disclosed in SEQ ID NO: 5-20; 66-181, and one or more substances taken from the group comprising: 1-5 mmol/l Magnesium Chloride, 100-500 μmol/l dNTP, 0.5-5 units of taq polymerase, bovine serum albumen, an oligomer in particular an oligonucleotide or peptide nucleic acid (PNA)-oligomer, said oligomer comprising in each case at least one base sequence having a length of at least 9 nucleotides which is complementary to, or hybridizes under moderately stringent or stringent conditions to a pretreated genomic DNA according to one of the SEQ ID NO: 5-20; 66-181 and sequences complementary thereto. It is preferred that said composition of matter comprises a buffer solution appropriate for the stabilization of said nucleic acid in an aqueous solution and enabling polymerase based reactions within said solution. Suitable buffers are known in the art and commercially available.

In further preferred embodiments of the invention said at least one nucleic acid is at least 50, 100, 150, 200, 250 or 500 base pairs in length of a segment of the nucleic acid sequence disclosed in SEQ ID NO: 5-20; 66-181.

Subject matter of the invention is a method for detecting prostate cell proliferative disorders in a subject comprising determining the expression levels of at least one gene selected from the group consisting of RASSF2A; TFAP2E; HIST1H4K; GSTPi; MME; RARB; NKX3-1; NPAL3; ACVR2A; ARL4C; PDE4D; CARTPT; HIST1H2BD; CUL1; MOBKL2B; IFLTD1 (KRAS); ANXA2; NFATC3; MN1; KLF8; FGF13; NKX2-6; SPG20; DSE/SART2; GJA1 (CX43); SPATA6; MCC; GPR68 (INTRON 1 AND/OR EXON 2 REGION) in a biological sample isolated from said subject wherein underexpression and/or CpG methylation is indicative of the presence of said disorder.

A preferred embodiment further comprises determining the expression level of PSA in said biological sample isolated from said subject wherein underexpression and/or CpG methylation is indicative of the presence of said disorder.

According to a preferred embodiment, said expression level is determined by detecting the presence, absence or level of mRNA transcribed from said gene.

According to a preferred embodiment, said expression level is determined by detecting the presence, absence or level of a polypeptide encoded by said gene or sequence thereof.

According to a preferred embodiment, said polypeptide is detected by one or more means selected from the group comprising western blot analysis, chromatography, immunoassay, ELISA immunoassay, radioimmunoassay, antibody and combinations thereof.

According to a preferred embodiment, said expression is determined by detecting the presence or absence of CpG methylation within said gene, wherein the presence of methylation indicates the presence of a carcinoma.

Subject matter of the invention is further a method for detecting prostate cell proliferative disorders in a subject, comprising contacting genomic DNA isolated from a biological sample obtained from said subject with at least one reagent, or series of reagents that distinguishes between methylated and non-methylated CpG dinucleotides within at least one target region of the genomic DNA, wherein the target region comprises, or hybridizes under stringent conditions to a sequence of at least 16 contiguous nucleotides of a sequence selected from the group consisting of SEQ ID NO: 1-4; 37-65 respectively, wherein said contiguous nucleotides comprise at least one CpG dinucleotide sequence, and whereby detecting carcinoma is, at least in part, afforded.

A preferred embodiment comprises further determining the expression level of PSA in said biological sample isolated from said subject wherein underexpression and/or CpG methylation is indicative of the presence of said disorder.

Subject matter of the invention is further a method for detecting prostate cell proliferative disorders, comprising:

a) extracting or otherwise isolating genomic DNA from a biological sample obtained from the subject;
b) treating the genomic DNA of a), or a fragment thereof, with one or more reagents to convert cytosine bases that are unmethylated in the 5-position thereof to uracil or to another base that is detectably dissimilar to cytosine in terms of hybridization properties;
c) contacting the treated genomic DNA, or the treated fragment thereof, with an amplification enzyme and at least one primer comprising, a contiguous sequence of at least 9 nucleotides that is complementary to, or hybridizes under moderately stringent or stringent conditions to a sequence selected from the group consisting of SEQ ID NO: 5-20; 66-181, and complements thereof, wherein the treated genomic DNA or the fragment thereof is either amplified to produce at least one amplificate, or is not amplified; and
d) determining, based on a presence or absence of, or on a property of said amplificate, the methylation state or level of at least one CpG dinucleotide of a sequence selected from the group consisting SEQ ID NO: 1-4; 37-65, or an average, or a value reflecting an average methylation state or level of a plurality of CpG dinucleotides of a sequence selected from the group consisting of SEQ ID NO: 1-4; 37-65, whereby at least one of detecting and diagnosing cancer is, at least in part, afforded.

A preferred embodiment comprises further determining the expression level of PSA in said biological sample isolated from said subject wherein underexpression and/or CpG methylation is indicative of the presence of said disorder.

According to a preferred embodiment, treating the genomic DNA, or the fragment thereof in b), comprises use of a reagent selected from the group comprising of bisulfite, hydrogen sulfite, disulfite, and combinations thereof.

According to a preferred embodiment, contacting or amplifying in c) comprises use of at least one method selected from the group comprising: use of a heat-resistant DNA polymerase as the amplification enzyme; use of a polymerase lacking 5′-3′ exonuclease activity; use of a polymerase chain reaction (PCR); generation of an amplificate nucleic acid molecule carrying a detectable label.

According to a preferred embodiment, the biological sample obtained from the subject is selected from the group comprising cell lines, histological slides, biopsies, paraffin-embedded tissue, body fluids, ejaculate, ejaculate, urine, blood plasma, blood serum, whole blood, isolated blood cells, cells isolated from the blood and combinations thereof.

A preferred embodiment comprises further in step d) the use of at least one nucleic acid molecule or peptide nucleic acid molecule comprising in each case a contiguous sequence at least 9 nucleotides in length that is complementary to, or hybridizes under moderately stringent or stringent conditions to a sequence selected from the group consisting of SEQ ID NO: 5-20; 66-181, and complements thereof, wherein said nucleic acid molecule or peptide nucleic acid molecule suppresses amplification of the nucleic acid to which it is hybridized.

According to a preferred embodiment, determining in d) comprises hybridization of at least one nucleic acid molecule or peptide nucleic acid molecule in each case comprising a contiguous sequence at least 9 nucleotides in length that is complementary to, or hybridizes under moderately stringent or stringent conditions to a sequence selected from the group consisting of SEQ ID NO: 5-20; 66-181, and complements thereof.

According to a preferred embodiment, at least one such hybridizing nucleic acid molecule or peptide nucleic acid molecule is bound to a solid phase.

A preferred embodiment comprises further extending at least one such hybridized nucleic acid molecule by at least one nucleotide base.

According to a preferred embodiment, determining in d), comprises sequencing of the amplificate.

According to a preferred embodiment, contacting or amplifying in c), comprises use of methylation-specific primers.

Subject matter of the invention is further a method for detecting prostate cell proliferative disorders, comprising:

a) extracting or otherwise isolating genomic DNA from a biological sample obtained from the subject;
b) digesting the genomic DNA of a), or a fragment thereof, with one or more methylation sensitive restriction enzymes;
c) contacting the DNA restriction enzyme digest of b), with an amplification enzyme and at least two primers suitable for the amplification of a sequence comprising at least one CpG dinucleotide of a sequence selected from the group consisting of SEQ ID NO: 1-4, 37-65;
d) determining, based on a presence or absence of an amplificate the methylation state or level of at least one CpG dinucleotide of a sequence selected from the group consisting SEQ ID NO: 1-4, 37-65, whereby at least one of detecting and classifying cancer is, at least in part, afforded.

A preferred embodiment comprises further determining the expression level of PSA in said biological sample isolated from said subject wherein underexpression and/or CpG methylation is indicative of the presence of said disorder.

According to a preferred embodiment, the presence or absence of an amplificate is determined by means of hybridization to at least one nucleic acid or peptide nucleic acid which is identical, complementary, or hybridizes under stringent or highly stringent conditions to an at least 16 base long segment of a sequence selected from the group consisting of SEQ ID NO: 1-4; 37-65.

Subject matter of the invention is further a treated nucleic acid for use in the detection of prostate cell proliferative disorders derived from genomic SEQ ID NO: 1-4, 37-65 wherein the treatment is suitable to convert at least one unmethylated cytosine base of the genomic DNA sequence to uracil or another base that is detectably dissimilar to cytosine in terms of hybridization.

Subject matter of the invention is further a nucleic acid for use in the detection of prostate cell proliferative disorders, comprising at least 9 or at least 16 contiguous nucleotides of a treated genomic DNA sequence selected from the group consisting of SEQ ID NO: 5-20, 66-181, and sequences complementary thereto, wherein the treatment is suitable to convert at least one unmethylated cytosine base of the genomic DNA sequence selected from the group consisting of SEQ ID NO: 1-4, 37-65 to uracil or another base that is detectably dissimilar to cytosine in terms of hybridization.

Subject matter of the invention is further a nucleic acid for use in the detection of prostate cell proliferative disorders, comprising at least 50 contiguous nucleotides of a DNA sequence selected from the group consisting of SEQ ID NO: 5-20, 66-181, and sequences complementary thereto.

According to a preferred embodiment, the contiguous base sequence comprises at least one CpG, TpG or CpA dinucleotide sequence.

Subject matter of the invention is further a nucleic acid for use in the detection of prostate cell proliferative disorders, comprising at least 9 or at least 16 contiguous nucleotides of a treated genomic DNA sequence selected from the group consisting of SEQ ID NO: 5-20, 66-181 and sequences complementary thereto as a diagnostic means.

Subject matter of the invention is further a kit suitable for determining the expression level by detecting the presence, absence or level of mRNA transcribed from a gene selected from the group consisting of RASSF2A; TFAP2E; HIST1H4K; GSTPi; MME; RARB; NKX3-1; NPAL3; ACVR2A; ARL4C; PDE4D; CARTPT; HIST1H2BD; CUL1; MOBKL2B; IFLTD1 (KRAS); ANXA2; NFATC3; MN1; KLF8; FGF13; NKX2-6; SPG20; DSE/SART2; GJA1 (CX43); SPATA6; MCC; GPR68 (INTRON 1 AND/OR EXON 2 REGION), comprising

a) a plurality of oligonucleotides or polynucleotides able to hybridise under stringent or moderately stringent conditions to the transcription products of at least one gene selected from the group consisting of RASSF2A; TFAP2E; HIST1H4K; GSTPi; MME; RARB; NKX3-1; NPAL3; ACVR2A; ARL4C; PDE4D; CARTPT; HIST1H2BD; CUL1; MOBKL2B; IFLTD1 (KRAS); ANXA2; NFATC3; MN1; KLF8; FGF13; NKX2-6; SPG20; DSE/SART2; GJA1 (CX43); SPATA6; MCC; GPR68 (INTRON 1 AND/OR EXON 2 REGION);
(b) a container suitable for containing the oligonucleotides or polynucleotides and a biological sample of the patient comprising the transcription products wherein the oligonucleotides or polynucleotides can hybridise under stringent or moderately stringent conditions to the transcription products;
(c) means to detect the hybridisation of (b); and optionally
(d) instructions for use and interpretation of the kit results.

Subject matter of the invention is further a kit suitable for determining the expression level by detecting the presence, absence or level of a polypeptide encoded by a gene selected from the group consisting of RASSF2A; TFAP2E; HIST1H4K; GSTPi; MME; RARB; NKX3-1; NPAL3; ACVR2A; ARL4C; PDE4D; CARTPT; HIST1H2BD; CUL1; MOBKL2B; IFLTD1 (KRAS); ANXA2; NFATC3; MN1; KLF8; FGF13; NKX2-6; SPG20; DSE/SART2; GJA1 (CX43); SPATA6; MCC; GPR68 (INTRON 1 AND/OR EXON 2 REGION), or sequence thereof, comprising

(a) a means for detecting polypeptides of a gene selected from the group consisting of RASSF2A; TFAP2E; HIST1H4K; GSTPi; MME; RARB; NKX3-1; NPAL3; ACVR2A; ARL4C; PDE4D; CARTPT; HIST1H2BD; CUL1; MOBKL2B; IFLTD1 (KRAS); ANXA2; NFATC3; MN1; KLF8; FGF13; NKX2-6; SPG20; DSE/SART2; GJA1 (CX43); SPATA6; MCC; GPR68 (INTRON 1 AND/OR EXON 2 REGION);
(b) a container suitable for containing the said means and the biological sample of the patient comprising the polypeptides wherein the means can form complexes with the polypeptides; (c) a means to detect the complexes of (b).

Subject matter of the invention is further a kit suitable for determining the expression level by detecting the presence or absence of CpG methylation within a gene selected from the group consisting of RASSF2A; TFAP2E; HIST1H4K; GSTPi; MME; RARB; NKX3-1; NPAL3; ACVR2A; ARL4C; PDE4D; CARTPT; HIST1H2BD; CUL1; MOBKL2B; IFLTD1 (KRAS); ANXA2; NFATC3; MN1; KLF8; FGF13; NKX2-6; SPG20; DSE/SART2; GJA1 (CX43); SPATA6; MCC; GPR68 (INTRON 1 AND/OR EXON 2 REGION), wherein the presence of methylation indicates the presence of a carcinoma, comprising

(a) a bisulfite reagent;
(b) a container suitable for containing the said bisulfite reagent and the biological sample of the patient;
(c) at least one set of oligonucleotides containing two oligonucleotides whose sequences in each case are identical, are complementary, or hybridize under stringent or highly stringent conditions to a 9 or more preferably 18 base long segment of a sequence selected from SEQ ID NO: 5-20, 66-181.

Subject matter of the invention is further a kit suitable for determining the expression level by detecting the presence or absence of CpG methylation within a gene selected from the group consisting of RASSF2A; TFAP2E; HIST1H4K; GSTPi; MME; RARE; NKX3-1; NPAL3; ACVR2A; ARL4C; PDE4D; CARTPT; HIST1H2BD; CUL1; MOBKL2B; IFLTD1 (KRAS); ANXA2; NFATC3; MN1; KLF8; FGF13; NKX2-6; SPG20; DSE/SART2; GJA1 (CX43); SPATA6; MCC; GPR68 (INTRON 1 AND/OR EXON 2 REGION), wherein the presence of methylation indicates the presence of a carcinoma, comprising

(a) a methylation sensitive restriction enzyme reagent;
(b) a container suitable for containing the said reagent and the biological sample of the patient;
(c) at least one set of oligonucleotides one or a plurality of nucleic acids or peptide nucleic acids which are identical, are complementary, or hybridize under stringent or highly stringent conditions to an at least 9 base long segment of a sequence selected from the group consisting of SEQ ID NO: 1-4, 37-65; and optionally
(d) instructions for use and interpretation of the kit results.

Subject matter of the invention is further the use of a method of the invention, an inventive nucleic acid and/or a kit of the invention in the diagnosis and/or detection of prostate cell proliferative disorders.

While the present invention has been described with specificity in accordance with certain of its preferred embodiments, the following examples serve only to illustrate the invention and are not intended to limit the invention within the principles and scope of the broadest interpretations and equivalent configurations thereof.

### Example 1

The aim of the present study was to determine the feasibility of measuring DNA methylation markers for prostate cancer (hereinafter also referred to as PCa) in remote body fluids. In this process a high quality workflow flow for urine was utilized, candidate markers were analyzed by HeavyMethyl™ (HM) technology (Cottrell et al., Nucleic Acids Res. 2004 Jan. 13; 32(1):e10) and it was demonstrated that PCa sheds DNA that can be detected by means of methylation analysis in both plasma and urine with high sensitivity. It was thus established that the analyzed markers were suitable for the development of a screening test for PCa based on DNA methylation analysis.

### Study Objectives

The purpose of the present study was to conduct an investigation into whether DNA methylation markers of PCa can be measured in a remote body fluid. The study was designed to identify the optimal analyte for such a test and to generate specificity and analytical performance data for marker candidates.

Candidate markers and location of assays

The markers RASSF2 and TFAP2E were identified on the basis of their methylation in prostate cancer tissues, as determined in a preliminary study (not described herein). The markers GSTPi and HIST1H4K had been previously identified in a study by the applicant as published in patent application WO 2005/054517.

Methylation analysis was performed by means of the HeavyMethyl™. Isolated genomic is bisulfite treated to convert non-methylated cytosines to uracil, wherein methylated cyctosines are conserved. Fragments of the bisulfite treated DNA comprising potentially methylated CpG dinucleotides are then amplified by means of PCR. The primers do not cover any potentially methylated cytosine positions (i.e. do not hybridise to genomic CpG dinucleotides). Amplification of fragments comprising unmethylated CpG dinucleotides is suppressed by means of a blocking oligonucleotide that hybridises to TG dinucleotides. Accordingly only DNA that was methylated in the genomic sample is amplified. Amplificate fragments are detected by means of detectably labelled probes suitable for use in PCR reactions such as RealTime detection probes. Assay primer and probes are provided in the accompanying sequence listing as according to Table 2.

### GSTPi

### Chromosomal Location: 11q13

Nearby Gene(s): GSTPi HM forward primer is just upstream of exon 1 and the reverse primer is just downstream of exon 1 of GSTP1.

### RASSF2A

Chromosomal Location: 20pter-p12.1
Nearby Gene(s): w/i the CpG island of intron 1 of the v. 1 transcript of RASSF2.

### HIST1H4K

Chromosomal Location: 6p22-21.3
Nearby Gene(s): overlaps intronless HIST1H4K

### TFAP2E

### Chromosomal Location: 1p34.3

Nearby Gene(s): w/i intron 3 of TFAP2E (˜11 kb downstream of txn start) and ˜20 kb upstream of KIAA0319L txn start (PKD-1 like gene).

### Tissue Study

Assays were initially tested in normal tissues, NAT and PCa. The marker candidates that were analyzed in the HM tissue test were all very specific for normal blood and normal prostate tissue. In contrast to previous studies it was observed that DNA from prostate normal adjacent tumor (NAT) is nearly as methylated as prostate tumor DNA. NAT (which may contain BPH) is clearly distinct from BPH tissue that has been derived from non-prostate-tumor-bearing patients without elevated PSA (the origin of many BPH tissue samples in the MSP tissue test). In fact, there is evidence in the literature that GSTP1 in NAT is methylated (Hanson et al., 2006).

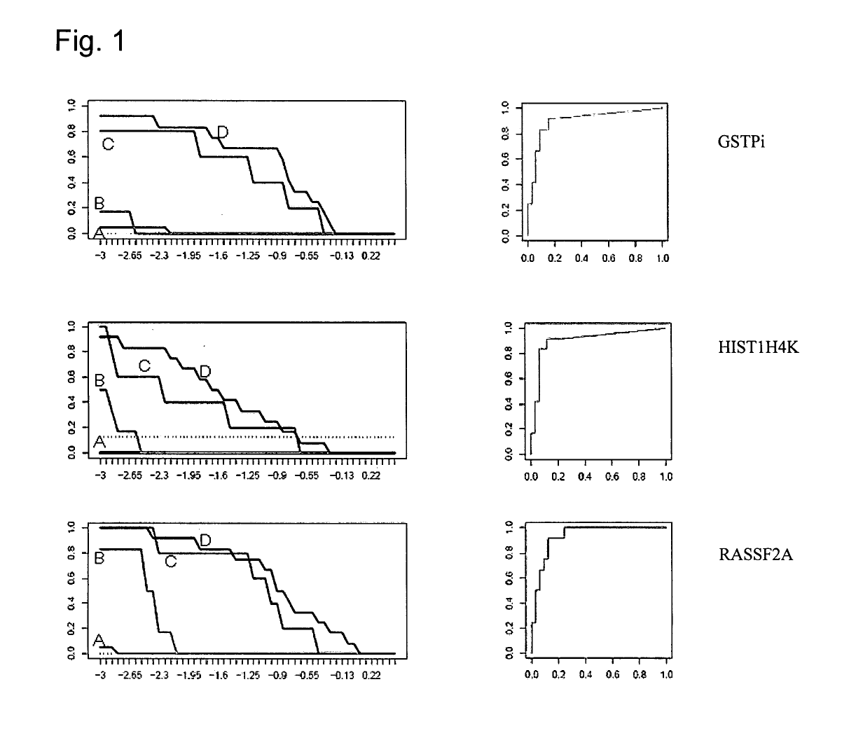

Performance of the markers in normal +BPH as compared to PCa is provided in Table 3 and FIG. 1.

### Remote Analyte Analysis

In order to maximize the analyte equivalent in real-time PCR assays (1.5 ml equivalents), the maximum number of assays in the study was capped at four, with each assay run in duplicate for each sample.

### Sample Collection

For this study, the inventors collected matched plasma and urine from a total of 191 men, including 91 males with biopsy-confirmed prostate cancer, 51 males with no cancer detected by biopsy (subsequently diagnosed with BPH), and 50 young asymptomatic males. In all analyses, the positive class is comprised of the PCa samples. In designing the present study, the definition of the negative class was an issue as there is no detection method that excludes presence of PCa with 100% certainty. Biopsy has a false negative diagnosis rate of at least 10% (Djavan et al., 2000; Mian et al., 2002; Gupta et al., 2005; Hanley et al., 2006) while PSA measurement is prone to both false negatives and false positives. Because the primary objective of the study was to demonstrate the feasibility of measuring methylated markers of PCa in a remote body fluid, the inventors focused on a negative class that minimized the probability of false positives. Consequently, young asymptomatic males were chosen as the “true” negative class. It was reasoned that young asymptomatic males with no family history of prostate cancer should be truly negative for PCa. Because one embodiment of the PCa test is as a diagnostic follow-on to PSA, the inventors also included a second negative class of biopsy negative, BPH samples. A potentially confounding factor in this class is the likely presence of false negative biopsies. In five PCa cases, only a plasma sample was collected and in ten additional cases only a urine sample was collected. The samples were collected at multiple sites. The urine was collected after a prostatic massage, both plasma and urine samples were obtained before any treatment for PCa. Inclusion and exclusion criteria were designed to ensure that the patients analyzed reflect the potential patients who would use PCa screening tests.

The following inclusion and exclusion criteria applied to the patients undergoing biopsy:

Inclusion criteria:
Indication for biopsy (elevated PSA and/or suspicious DRE)
Biopsy scheduled within 1 week after sample collection
Age 40-80

Exclusion criteria:
Any prior treatment for prostate cancer
History of cancer or serious illness in the past 5 years
Symptoms of urinary tract infection

The following criteria applied to the asymptomatic men of the control group:

Inclusion criteria:
Male
Age 18-30

Exclusion criteria:
Any prior treatment for or symptoms of prostate cancer or prostate disease
History of cancer or serious illness in the past 5 years
Symptoms of urinary tract infection
Patient data and tumor characteristics

The Gleason score (where appropriate) of the patient samples are listed Table 4. The mean PSA values for the prostate cancer, HGPIN and biopsy negative samples were 18.2±33.1, 7.0±3.0 and 8.8±5.2 respectively. The prostate cancer, HGPIN and biopsy negative classes were diagnosed after sample collection via prostate biopsy. The mean number of biopsy cores for all sample classes was 8, although there was some variation between providers.

DNA extraction and bisulfate treatment was carried out according to standardised protocols. For each assay, 1.5 ml analyte equivalent was run in duplicate.

### Marker Performance, General Considerations

The initial objective of the study was to develop a panel of markers targeted as a diagnostic follow-on to PSA tests of 2.5 ng/ml or more for men over 50 years of age to discriminate prostate cancer from non-cancerous conditions. Such a test could be further expanded as a more specific prostate cancer screening test that would compete with PSA testing because of superior performance. In the present study the inventors analyzed the data in two different ways: (i) the inventors used prostate cancer and biopsy-negative samples to assess markers performance in the follow-on to PSA test (diagnostic application) and (ii) the inventors used prostate cancer and all the non-cancer (biopsy-negative and asymptomatic) samples to measure markers performance in screening test (screening application). The inventors report marker performance for plasma and urine separately, the inventors also provide data analysis for individual markers and marker panels. All data are reported as logmean raw mathylation values.

As a primary screening test, the marker panel would preferably identify PCa in men over age 50 years with improved specificity relative to PSA. All screening application analyses use the PCa samples as the positive class. For the purposes of the present study, the inventors analyzed data for the screening application with two alternative negative classes. The first negative class analyzed the 50 young asymptomatic males with minimal likelihood of undetected PCa. While this negative class represents a “true” test negative, it is not age-matched to the target PCa screening population and does not include any likely false positive classes, e.g. BPH. Therefore, the inventors performed a second analysis in which all 50 asymptomatic young controls and all 51 biopsy negative controls were analyzed as a 101 sample size negative class.

On average, approximately 20,000,000 PSA tests are performed every year in the US with only approximately 1,000,000 cases moving forward to biopsy (of which approximately 750,000 biopsies are unnecessary). Therefore, less than 5% of individuals that are currently screened by PSA fall in the negative class that is represented by elevated-PSA-BPH-positive whereas as the vast majority of the target screening population fall into the PSA-low negative class. Whereas the negative class of only asymptomatic young males may represent an overestimation of the discriminatory capacity of our markers, the combined negative class of asymptomatic young males plus age-matched biopsy negative males may represent an underestimation of the discriminatory capacity of our markers.

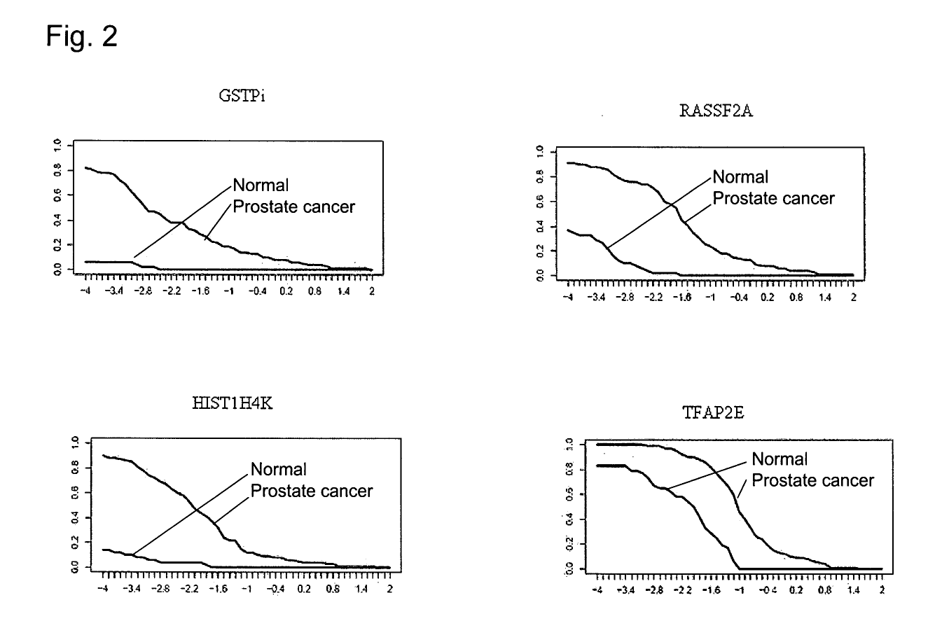

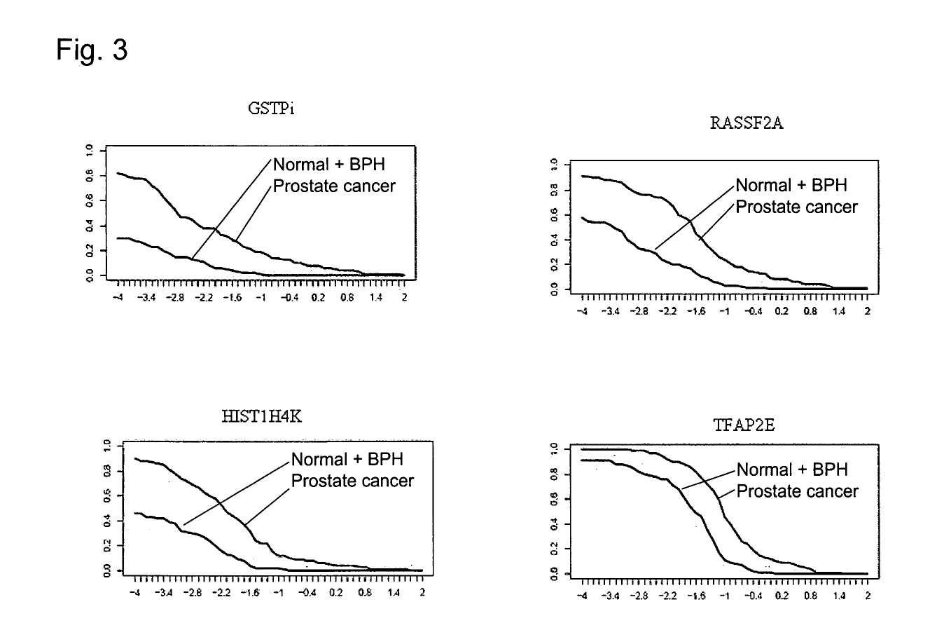

Sensitivity and specificity of individual (single) markers tested by real-time PCR in post-prostatic massage urine from prostate cancer patients vs. biopsy negative patients and asymptomatic control individualsis shown in Table 5. FIG. 2 shows the HM real-time PCR assays of post-prostatic massage urine of PCa and negative class I (asymptomatic individuals). FIG. 3 shows the HM real-time PCR assays of post-prostatic massage urine of PCa and negative class II (asymptomatic plus biopsy negative individuals).

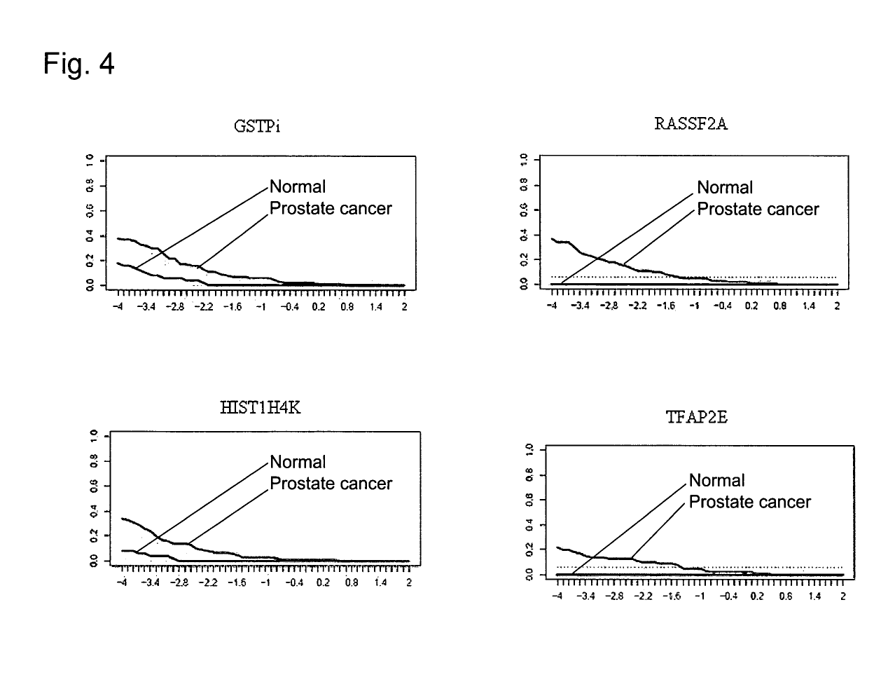

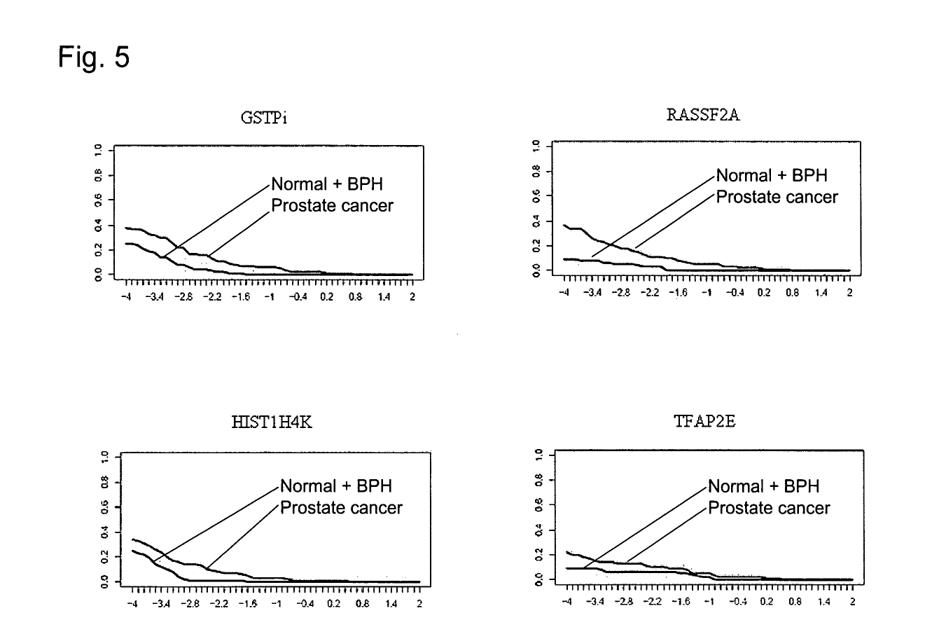

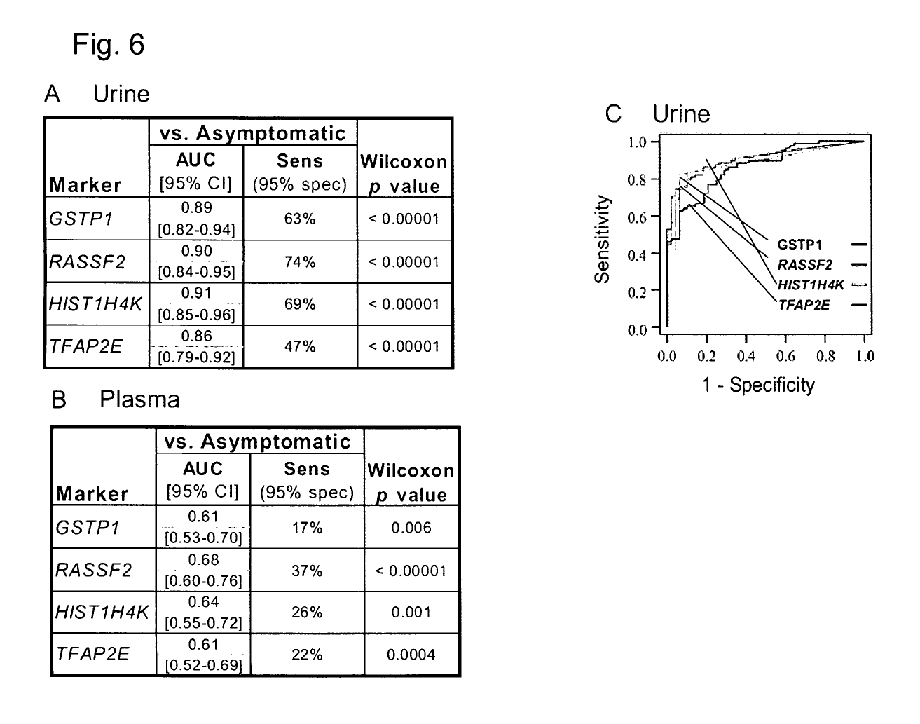

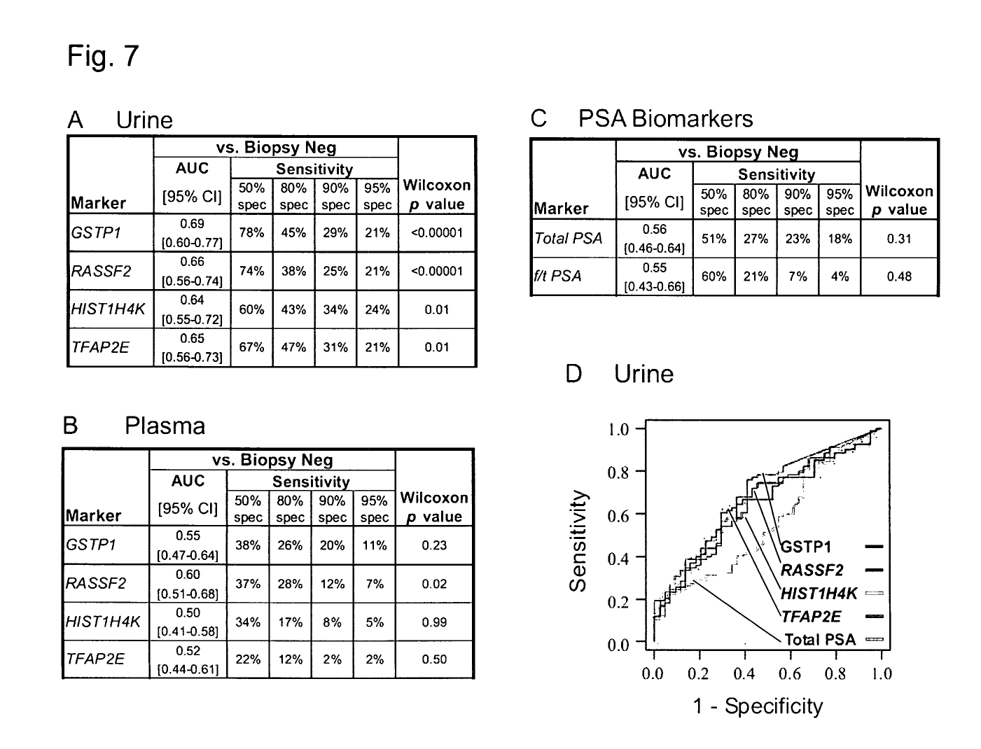

Sensitivity and specificity of individual (single) markers tested by real-time PCR in plasma from prostate cancer patients vs. biopsy negative patients and asymptomatic control individualsis shown in Table 6. FIG. 4 shows the HM real-time PCR assays of plasma of PCa and negative class I (asymptomatic individuals). FIG. 5 shows the HM real-time PCR assays of plasma of PCa and negative class II (asymptomatic plus biopsy negative individuals). FIG. 6 shows the performance of the biomarker assays for discrimination of PCa patients from asymptomatic young males in both urine and plasma. FIG. 7 shows the performance of the biomarker assays for discrimination of PCa from biopsy negative patients in both urine and plasma.

As illustrated in Table 7, in all negative class comparisons and for all markers, urine was the more sensitive analyte.

Correlation of Markers with Gleason Score

Increasing amounts of methylated marker DNA correlated with increasing Gleason score for all markers in plasma. This was true for samples with high amounts of methylated marker DNA in urine (see especially markers TFAP2E and RASSF2A), but in general the correlation was less strong in DNA from urine than in DNA from plasma. PSA as a marker of PCa in patients with elevated PSA (>4 ng/ml) also correlated with increasing Gleason score.

Performance of screening marker panels to distinguish PCa from negative class I (asymptomatic males) in urine is provided in Table 8.

Performance of screening marker panels to distinguish PCa from negative class II (asymptomatic males plus biopsy negative) in urine is provided in Table 9.

Performance of screening marker panels to distinguish PCa from negative class I (asymptomatic males) in plasma is provided in Table 10.

Performance of screening marker panels to distinguish PCa from negative class II (asymptomatic males plus biopsy negative) in plasma is provided in in Table 11.

### Marker Performance in Diagnostic Application: Follow-on to PSA

As a diagnostic follow-on to PSA testing, the markers would preferably identify PCa in men over age 50 years who have been classified as high-risk individuals due to elevated PSA (>2.5 ng/ml). This is a distinct application and analysis and requires increased discrimination as compared to the screening test. False positives in this application arise from the elevated PSA, biopsy negative BPH class. Again, the PCa samples represent the positive class. For the purposes of a diagnostic follow-on application, the inventors analyzed the data using a single negative class comprised of the 51 biopsy negative samples. AUC of markers tested by real-time PCR in post-prostatic massage urine and plasma from prostate cancer patients and biopsy negative patients is provided in Table 12.

From said table it can be seen that for all methylation markers analyzed, urine was the more sensitive analyte. For total PSA (treated here as an additional marker to determine if there is any further information provided past the >4 ng/ml indication for biopsy), there was no difference in sensitivity between urine and plasma.

### Performance of Marker Panels

In order to provide improved accuracy, combinations of markers were assessed both qualitatively and quantitatively.

Table 13 provides the performance of diagnostic marker panels to distinguish PCa from biopsy negative in urine.

Table 14 provides the performance of diagnostic marker panels to distinguish PCa from biopsy negative in plasma.

### Discussion

The study was conducted on plasma and/or urine samples collected from 91 PCa patients, 51 biopsy-negative patients (diagnosed with BPH) and 50 young asymptomatic males. HM™ real-time PCR assays were used to measure DNA methylation of the candidate markers. The amount of methylated marker DNA was correlated with PCa in both plasma and urine, with urine DNA showing greater sensitivity. As a screening test (discrimination of PCa cancer from asymptomatic controls using urine analyte), anchor marker candidates GSTPi, RASSF2A, HIST1H4K and TFAP2E have 63%, 74%, 69% and 47% sensitivity at 96% specificity respectively. As a diagnostic follow-on to PSA test (discrimination of PCa from biopsy negative controls, all with elevated PSA), the markers have 23%, 18%, 28% & 23% sensitivity at 95% specificity, respectively. A quantitative screening panel of markers RASSF2A and HIST1H4K yielded 94% sensitivity at 88% specificity against asymptomatic individuals. A quantitative diagnostic panel of markers GSTPi and PSA yielded 83% sensitivity at 45% specificity. The performance of these marker compares well with the performance of PSA (18% sensitivity at 98% specificity for men <60 years and 19% sensitivity at 94% specificity for men >60 years) in the screening population (Punglia et al., 2003). Methylation of all markers correlated well with Gleason score in plasma DNA, but the correlation was less strong in urine DNA.

### Conclusions

At the completion of the present investigation, it was demonstrated that prostate cancer biomarkers based on methylated DNA can be measured in plasma and urine, with urine DNA showing greater sensitivity than plasma. Additionally, DNA methylation markers that discriminate PCa patients from asymptomatic controls and those with benign prostatic hyperplasia (BPH) were identified. The major conclusions of the present study are as follows:

1. Methylated markers of prostate cancer can be measured in both plasma and urine from PCa patients.
2. Identification of markers that discriminate PCa patients from those without PCa.

### Example 2

The aim of the present study was to determine the sensitivity and specificity of DNA methylation markers for prostate cancer (hereinafter also referred to as PCa) in tissues. In this study, 84 PCa, 34 BPH and 35 normal prostate tissues were analyzed using high sensitivity real-time PCR assays. It was thus established that the analyzed markers were suitable for the development of a screening test for PCa based on DNA methylation analysis.

### Study Objectives

The purpose of the present study was to validate on an independent sample set the DNA methylation markers of PCa identified by our microarray discovery technology. The study was designed to generate specificity and analytical performance data for the marker candidates.

Introduction: A PCa screening biomarker with the diagnostic ability to discriminate PCa from benign prostatic hyperplasia (BPH) in patients with elevated PSA would offer a valuable tool for the public health management of PCa. Aberrant DNA methylation occurs early in tumorigenesis, is stable, and can be assayed in tissues and body fluids, making targets of aberrant DNA methylation attractive biomarker candidates. The inventors have previously demonstrated that hypermethylation of GSTP1 can be detected in urine from PCa patients and serves as a sensitive marker for early identification of PCa. The inventors have now conducted a genome-wide screen to identify markers that augment the specificity of remote sample PCa detection in a diagnostic setting.

Methods: Custom Affymetrix microarrays with >50,000 features were used to explore methylation differences between PCa (n=20), BPH (n=17) and age-matched normal (n=12) tissues. Markers with >25% median methylation in bladder cancer were discarded. The markers were ranked by p statistic, median % PCa methylation and median % BPH methylation. Top candidates were chosen for real-time PCR assay development and validated using PCa (n=84), BPH (n=34) and age-matched normal (n=35) tissues.

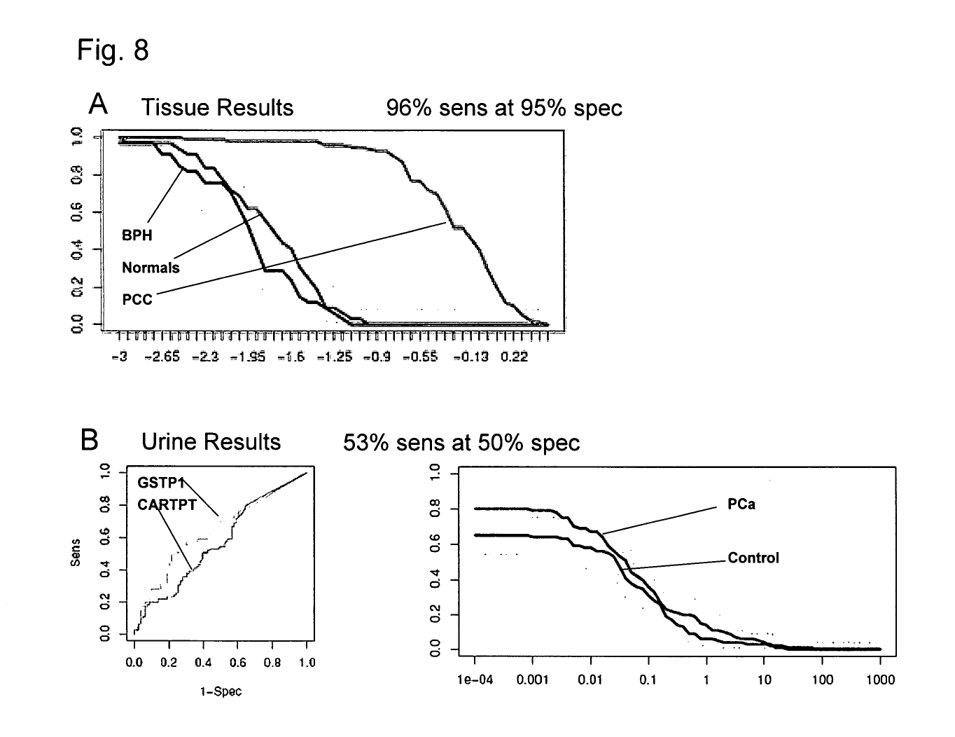

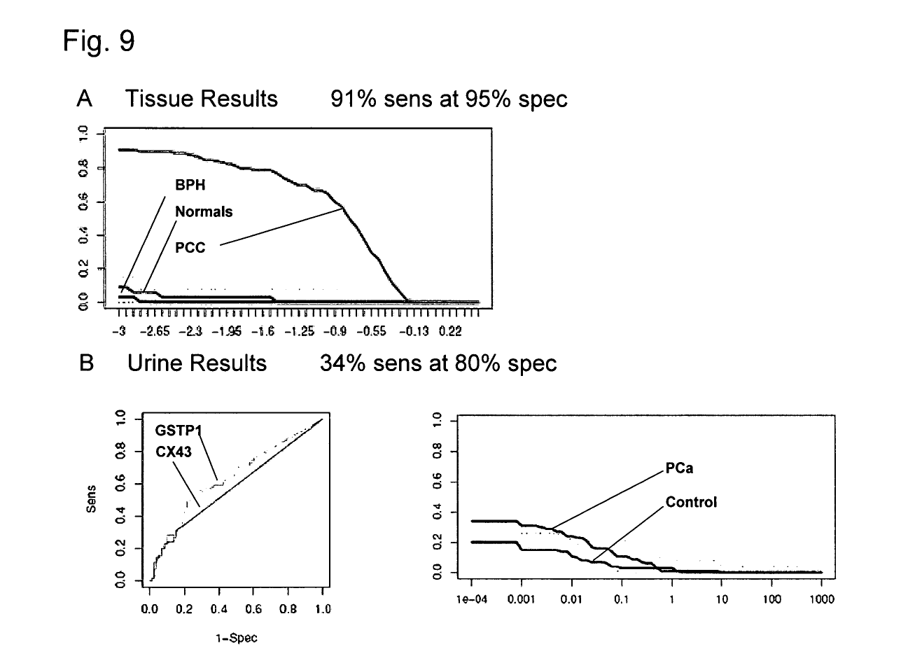

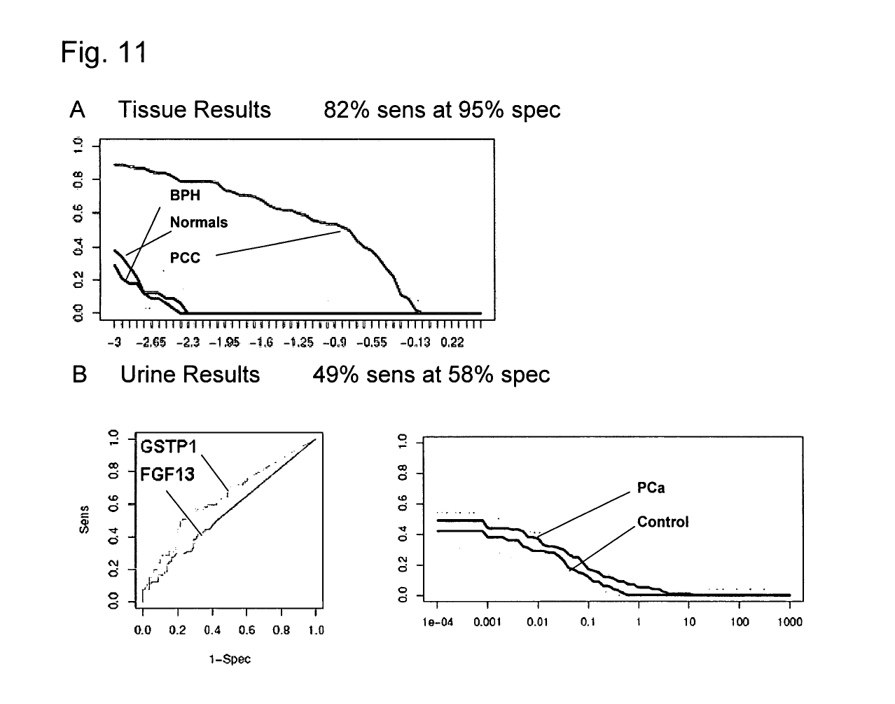

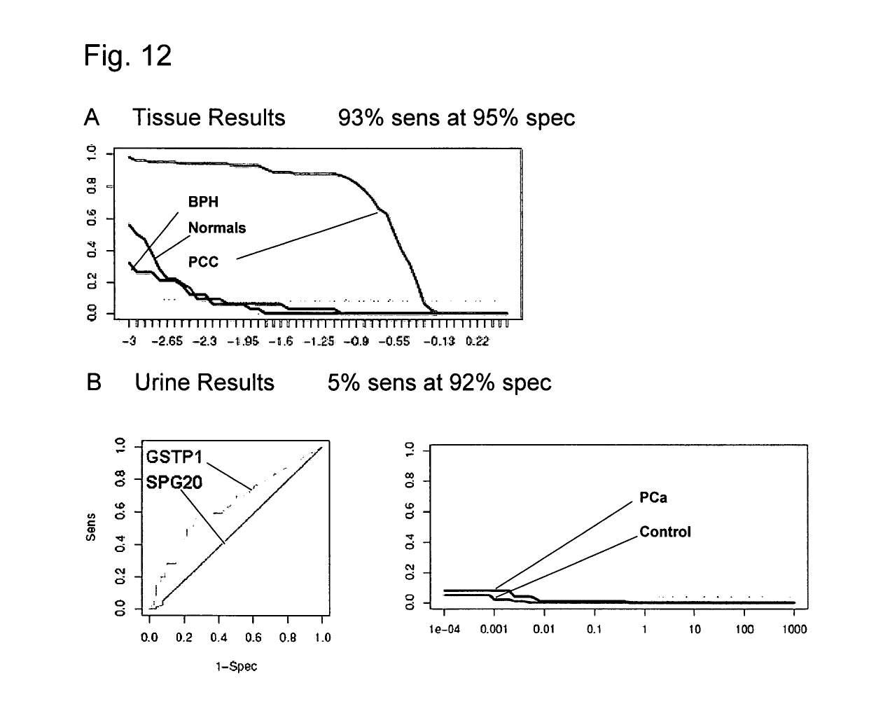

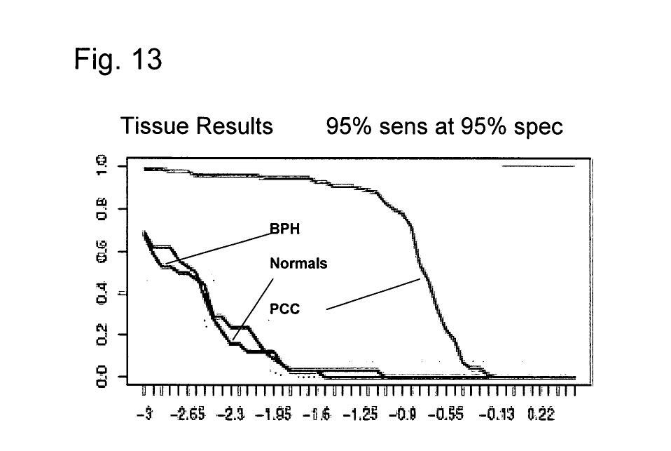

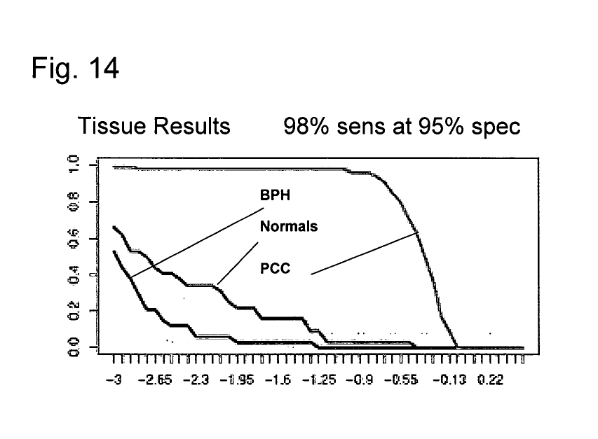

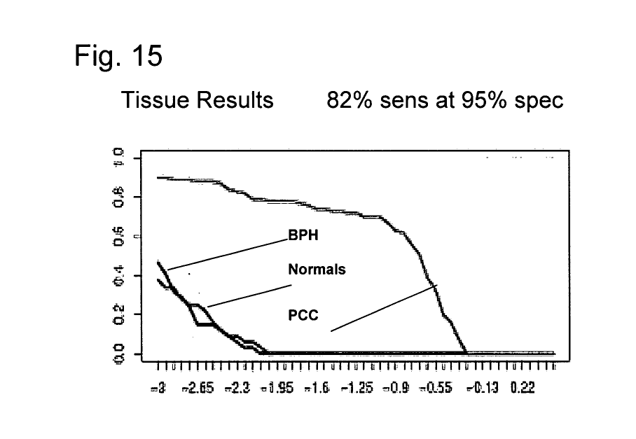

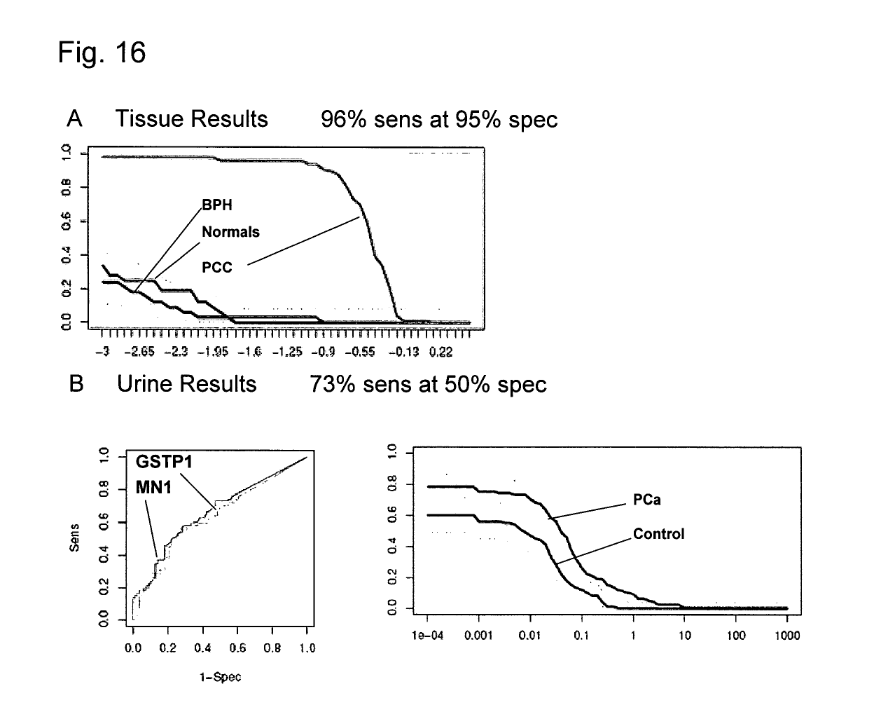

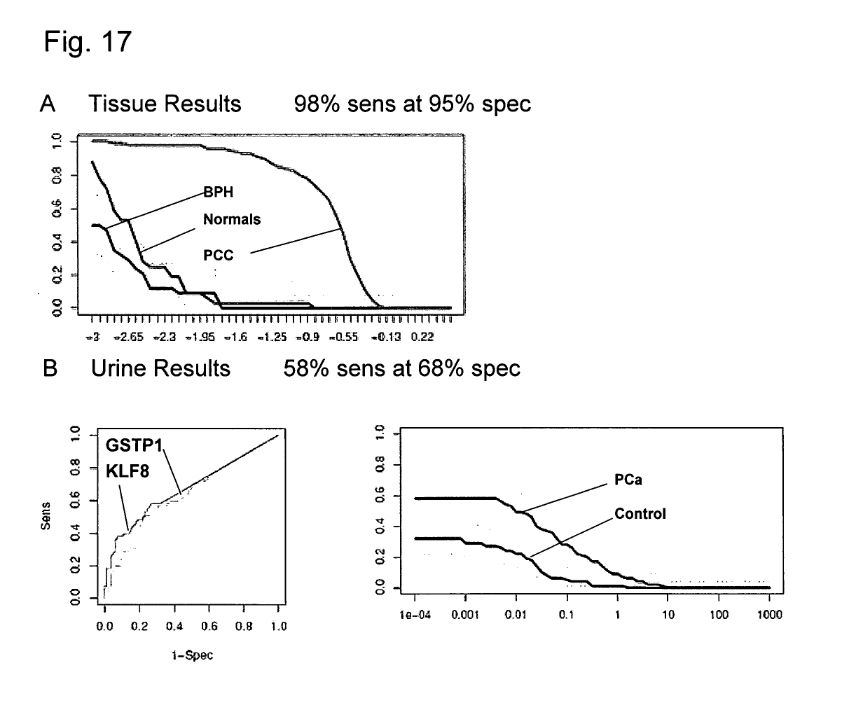

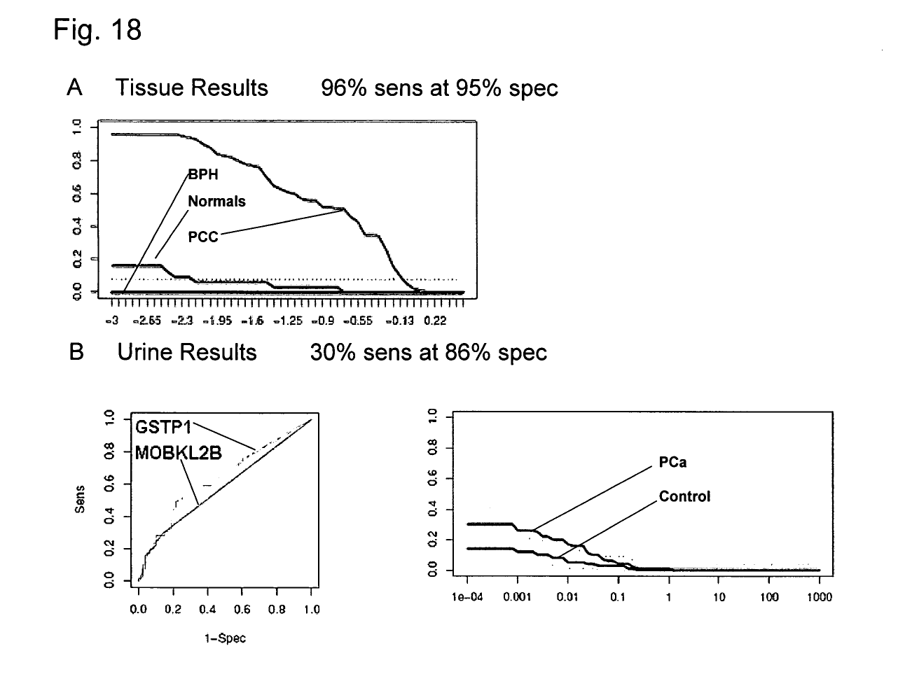

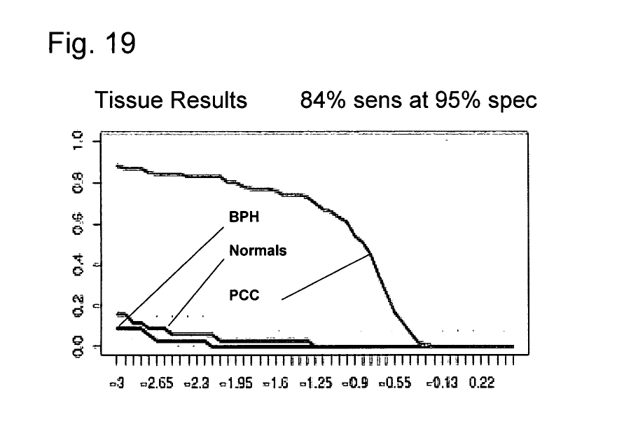

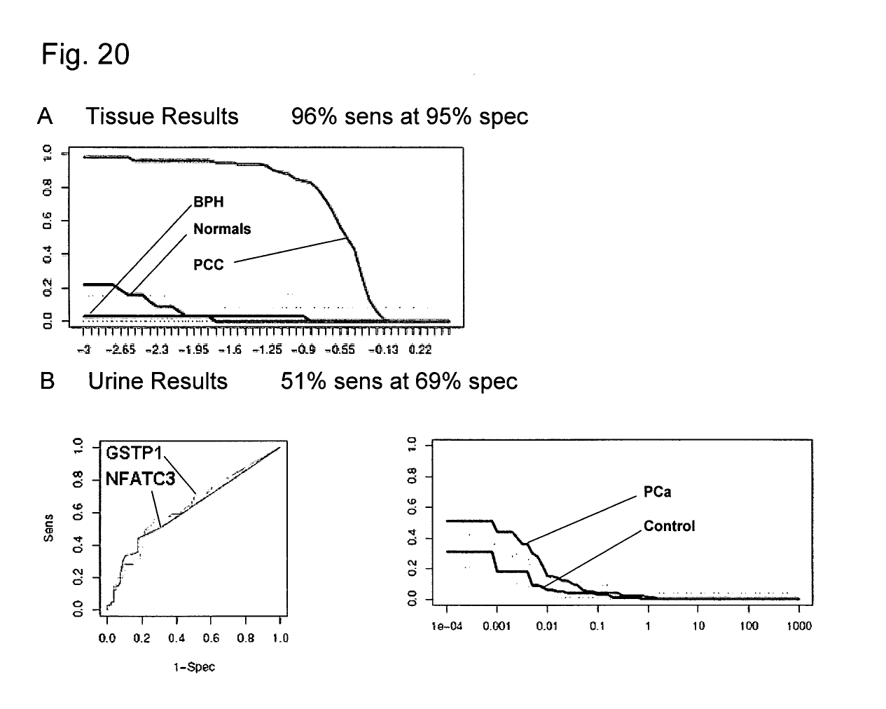

Results: Comparison yielded a large number of statistically significant markers after Bonferroni correction: 145 markers for PCa vs. BPH and normal, 148 markers for PCa vs. normal, 121 markers for PCa vs. BPH, and 61 markers for PCa vs. BPH associated with PSA values of >4 ng/ml. The top 38 markers were chosen for real-time PCR assay development. Results of real-time PCR analysis on prostate tissue (Table 16) validated the markers and revealed 4 marker categories: high sensitivity and high specificity for PCa, high specificity but low sensitivity due to low median PMR in PCa, high specificity but low sensitivity due to background methylation in BPH and finally markers that may correlate with features of aggressive PCa. Performance results for selected markers are shown in FIGS. 8-20.

### Conclusions

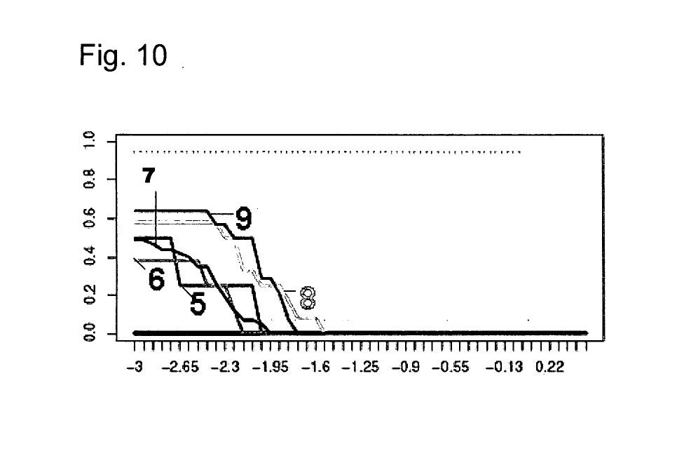

At the completion of the present investigation, it was demonstrated that the inventors identified prostate cancer biomarkers based on methylated DNA that are highly specific and sensitive for PCa. Additionally, at least one DNA methylation marker that discriminates high Gleason score PCa patients (Gleason 8-10) from benign tissues was identified (GPR68, see FIG. 10). The major conclusions of the present study are as follows:

1. The inventors have identified several previously undescribed DNA methylation markers of PCa.
2. Several of these markers show performance at least equivalent to GSTP1 for discriminating PCa tissues from BPH tissues.
3. The inventors have identified marker(s) that may distinguish pathologically aggressive PCa from indolent PCa.

TABLE 1
Genes and sequences according to the present invention

| | | | Methylated | | Unmethylated |
|---|---|---|---|---|---|
| | | Methylated | bisulfite | Unmethylated | bisulfite |
| | Genomics | bisulfite | converted | bisulfite | converted |
| | SEQ | converted | antsense | converted | antisense |
| Gene | ID NO: | sense strand | strand | sense strand | strand |
| RASSF2A | 1 | 5 | 6 | 13 | 14 |
| TFAP2E | 2 | 7 | 8 | 15 | 16 |
| HIST1H4K | 3 | 9 | 10 | 17 | 18 |
| GSTPi | 4 | 11 | 12 | 19 | 20 |
| MME | 37 | 66 | 67 | 124 | 125 |
| RARB | 38 | 68 | 69 | 126 | 127 |
| NKX3-1 | 39 | 70 | 71 | 128 | 129 |
| NPAL3 | 40 | 72 | 73 | 130 | 131 |
| ACVR2A | 41 | 74 | 75 | 132 | 133 |
| ARL4C | 42 | 76 | 77 | 134 | 135 |
| PDE4D | 43 | 78 | 79 | 136 | 137 |
| CARTPT | 44 | 80 | 81 | 138 | 139 |
| HIST1H2BD | 45 | 82 | 83 | 140 | 141 |
| CUL1 | 46 | 84 | 85 | 142 | 143 |
| MOBKL2B | 47 | 86 | 87 | 144 | 145 |
| IFLTD1; | 48 | 88 | 89 | 146 | 147 |
| KRAS | | | | | |
| ANXA2 | 49 | 90 | 91 | 148 | 149 |
| NFATC3 | 50 | 92 | 93 | 150 | 151 |
| NFATC3 | 51 | 94 | 95 | 152 | 153 |
| MN1 | 52 | 96 | 97 | 154 | 155 |
| KLF8 | 53 | 98 | 99 | 156 | 157 |
| FGF13 | 54 | 100 | 101 | 158 | 159 |
| NKX2-6 | 55 | 102 | 103 | 160 | 161 |
| SPG20 | 56 | 104 | 105 | 162 | 163 |
| DSE/SART2 | 57 | 106 | 107 | 164 | 165 |
| GJA1 (aka | 58 | 108 | 109 | 166 | 167 |
| CX43) | | | | | |
| GJA1 (aka | 59 | 110 | 111 | 168 | 169 |
| CX43) | | | | | |
| SPATA6 | 60 | 112 | 113 | 170 | 171 |
| MCC | 61 | 114 | 115 | 172 | 173 |
| GPR68 (Intron | 62 | 116 | 117 | 174 | 175 |
| 1 region) | | | | | |

TABLE 2
Assay components according to Example 1

| | Forward | Reverse | | Detection |
|---|---|---|---|---|
| Gene | Primer | Primer | Blocker | Oligo |
| GSTPi | 21 | 22 | 23 | 24 |
| HIST1H4K | 25 | 26 | 27 | 28 |
| RASSF2A | 29 | 30 | 31 | 32 |
| TFAP2E | 34 | 35 | 36 | 37 |

TABLE 3:
Performance analysis of markers
(normal plus BPH vs. PCa)
in tissue test according to Example 1.

| Marker | AUC | Sensitivity | Specificity |
|---|---|---|---|
| GSTPi | 0.90 | 0.83 | 0.91 |
| HIST1H4K | 0.91 | 0.83 | 0.91 |
| RASSF2A | 0.93 | 0.75 | 0.91 |
| TFAP2E | NA | NA | NA |

TABLE 4
Remote samples according to Example 1.

| Sample type | No. of samples |
|---|---|
| Prostate Cancer - | |
| Gleason Score | |
| 4 | 1 |
| 5 | 5 |
| 6 | 33 |
| 7 | 39 |
| 8 | 8 |
| 9 | 4 |
| No score available | 1 |
| Total Prostate Cancer: | 91 |
| Biopsy Negative | 51 |
| Asymptomatic Control | 50 |

TABLE 5
Sensitivity and specificity of individual markers tested by real-time
PCR in post-prostatic massage urine from prostate cancer patients,
biopsy negative patients and asymptomatic control individuals.

| | Negative Class I: | | | Negative Class II: | | |
|---|---|---|---|---|---|---|
| | Asymptomatic | | | Asymptomatic + Biopsy (−) | | |
| | | | Wilcoxon | | | Wilcoxon |
| Marker | AUC | Sens/Spec | p value | AUC | Sens/Spec | p value |
| GSTPi | 0.89 | 0.63/0.96 | 0 | 0.79 | 0.31/0.96 | 0 |
| RASSF2A | 0.90 | 0.74/0.96 | 0 | 0.79 | 0.24/0.96 | 0 |
| HIST1H4K | 0.91 | 0.69/0.96 | 0 | 0.77 | 0.36/0.96 | 0 |
| TFAP2E | 0.86 | 0.47/0.96 | 0 | 0.77 | 0.27/0.96 | 0 |

TABLE 6
Sensitivity and specificity of individual markers tested by real-time
PCR in plasma from prostate cancer patients, biopsy negative
patients and asymptomatic control individuals.

| | | | | Negative Class II: | | |
|---|---|---|---|---|---|---|
| | Negative Class I: | | | Asymptomatic + | | |
| | Asymptomatic | | | Biopsy (−) | | |
| | | Sens/ | Wilcoxon | | Sens/ | Wilcoxon |
| Marker | AUC | Spec | p value | AUC | Spec | p value |
| GSTPi/ | 0.61 | 0.17/0.96 | 0.0063 | 0.58 | 0.17/0.95 | 0.0183 |
| GSTP1 | | | | | | |
| RASSF2A | 0.68 | 0.37/1.00 | 0 | 0.64 | 0.20/0.95 | 0 |
| HIST1H4K | 0.64 | 0.26/0.96 | 5e⁻⁰⁴ | 0.56 | 0.16/0.95 | 0.0572 |
| TFAP2E | 0.61 | 0.22/1.00 | 4e⁻⁰⁴ | 0.56 | 0.09/0.95 | 0.0128 |

TABLE 7

| | Negative Class I: | | Negative Class II: | |
|---|---|---|---|---|
| | Asymptomatic | | Asymptomatic + Biopsy (—) | |
| Marker | Urine AUC | Plasma AUC | Urine AUC | Plasma AUC |
| GSTPi/ | 0.89 | 0.61 | 0.69 | 0.55 |
| GSTP1 | | | | |
| RASSF2A | 0.90 | 0.68 | 0.66 | 0.60 |
| HIST1H4K | 0.91 | 0.64 | 0.64 | 0.50 |
| TFAP2E | 0.86 | 0.61 | 0.65 | 0.52 |

TABLE 8
Performance of screening marker panels to distinguish
PCa from negative class I (asymptomatic males) in urine

| | | % Spec |
|---|---|---|
| Marker Panel | % Sens PCa | Asymptomatic |
| Quantitative Single Markers: | | |
| RASSF2A | 74 | 96 |
| HIST1H4K | 69 | 96 |
| GSTPi | 63 | 96 |
| TFAP2E | 46 | 100 |
| Qualitative Panels: | | |
| GSTPi + HIST1H4K | 79 | 98 |
| RASSF2A + HIST1H4K | 94 | 88 |
| Quantitative Panels: | | |
| RASSF2A + HIST1H4K | 94 | 88 |
| quadSVM | 79 | 98 |
| (all markers, no PSA) | | |

TABLE 9

| | | % Spec |
|---|---|---|
| | | Asymptomatic + |
| Marker Panel | % Sens PCa | Biopsy (—) |
| Quantitative Single Markers: | | |
| RASSF2A | 74 | 76 |
| HIST1H4K | 69 | 68 |
| GSTPi | 63 | 80 |
| TFAP2E | 46 | 88 |
| Qualitative Panels: | | |
| GSTPi + HIST1H4K | 79 | 72 |
| RASSF2A + HIST1H4K | 94 | 54 |
| Quantitative Panels: | | |
| RASSF2A + HIST1H4K | 94 | 58 |
| quadSVM | 79 | 76 |
| (all markers, no PSA) | | |

TABLE 10
Performance of screening marker panels to distinguish
PCa from negative class I (asymptomatic males) in plasma

| | | % Spec |
|---|---|---|
| Marker Panel | % Sens PCa | Asymptomatic |
| Quantitative Single Markers: | | |
| RASSF2A | 37 | 100 |
| HIST1H4K | 26 | 96 |
| GSTPi | 17 | 94 |
| TFAP2E | 22 | 100 |
| Qualitative Panels: | | |
| RASSF2A + HIST1H4K | 41 | 98 |
| Quantitative Panels: | | |
| RASSF2A + TFAP2E | 32 | 100 |
| (TFAP2E used to normalize) | | |
| quadSVM | 39 | 96 |
| (all markers, no PSA) | | |

TABLE 11

| | | % Spec |
|---|---|---|
| | | Asymptomatic + |
| Marker Panel | % Sens PCa | Biopsy (—) |
| Quantitative Single Markers: | | |
| RASSF2A | 37 | 91 |
| HIST1H4K | 26 | 88 |
| GSTPi | 17 | 95 |
| TFAP2E | 22 | 92 |
| Qualitative Panels: | | |
| RASSF2A + HIST1H4K | 41 | 88 |
| Quantitative Panels: | | |
| RASSF2A + TFAP2E | 32 | 92 |
| (TFAP2E used to normalize) | | |
| quadSVM | 39 | 94 |
| (all markers, no PSA) | | |

TABLE 12

PCa vs. Biopsy (—)

| Marker | Urine AUC | Plasma AUC |
|---|---|---|
| GSTPi/ | 0.69 | 0.55 |
| GSTP1 | | |
| RASSF2A | 0.66 | 0.60 |
| HIST1H4K | 0.64 | 0.50 |
| TFAP2E | 0.65 | 0.52 |
| ***PSA | 0.56 | 0.56 |

***Tests whether PSA contains further information beyond what was contributed by the >4 ng/ml cut-off indication for prostate biopsy.

TABLE 13

| Marker Panel | % Sens PCa | % Spec Biopsy (—) |
|---|---|---|
| Quantitative Single Markers: | | |
| RASSF2A | 74 | 55 |
| HIST1H4K | 69 | 41 |
| GSTPi | 63 | 64 |
| TFAP2E | 46 | 77 |
| Qualitative Panels: | | |
| GSTPi + HIST1H4K | 79 | 46 |
| RASSF2A + HIST1H4K | 94 | 21 |
| Quantitative Panels: | | |
| RASSF2A + HIST1H4K | 94 | 27 |
| GSTPi + PSA | 83 | 45 |
| quadSVM | 79 | 55 |
| (all markers, no PSA) | | |

TABLE 14

| Marker Panel | % Sens PCa | % Spec Biopsy (—) |
|---|---|---|
| Quantitative Single Markers: | | |
| RASSF2A | 37 | 82 |
| HIST1H4K | 26 | 79 |
| GSTPi | 17 | 96 |
| TFAP2E | 22 | 84 |
| Qualitative Panels: | | |
| RASSF2A + HIST1H4K | 41 | 79 |
| Quantitative Panels: | | |
| RASSF2A + TFAP2E | 32 | 85 |
| (TFAP2E used to normalize) | | |
| RASSF2A + TFAP2E + PSA | 94 | 22 |
| (TFAP2E used to normalize) | | |
| quadSVM | 39 | 91 |
| (all markers, no PSA) | | |
| quadSVM | 48 | 87 |
| (all markers + PSA) | | |

TABLE 15
Assay components according to Example 2

| | Genomics | Forward | Reverse | Detection |
|---|---|---|---|---|
| | SEQ ID | Primer | Primer | Probe |
| Gene | NO: | SEQ ID NO: | SEQ ID NO: | SEQ ID NO: |
| MME | 37 | 182 | 183 | 184 |
| RARB | 38 | 185 | 186 | 187 |
| NKX3-1 | 39 | 188 | 189 | 190 |
| NPAL3 | 40 | 191 | 192 | 193 |
| ACVR2A | 41 | 194 | 195 | 196 |
| ARL4C | 42 | 197 | 198 | 199 |
| PDE4D | 43 | 200 | 201 | 202 |
| CARTPT | 44 | 203 | 204 | 205 |
| HIST1H2BD | 45 | 206 | 207 | 208 |
| CUL1 | 46 | 209 | 210 | 211 |
| MOBKL2B | 47 | 212 | 213 | 214 |
| IFLTD1; KRAS | 48 | 215 | 216 | 217 |
| ANXA2 | 49 | 218 | 219 | 220 |
| NFATC3 | 50 | 221 | 222 | 223 |
| NFATC3 | 51 | 224 | 225 | 226 |
| MN1 | 52 | 227 | 228 | 229 |
| KLF8 | 53 | 230 | 231 | 232 |
| FGF13 | 54 | 233 | 234 | 235 |
| NKX2-6 | 55 | 236 | 237 | 238 |
| SPG20 | 56 | 239 | 240 | 241 |
| DSE/SART2 | 57 | 242 | 243 | 244 |
| GJA1 (aka CX43) | 58 | 245 | 246 | 247 |
| GJA1 (aka CX43) | 59 | 248 | 249 | 250 |
| SPATA6 | 60 | 251 | 252 | 253 |
| MCC | 61 | 254 | 255 | 256 |
| GPR68 (Intron 1 | 62 | 257 | 258 | 259 |
| region) | | | | |

TABLE 16:
AUC of assay according to Example 2

| For- | Re- | Detec- | | | | | |
|---|---|---|---|---|---|---|---|
| ward | verse | tion | | | | | |
| Primer | Primer | Probe | | | | Median | Median |
| SEQ | SEQ | SEQ | | Sensi- | Speci- | PCa | BPH |
| ID NO: | ID NO: | ID NO: | AUC | tivity | ficity | PMR | PMR |
| 182 | 183 | 184 | 0.82 | 0.66 | 0.97 | 0.004 | 0 |
| | | | [0.75, 0.88] | | | | |
| 185 | 186 | 187 | 0.98 | 0.98 | 0.95 | 0.523 | 0.001 |
| | | | [0.94, 1.00] | | | | |
| 188 | 189 | 190 | 0.76 | 0.51 | 1.00 | 0.002 | 0 |
| | | | [0.68, 0.82] | | | | |
| 191 | 192 | 193 | 0.76 | 0.51 | 1.00 | 0.002 | 0 |
| | | | [0.68, 0.82] | | | | |
| 194 | 195 | 196 | 0.92 | 0.85 | 0.95 | 0.025 | 0 |
| | | | [0.87, 0.96] | | | | |
| 197 | 198 | 199 | 0.95 | 0.90 | 0.98 | 0.013 | 0 |
| | | | [0.90, 0.98] | | | | |
| 200 | 201 | 202 | 0.92 | 0.82 | 0.95 | 0.212 | 0.001 |
| | | | [0.85, 0.95] | | | | |
| 203 | 204 | 205 | 0.98 | 0.96 | 0.95 | 0.718 | 0.012 |
| | | | [0.94, 1.00] | | | | |
| 206 | 207 | 208 | 0.93 | 0.84 | 0.95 | 0.154 | 0 |
| | | | [0.87, 0.96] | | | | |
| 209 | 210 | 211 | 0.71 | 0.41 | 1.00 | 0.000 | 0 |
| | | | [0.63, 0.78] | | | | |
| 212 | 213 | 214 | 0.97 | 0.96 | 0.95 | 0.218 | 0 |
| | | | [0.92, 0.99] | | | | |
| 215 | 216 | 217 | 0.75 | 0.50 | 1.00 | 0.001 | 0 |
| | | | [0.67, 0.82] | | | | |
| 218 | 219 | 220 | 0.77 | 0.55 | 0.98 | 0.004 | 0.000 |
| | | | [0.69, 0.84] | | | | |
| 221 | 222 | 223 | 0.98 | 0.96 | 0.95 | 0.329 | 0 |
| | | | [0.95, 1.00] | | | | |
| 224 | 225 | 226 | 0.98 | 0.96 | 0.95 | 0.317 | 0 |
| | | | [0.94, 1.00] | | | | |
| 227 | 228 | 229 | 0.98 | 0.96 | 0.95 | 0.260 | 0.001 |
| | | | [0.95, 1.00] | | | | |
| 230 | 231 | 232 | 0.98 | 0.98 | 0.95 | 0.357 | 0 |
| | | | [0.94, 1.00] | | | | |
| 233 | 234 | 235 | 0.92 | 0.82 | 0.95 | 0.173 | 0 |
| | | | [0.86, 0.96] | | | | |
| 236 | 237 | 238 | 0.97 | 0.95 | 0.95 | 0.187 | 0.002 |
| | | | [0.95, 0.99] | | | | |
| 239 | 240 | 241 | 0.97 | 0.93 | 0.95 | 0.297 | 0.001 |
| | | | [0.92, 0.99] | | | | |
| 242 | 243 | 244 | 0.96 | 0.94 | 0.97 | 0.089 | 0 |
| | | | [0.92, 0.99] | | | | |
| 245 | 246 | 247 | 0.95 | 0.91 | 0.95 | 0.171 | 0 |
| | | | [0.90, 0.98] | | | | |
| 248 | 249 | 250 | 0.97 | 0.93 | 0.95 | 0.173 | 0.001 |
| | | | [0.93, 0.99] | | | | |
| 251 | 252 | 253 | 0.94 | 0.88 | 0.98 | 0.033 | 0 |
| | | | [0.89, 0.97] | | | | |
| 254 | 255 | 256 | 0.77 | 0.55 | 1.00 | 0.003 | 0 |
| | | | [0.70, 0.84] | | | | |
| 257 | 258 | 259 | 0.76 | 0.51 | 1.00 | 0.001 | 0.000 |
| | | | [0.68, 0.82] | | | | |

### Example 3

The aim of the present study was to determine the feasibility of measuring DNA methylation biomarkers for prostate cancer (hereinafter also referred to as PCa) in post-prostatic massage urine. In this process a well-characterized workflow for urine was used and 8 candidate biomarkers plus GSTP1 were analyzed by real-time PCR using both hybridization probes and HeavyMethyl™ (HM) (Cottrell et al., Nucleic Acids Res. 2004 Jan. 13; 32(1):e10) technology following bisulfite treatment of genomic DNA (see Table 1).

It was demonstrated that PCa sheds DNA that can be detected by means of methylation analysis in urine with high sensitivity. It was also demonstrated that the analyzed biomarkers were suitable for the development of screening and diagnostic tests for PCa.

### Study Objectives

The purpose of the present study was to conduct an investigation into whether the candidate PCa-specific DNA methylation biomarkers identified by differential methylation hybridization (DMH) and validated in prostate tissues can be measured in post-prostatic massage urine. The study was designed to generate performance data for the biomarker candidates when analyzed individually and in panels.

Candidate Biomarkers and Location of qPCR Assays

The PCa biomarker GSTP1 had been previously identified in a study by the applicant as published in patent application WO 2005/054517. The 8 candidate biomarkers were identified as described in WO 2005/054517 (P185WO) and subsequently validated in prostate tissues as described in Example 2. This was the first analysis of these candidate biomarkers in post-prostatic massage urine.

Methylation analysis was performed by means of real-time PCR using both hybridization probes (8 newly identified candidate biomarkers) and HeavyMethyl™ (GSTP1 biomarker) technology. Isolated genomic DNA was bisulfite treated to convert non-methylated cytosines to uracil, wherein methylated cyctosines are conserved. Fragments of the bisulfite treated DNA comprising potentially methylated CpG dinucleotides are then amplified by means of PCR. The primers do not cover any potentially methylated cytosine positions (i.e. do not hybridise to genomic CpG dinucleotides). Amplification of fragments comprising unmethylated CpG dinucleotides is suppressed by means of a blocking oligonucleotide that hybridises to TG dinucleotides. Accordingly only DNA that was methylated in the genomic sample is amplified. Amplificate fragments are detected by means of detectably labelled probes suitable for use in PCR reactions such as real-time detection probes. Assay primer and probe oligos are provided in Table 2.

### GSTP1

### Chromosomal Location: 11q13

Nearby Gene(s): GSTP1 HM forward primer is just upstream of exon 1 and the reverse primer is just downstream of exon 1 of GSTP1.

### KLF8

### Chromosomal Location: Xp11.21

Nearby Gene(s): w/i exon 1 of KLF8

### NFATC3

### Chromosomal Location: 16q22.1

Nearby Gene(s): w/i promoter of NFATC3

### MOBKL2B

### Chromosomal Location: 9p21.2

Nearby Gene(s): w/i intron 1 of MOBKL2B

### GJA1 (aka CX43)

### Chromosomal Location: 6q22.31

Nearby Gene(s): w/i intron 1 of GJA 1

### MN1

### Chromosomal Location: 22q 12.1

Nearby Gene(s): w/i the promoter of MN1

### FGF13

### Chromosomal Location Xq26.3

Nearby Gene(s): w/i intron 1 of FGF 13

### CARTPT

### Chromosomal Location: 5q13.2

Nearby Gene(s): w/i exon 1 of CARTPT

### SPG20

### Chromosomal Location: 13q13.3

Nearby Gene(s): w/i intron 1 of SPG20

### Study Design

In order to maximize the urine volume interrogated by a single real-time PCR assay (1.4 ml equivalents), the maximum number of candidate biomarkers previously validated in prostate tissues that could be analyzed in this study was nine, with each assay run once for each sample.

### Sample Collection

For this study, the inventors collected urine from a total of 159 men, including 81 males with biopsy-confirmed prostate cancer and 78 males with no cancer detected by prostate biopsy. In designing the present study, the definition of the negative class was an issue as there is no detection method that excludes presence of PCa with 100% certainty. Biopsy has a false negative diagnosis rate of at least 10% (Djavan et al., 2000; Mian et al., 2002; Gupta et al., 2005; Hanley et al., 2006) while PSA measurement is prone to both false negatives and false positives.

The samples were collected at six sites in the United States and Germany. Urine was collected after a 20-60 s prostatic massage prior to any treatment for PCa. Inclusion and exclusion criteria were designed to ensure that the patients analyzed reflect the potential patients who would use PCa screening and diagnostic tests.

The following inclusion and exclusion criteria applied to the patients undergoing prostate biopsy:

Inclusion criteria:
Indication for biopsy (elevated PSA and/or suspicious DRE)
Biopsy scheduled within 1 week after sample collection
Age 40-80

Exclusion criteria:
Any prior treatment for prostate cancer
History of cancer or serious illness in the past 5 years
Symptoms of urinary tract infection

### Patient Data and Tumor Characteristics

The Gleason scores (where appropriate) of the patient samples are listed Table 3. The median total PSA values for the prostate cancer and biopsy negative patients were 8.3±1.6 and 5.4±0.6, respectively. The prostate cancer and biopsy negative classes were defined after sample collection via prostate biopsy. The median number of previous biopsies for prostate cancer and biopsy negative patients were 0.5±0.1 and 0.0±0.1, respectively.

### Workflow

DNA extraction and bisulfite treatment was carried out according to standardised protocols (Lofton-Day et al., Clin. Chem. 2008 54(2):1-10). For each qPCR assay, 1.4 ml urine equivalent was analyzed once.

### Biomarker Performance, General Considerations

The objective of the study was to determine the performance of the candidate biomarkers in men over 50 years of age in discriminating prostate cancer from non-cancerous conditions. The data was analyzed using prostate cancer patients as the positive class and biopsy negative patients as the negative class. In this example, the inventors report biomarker performance for urine only (data on tissue samples is provided in a Example 2). The inventors provide performance data for the biomarkers individually and in panels.

Performance data (AUC, sensitivity and specificity) of the individual biomarkers GSTPi, KLF8, NFATC3, MOBKL2B, GJA1/CX23, MN1, FGF13, CARTPT, and SPG20 is shown in Table 4. Performance data (AUC, sensitivity and specificity) for the biomarker panels is shown in Table 5. ROC curves for the markers compared to GSTP1 are shown in FIGS. 8-9, 11-12, 16-18 and 20.

### Discussion

The study was conducted on urine samples collected from 81 PCa and 78 biopsy negative patients. Real-time PCR assays were used to measure DNA methylation of the candidate biomarkers. The amount of methylated biomarker DNA was significantly correlated with PCa for GSTP1 and five of the newly identified candidate biomarkers (KLF8, NFATC3, MOBKL2B, GJA1 and MN1). The performances of these biomarkers compare well with the performance of PSA (18% sensitivity at 98% specificity for men <60 years and 19% sensitivity at 94% specificity for men >60 years) in the screening population (Punglia et al., 2003). Sensitivity of the six significant biomarkers at 95% specificity ranges from 12% for NFATC3 to 26% for KLF8(Table 6).

### Conclusions

The present investigation has demonstrated that GSTP1 and all of the newly identified prostate cancer biomarkers are measurable in urine from prostate cancer patients. Further, GSTP1 and five of the newly identified candidate biomarkers significantly discriminate PCa patients from biopsy negative patients. The major conclusion of the present study is: The DNA methylation-based biomarkers of prostate cancer KLF8, NFATC3, MOBKL2B, GJA1 and MN1 are measurable in urine from PCa patients and can significantly discriminate PCa from biopsy negative patients.

TABLE 17
Genes and sequences

| | | Methyl- | Methyl- | Unmethyl- | Unmethyl- |
|---|---|---|---|---|---|
| | | ated | ated | ated | ated |
| | | bisulfite | bisulfite | bisulfite | bisulfite |
| | Genomics | converted | converted | converted | converted |
| | SEQ | sense | antsense | sense | antisense |
| Gene | ID NO: | strand | strand | strand | strand |
| GSTP1 | 4 | 11 | 12 | 19 | 20 |
| KLF8 | 53 | 98 | 99 | 156 | 157 |
| NFATC3 | 50 | 92 | 93 | 150 | 151 |
| MOBKL2B | 47 | 86 | 87 | 144 | 145 |
| GJA1 (aka | 58 | 108 | 109 | 166 | 167 |
| CX43) | | | | | |
| MN1 | 52 | 96 | 97 | 154 | 155 |
| FGF13 | 54 | 100 | 101 | 158 | 159 |
| CARTPT | 44 | 80 | 81 | 138 | 139 |
| SPG20 | 56 | 104 | 105 | 162 | 163 |

TABLE 18
Assay components

| Gene | Forward Primer | Reverse Primer | Blocker | Detection Oligo |
|---|---|---|---|---|
| GSTP1 | SEQ ID NO: 21 | SEQ ID NO: 22 | SEQ ID NO: 23 | SEQ ID NO: 24 |
| | GGGATTATTT | CCCATACTAA | CTAAACCCCA | AGTTTCGTCGTCG |
| | TTATAAGGTT | AAACTCTAAA | TCCCCAAAAA | TAGTTTTCGTT- |
| | | C | CACAAACCAC | FLUOR |
| | | | ACA-C3′ | LCRED640- |
| | | | | SEQ ID NO: 269 |
| | | | | TAGTGAGTACGC |
| | | | | GCGGTTCG-PH |
| KLF8 | SEQ ID NO: 230 | SEQ ID NO: 231 | NA | SEQ ID NO: 232 |
| | GTATTTTAGGTTT | ACTCGTACGAAT | | ACTACCCCACGAAAT |
| | TGATTCGCGTG | CCTCTAAAACGA | | ACCTCGCGC |
| | | A | | |
| NFATC3 | SEQ ID NO: 221 | SEQ ID NO: 222 | NA | SEQ ID NO: 223 |
| | GCGAAAGTTTCGT | ACAAAAACCGAA | | TCCGAACTCTAAACTC |
| | TTCGTAGA | CCGAAAATC | | GCGATCGC |
| MOBKL2B | SEQ ID NO: 212 | SEQ ID NO: 213 | NA | SEQ ID NO: 214 |
| | TATTTTGTAGGCG | CCGACAAAAACA | | ATCGTCCTTCTTTCGA |
| | TTCGCGG | AAAATCGC | | AATACAAAACTCCGA |
| GJA1 | SEQ ID NO: 245 | SEQ ID NO: 246 | NA | SEQ ID NO: 247 |
| (aka | TTTTTCGAGAATG | AAATAAACGCAA | | CGAACGCGTCATCAA |
| CX43) | AGGCGG | CGCTACTACGA | | CTTCCCGA |
| MN1 | SEQ ID NO: 227 | SEQ ID NO: 228 | NA | SEQ ID NO: 229 |
| | TAGTAATCGGAG | CAAAATATACGA | | CGTACGTATCTTCTCG |
| | CGATTTGCG | CCCGACGAATA | | AAATTCCCAACCGC |
| FGF13 | SEQ ID NO: 233 | SEQ ID NO: 234 | NA | SEQ ID NO: 235 |
| | GAGGGGGTTTAG | CGAAACTATAAC | | AAAACCGAACCGCCG |
| | TATGTCGTTCG | GTCGACTCCGA | | CCGAAAA |
| CARTPT | SEQ ID NO: 203 | SEQ ID NO: 204 | NA | SEQ ID NO: 205 |
| | GCGTTTGGAATTC | GAATCAACCGTC | | TCCGCCGACGCTTAA |
| | GGCG | TATAAACAACGC | | CGTCAATACC |
| SPG20 | SEQ ID NO: 239 | SEQ ID NO: 240 | NA | SEQ ID NO: 241 |
| | AAACAACGAAAT | GAGGGAATTTTTT | | ACGATACTCCGAAAA |
| | TAAAACGACGAA | TCGTTCGAT | | AACTCCGCGATAAA |

TABLE 19
Patient samples.

| Sample type | No. of samples |
|---|---|
| Prostate Cancer - | |
| Gleason Score | |
| 4 | 2 |
| 5 | 3 |
| 6 | 14 |
| 7 | 32 |
| 8 | 3 |
| 9 | 4 |
| 10 | 3 |
| No score available | 20 |
| Total Prostate Cancer: | 81 |
| Biopsy Negative | 78 |

TABLE 20
Performance analysis of individual biomarkers analysed in
post-prostatic massage urine from prostate cancer and
biopsy negative patients.

| Biomarker | AUC | Sensitivity | Specificity | Wilcoxon p value |
|---|---|---|---|---|
| GSTP1 | 0.64 | 0.67 | 0.50 | 0.0027 |
| | [0.56-0.71] | | | |
| KLF8 | 0.67 | 0.58 | 0.68 | 0.0000 |
| | [0.59-0.75] | | | |
| NFATC3 | 0.62 | 0.51 | 0.69 | 0.0026 |
| | [0.54-0.70] | | | |
| MOBKL2B | 0.58 | 0.30 | 0.86 | 0.0150 |
| | [0.50-0.66] | | | |
| GJA1 (aka | 0.58 | 0.34 | 0.80 | 0.0385 |
| CX43) | [0.50-0.66] | | | |
| MN1 | 0.66 | 0.73 | 0.50 | 0.0003 |
| | [0.58-0.73] | | | |
| FGF13 | 0.55 | 0.49 | 0.58 | 0.2406 |
| | [0.47-0.63] | | | |
| CARTPT | 0.57 | 0.53 | 0.50 | 0.1345 |
| | [0.49-0.64] | | | |
| SPG20 | 0.6494 | 0.05 | 0.92 | 0.4415 |
| | [0.40-0.56] | | | |

TABLE 21
Performance of biomarker panels to distinguish PCa from biopsy
negative patients in post-prostatic massage urine

| | | | | Wilcoxon |
|---|---|---|---|---|
| Biomarker Panel | AUC | Sensitivity | Specificity | p value |
| GSTP1 + Total PSA | 0.76 | 0.88 | 0.52 | 0.0000 |
| | [0.68-0.83] | | | |
| KLF8 + Total PSA | 0.79 | 0.88 | 0.52 | 0.0000 |
| | [0.71-0.86] | | | |
| MN1 + Total PSA | 0.77 | 0.84 | 0.52 | 0.0000 |
| | [0.69-0.84] | | | |
| GSTP1 + KLF8 | 0.67 | 0.57 | 0.72 | 0.0001 |
| | [0.57-0.72] | | | |
| KLF8 + GJA1 | 0.71 | 0.57 | 0.73 | 0.0000 |
| (aka CX43) | [0.63-0.78] | | | |
| GSTP1 + KLF8 + | 0.70 | 0.56 | 0.75 | 0.0000 |
| GJA1 (aka CX43) | [0.62-0.78] | | | |
| KLF8 + MOBKL2B | 0.66 | 0.60 | 0.60 | 0.0003 |
| | [0.57-0.73] | | | |
| GSTP1 + KLF8 + | 0.68 | 0.64 | 0.64 | 0.0001 |
| NFATC3 | [0.60-0.75] | | | |
| KLF8 + NFATC3 | 0.69 | 0.74 | 0.53 | 0.0000 |
| | [0.61-0.76] | | | |
| MN1 + MOBKL2B | 0.67 | 0.73 | 0.51 | 0.0002 |
| | [0.59-0.74] | | | |
| KLF8 + MN1 | 0.68 | 0.73 | 0.51 | 0.0001 |
| | [0.60-0.75] | | | |
| MN1 + GJA1 | 0.68 | 0.73 | 0.51 | 0.0002 |
| (aka CX43) | [0.60-0.75] | | | |
| GSTP1 + MN1 | 0.66 | 0.72 | 0.51 | 0.0003 |
| | [0.59-0.74] | | | |

TABLE 22
Performance analysis of individual biomarkers at
high fixed specificity (95%).

| Biomarker | AUC | Sensitivity | Specificity | Wilcoxon p value |
|---|---|---|---|---|
| GSTP1 | 0.64 | 0.15 | 0.95 | 0.0027 |
| | [0.56-0.71] | | | |
| KLF8 | 0.67 | 0.26 | 0.95 | 0.0000 |
| | [0.59-0.75] | | | |
| NFATC3 | 0.62 | 0.12 | 0.95 | 0.0026 |
| | [0.54-0.70] | | | |
| MOBKL2B | 0.58 | 0.16 | 0.95 | 0.0150 |
| | [0.50-0.66] | | | |
| GJA1 (aka | 0.58 | 0.14 | 0.95 | 0.0385 |
| CX43) | [0.50-0.66] | | | |
| MN1 | 0.66 | 0.19 | 0.95 | 0.0003 |
| | [0.58-0.73] | | | |
| FGF13 | 0.55 | 0.11 | 0.95 | 0.2406 |
| | [0.47-0.63] | | | |
| CARTPT | 0.57 | 0.10 | 0.95 | 0.1345 |
| | [0.49-0.64] | | | |
| SPG20 | 0.6494 | 0.01 | 0.95 | 0.4415 |
| | [0.40-0.56] | | | |

1. A method for detecting prostate cell proliferative disorders in a subject comprising determining the expression levels of at least one gene selected from the group consisting of RASSF2A; TFAP2E; HIST1H4K; GSTPi; MME; RARB; NKX3-1; NPAL3; ACVR2A; ARL4C; PDE4D; CARTPT; HIST1H2BD; CUL1; MOBKL2B; IFLTD1 (KRAS); ANXA2; NFATC3; MN1; KLF8; FGF13; NKX2-6; SPG20; DSE/SART2; GJA1 (CX43); SPATA6; MCC; GPR68 (INTRON 1 AND/OR EXON 2 REGION) in a biological sample isolated from said subject wherein underexpression and/or CpG methylation is indicative of the presence of said disorder.

2. The method according to claim 1 further comprising determining the expression level of PSA in said biological sample isolated from said subject wherein underexpression and/or CpG methylation is indicative of the presence of said disorder.

3. The method according to claim 1 wherein said expression level is determined by detecting the presence, absence or level of mRNA transcribed from said gene.

4. The method according to claim 1 wherein said expression level is determined by detecting the presence, absence or level of a polypeptide encoded by said gene or sequence thereof.

5. The method according to claim 1 wherein said expression is determined by detecting the presence or absence of CpG methylation within said gene, wherein the presence of methylation indicates the presence of a carcinoma.

6. A method for detecting prostate cell proliferative disorders in a subject, comprising: contacting genomic DNA isolated from a biological sample obtained from said subject with at least one reagent, or series of reagents that distinguishes between methylated and non-methylated CpG dinucleotides within at least one target region of the genomic DNA, wherein the target region comprises, or hybridizes under stringent conditions to a sequence of at least 16 contiguous nucleotides of a sequence selected from the group consisting of SEQ ID NOS:1-4 and SEQ ID NOS:37 65, wherein said contiguous nucleotides comprise at least one CpG dinucleotide sequence; and determining, based on said contacting, a methylation state or level of the at least one CpG dinucleotide, wherein detecting carcinoma is, at least in part, afforded.

7. The method of claim 6, comprising:
a) obtaining extracted or otherwise isolated genomic DNA from a biological sample obtained from the subject;
b) treating the genomic DNA of a), or a fragment thereof, with one or more reagents to convert cytosine bases that are unmethylated in the 5-position thereof to uracil or to another base that is detectably dissimilar to cytosine in terms of hybridization properties;
c) contacting the treated genomic DNA, or the treated fragment thereof, with an amplification enzyme and at least one primer comprising, a contiguous sequence of at least 9 nucleotides that is complementary to, or hybridizes under moderately stringent or stringent conditions to a sequence selected from the group consisting of SEQ ID NOS:5-20, SEQ ID NOS:66-181, and complements thereof, wherein the treated genomic DNA or the fragment thereof is either amplified to produce at least one amplificate, or is not amplified; and
d) determining, based on a presence or absence of, or on a property of said amplificate, the methylation state or level of the at least one CpG dinucleotide of the sequence selected from the group consisting SEQ ID NOS:1-4 and SEQ ID NOS:37, or an average, or a value reflecting an average methylation state or level of a plurality of CpG dinucleotides of a sequence selected from the group consisting of SEQ ID NOS:1-4 and SEQ ID NOS:37 65, wherein at least one of detecting and diagnosing cancer is, at least in part, afforded.

8. The method of claim 6, comprising:
a) obtaining extracted or otherwise isolated genomic DNA from a biological sample obtained from the subject;
b) digesting the genomic DNA of a), or a fragment thereof, with one or more methylation sensitive restriction enzymes;
c) contacting the DNA restriction enzyme digest of b), with an amplification enzyme and at least two primers suitable for the amplification of a sequence comprising at least one CpG dinucleotide of a sequence selected from the group consisting of SEQ ID NOS:1-4 and SEQ ID NOS:37-65;
d) determining, based on a presence or absence of an amplificate the methylation state or level of the at least one CpG dinucleotide of the sequence selected from the group consisting of SEQ ID NOS:1-4 and SEQ ID NOS:37-65, whereby at least one of detecting and classifying cancer is, at least in part, afforded.

9. A treated nucleic acid for use in the detection of prostate cell proliferative disorders derived from a genomic DNA sequence selected from the group consisting of SEQ ID NOS:1-4 and SEQ ID NOS:37-65, wherein the treatment is suitable to convert at least one unmethylated cytosine base of the genomic DNA sequence to uracil or another base that is detectably dissimilar to cytosine in terms of hybridization.

10. The treated nucleic acid of claim 9, comprising at least 9 or at least 16 contiguous nucleotides of a treated genomic DNA sequence selected from the group consisting of SEQ ID NOS:5-20, SEQ ID NOS:66-181, and sequences complementary thereto.

11. The treated nucleic acid of claim 10, comprising at least 50 contiguous nucleotides of a DNA sequence selected from the group consisting of SEQ ID NOS:5-20, SEQ ID NOS:66-181, and sequences complementary thereto.

12. A kit, comprising:
a nucleic acid detection component having
a plurality of oligonucleotides or polynucleotides able to hybridise under stringent or moderately stringent conditions to the transcription products of at least one gene selected from the group consisting of RASSF2A; TFAP2E; HIST1H4K; GSTPi; MME; RARB; NKX3-1; NPAL3; ACVR2A; ARL4C; PDE4D; CARTPT; HIST1H2BD; CUL1; MOBKL2B; IFLTD1 (KRAS); ANXA2; NFATC3; MN1; KLF8; FGF13; NKX2-6; SPG20; DSE/SART2; GJA1 (CX43); SPATA6; MCC; GPR68 (INTRON 1 AND/OR EXON 2 REGION);
a container suitable for containing the oligonucleotides or polynucleotides and a biological sample of the patient comprising the transcription products wherein the oligonucleotides or polynucleotides can hybridise under stringent or moderately stringent conditions to the transcription products; and
means to detect the hybridisation; or
a protein detection component having
means for detecting polypeptides of a gene selected from the group consisting of RASSF2A; TFAP2E; HIST1H4K; GSTPi; MME; RARB; NKX3-1; NPAL3; ACVR2A; ARL4C; PDE4D; CARTPT; HIST1H2BD; CUL1; MOBKL2B; IFLTD1 (KRAS); ANXA2; NFATC3; MN1; KLF8; FGF13; NKX2-6; SPG20; DSE/SART2; GJA1 (CX43); SPATA6; MCC; GPR68 (INTRON 1 AND/OR EXON 2 REGION);
a container suitable for containing the said means and the biological sample of the patient comprising the polypeptides wherein the means can form complexes with the polypeptides; and
means to detect the complexes; and optionally
instructions for use and interpretation of the kit results.

13. (canceled)

14. A kit, comprising either:
a) a bisulfite reagent component having
a bisulfite reagent;
a container suitable for containing the said bisulfite reagent and the biological sample of the patient; and
at least one set of oligonucleotides containing two oligonucleotides whose sequences in each case are identical, are complementary, or hybridize under stringent or highly stringent conditions to a 9 or more preferably 18 base long segment of a sequence selected from the group consisting of SEQ ID NOS:5-20 and SEQ ID NOS:66-181; or
b) a methylation sensitive restriction enzyme reagent component having
a methylation sensitive restriction enzyme reagent;
a container suitable for containing the said reagent and the biological sample of the patient; and
at least one set of oligonucleotides one or a plurality of nucleic acids or peptide nucleic acids which are identical, are complementary, or hybridize under stringent or highly stringent conditions to an at least 9 base long segment of a sequence selected from the group consisting of SEQ ID NOS:1-4 and SEQ ID NOS:37-65; and optionally
c) instructions for use and interpretation of the kit results.

15. (canceled)

16. (canceled)


SEQUENCE LISTING (269 sequences)

SEQ ID NO: 1 - DNA, 1920 bp, Homo Sapiens
ccaggctgccgtagacacagcctttgctctcccgaaaaacacgttctaggcgccgggattccagatacctgggaaatagagtgcacgcagctgttgagaggcctcgcgcttggcttctcctatcactgaggcgcagaggtgctgtggacagcccagacccacacggcgcccgaggtgaaacagaaccctcagtctccctatgaggccactggcactctcggctgtccccagagctctccgacttagagctgaatgcaaagtaagcgctcgaaatgcagaagtagccggggccgcccacggcacctgcctcgctcggggcgagagaagacgccaggctgaggtcccagcgacctcaggcaccagctccgaaggagggcggggagaccgcaaaggggaagtgcccggagggccaacggcccccgcgcaccctgcgcccctctgaagcgcgccgcctccccgcgccggggactgggacctgcctctggggaatccgcctagaagacggcggcggactggggtcgggcactctccagggctgtcaggccctccccagccctgcacctgccgcgccgccccacctcgccaggaagtctcagagaccccggggatggggtgggagcgccttcccatcgcgggctcaaaaagaaggaaggacgcccccaggggtcgtagaaggaggactagctccaagccacaactttcttcggacccaaggcaggccggctggggctccgcgcctacacggcccctggcgggggtccgcgcgccccgggagccccgcggctcggggaggaaagaggagacaagagacaggcgaggattacggggctgacccagccggggtagggaccatcgtggaaaaactttggcgaggtggggggacgcggaaagagagcggcccgcgccctgcaccttgcgccgggcatcccgcgccagtgcctcgctcccagtgccccgcgccccgcgccccgcgccttgccttcaccccgggccagctgcatcgcgcccgcgccgcaggaaccgtggagttggaaagtgggggcgccgcggctggggggctgcttcagctgcgcctcggccagcgatcggcgggccgggctcaaatccagccaggctgggcaggcggtggccgcgcgactggggaccgggcgccccgccctcctcgctcccctcctccttcctctccctccctccagccccttggcctttttcagcccctaccggatctgctcgtccgctgtcctctcttttctctcgctcttcatatcactctccaccccttcgccttgccttcgcctttcttcctccccttgtctcctgccccctcctcttctcccctcccctctaggggcggagcttctcccctccctcccagacaatgctgtggctgcgtccccttccccgccagctcgtccaggctcccgccgccagcgattcttccgggctgggggtggggaggtggggggggagtgcagggttggggaggatgagctggctcccctcacctccttgctgctgccctctccaagagggatggagacttggcccaagctcctcggttcacccggagctgtgacagccactcccagggaacagtcacgctgccctaccaagcccacctccagcggcctggattccccaggcagaggttgtgggattttgttttttctaacatcccagcttattcccaaaagggtttgagccggacaggggctaaacaggccccttcgacttggcgggccggccagacgtgacagcaatgccaaggaggccaagtttctttgtccatttctcacctcccccttttccatccctggacctcctggcgcccccagtacacagaggcccttgagcagcccggctgcaggttccctatctactcagagttctccccctcacgtgcctatccccaaccctgca

SEQ ID NO: 2 - DNA, 6096 bp, Homo Sapiens
taccagtgtaagattcaaaattccctttttgcactgcacagtgagatgcccagggctccagctcagtgcctggacatagcgattcctgggcctgcccgtcgccgccccaagcgaagctggtgcgccttgggcggagcagacagagaccctgggtggcaggggcttgggaagacatgggcggctagggctttatgcgccctcaccgctgccctctgctatttgcaggcaatggacgagccgggaatgagcctcctagaccagtccgtgatcaagaaaggtaaggaatggtctgtcagggcagagcccggcgagatggtgcaggcccttggtgcacagatccattttcttcaccggccgtgcctcctgtgtgtcgccaggctgggtgtccaccaggcactcttcctggcccagccagatgttaggcagacgtgcgggcttggtgagtttgcccagcaccctgtggcctggggtgggcctcagcggatcagcattcactgggctgcagcactgggagcctggcctctccccgccgagggggagggcactcttgtggatctggagttgatttgcagaacgagttaaaccacttccctgtttccctaagagatgggaatggaagtgctgttcccacggagttggggaaatgattttcactttacagtgccttagcatttcggtgcctggcgggcactttcttcctcttccttccaggcagggccttggaggcctctgggggaattttctttctgtgggagtctcttgcggcatttagacttaggggagcttgtgtgtgagtactgtgtgttaggctgtgtgcacctgagtcagggcccacctgctcctgggtgtctgtgtccatgtgagttcagggtcctgtgcatgtctgaaatgttcccttcatgggtgtcttagtatttcttggagtgtgagtgtgtctgtttctgtgaatgtgtttgtgaggtgtgtctctgtatgttggtgtgcatttctctgcatttgggggatgtacacatttctcaatatgtacagtatctctgttgtgtcctgcactttgttctttggtatctgaggatttccaagcatgcgcgggccctctctgtgtatatataggagtatttatgtgactcctggcattagtaaaatccagggacacgggatccaccttttctggcctgaggaccaagtactggccatgacaggggaaggtgagagacgacaaaaacagagagacagccagagaggagcagagagtcagaggggcccaggcattgggtagcagcctctttacatttggggcaggtgcccgaaagaattcagaggtgcacatgagcctgaggtgccccaggcaggcactgctcccacagggtttggcttgagttgtttttcaaacgagtgaattcaagcctgggctctatttgccctccacttgttctcaggggaggccaaggtggaagtggtggtagcagggctggggctggacttccaggagctggggctgagttaccaggagctgggggttgggtggatgacttggagtgtgtagcagggaagatgaggcaacagggcaggaagtgggtggggggaggtggaattggggctgtgtcctgtgtcgcttggaactgggagtgtgggaaagacactaggaacctggttgcagcgcagctctgctggtggggcttggttggcttactgtacagagcctttcttgacccctgaagaaagagatccgtctgcagtgggcaaaagcctgcctggacttcctggccaccagaaatatgagcatggtggtggtccccagttccctattcatgcttgggctcaagagactgggagtctaggttcactgactccctgagaaagactaagaccctgcattttagaaagaggtttggggatctctgccctgcgcaagggtagaaggatcagctgttcctctgagcaccttaacccggaaccccggtccgaagccgagacaggagactggatgcgaggccctcccagagctggtttctctcaaacaacttccaaaactcctagatcctaggggtacgccgaaatcccccaaagcagtccaaagaacacaacgagagtcctaacatcccaggtggcggcgcgctggctccctggagcggggcgggacgcggccgcgcggactcacgtgcacaaccgcgcgggacggggccacgcggactcacgtgcacaaccgcgggaccccagcgccagcgggaccccagcgccagcgggaccccagcgccagcgggaccccagcgccagcgggaccccagcgccagcgggaccccagcgccagcgggaccccagcgccagcgggtctgtggcccagtggagcgagtggagcgctggcgacctgagcggagactgcgccctggacgccccagcctagacgtcaagttacagcccgcgcagcagcagcaaaggggaaggggcaggagccgggcacagttggatccggaggtcgtgacccaggggaaagcgtgggcggtcgacccagggcagctgcggcggcgaggcaggtgggctccttgctccctggagccgcccctccccacacctgccctcggcgcccccagcagttttcaccttggccctccgcggtcactgcgggattcggcgttgccgccagcccagtggggagtgaattagcgccctccttcgtcctcggcccttccgacggcacgaggaactcctgtcctgccccacagaccttcggcctccgccgagtgcggtactggagcctgccccgccagggccctggaatcagagaaagtcgctctttggccacctgaagcgtcggatccctacagtgcctcccagcctgggcgggagcggcggctgcgtcgctgaaggttggggtccttggtgcgaaagggaggcagctgcagcctcagccccaccccagaagcggccttcgcatcgctgcggtgggcgttctcgggcttcgacttcgccagcgccgcggggcagaggcacctggagctcgcagggcccagacctgggttggaaaagcttcgctgactgcaggcaagcgtccgggaggggcggccaggcgaagccccggcgctttaccacacacttccgggtcccatgccagttgcatccgcggtattgggcaggaaatggcagggctgaggccgaccctaggagtataagggagccctccatttcctgcccacatttgtcacctccagttttgcaacctatcccagacacacagaaagcaagcaggactggtggggagacggagcttaacaggaatattttccagcagtgagcaggggctgtatgggacgcgggaggagctcagaggaggcgcggagagtgcccgaggttgggtgagtgcctagaggggagatagttgaaccgggttcaagaggtgcttagtgggtgtttgttgaatgaatgagtgatgggctttgaagtctgagtgcattgaaagagggggtgtgtaaaaagggctcctttcatcacacaggacacagcatatgcaaatcctctccctgtggaaaagccagacaggttaaaaaggttacaaacaaattagccgggcatggtggtgcgcgtctgtagtcccagctactagggaggctgagccaggggaatcgcttgaacccgggaggcggagattgcagtgagccaagatcgcgccactgcactccagcctggaaacagagcgagactccgtctcggaaaaaaaaaaaaaaagttacaaaccgtgtgtgggtttcaggttatacaatcagagctggaggggagtggtcaaggatgagaactgagatggatccctcgttccctctggaggagagtgggtggttgcctacttgggggtggggaatccctctccacgggctcagctgtccaatctcaggggatctctaggacaggagctgatgtaaacagtcgccctattccttgctgtctttggccctggagaaggaggagggagctggggagggtctccacttcccagacaatctctaagcagccaggacatgggtgagatgagtgagatactgacttctgggacagaatttgagagggtgccaaaaaactcagtaatcaagataaataggccgggcgcagtggctcacgtctgtaatcccagcactttgggaggccggatcacttgaggtcaagagttcgagaccagcctggccaagatggtgaaaccccatctctactaaaaatacaaaaattagcccagtgtggtggcgctagcctgtaatcccagccactcaggaggctgaggcaagagaattgcttgacccaggaggcagaggttgcagtgagccgagatcatgccactgtactccagcctggacaacagagggagactatctcaaaaaaaaaaaaaaaaaaaaaaaaaaaaaagaggccgggcggcggtggctcacaccatgtgatcccagcactttgggaggccgaggcgggtggatcacctgaggtctggagttcgagaccagcctggccaatatggtgaaaccccgtttctactaaaaatacaaaaattagctgggtggggtggcaggcacctgtaatcccagctactccggaggctgaggcaggagaatcccttgaacctgcggggcggaggttgcagtgaaccaagatcacaccattgcactccagcctggacaacaacagcaaaactctgtctcaaaaaaaaaaaaaatcttttttttcgagacacagttttactctctcgcccaggttggggtgcagcaccacgatctcagctcactgcaacctctgcctctcagattctcgtacctcagcctcccaagtagctgggattacaggtacctgtcaccacgcccagctaatttttgtatttttagtaggggcgtggtttcaccatgttggccaggctggtcttgaactcctgacctcaagtgacctgcccgcttcagccacccaaagtgctgggattacaggcgtgagccaccacgcttggcctttttaaatgaaaatagtgcaaaaatccacgataaacaaaatatcaaaaatttactgaacttgcacttccacaaccctttctcacctgcctcccaggctactctctgccccagaaagcaacttaaaaaatgtgcagatggagtttggactttacctgaaaatggtgggagctatggaaaaccttggagcaggggagtgaaggatagaaattatatgtaaaagaaaccctgggccgggcgcagtggcttatgcctgtaatcccagcactttgggaggccgaggcaggtggattacctgaggtcaggagattgagaccagcctgaccaacatggtgaaatgtcatctctactaaaaatacaaaaaaaattagccaggcatggtggtgcacgcctgtagtcccagctactccggaggctgagacaggaaaatcgcttgaacccgggaggcggaggttgcagtgagccaagattgtgccattgcactccagcctgggcaacaagagcaaaactccatcttaaaaaaaaagaaagaaagaaaccctctggcagttgatgagaaggaaacttaatcggcaggtcccagcaggggagatgaggagactctagggagggcatttgcacatgctgtgccccagtgtgggccagggagcaggtcactactcctcccgtctaccttcctcttgctccaaccccttcaagctttggaccagtggtaccctaagtgtagtccaaggaaccacatgcatcaggacccccagggggtgcttgttaaaaatgcaaattttggccaggtgcagtggctcacacctgtaatcccagcactttgggaggccgaggcgggtggatcacgaggtcaggagatcgagaccatcctggcaaacacggtgaaaccccatctctactaaaaaaacaaaaacaaaccaaaaaaaacattagctgggcgtggtggcgggcgcctgtagtcccagctactcgggaggctgaggcaggagaatggcgtgaacccgggaggcggagcttgcagtgagctgagattgcgccactgcaccccagcctgggcgacagagcgagactctgcctcaaaaaaaaaaaagcaaatttcttgggcaccaccccacattgactg

SEQ ID NO: 3 - DNA, 2501 bp, Homo Sapiens
tttgcaaatggagacatcttcattattcctatagtatcatatgtttttaaagtttgtactcacactttgggtgataaatgaaggacaagatccttccctatccttgtgaggatgactacagcatgactggatgggcttgctatgatttttatctttccctgtgttctcactaccgttttattaatctcagttctttttcacagggtagcacagaatttaactagcagaaagagatccagccatgtagaccagagatttgtctaagtgacggcatgtaagaatcaggaaggaaagttttttgtttaaataccaacaggttccttccttaaagcaattattatttttcaaatctaacccacaaggtgatagtatccttaaaccaattaaatcagaatctcgggttggataacctcaaatatgacttattagcacttcccattaatcactggtccttcaggcctttaagtttacttactaggaatctcacttttaataccatcttatcaacttcagttgtaaataagagaacactcaaaggctgaggaattctcagcggtaaagctctgcccacgttaagtaacaaaggataagttagtctttgttgtgatcactttgttgtactgataagctacgtatttctactcaaggattcaaattctcacctttctcaagaattgggccaaaaccgataaactaaacttatttacggtccactgattaaaggttgttgcataataagttcttgctatgttcagcagttggattcacagcgccagaaacctataactgcttgactttcctccccactacactgcgaaaattgccccttaaatgtaactaaccctaaaacctcaacagtatcgtggccaggcgtggtggctcactactgtaataccaacattaggcataggcgaggggattgaggccaggatatcgaaactagcctgggaaacacacggagacccggtctttggaaaaataattagccttgcgtggtggtgggcgcgaggttccggctaatcgggaggctacagtgagccatgatgacactgcactacagtctgcgcgacggcccatgtcagtaagctctggagcacctgaaacaagttgtgttgggtattttatttactggagagcgattagtgactgatgcctacttacagcgactagagacgcatgctccgatagcagcacaaactcagcaggcgcgaacaaatggtaaagagaaactgggcaaacaagcatcacggctcctcagctgagaaagtgggggccctaaaaagggccttttgttgatagaaagggacgctcaaccaccgaaaccgtagagggtgcggccctggcgcttgagcgcgtagaccacatccatggcggtgaccgtcttgcgcttggcgtgctctgtataggtcacggcgtcccggatcacgttctccaggaacaccttcagcaccccgcgagtctcctcgtagatgaggccggagatgcgcttcacgccgccgcggcgagcaaggcgccggatggccggcttggtgatgccctggatattgtcgcgcagtactttacggtggcgcttagcgccgcctttgccaagacccttcccgcctttgccgcggccagacatgacgagcaagaggagtctcacccaacgctttgtgaggactctggcctgaggcagcgcctttatacgacagttggcggaccgaactgagaacctgaaagaagtcggcgggaagtcccgccccggtgggggaggggaaatctaaagggccaaaccgaaatagggggaaaaaaaaagcgagcttcttgtttccgtgttctgaattttgtaacgtgcatagtattttgttaccacgttatgaggctttaaaaaattgcttttgaacgcagaagatatacatcaatactgtgggaaatacaagaaaggacaagaaattaagaaactacaatgttatcccatcacacaggctagttaatcatgtattttgcagagcagttgcacatatttttccaagaaaatgtatacagtgttgtatatggagttttgtaacctccttatattgattataatttaaccaatttctattaaagagataaaagtgatgttttggtgtctatgtttcttaggaattatcaatagttataatcagttccccagcaattttttaatcggctgtattttaaaaataatgttttccacattcaacataaatgtactttttctctatacttgggaccaatattgaaatttatgattttattacaccaaaatttaaattttattacattaatatttaaaattgtattagaggtctcatgatttggtactacgggtctccgcattatttcctttccaaatttcctaatctgtttcaccaaggtttctggacaactttagagaccttttgtgaagtttgaataaaatctcttcgagattttgataattgcattagctttaggacttaattggaatagaattaaaatccttaaaacaagctcttata

SEQ ID NO: 4 - DNA, 2501 bp, Homo Sapiens
ttgttgtacagaatatttcatcacccaggtattatgccgagtacccaatagttctcttttctgctcctctccttcctcccatcctgcaccctggagtcaaccacagtgtctgttgtttccttgtttgtgttataagttctcatcatttagctcccacttacaagtgagaacatccagtatttggatttctgttcctgcattagtttgctaaggataatagcctctagctccatccatgttcccacaaaagacatgatctagttctttttaatggctgcattaaatgaagttttaaagatacaacataaacaccaacctcttccccaccacaaaaatcccttgctgaatttgattacacttaaattaacgagttttgtttcatgaaagactccttggacaaacttgacagttgatggaataggagaagctgtctgtcatgtctaaagccaacaagagatcaatatctagaataaatggagatctgcaaatcaacagaaagtaggcagcaaagccaaagaaaatagcctaaggcacagccactaaaaggaacgtgatcatgtcctttgcagggacatgggtggagctggaagccgttagcctcagcaaactcacacaggaacagaaaaccagcgagaccgcatggtctcacttataagtgggagctgaacaatgagaacacatggtcacatggcggcgatcaacacacactggtgcctgttgagcggggtgctggggagggagagtaccaggaagaatagctaagggatactgggcttaatacctgggtgatgggatgatctgtacagcaaaccatcatggcgcacacacctatgtaacaaacctgcacatcctctacatgtaccccagaacttcaaataaaagttggacggccaggcgtggtggctcacgcctgtaatcccagcactttgggaagccgaggcgtgcagatcacctaaggtcaggagttcgagaccagcccggccaacatggtgaaaccccgtctctactaaaaatacaaaaatcagccagatgtggcacgcacctataattccacctactcgggaggctgaagcagaattgcttgaacccgagaggcggaggttgcagtgagccgccgagatcgcgccactgcactccagcctgggccacagcgtgagactacgtcataaaataaaataaaataacacaaaataaaataaaataaaataaaataaaataaaataaaataaaataaaataaaataaaaaaataaaataaaataaaataaaataaagcaatttcctttcctctaagcggcctccacccctctcccctgccctgtgaagcgggtgtgcaagctccgggatcgcagcggtcttagggaatttccccccgcgatgtcccggcgcgccagttcgctgcgcacacttcgctgcggtcctcttcctgctgtctgtttactccctaggccccgctggggacctgggaaagagggaaaggcttccccggccagctgcgcggcgactccggggactccagggcgcccctctgcggccgacgcccggggtgcagcggccgccggggctggggccggcgggagtccgcgggaccctccagaagagcggccggcgccgtgactcagcactggggcggagcggggcgggaccacccttataaggctcggaggccgcgaggccttcgctggagtttcgccgccgcagtcttcgccaccagtgagtacgcgcggcccgcgtccccggggatggggctcagagctcccagcatggggccaacccgcagcatcaggcccgggctcccggcagggctcctcgcccacctcgagacccgggacgggggcctaggggacccaggacgtccccagtgccgttagcggctttcagggggcccggagcgcctcggggagggatgggaccccgggggcggggagggggggcagactgcgctcaccgcgccttggcatcctcccccgggctccagcaaacttttctttgttcgctgcagtgccgccctacaccgtggtctatttcccagttcgaggtaggagcatgtgtctggcagggaagggaggcaggggctggggctgcagcccacagcccctcgcccacccggagagatccgaacccccttatccctccgtcgtgtggcttttaccccgggcctccttcctgttccccgcctctcccgccatgcctgctccccgccccagtgttgtgtgaaatcttcggaggaacctgtttccctgttccctccctgcactcctgacccctccccgggttgctgcgaggcggagtcggcccggtccccacatctcgtacttctccctccccgcaggccgctgcgcggccctgcgcatgctgctggcagatcagggccagagctggaaggaggaggtggtgaccgtggagacgtggcaggagggctcactcaaagcctcctgcgtaagtgaccatgcccgggcaaggggagggggtgctgggccttagggggctgtgactaggatc

SEQ ID NO: 5 - DNA, 1920 bp, Artificial Sequence
  chemically treated genomic DNA (Homo sapiens)
ttaggttgtcgtagatatagtttttgttttttcgaaaaatacgttttaggcgtcgggattttagatatttgggaaatagagtgtacgtagttgttgagaggtttcgcgtttggttttttttattattgaggcgtagaggtgttgtggatagtttagatttatacggcgttcgaggtgaaatagaatttttagtttttttatgaggttattggtattttcggttgtttttagagtttttcgatttagagttgaatgtaaagtaagcgttcgaaatgtagaagtagtcggggtcgtttacggtatttgtttcgttcggggcgagagaagacgttaggttgaggttttagcgattttaggtattagtttcgaaggagggcggggagatcgtaaaggggaagtgttcggagggttaacggttttcgcgtattttgcgtttttttgaagcgcgtcgttttttcgcgtcggggattgggatttgtttttggggaattcgtttagaagacggcggcggattggggtcgggtattttttagggttgttaggttttttttagttttgtatttgtcgcgtcgttttatttcgttaggaagttttagagatttcggggatggggtgggagcgtttttttatcgcgggtttaaaaagaaggaaggacgtttttaggggtcgtagaaggaggattagttttaagttataatttttttcggatttaaggtaggtcggttggggtttcgcgtttatacggtttttggcgggggttcgcgcgtttcgggagtttcgcggttcggggaggaaagaggagataagagataggcgaggattacggggttgatttagtcggggtagggattatcgtggaaaaattttggcgaggtggggggacgcggaaagagagcggttcgcgttttgtattttgcgtcgggtatttcgcgttagtgtttcgtttttagtgtttcgcgtttcgcgtttcgcgttttgtttttatttcgggttagttgtatcgcgttcgcgtcgtaggaatcgtggagttggaaagtgggggcgtcgcggttggggggttgttttagttgcgtttcggttagcgatcggcgggtcgggtttaaatttagttaggttgggtaggcggtggtcgcgcgattggggatcgggcgtttcgtttttttcgtttttttttttttttttttttttttttttagttttttggttttttttagtttttatcggatttgttcgttcgttgtttttttttttttttcgttttttatattattttttattttttcgttttgttttcgtttttttttttttttttgttttttgttttttttttttttttttttttttttaggggcggagtttttttttttttttttagataatgttgtggttgcgttttttttttcgttagttcgtttaggttttcgtcgttagcgattttttcgggttgggggtggggaggtggggggggagtgtagggttggggaggatgagttggttttttttatttttttgttgttgttttttttaagagggatggagatttggtttaagtttttcggtttattcggagttgtgatagttatttttagggaatagttacgttgttttattaagtttatttttagcggtttggattttttaggtagaggttgtgggattttgttttttttaatattttagtttatttttaaaagggtttgagtcggataggggttaaataggttttttcgatttggcgggtcggttagacgtgatagtaatgttaaggaggttaagtttttttgtttattttttattttttttttttttatttttggattttttggcgtttttagtatatagaggtttttgagtagttcggttgtaggttttttatttatttagagtttttttttttacgtgtttatttttaattttgta

SEQ ID NO: 6 - DNA, 1920 bp, Artificial Sequence
  chemically treated genomic DNA (Homo sapiens)
tgtagggttggggataggtacgtgagggggagaattttgagtagatagggaatttgtagtcgggttgtttaagggtttttgtgtattgggggcgttaggaggtttagggatggaaaagggggaggtgagaaatggataaagaaatttggtttttttggtattgttgttacgtttggtcggttcgttaagtcgaaggggtttgtttagtttttgttcggtttaaatttttttgggaataagttgggatgttagaaaaaataaaattttataatttttgtttggggaatttaggtcgttggaggtgggtttggtagggtagcgtgattgttttttgggagtggttgttatagtttcgggtgaatcgaggagtttgggttaagtttttattttttttggagagggtagtagtaaggaggtgaggggagttagtttattttttttaattttgtattttttttttatttttttatttttagttcggaagaatcgttggcggcgggagtttggacgagttggcggggaaggggacgtagttatagtattgtttgggagggaggggagaagtttcgtttttagaggggaggggagaagaggagggggtaggagataaggggaggaagaaaggcgaaggtaaggcgaaggggtggagagtgatatgaagagcgagagaaaagagaggatagcggacgagtagattcggtaggggttgaaaaaggttaaggggttggagggagggagaggaaggaggaggggagcgaggagggcggggcgttcggtttttagtcgcgcggttatcgtttgtttagtttggttggatttgagttcggttcgtcgatcgttggtcgaggcgtagttgaagtagttttttagtcgcggcgtttttattttttaattttacggtttttgcggcgcgggcgcgatgtagttggttcggggtgaaggtaaggcgcggggcgcggggcgcggggtattgggagcgaggtattggcgcgggatgttcggcgtaaggtgtagggcgcgggtcgtttttttttcgcgtttttttatttcgttaaagtttttttacgatggtttttatttcggttgggttagtttcgtaattttcgtttgttttttgttttttttttttttttcgagtcgcggggttttcggggcgcgcggattttcgttaggggtcgtgtaggcgcggagttttagtcggtttgttttgggttcgaagaaagttgtggtttggagttagttttttttttacgatttttgggggcgtttttttttttttttgagttcgcgatgggaaggcgtttttattttattttcggggtttttgagattttttggcgaggtggggcggcgcggtaggtgtagggttggggagggtttgatagttttggagagtgttcgattttagttcgtcgtcgttttttaggcggattttttagaggtaggttttagttttcggcgcggggaggcggcgcgttttagaggggcgtagggtgcgcgggggtcgttggtttttcgggtattttttttttgcggttttttcgttttttttcggagttggtgtttgaggtcgttgggattttagtttggcgttttttttcgtttcgagcgaggtaggtgtcgtgggcggtttcggttatttttgtatttcgagcgtttattttgtatttagttttaagtcggagagttttggggatagtcgagagtgttagtggttttatagggagattgagggttttgttttatttcgggcgtcgtgtgggtttgggttgtttatagtatttttgcgttttagtgataggagaagttaagcgcgaggttttttaatagttgcgtgtattttattttttaggtatttggaatttcggcgtttagaacgtgtttttcgggagagtaaaggttgtgtttacggtagtttgg

SEQ ID NO: 7 - DNA, 6096 bp, Artificial Sequence
  chemically treated genomic DNA (Homo sapiens)
tattagtgtaagatttaaaattttttttttgtattgtatagtgagatgtttagggttttagtttagtgtttggatatagcgatttttgggtttgttcgtcgtcgttttaagcgaagttggtgcgttttgggcggagtagatagagattttgggtggtaggggtttgggaagatatgggcggttagggttttatgcgtttttatcgttgttttttgttatttgtaggtaatggacgagtcgggaatgagttttttagattagttcgtgattaagaaaggtaaggaatggtttgttagggtagagttcggcgagatggtgtaggtttttggtgtatagatttattttttttatcggtcgtgttttttgtgtgtcgttaggttgggtgtttattaggtattttttttggtttagttagatgttaggtagacgtgcgggtttggtgagtttgtttagtattttgtggtttggggtgggttttagcggattagtatttattgggttgtagtattgggagtttggttttttttcgtcgagggggagggtatttttgtggatttggagttgatttgtagaacgagttaaattatttttttgtttttttaagagatgggaatggaagtgttgtttttacggagttggggaaatgatttttattttatagtgttttagtatttcggtgtttggcgggtatttttttttttttttttttaggtagggttttggaggtttttgggggaattttttttttgtgggagttttttgcggtatttagatttaggggagtttgtgtgtgagtattgtgtgttaggttgtgtgtatttgagttagggtttatttgtttttgggtgtttgtgtttatgtgagtttagggttttgtgtatgtttgaaatgttttttttatgggtgttttagtattttttggagtgtgagtgtgtttgtttttgtgaatgtgtttgtgaggtgtgtttttgtatgttggtgtgtatttttttgtatttgggggatgtatatattttttaatatgtatagtatttttgttgtgttttgtattttgttttttggtatttgaggatttttaagtatgcgcgggttttttttgtgtatatataggagtatttatgtgatttttggtattagtaaaatttagggatacgggatttattttttttggtttgaggattaagtattggttatgataggggaaggtgagagacgataaaaatagagagatagttagagaggagtagagagttagaggggtttaggtattgggtagtagtttttttatatttggggtaggtgttcgaaagaatttagaggtgtatatgagtttgaggtgttttaggtaggtattgtttttatagggtttggtttgagttgttttttaaacgagtgaatttaagtttgggttttatttgttttttatttgtttttaggggaggttaaggtggaagtggtggtagtagggttggggttggatttttaggagttggggttgagttattaggagttgggggttgggtggatgatttggagtgtgtagtagggaagatgaggtaatagggtaggaagtgggtggggggaggtggaattggggttgtgttttgtgtcgtttggaattgggagtgtgggaaagatattaggaatttggttgtagcgtagttttgttggtggggtttggttggtttattgtatagagttttttttgatttttgaagaaagagattcgtttgtagtgggtaaaagtttgtttggattttttggttattagaaatatgagtatggtggtggtttttagttttttatttatgtttgggtttaagagattgggagtttaggtttattgattttttgagaaagattaagattttgtattttagaaagaggtttggggatttttgttttgcgtaagggtagaaggattagttgtttttttgagtattttaattcggaatttcggttcgaagtcgagataggagattggatgcgaggtttttttagagttggttttttttaaataatttttaaaatttttagattttaggggtacgtcgaaattttttaaagtagtttaaagaatataacgagagttttaatattttaggtggcggcgcgttggttttttggagcggggcgggacgcggtcgcgcggatttacgtgtataatcgcgcgggacggggttacgcggatttacgtgtataatcgcgggattttagcgttagcgggattttagcgttagcgggattttagcgttagcgggattttagcgttagcgggattttagcgttagcgggattttagcgttagcgggattttagcgttagcgggtttgtggtttagtggagcgagtggagcgttggcgatttgagcggagattgcgttttggacgttttagtttagacgttaagttatagttcgcgtagtagtagtaaaggggaaggggtaggagtcgggtatagttggattcggaggtcgtgatttaggggaaagcgtgggcggtcgatttagggtagttgcggcggcgaggtaggtgggttttttgttttttggagtcgtttttttttatatttgttttcggcgtttttagtagtttttattttggtttttcgcggttattgcgggattcggcgttgtcgttagtttagtggggagtgaattagcgttttttttcgttttcggttttttcgacggtacgaggaatttttgttttgttttatagattttcggttttcgtcgagtgcggtattggagtttgtttcgttagggttttggaattagagaaagtcgttttttggttatttgaagcgtcggatttttatagtgttttttagtttgggcgggagcggcggttgcgtcgttgaaggttggggtttttggtgcgaaagggaggtagttgtagttttagttttattttagaagcggttttcgtatcgttgcggtgggcgttttcgggtttcgatttcgttagcgtcgcggggtagaggtatttggagttcgtagggtttagatttgggttggaaaagtttcgttgattgtaggtaagcgttcgggaggggcggttaggcgaagtttcggcgttttattatatattttcgggttttatgttagttgtattcgcggtattgggtaggaaatggtagggttgaggtcgattttaggagtataagggagttttttattttttgtttatatttgttatttttagttttgtaatttattttagatatatagaaagtaagtaggattggtggggagacggagtttaataggaatattttttagtagtgagtaggggttgtatgggacgcgggaggagtttagaggaggcgcggagagtgttcgaggttgggtgagtgtttagaggggagatagttgaatcgggtttaagaggtgtttagtgggtgtttgttgaatgaatgagtgatgggttttgaagtttgagtgtattgaaagagggggtgtgtaaaaagggttttttttattatataggatatagtatatgtaaattttttttttgtggaaaagttagataggttaaaaaggttataaataaattagtcgggtatggtggtgcgcgtttgtagttttagttattagggaggttgagttaggggaatcgtttgaattcgggaggcggagattgtagtgagttaagatcgcgttattgtattttagtttggaaatagagcgagatttcgtttcggaaaaaaaaaaaaaaagttataaatcgtgtgtgggttttaggttatataattagagttggaggggagtggttaaggatgagaattgagatggatttttcgttttttttggaggagagtgggtggttgtttatttgggggtggggaatttttttttacgggtttagttgtttaattttaggggatttttaggataggagttgatgtaaatagtcgttttattttttgttgtttttggttttggagaaggaggagggagttggggagggtttttattttttagataatttttaagtagttaggatatgggtgagatgagtgagatattgatttttgggatagaatttgagagggtgttaaaaaatttagtaattaagataaataggtcgggcgtagtggtttacgtttgtaattttagtattttgggaggtcggattatttgaggttaagagttcgagattagtttggttaagatggtgaaattttatttttattaaaaatataaaaattagtttagtgtggtggcgttagtttgtaattttagttatttaggaggttgaggtaagagaattgtttgatttaggaggtagaggttgtagtgagtcgagattatgttattgtattttagtttggataatagagggagattattttaaaaaaaaaaaaaaaaaaaaaaaaaaaaaagaggtcgggcggcggtggtttatattatgtgattttagtattttgggaggtcgaggcgggtggattatttgaggtttggagttcgagattagtttggttaatatggtgaaatttcgtttttattaaaaatataaaaattagttgggtggggtggtaggtatttgtaattttagttatttcggaggttgaggtaggagaattttttgaatttgcggggcggaggttgtagtgaattaagattatattattgtattttagtttggataataatagtaaaattttgttttaaaaaaaaaaaaaattttttttttcgagatatagttttattttttcgtttaggttggggtgtagtattacgattttagtttattgtaatttttgttttttagattttcgtattttagttttttaagtagttgggattataggtatttgttattacgtttagttaatttttgtatttttagtaggggcgtggttttattatgttggttaggttggttttgaatttttgattttaagtgatttgttcgttttagttatttaaagtgttgggattataggcgtgagttattacgtttggtttttttaaatgaaaatagtgtaaaaatttacgataaataaaatattaaaaatttattgaatttgtatttttataatttttttttatttgttttttaggttattttttgttttagaaagtaatttaaaaaatgtgtagatggagtttggattttatttgaaaatggtgggagttatggaaaattttggagtaggggagtgaaggatagaaattatatgtaaaagaaattttgggtcgggcgtagtggtttatgtttgtaattttagtattttgggaggtcgaggtaggtggattatttgaggttaggagattgagattagtttgattaatatggtgaaatgttatttttattaaaaatataaaaaaaattagttaggtatggtggtgtacgtttgtagttttagttatttcggaggttgagataggaaaatcgtttgaattcgggaggcggaggttgtagtgagttaagattgtgttattgtattttagtttgggtaataagagtaaaattttattttaaaaaaaaagaaagaaagaaattttttggtagttgatgagaaggaaatttaatcggtaggttttagtaggggagatgaggagattttagggagggtatttgtatatgttgtgttttagtgtgggttagggagtaggttattatttttttcgtttattttttttttgttttaatttttttaagttttggattagtggtattttaagtgtagtttaaggaattatatgtattaggatttttagggggtgtttgttaaaaatgtaaattttggttaggtgtagtggtttatatttgtaattttagtattttgggaggtcgaggcgggtggattacgaggttaggagatcgagattattttggtaaatacggtgaaattttatttttattaaaaaaataaaaataaattaaaaaaaatattagttgggcgtggtggcgggcgtttgtagttttagttattcgggaggttgaggtaggagaatggcgtgaattcgggaggcggagtttgtagtgagttgagattgcgttattgtattttagtttgggcgatagagcgagattttgttttaaaaaaaaaaaagtaaattttttgggtattattttatattgattg

SEQ ID NO: 8 - DNA, 6096 bp, Artificial Sequence
  chemically treated genomic DNA (Homo sapiens)
tagttaatgtggggtggtgtttaagaaatttgtttttttttttttgaggtagagtttcgttttgtcgtttaggttggggtgtagtggcgtaattttagtttattgtaagtttcgtttttcgggtttacgttatttttttgttttagtttttcgagtagttgggattataggcgttcgttattacgtttagttaatgttttttttggtttgtttttgtttttttagtagagatggggttttatcgtgtttgttaggatggtttcgattttttgatttcgtgatttattcgtttcggttttttaaagtgttgggattataggtgtgagttattgtatttggttaaaatttgtatttttaataagtattttttgggggttttgatgtatgtggttttttggattatatttagggtattattggtttaaagtttgaaggggttggagtaagaggaaggtagacgggaggagtagtgatttgttttttggtttatattggggtatagtatgtgtaaatgttttttttagagtttttttattttttttgttgggatttgtcgattaagttttttttttattaattgttagagggtttttttttttttttttttttaagatggagttttgtttttgttgtttaggttggagtgtaatggtataattttggtttattgtaattttcgtttttcgggtttaagcgatttttttgttttagttttcggagtagttgggattataggcgtgtattattatgtttggttaattttttttgtatttttagtagagatgatattttattatgttggttaggttggttttaattttttgattttaggtaatttatttgtttcggttttttaaagtgttgggattataggtataagttattgcgttcggtttagggttttttttatatataatttttattttttatttttttgttttaaggttttttatagtttttattatttttaggtaaagtttaaattttatttgtatattttttaagttgttttttggggtagagagtagtttgggaggtaggtgagaaagggttgtggaagtgtaagtttagtaaatttttgatattttgtttatcgtggatttttgtattatttttatttaaaaaggttaagcgtggtggtttacgtttgtaattttagtattttgggtggttgaagcgggtaggttatttgaggttaggagtttaagattagtttggttaatatggtgaaattacgtttttattaaaaatataaaaattagttgggcgtggtgataggtatttgtaattttagttatttgggaggttgaggtacgagaatttgagaggtagaggttgtagtgagttgagatcgtggtgttgtattttaatttgggcgagagagtaaaattgtgtttcgaaaaaaaagattttttttttttttgagatagagttttgttgttgttgtttaggttggagtgtaatggtgtgattttggtttattgtaattttcgtttcgtaggtttaagggatttttttgttttagttttcggagtagttgggattataggtgtttgttattttatttagttaatttttgtatttttagtagaaacggggttttattatattggttaggttggtttcgaattttagattttaggtgatttattcgtttcggttttttaaagtgttgggattatatggtgtgagttatcgtcgttcggttttttttttttttttttttttttttttttttttgagatagtttttttttgttgtttaggttggagtatagtggtatgatttcggtttattgtaatttttgttttttgggttaagtaatttttttgttttagttttttgagtggttgggattataggttagcgttattatattgggttaatttttgtatttttagtagagatggggttttattattttggttaggttggtttcgaatttttgattttaagtgattcggttttttaaagtgttgggattatagacgtgagttattgcgttcggtttatttattttgattattgagttttttggtatttttttaaattttgttttagaagttagtattttatttattttatttatgttttggttgtttagagattgtttgggaagtggagattttttttagtttttttttttttttttagggttaaagatagtaaggaatagggcgattgtttatattagtttttgttttagagattttttgagattggatagttgagttcgtggagagggattttttatttttaagtaggtaattatttatttttttttagagggaacgagggatttattttagtttttatttttgattatttttttttagttttgattgtataatttgaaatttatatacggtttgtaatttttttttttttttttcgagacggagtttcgttttgtttttaggttggagtgtagtggcgcgattttggtttattgtaattttcgtttttcgggtttaagcgatttttttggtttagtttttttagtagttgggattatagacgcgtattattatgttcggttaatttgtttgtaatttttttaatttgtttggtttttttatagggagaggatttgtatatgttgtgttttgtgtgatgaaaggagttttttttatatatttttttttttaatgtatttagattttaaagtttattatttatttatttaataaatatttattaagtattttttgaattcggtttaattattttttttttaggtatttatttaatttcgggtatttttcgcgttttttttgagtttttttcgcgttttatatagtttttgtttattgttggaaaatatttttgttaagtttcgtttttttattagttttgtttgttttttgtgtgtttgggataggttgtaaaattggaggtgataaatgtgggtaggaaatggagggtttttttatatttttagggtcggttttagttttgttattttttgtttaatatcgcggatgtaattggtatgggattcggaagtgtgtggtaaagcgtcggggtttcgtttggtcgtttttttcggacgtttgtttgtagttagcgaagtttttttaatttaggtttgggttttgcgagttttaggtgtttttgtttcgcggcgttggcgaagtcgaagttcgagaacgtttatcgtagcgatgcgaaggtcgtttttggggtggggttgaggttgtagttgtttttttttcgtattaaggattttaatttttagcgacgtagtcgtcgttttcgtttaggttgggaggtattgtagggattcgacgttttaggtggttaaagagcgattttttttgattttagggttttggcggggtaggttttagtatcgtattcggcggaggtcgaaggtttgtggggtaggataggagtttttcgtgtcgtcggaagggtcgaggacgaaggagggcgttaatttattttttattgggttggcggtaacgtcgaatttcgtagtgatcgcggagggttaaggtgaaaattgttgggggcgtcgagggtaggtgtggggaggggcggttttagggagtaaggagtttatttgtttcgtcgtcgtagttgttttgggtcgatcgtttacgtttttttttgggttacgattttcggatttaattgtgttcggtttttgtttttttttttttgttgttgttgcgcgggttgtaatttgacgtttaggttggggcgtttagggcgtagttttcgtttaggtcgttagcgttttattcgttttattgggttatagattcgttggcgttggggtttcgttggcgttggggtttcgttggcgttggggtttcgttggcgttggggtttcgttggcgttggggtttcgttggcgttggggtttcgttggcgttggggtttcgcggttgtgtacgtgagttcgcgtggtttcgtttcgcgcggttgtgtacgtgagttcgcgcggtcgcgtttcgtttcgttttagggagttagcgcgtcgttatttgggatgttaggattttcgttgtgttttttggattgttttgggggatttcggcgtatttttaggatttaggagttttggaagttgtttgagagaaattagttttgggagggtttcgtatttagttttttgtttcggtttcggatcggggtttcgggttaaggtgtttagaggaatagttgatttttttatttttgcgtagggtagagatttttaaatttttttttaaaatgtagggttttagttttttttagggagttagtgaatttagatttttagttttttgagtttaagtatgaatagggaattggggattattattatgtttatatttttggtggttaggaagtttaggtaggtttttgtttattgtagacggatttttttttttaggggttaagaaaggttttgtatagtaagttaattaagttttattagtagagttgcgttgtaattaggtttttagtgttttttttatatttttagttttaagcgatataggatatagttttaattttatttttttttatttattttttgttttgttgttttattttttttgttatatattttaagttatttatttaatttttagtttttggtaatttagttttagtttttggaagtttagttttagttttgttattattatttttattttggttttttttgagaataagtggagggtaaatagagtttaggtttgaatttattcgtttgaaaaataatttaagttaaattttgtgggagtagtgtttgtttggggtattttaggtttatgtgtatttttgaattttttcgggtatttgttttaaatgtaaagaggttgttatttaatgtttgggtttttttgattttttgtttttttttggttgtttttttgtttttgtcgttttttatttttttttgttatggttagtatttggtttttaggttagaaaaggtggatttcgtgtttttggattttattaatgttaggagttatataaatatttttatatatatatagagagggttcgcgtatgtttggaaatttttagatattaaagaataaagtgtaggatataatagagatattgtatatattgagaaatgtgtatattttttaaatgtagagaaatgtatattaatatatagagatatattttataaatatatttatagaaatagatatatttatattttaagaaatattaagatatttatgaagggaatattttagatatgtataggattttgaatttatatggatatagatatttaggagtaggtgggttttgatttaggtgtatatagtttaatatatagtatttatatataagtttttttaagtttaaatgtcgtaagagatttttatagaaagaaaatttttttagaggtttttaaggttttgtttggaaggaagaggaagaaagtgttcgttaggtatcgaaatgttaaggtattgtaaagtgaaaattatttttttaatttcgtgggaatagtatttttatttttattttttagggaaatagggaagtggtttaattcgttttgtaaattaattttagatttataagagtgttttttttttcggcggggagaggttaggtttttagtgttgtagtttagtgaatgttgattcgttgaggtttattttaggttatagggtgttgggtaaatttattaagttcgtacgtttgtttaatatttggttgggttaggaagagtgtttggtggatatttagtttggcgatatataggaggtacggtcggtgaagaaaatggatttgtgtattaagggtttgtattatttcgtcgggttttgttttgatagattattttttattttttttgattacggattggtttaggaggtttattttcggttcgtttattgtttgtaaatagtagagggtagcggtgagggcgtataaagttttagtcgtttatgtttttttaagtttttgttatttagggtttttgtttgtttcgtttaaggcgtattagtttcgtttggggcggcgacgggtaggtttaggaatcgttatgtttaggtattgagttggagttttgggtattttattgtgtagtgtaaaaagggaattttgaattttatattggta

SEQ ID NO: 9 - DNA, 2501 bp, Artificial Sequence
  chemically treated genomic DNA (Homo sapiens)
tttgtaaatggagatatttttattatttttatagtattatatgtttttaaagtttgtatttatattttgggtgataaatgaaggataagatttttttttatttttgtgaggatgattatagtatgattggatgggtttgttatgatttttatttttttttgtgtttttattatcgttttattaattttagtttttttttatagggtagtatagaatttaattagtagaaagagatttagttatgtagattagagatttgtttaagtgacggtatgtaagaattaggaaggaaagttttttgtttaaatattaataggttttttttttaaagtaattattattttttaaatttaatttataaggtgatagtatttttaaattaattaaattagaatttcgggttggataattttaaatatgatttattagtattttttattaattattggttttttaggtttttaagtttatttattaggaattttatttttaatattattttattaattttagttgtaaataagagaatatttaaaggttgaggaatttttagcggtaaagttttgtttacgttaagtaataaaggataagttagtttttgttgtgattattttgttgtattgataagttacgtatttttatttaaggatttaaatttttattttttttaagaattgggttaaaatcgataaattaaatttatttacggtttattgattaaaggttgttgtataataagtttttgttatgtttagtagttggatttatagcgttagaaatttataattgtttgatttttttttttattatattgcgaaaattgttttttaaatgtaattaattttaaaattttaatagtatcgtggttaggcgtggtggtttattattgtaatattaatattaggtataggcgaggggattgaggttaggatatcgaaattagtttgggaaatatacggagattcggtttttggaaaaataattagttttgcgtggtggtgggcgcgaggtttcggttaatcgggaggttatagtgagttatgatgatattgtattatagtttgcgcgacggtttatgttagtaagttttggagtatttgaaataagttgtgttgggtattttatttattggagagcgattagtgattgatgtttatttatagcgattagagacgtatgtttcgatagtagtataaatttagtaggcgcgaataaatggtaaagagaaattgggtaaataagtattacggttttttagttgagaaagtgggggttttaaaaagggttttttgttgatagaaagggacgtttaattatcgaaatcgtagagggtgcggttttggcgtttgagcgcgtagattatatttatggcggtgatcgttttgcgtttggcgtgttttgtataggttacggcgtttcggattacgttttttaggaatatttttagtatttcgcgagttttttcgtagatgaggtcggagatgcgttttacgtcgtcgcggcgagtaaggcgtcggatggtcggtttggtgatgttttggatattgtcgcgtagtattttacggtggcgtttagcgtcgtttttgttaagatttttttcgtttttgtcgcggttagatatgacgagtaagaggagttttatttaacgttttgtgaggattttggtttgaggtagcgtttttatacgatagttggcggatcgaattgagaatttgaaagaagtcggcgggaagtttcgtttcggtgggggaggggaaatttaaagggttaaatcgaaatagggggaaaaaaaaagcgagttttttgttttcgtgttttgaattttgtaacgtgtatagtattttgttattacgttatgaggttttaaaaaattgtttttgaacgtagaagatatatattaatattgtgggaaatataagaaaggataagaaattaagaaattataatgttattttattatataggttagttaattatgtattttgtagagtagttgtatatatttttttaagaaaatgtatatagtgttgtatatggagttttgtaatttttttatattgattataatttaattaatttttattaaagagataaaagtgatgttttggtgtttatgttttttaggaattattaatagttataattagttttttagtaattttttaatcggttgtattttaaaaataatgttttttatatttaatataaatgtattttttttttatatttgggattaatattgaaatttatgattttattatattaaaatttaaattttattatattaatatttaaaattgtattagaggttttatgatttggtattacgggttttcgtattattttttttttaaattttttaatttgttttattaaggtttttggataattttagagattttttgtgaagtttgaataaaattttttcgagattttgataattgtattagttttaggatttaattggaatagaattaaaatttttaaaataagtttttata

SEQ ID NO: 10 - DNA, 2501 bp, Artificial Sequence
  chemically treated genomic DNA (Homo sapiens)
tataagagtttgttttaaggattttaattttattttaattaagttttaaagttaatgtaattattaaaatttcgaagagattttatttaaattttataaaaggtttttaaagttgtttagaaattttggtgaaatagattaggaaatttggaaaggaaataatgcggagattcgtagtattaaattatgagatttttaatataattttaaatattaatgtaataaaatttaaattttggtgtaataaaattataaattttaatattggttttaagtatagagaaaaagtatatttatgttgaatgtggaaaatattatttttaaaatatagtcgattaaaaaattgttggggaattgattataattattgataatttttaagaaatatagatattaaaatattatttttatttttttaatagaaattggttaaattataattaatataaggaggttataaaattttatatataatattgtatatatttttttggaaaaatatgtgtaattgttttgtaaaatatatgattaattagtttgtgtgatgggataatattgtagttttttaattttttgtttttttttgtattttttatagtattgatgtatattttttgcgtttaaaagtaattttttaaagttttataacgtggtaataaaatattatgtacgttataaaatttagaatacggaaataagaagttcgttttttttttttttttatttcggtttggttttttagatttttttttttttatcggggcgggatttttcgtcgattttttttaggtttttagttcggttcgttaattgtcgtataaaggcgttgttttaggttagagtttttataaagcgttgggtgagattttttttgttcgttatgtttggtcgcggtaaaggcgggaagggttttggtaaaggcggcgttaagcgttatcgtaaagtattgcgcgataatatttagggtattattaagtcggttattcggcgttttgttcgtcgcggcggcgtgaagcgtattttcggttttatttacgaggagattcgcggggtgttgaaggtgtttttggagaacgtgattcgggacgtcgtgatttatatagagtacgttaagcgtaagacggttatcgttatggatgtggtttacgcgtttaagcgttagggtcgtattttttacggtttcggtggttgagcgtttttttttattaataaaaggttttttttagggtttttatttttttagttgaggagtcgtgatgtttgtttgtttagtttttttttattatttgttcgcgtttgttgagtttgtgttgttatcggagtatgcgtttttagtcgttgtaagtaggtattagttattaatcgttttttagtaaataaaatatttaatataatttgttttaggtgttttagagtttattgatatgggtcgtcgcgtagattgtagtgtagtgttattatggtttattgtagtttttcgattagtcggaatttcgcgtttattattacgtaaggttaattatttttttaaagatcgggttttcgtgtgttttttaggttagtttcgatattttggttttaattttttcgtttatgtttaatgttggtattatagtagtgagttattacgtttggttacgatattgttgaggttttagggttagttatatttaaggggtaattttcgtagtgtagtggggaggaaagttaagtagttataggtttttggcgttgtgaatttaattgttgaatatagtaagaatttattatgtaataatttttaattagtggatcgtaaataagtttagtttatcggttttggtttaatttttgagaaaggtgagaatttgaatttttgagtagaaatacgtagtttattagtataataaagtgattataataaagattaatttattttttgttatttaacgtgggtagagttttatcgttgagaattttttagtttttgagtgtttttttatttataattgaagttgataagatggtattaaaagtgagatttttagtaagtaaatttaaaggtttgaaggattagtgattaatgggaagtgttaataagttatatttgaggttatttaattcgagattttgatttaattggtttaaggatattattattttgtgggttagatttgaaaaataataattgttttaaggaaggaatttgttggtatttaaataaaaaatttttttttttgatttttatatgtcgttatttagataaatttttggtttatatggttggatttttttttgttagttaaattttgtgttattttgtgaaaaagaattgagattaataaaacggtagtgagaatatagggaaagataaaaattatagtaagtttatttagttatgttgtagttatttttataaggatagggaaggattttgttttttatttattatttaaagtgtgagtataaattttaaaaatatatgatattataggaataatgaagatgtttttatttgtaaa

SEQ ID NO: 11 - DNA, 2501 bp, Artificial Sequence
  chemically treated genomic DNA (Homo sapiens)
ttgttgtatagaatattttattatttaggtattatgtcgagtatttaatagtttttttttttgtttttttttttttttttattttgtattttggagttaattatagtgtttgttgtttttttgtttgtgttataagtttttattatttagtttttatttataagtgagaatatttagtatttggatttttgtttttgtattagtttgttaaggataatagtttttagttttatttatgtttttataaaagatatgatttagttttttttaatggttgtattaaatgaagttttaaagatataatataaatattaattttttttttattataaaaattttttgttgaatttgattatatttaaattaacgagttttgttttatgaaagattttttggataaatttgatagttgatggaataggagaagttgtttgttatgtttaaagttaataagagattaatatttagaataaatggagatttgtaaattaatagaaagtaggtagtaaagttaaagaaaatagtttaaggtatagttattaaaaggaacgtgattatgttttttgtagggatatgggtggagttggaagtcgttagttttagtaaatttatataggaatagaaaattagcgagatcgtatggttttatttataagtgggagttgaataatgagaatatatggttatatggcggcgattaatatatattggtgtttgttgagcggggtgttggggagggagagtattaggaagaatagttaagggatattgggtttaatatttgggtgatgggatgatttgtatagtaaattattatggcgtatatatttatgtaataaatttgtatattttttatatgtattttagaattttaaataaaagttggacggttaggcgtggtggtttacgtttgtaattttagtattttgggaagtcgaggcgtgtagattatttaaggttaggagttcgagattagttcggttaatatggtgaaatttcgtttttattaaaaatataaaaattagttagatgtggtacgtatttataattttatttattcgggaggttgaagtagaattgtttgaattcgagaggcggaggttgtagtgagtcgtcgagatcgcgttattgtattttagtttgggttatagcgtgagattacgttataaaataaaataaaataatataaaataaaataaaataaaataaaataaaataaaataaaataaaataaaataaaataaaaaaataaaataaaataaaataaaataaagtaatttttttttttttaagcggtttttattttttttttttgttttgtgaagcgggtgtgtaagtttcgggatcgtagcggttttagggaattttttttcgcgatgtttcggcgcgttagttcgttgcgtatatttcgttgcggttttttttttgttgtttgtttattttttaggtttcgttggggatttgggaaagagggaaaggttttttcggttagttgcgcggcgatttcggggattttagggcgtttttttgcggtcgacgttcggggtgtagcggtcgtcggggttggggtcggcgggagttcgcgggattttttagaagagcggtcggcgtcgtgatttagtattggggcggagcggggcgggattatttttataaggttcggaggtcgcgaggttttcgttggagtttcgtcgtcgtagttttcgttattagtgagtacgcgcggttcgcgttttcggggatggggtttagagtttttagtatggggttaattcgtagtattaggttcgggttttcggtagggtttttcgtttatttcgagattcgggacgggggtttaggggatttaggacgtttttagtgtcgttagcggtttttagggggttcggagcgtttcggggagggatgggatttcgggggcggggagggggggtagattgcgtttatcgcgttttggtattttttttcgggttttagtaaatttttttttgttcgttgtagtgtcgttttatatcgtggtttattttttagttcgaggtaggagtatgtgtttggtagggaagggaggtaggggttggggttgtagtttatagtttttcgtttattcggagagattcgaatttttttattttttcgtcgtgtggtttttatttcgggttttttttttgtttttcgtttttttcgttatgtttgtttttcgttttagtgttgtgtgaaattttcggaggaatttgtttttttgttttttttttgtatttttgattttttttcgggttgttgcgaggcggagtcggttcggtttttatatttcgtattttttttttttcgtaggtcgttgcgcggttttgcgtatgttgttggtagattagggttagagttggaaggaggaggtggtgatcgtggagacgtggtaggagggtttatttaaagttttttgcgtaagtgattatgttcgggtaaggggagggggtgttgggttttagggggttgtgattaggatt

SEQ ID NO: 12 - DNA, 2501 bp, Artificial Sequence
  chemically treated genomic DNA (Homo sapiens)
gattttagttatagttttttaaggtttagtattttttttttttgttcgggtatggttatttacgtaggaggttttgagtgagtttttttgttacgtttttacggttattattttttttttttagttttggttttgatttgttagtagtatgcgtagggtcgcgtagcggtttgcggggagggagaagtacgagatgtggggatcgggtcgatttcgtttcgtagtaattcggggaggggttaggagtgtagggagggaatagggaaataggttttttcgaagattttatataatattggggcggggagtaggtatggcgggagaggcggggaataggaaggaggttcggggtaaaagttatacgacggagggataagggggttcggattttttcgggtgggcgaggggttgtgggttgtagttttagtttttgttttttttttttgttagatatatgtttttatttcgaattgggaaatagattacggtgtagggcggtattgtagcgaataaagaaaagtttgttggagttcgggggaggatgttaaggcgcggtgagcgtagtttgtttttttttttcgttttcggggttttattttttttcgaggcgtttcgggttttttgaaagtcgttaacggtattggggacgttttgggttttttaggttttcgtttcgggtttcgaggtgggcgaggagttttgtcgggagttcgggtttgatgttgcgggttggttttatgttgggagttttgagttttattttcggggacgcgggtcgcgcgtatttattggtggcgaagattgcggcggcgaaattttagcgaaggtttcgcggttttcgagttttataagggtggtttcgtttcgtttcgttttagtgttgagttacggcgtcggtcgtttttttggagggtttcgcggattttcgtcggttttagtttcggcggtcgttgtatttcgggcgtcggtcgtagaggggcgttttggagttttcggagtcgtcgcgtagttggtcggggaagttttttttttttttttaggtttttagcggggtttagggagtaaatagatagtaggaagaggatcgtagcgaagtgtgcgtagcgaattggcgcgtcgggatatcgcggggggaaattttttaagatcgttgcgatttcggagtttgtatattcgttttatagggtaggggagaggggtggaggtcgtttagaggaaaggaaattgttttattttattttattttattttatttttttattttattttattttattttattttattttattttattttattttattttattttgtgttattttattttattttatgacgtagttttacgttgtggtttaggttggagtgtagtggcgcgatttcggcggtttattgtaattttcgtttttcgggtttaagtaattttgttttagtttttcgagtaggtggaattataggtgcgtgttatatttggttgatttttgtatttttagtagagacggggttttattatgttggtcgggttggtttcgaatttttgattttaggtgatttgtacgtttcggttttttaaagtgttgggattataggcgtgagttattacgtttggtcgtttaatttttatttgaagttttggggtatatgtagaggatgtgtaggtttgttatataggtgtgtgcgttatgatggtttgttgtatagattattttattatttaggtattaagtttagtattttttagttattttttttggtattttttttttttagtatttcgtttaataggtattagtgtgtgttgatcgtcgttatgtgattatgtgtttttattgtttagtttttatttataagtgagattatgcggtttcgttggttttttgtttttgtgtgagtttgttgaggttaacggtttttagttttatttatgtttttgtaaaggatatgattacgttttttttagtggttgtgttttaggttattttttttggttttgttgtttattttttgttgatttgtagatttttatttattttagatattgattttttgttggttttagatatgatagatagttttttttattttattaattgttaagtttgtttaaggagttttttatgaaataaaattcgttaatttaagtgtaattaaatttagtaagggatttttgtggtggggaagaggttggtgtttatgttgtatttttaaaattttatttaatgtagttattaaaaagaattagattatgttttttgtgggaatatggatggagttagaggttattatttttagtaaattaatgtaggaatagaaatttaaatattggatgtttttatttgtaagtgggagttaaatgatgagaatttataatataaataaggaaataatagatattgtggttgattttagggtgtaggatgggaggaaggagaggagtagaaaagagaattattgggtattcggtataatatttgggtgatgaaatattttgtataataa

SEQ ID NO: 13 - DNA, 1920 bp, Artificial Sequence
  chemically treated genomic DNA (Homo sapiens)
ttaggttgttgtagatatagtttttgtttttttgaaaaatatgttttaggtgttgggattttagatatttgggaaatagagtgtatgtagttgttgagaggttttgtgtttggttttttttattattgaggtgtagaggtgttgtggatagtttagatttatatggtgtttgaggtgaaatagaatttttagtttttttatgaggttattggtatttttggttgtttttagagttttttgatttagagttgaatgtaaagtaagtgtttgaaatgtagaagtagttggggttgtttatggtatttgttttgtttggggtgagagaagatgttaggttgaggttttagtgattttaggtattagttttgaaggagggtggggagattgtaaaggggaagtgtttggagggttaatggtttttgtgtattttgtgtttttttgaagtgtgttgtttttttgtgttggggattgggatttgtttttggggaatttgtttagaagatggtggtggattggggttgggtattttttagggttgttaggttttttttagttttgtatttgttgtgttgttttattttgttaggaagttttagagattttggggatggggtgggagtgtttttttattgtgggtttaaaaagaaggaaggatgtttttaggggttgtagaaggaggattagttttaagttataattttttttggatttaaggtaggttggttggggttttgtgtttatatggtttttggtgggggtttgtgtgttttgggagttttgtggtttggggaggaaagaggagataagagataggtgaggattatggggttgatttagttggggtagggattattgtggaaaaattttggtgaggtggggggatgtggaaagagagtggtttgtgttttgtattttgtgttgggtattttgtgttagtgttttgtttttagtgttttgtgttttgtgttttgtgttttgtttttattttgggttagttgtattgtgtttgtgttgtaggaattgtggagttggaaagtgggggtgttgtggttggggggttgttttagttgtgttttggttagtgattggtgggttgggtttaaatttagttaggttgggtaggtggtggttgtgtgattggggattgggtgttttgttttttttgtttttttttttttttttttttttttttttagttttttggttttttttagtttttattggatttgtttgtttgttgttttttttttttttttgttttttatattattttttatttttttgttttgtttttgtttttttttttttttttgttttttgttttttttttttttttttttttttttaggggtggagtttttttttttttttttagataatgttgtggttgtgtttttttttttgttagtttgtttaggtttttgttgttagtgatttttttgggttgggggtggggaggtggggggggagtgtagggttggggaggatgagttggttttttttatttttttgttgttgttttttttaagagggatggagatttggtttaagttttttggtttatttggagttgtgatagttatttttagggaatagttatgttgttttattaagtttatttttagtggtttggattttttaggtagaggttgtgggattttgttttttttaatattttagtttatttttaaaagggtttgagttggataggggttaaataggtttttttgatttggtgggttggttagatgtgatagtaatgttaaggaggttaagtttttttgtttattttttattttttttttttttatttttggattttttggtgtttttagtatatagaggtttttgagtagtttggttgtaggttttttatttatttagagtttttttttttatgtgtttatttttaattttgta

SEQ ID NO: 14 - DNA, 1920 bp, Artificial Sequence
  chemically treated genomic DNA (Homo sapiens)
tgtagggttggggataggtatgtgagggggagaattttgagtagatagggaatttgtagttgggttgtttaagggtttttgtgtattgggggtgttaggaggtttagggatggaaaagggggaggtgagaaatggataaagaaatttggtttttttggtattgttgttatgtttggttggtttgttaagttgaaggggtttgtttagtttttgtttggtttaaatttttttgggaataagttgggatgttagaaaaaataaaattttataatttttgtttggggaatttaggttgttggaggtgggtttggtagggtagtgtgattgttttttgggagtggttgttatagttttgggtgaattgaggagtttgggttaagtttttattttttttggagagggtagtagtaaggaggtgaggggagttagtttattttttttaattttgtattttttttttatttttttatttttagtttggaagaattgttggtggtgggagtttggatgagttggtggggaaggggatgtagttatagtattgtttgggagggaggggagaagttttgtttttagaggggaggggagaagaggagggggtaggagataaggggaggaagaaaggtgaaggtaaggtgaaggggtggagagtgatatgaagagtgagagaaaagagaggatagtggatgagtagatttggtaggggttgaaaaaggttaaggggttggagggagggagaggaaggaggaggggagtgaggagggtggggtgtttggtttttagttgtgtggttattgtttgtttagtttggttggatttgagtttggtttgttgattgttggttgaggtgtagttgaagtagttttttagttgtggtgtttttattttttaattttatggtttttgtggtgtgggtgtgatgtagttggtttggggtgaaggtaaggtgtggggtgtggggtgtggggtattgggagtgaggtattggtgtgggatgtttggtgtaaggtgtagggtgtgggttgttttttttttgtgtttttttattttgttaaagtttttttatgatggtttttattttggttgggttagttttgtaatttttgtttgttttttgtttttttttttttttttgagttgtggggtttttggggtgtgtggatttttgttaggggttgtgtaggtgtggagttttagttggtttgttttgggtttgaagaaagttgtggtttggagttagttttttttttatgatttttgggggtgtttttttttttttttgagtttgtgatgggaaggtgtttttattttatttttggggtttttgagattttttggtgaggtggggtggtgtggtaggtgtagggttggggagggtttgatagttttggagagtgtttgattttagtttgttgttgttttttaggtggattttttagaggtaggttttagtttttggtgtggggaggtggtgtgttttagaggggtgtagggtgtgtgggggttgttggttttttgggtattttttttttgtggtttttttgtttttttttggagttggtgtttgaggttgttgggattttagtttggtgtttttttttgttttgagtgaggtaggtgttgtgggtggttttggttatttttgtattttgagtgtttattttgtatttagttttaagttggagagttttggggatagttgagagtgttagtggttttatagggagattgagggttttgttttattttgggtgttgtgtgggtttgggttgtttatagtatttttgtgttttagtgataggagaagttaagtgtgaggttttttaatagttgtgtgtattttattttttaggtatttggaattttggtgtttagaatgtgttttttgggagagtaaaggttgtgtttatggtagtttgg

SEQ ID NO: 15 - DNA, 6096 bp, Artificial Sequence
  chemically treated genomic DNA (Homo sapiens)
tattagtgtaagatttaaaattttttttttgtattgtatagtgagatgtttagggttttagtttagtgtttggatatagtgatttttgggtttgtttgttgttgttttaagtgaagttggtgtgttttgggtggagtagatagagattttgggtggtaggggtttgggaagatatgggtggttagggttttatgtgtttttattgttgttttttgttatttgtaggtaatggatgagttgggaatgagttttttagattagtttgtgattaagaaaggtaaggaatggtttgttagggtagagtttggtgagatggtgtaggtttttggtgtatagatttattttttttattggttgtgttttttgtgtgttgttaggttgggtgtttattaggtattttttttggtttagttagatgttaggtagatgtgtgggtttggtgagtttgtttagtattttgtggtttggggtgggttttagtggattagtatttattgggttgtagtattgggagtttggtttttttttgttgagggggagggtatttttgtggatttggagttgatttgtagaatgagttaaattatttttttgtttttttaagagatgggaatggaagtgttgtttttatggagttggggaaatgatttttattttatagtgttttagtattttggtgtttggtgggtatttttttttttttttttttaggtagggttttggaggtttttgggggaattttttttttgtgggagttttttgtggtatttagatttaggggagtttgtgtgtgagtattgtgtgttaggttgtgtgtatttgagttagggtttatttgtttttgggtgtttgtgtttatgtgagtttagggttttgtgtatgtttgaaatgttttttttatgggtgttttagtattttttggagtgtgagtgtgtttgtttttgtgaatgtgtttgtgaggtgtgtttttgtatgttggtgtgtatttttttgtatttgggggatgtatatattttttaatatgtatagtatttttgttgtgttttgtattttgttttttggtatttgaggatttttaagtatgtgtgggttttttttgtgtatatataggagtatttatgtgatttttggtattagtaaaatttagggatatgggatttattttttttggtttgaggattaagtattggttatgataggggaaggtgagagatgataaaaatagagagatagttagagaggagtagagagttagaggggtttaggtattgggtagtagtttttttatatttggggtaggtgtttgaaagaatttagaggtgtatatgagtttgaggtgttttaggtaggtattgtttttatagggtttggtttgagttgttttttaaatgagtgaatttaagtttgggttttatttgttttttatttgtttttaggggaggttaaggtggaagtggtggtagtagggttggggttggatttttaggagttggggttgagttattaggagttgggggttgggtggatgatttggagtgtgtagtagggaagatgaggtaatagggtaggaagtgggtggggggaggtggaattggggttgtgttttgtgttgtttggaattgggagtgtgggaaagatattaggaatttggttgtagtgtagttttgttggtggggtttggttggtttattgtatagagttttttttgatttttgaagaaagagatttgtttgtagtgggtaaaagtttgtttggattttttggttattagaaatatgagtatggtggtggtttttagttttttatttatgtttgggtttaagagattgggagtttaggtttattgattttttgagaaagattaagattttgtattttagaaagaggtttggggatttttgttttgtgtaagggtagaaggattagttgtttttttgagtattttaatttggaattttggtttgaagttgagataggagattggatgtgaggtttttttagagttggttttttttaaataatttttaaaatttttagattttaggggtatgttgaaattttttaaagtagtttaaagaatataatgagagttttaatattttaggtggtggtgtgttggttttttggagtggggtgggatgtggttgtgtggatttatgtgtataattgtgtgggatggggttatgtggatttatgtgtataattgtgggattttagtgttagtgggattttagtgttagtgggattttagtgttagtgggattttagtgttagtgggattttagtgttagtgggattttagtgttagtgggattttagtgttagtgggtttgtggtttagtggagtgagtggagtgttggtgatttgagtggagattgtgttttggatgttttagtttagatgttaagttatagtttgtgtagtagtagtaaaggggaaggggtaggagttgggtatagttggatttggaggttgtgatttaggggaaagtgtgggtggttgatttagggtagttgtggtggtgaggtaggtgggttttttgttttttggagttgtttttttttatatttgtttttggtgtttttagtagtttttattttggttttttgtggttattgtgggatttggtgttgttgttagtttagtggggagtgaattagtgtttttttttgtttttggtttttttgatggtatgaggaatttttgttttgttttatagatttttggtttttgttgagtgtggtattggagtttgttttgttagggttttggaattagagaaagttgttttttggttatttgaagtgttggatttttatagtgttttttagtttgggtgggagtggtggttgtgttgttgaaggttggggtttttggtgtgaaagggaggtagttgtagttttagttttattttagaagtggtttttgtattgttgtggtgggtgtttttgggttttgattttgttagtgttgtggggtagaggtatttggagtttgtagggtttagatttgggttggaaaagttttgttgattgtaggtaagtgtttgggaggggtggttaggtgaagttttggtgttttattatatatttttgggttttatgttagttgtatttgtggtattgggtaggaaatggtagggttgaggttgattttaggagtataagggagttttttattttttgtttatatttgttatttttagttttgtaatttattttagatatatagaaagtaagtaggattggtggggagatggagtttaataggaatattttttagtagtgagtaggggttgtatgggatgtgggaggagtttagaggaggtgtggagagtgtttgaggttgggtgagtgtttagaggggagatagttgaattgggtttaagaggtgtttagtgggtgtttgttgaatgaatgagtgatgggttttgaagtttgagtgtattgaaagagggggtgtgtaaaaagggttttttttattatataggatatagtatatgtaaattttttttttgtggaaaagttagataggttaaaaaggttataaataaattagttgggtatggtggtgtgtgtttgtagttttagttattagggaggttgagttaggggaattgtttgaatttgggaggtggagattgtagtgagttaagattgtgttattgtattttagtttggaaatagagtgagattttgttttggaaaaaaaaaaaaaaagttataaattgtgtgtgggttttaggttatataattagagttggaggggagtggttaaggatgagaattgagatggattttttgttttttttggaggagagtgggtggttgtttatttgggggtggggaatttttttttatgggtttagttgtttaattttaggggatttttaggataggagttgatgtaaatagttgttttattttttgttgtttttggttttggagaaggaggagggagttggggagggtttttattttttagataatttttaagtagttaggatatgggtgagatgagtgagatattgatttttgggatagaatttgagagggtgttaaaaaatttagtaattaagataaataggttgggtgtagtggtttatgtttgtaattttagtattttgggaggttggattatttgaggttaagagtttgagattagtttggttaagatggtgaaattttatttttattaaaaatataaaaattagtttagtgtggtggtgttagtttgtaattttagttatttaggaggttgaggtaagagaattgtttgatttaggaggtagaggttgtagtgagttgagattatgttattgtattttagtttggataatagagggagattattttaaaaaaaaaaaaaaaaaaaaaaaaaaaaaagaggttgggtggtggtggtttatattatgtgattttagtattttgggaggttgaggtgggtggattatttgaggtttggagtttgagattagtttggttaatatggtgaaattttgtttttattaaaaatataaaaattagttgggtggggtggtaggtatttgtaattttagttattttggaggttgaggtaggagaattttttgaatttgtggggtggaggttgtagtgaattaagattatattattgtattttagtttggataataatagtaaaattttgttttaaaaaaaaaaaaaatttttttttttgagatatagttttatttttttgtttaggttggggtgtagtattatgattttagtttattgtaatttttgttttttagatttttgtattttagttttttaagtagttgggattataggtatttgttattatgtttagttaatttttgtatttttagtaggggtgtggttttattatgttggttaggttggttttgaatttttgattttaagtgatttgtttgttttagttatttaaagtgttgggattataggtgtgagttattatgtttggtttttttaaatgaaaatagtgtaaaaatttatgataaataaaatattaaaaatttattgaatttgtatttttataatttttttttatttgttttttaggttattttttgttttagaaagtaatttaaaaaatgtgtagatggagtttggattttatttgaaaatggtgggagttatggaaaattttggagtaggggagtgaaggatagaaattatatgtaaaagaaattttgggttgggtgtagtggtttatgtttgtaattttagtattttgggaggttgaggtaggtggattatttgaggttaggagattgagattagtttgattaatatggtgaaatgttatttttattaaaaatataaaaaaaattagttaggtatggtggtgtatgtttgtagttttagttattttggaggttgagataggaaaattgtttgaatttgggaggtggaggttgtagtgagttaagattgtgttattgtattttagtttgggtaataagagtaaaattttattttaaaaaaaaagaaagaaagaaattttttggtagttgatgagaaggaaatttaattggtaggttttagtaggggagatgaggagattttagggagggtatttgtatatgttgtgttttagtgtgggttagggagtaggttattattttttttgtttattttttttttgttttaatttttttaagttttggattagtggtattttaagtgtagtttaaggaattatatgtattaggatttttagggggtgtttgttaaaaatgtaaattttggttaggtgtagtggtttatatttgtaattttagtattttgggaggttgaggtgggtggattatgaggttaggagattgagattattttggtaaatatggtgaaattttatttttattaaaaaaataaaaataaattaaaaaaaatattagttgggtgtggtggtgggtgtttgtagttttagttatttgggaggttgaggtaggagaatggtgtgaatttgggaggtggagtttgtagtgagttgagattgtgttattgtattttagtttgggtgatagagtgagattttgttttaaaaaaaaaaaagtaaattttttgggtattattttatattgattg

SEQ ID NO: 16 - DNA, 6096 bp, Artificial Sequence
  chemically treated genomic DNA (Homo sapiens)
tagttaatgtggggtggtgtttaagaaatttgtttttttttttttgaggtagagttttgttttgttgtttaggttggggtgtagtggtgtaattttagtttattgtaagttttgttttttgggtttatgttatttttttgttttagttttttgagtagttgggattataggtgtttgttattatgtttagttaatgttttttttggtttgtttttgtttttttagtagagatggggttttattgtgtttgttaggatggttttgattttttgattttgtgatttatttgttttggttttttaaagtgttgggattataggtgtgagttattgtatttggttaaaatttgtatttttaataagtattttttgggggttttgatgtatgtggttttttggattatatttagggtattattggtttaaagtttgaaggggttggagtaagaggaaggtagatgggaggagtagtgatttgttttttggtttatattggggtatagtatgtgtaaatgttttttttagagtttttttattttttttgttgggatttgttgattaagttttttttttattaattgttagagggtttttttttttttttttttttaagatggagttttgtttttgttgtttaggttggagtgtaatggtataattttggtttattgtaatttttgttttttgggtttaagtgatttttttgttttagtttttggagtagttgggattataggtgtgtattattatgtttggttaattttttttgtatttttagtagagatgatattttattatgttggttaggttggttttaattttttgattttaggtaatttatttgttttggttttttaaagtgttgggattataggtataagttattgtgtttggtttagggttttttttatatataatttttattttttatttttttgttttaaggttttttatagtttttattatttttaggtaaagtttaaattttatttgtatattttttaagttgttttttggggtagagagtagtttgggaggtaggtgagaaagggttgtggaagtgtaagtttagtaaatttttgatattttgtttattgtggatttttgtattatttttatttaaaaaggttaagtgtggtggtttatgtttgtaattttagtattttgggtggttgaagtgggtaggttatttgaggttaggagtttaagattagtttggttaatatggtgaaattatgtttttattaaaaatataaaaattagttgggtgtggtgataggtatttgtaattttagttatttgggaggttgaggtatgagaatttgagaggtagaggttgtagtgagttgagattgtggtgttgtattttaatttgggtgagagagtaaaattgtgttttgaaaaaaaagattttttttttttttgagatagagttttgttgttgttgtttaggttggagtgtaatggtgtgattttggtttattgtaatttttgttttgtaggtttaagggatttttttgttttagtttttggagtagttgggattataggtgtttgttattttatttagttaatttttgtatttttagtagaaatggggttttattatattggttaggttggttttgaattttagattttaggtgatttatttgttttggttttttaaagtgttgggattatatggtgtgagttattgttgtttggttttttttttttttttttttttttttttttttttgagatagtttttttttgttgtttaggttggagtatagtggtatgattttggtttattgtaatttttgttttttgggttaagtaatttttttgttttagttttttgagtggttgggattataggttagtgttattatattgggttaatttttgtatttttagtagagatggggttttattattttggttaggttggttttgaatttttgattttaagtgatttggttttttaaagtgttgggattatagatgtgagttattgtgtttggtttatttattttgattattgagttttttggtatttttttaaattttgttttagaagttagtattttatttattttatttatgttttggttgtttagagattgtttgggaagtggagattttttttagtttttttttttttttttagggttaaagatagtaaggaatagggtgattgtttatattagtttttgttttagagattttttgagattggatagttgagtttgtggagagggattttttatttttaagtaggtaattatttatttttttttagagggaatgagggatttattttagtttttatttttgattatttttttttagttttgattgtataatttgaaatttatatatggtttgtaattttttttttttttttttgagatggagttttgttttgtttttaggttggagtgtagtggtgtgattttggtttattgtaatttttgttttttgggtttaagtgatttttttggtttagtttttttagtagttgggattatagatgtgtattattatgtttggttaatttgtttgtaatttttttaatttgtttggtttttttatagggagaggatttgtatatgttgtgttttgtgtgatgaaaggagttttttttatatatttttttttttaatgtatttagattttaaagtttattatttatttatttaataaatatttattaagtattttttgaatttggtttaattattttttttttaggtatttatttaattttgggtattttttgtgttttttttgagttttttttgtgttttatatagtttttgtttattgttggaaaatatttttgttaagttttgtttttttattagttttgtttgttttttgtgtgtttgggataggttgtaaaattggaggtgataaatgtgggtaggaaatggagggtttttttatatttttagggttggttttagttttgttattttttgtttaatattgtggatgtaattggtatgggatttggaagtgtgtggtaaagtgttggggttttgtttggttgttttttttggatgtttgtttgtagttagtgaagtttttttaatttaggtttgggttttgtgagttttaggtgtttttgttttgtggtgttggtgaagttgaagtttgagaatgtttattgtagtgatgtgaaggttgtttttggggtggggttgaggttgtagttgttttttttttgtattaaggattttaatttttagtgatgtagttgttgtttttgtttaggttgggaggtattgtagggatttgatgttttaggtggttaaagagtgattttttttgattttagggttttggtggggtaggttttagtattgtatttggtggaggttgaaggtttgtggggtaggataggagttttttgtgttgttggaagggttgaggatgaaggagggtgttaatttattttttattgggttggtggtaatgttgaattttgtagtgattgtggagggttaaggtgaaaattgttgggggtgttgagggtaggtgtggggaggggtggttttagggagtaaggagtttatttgttttgttgttgtagttgttttgggttgattgtttatgtttttttttgggttatgatttttggatttaattgtgtttggtttttgtttttttttttttgttgttgttgtgtgggttgtaatttgatgtttaggttggggtgtttagggtgtagtttttgtttaggttgttagtgttttatttgttttattgggttatagatttgttggtgttggggttttgttggtgttggggttttgttggtgttggggttttgttggtgttggggttttgttggtgttggggttttgttggtgttggggttttgttggtgttggggttttgtggttgtgtatgtgagtttgtgtggttttgttttgtgtggttgtgtatgtgagtttgtgtggttgtgttttgttttgttttagggagttagtgtgttgttatttgggatgttaggatttttgttgtgttttttggattgttttgggggattttggtgtatttttaggatttaggagttttggaagttgtttgagagaaattagttttgggagggttttgtatttagttttttgttttggttttggattggggttttgggttaaggtgtttagaggaatagttgatttttttatttttgtgtagggtagagatttttaaatttttttttaaaatgtagggttttagttttttttagggagttagtgaatttagatttttagttttttgagtttaagtatgaatagggaattggggattattattatgtttatatttttggtggttaggaagtttaggtaggtttttgtttattgtagatggatttttttttttaggggttaagaaaggttttgtatagtaagttaattaagttttattagtagagttgtgttgtaattaggtttttagtgttttttttatatttttagttttaagtgatataggatatagttttaattttatttttttttatttattttttgttttgttgttttattttttttgttatatattttaagttatttatttaatttttagtttttggtaatttagttttagtttttggaagtttagttttagttttgttattattatttttattttggttttttttgagaataagtggagggtaaatagagtttaggtttgaatttatttgtttgaaaaataatttaagttaaattttgtgggagtagtgtttgtttggggtattttaggtttatgtgtatttttgaatttttttgggtatttgttttaaatgtaaagaggttgttatttaatgtttgggtttttttgattttttgtttttttttggttgtttttttgtttttgttgttttttatttttttttgttatggttagtatttggtttttaggttagaaaaggtggattttgtgtttttggattttattaatgttaggagttatataaatatttttatatatatatagagagggtttgtgtatgtttggaaatttttagatattaaagaataaagtgtaggatataatagagatattgtatatattgagaaatgtgtatattttttaaatgtagagaaatgtatattaatatatagagatatattttataaatatatttatagaaatagatatatttatattttaagaaatattaagatatttatgaagggaatattttagatatgtataggattttgaatttatatggatatagatatttaggagtaggtgggttttgatttaggtgtatatagtttaatatatagtatttatatataagtttttttaagtttaaatgttgtaagagatttttatagaaagaaaatttttttagaggtttttaaggttttgtttggaaggaagaggaagaaagtgtttgttaggtattgaaatgttaaggtattgtaaagtgaaaattatttttttaattttgtgggaatagtatttttatttttattttttagggaaatagggaagtggtttaatttgttttgtaaattaattttagatttataagagtgtttttttttttggtggggagaggttaggtttttagtgttgtagtttagtgaatgttgatttgttgaggtttattttaggttatagggtgttgggtaaatttattaagtttgtatgtttgtttaatatttggttgggttaggaagagtgtttggtggatatttagtttggtgatatataggaggtatggttggtgaagaaaatggatttgtgtattaagggtttgtattattttgttgggttttgttttgatagattattttttattttttttgattatggattggtttaggaggtttatttttggtttgtttattgtttgtaaatagtagagggtagtggtgagggtgtataaagttttagttgtttatgtttttttaagtttttgttatttagggtttttgtttgttttgtttaaggtgtattagttttgtttggggtggtgatgggtaggtttaggaattgttatgtttaggtattgagttggagttttgggtattttattgtgtagtgtaaaaagggaattttgaattttatattggta

SEQ ID NO: 17 - DNA, 2501 bp, Artificial Sequence
  chemically treated genomic DNA (Homo sapiens)
tttgtaaatggagatatttttattatttttatagtattatatgtttttaaagtttgtatttatattttgggtgataaatgaaggataagatttttttttatttttgtgaggatgattatagtatgattggatgggtttgttatgatttttatttttttttgtgtttttattattgttttattaattttagtttttttttatagggtagtatagaatttaattagtagaaagagatttagttatgtagattagagatttgtttaagtgatggtatgtaagaattaggaaggaaagttttttgtttaaatattaataggttttttttttaaagtaattattattttttaaatttaatttataaggtgatagtatttttaaattaattaaattagaattttgggttggataattttaaatatgatttattagtattttttattaattattggttttttaggtttttaagtttatttattaggaattttatttttaatattattttattaattttagttgtaaataagagaatatttaaaggttgaggaatttttagtggtaaagttttgtttatgttaagtaataaaggataagttagtttttgttgtgattattttgttgtattgataagttatgtatttttatttaaggatttaaatttttattttttttaagaattgggttaaaattgataaattaaatttatttatggtttattgattaaaggttgttgtataataagtttttgttatgtttagtagttggatttatagtgttagaaatttataattgtttgatttttttttttattatattgtgaaaattgttttttaaatgtaattaattttaaaattttaatagtattgtggttaggtgtggtggtttattattgtaatattaatattaggtataggtgaggggattgaggttaggatattgaaattagtttgggaaatatatggagatttggtttttggaaaaataattagttttgtgtggtggtgggtgtgaggttttggttaattgggaggttatagtgagttatgatgatattgtattatagtttgtgtgatggtttatgttagtaagttttggagtatttgaaataagttgtgttgggtattttatttattggagagtgattagtgattgatgtttatttatagtgattagagatgtatgttttgatagtagtataaatttagtaggtgtgaataaatggtaaagagaaattgggtaaataagtattatggttttttagttgagaaagtgggggttttaaaaagggttttttgttgatagaaagggatgtttaattattgaaattgtagagggtgtggttttggtgtttgagtgtgtagattatatttatggtggtgattgttttgtgtttggtgtgttttgtataggttatggtgttttggattatgttttttaggaatatttttagtattttgtgagtttttttgtagatgaggttggagatgtgttttatgttgttgtggtgagtaaggtgttggatggttggtttggtgatgttttggatattgttgtgtagtattttatggtggtgtttagtgttgtttttgttaagattttttttgtttttgttgtggttagatatgatgagtaagaggagttttatttaatgttttgtgaggattttggtttgaggtagtgtttttatatgatagttggtggattgaattgagaatttgaaagaagttggtgggaagttttgttttggtgggggaggggaaatttaaagggttaaattgaaatagggggaaaaaaaaagtgagttttttgtttttgtgttttgaattttgtaatgtgtatagtattttgttattatgttatgaggttttaaaaaattgtttttgaatgtagaagatatatattaatattgtgggaaatataagaaaggataagaaattaagaaattataatgttattttattatataggttagttaattatgtattttgtagagtagttgtatatatttttttaagaaaatgtatatagtgttgtatatggagttttgtaatttttttatattgattataatttaattaatttttattaaagagataaaagtgatgttttggtgtttatgttttttaggaattattaatagttataattagttttttagtaattttttaattggttgtattttaaaaataatgttttttatatttaatataaatgtattttttttttatatttgggattaatattgaaatttatgattttattatattaaaatttaaattttattatattaatatttaaaattgtattagaggttttatgatttggtattatgggtttttgtattattttttttttaaattttttaatttgttttattaaggtttttggataattttagagattttttgtgaagtttgaataaaatttttttgagattttgataattgtattagttttaggatttaattggaatagaattaaaatttttaaaataagtttttata

SEQ ID NO: 18 - DNA, 2501 bp, Artificial Sequence
  chemically treated genomic DNA (Homo sapiens)
tataagagtttgttttaaggattttaattttattttaattaagttttaaagttaatgtaattattaaaattttgaagagattttatttaaattttataaaaggtttttaaagttgtttagaaattttggtgaaatagattaggaaatttggaaaggaaataatgtggagatttgtagtattaaattatgagatttttaatataattttaaatattaatgtaataaaatttaaattttggtgtaataaaattataaattttaatattggttttaagtatagagaaaaagtatatttatgttgaatgtggaaaatattatttttaaaatatagttgattaaaaaattgttggggaattgattataattattgataatttttaagaaatatagatattaaaatattatttttatttttttaatagaaattggttaaattataattaatataaggaggttataaaattttatatataatattgtatatatttttttggaaaaatatgtgtaattgttttgtaaaatatatgattaattagtttgtgtgatgggataatattgtagttttttaattttttgtttttttttgtattttttatagtattgatgtatattttttgtgtttaaaagtaattttttaaagttttataatgtggtaataaaatattatgtatgttataaaatttagaatatggaaataagaagtttgttttttttttttttttattttggtttggttttttagatttttttttttttattggggtgggattttttgttgattttttttaggtttttagtttggtttgttaattgttgtataaaggtgttgttttaggttagagtttttataaagtgttgggtgagattttttttgtttgttatgtttggttgtggtaaaggtgggaagggttttggtaaaggtggtgttaagtgttattgtaaagtattgtgtgataatatttagggtattattaagttggttatttggtgttttgtttgttgtggtggtgtgaagtgtatttttggttttatttatgaggagatttgtggggtgttgaaggtgtttttggagaatgtgatttgggatgttgtgatttatatagagtatgttaagtgtaagatggttattgttatggatgtggtttatgtgtttaagtgttagggttgtattttttatggttttggtggttgagtgtttttttttattaataaaaggttttttttagggtttttatttttttagttgaggagttgtgatgtttgtttgtttagtttttttttattatttgtttgtgtttgttgagtttgtgttgttattggagtatgtgtttttagttgttgtaagtaggtattagttattaattgttttttagtaaataaaatatttaatataatttgttttaggtgttttagagtttattgatatgggttgttgtgtagattgtagtgtagtgttattatggtttattgtagttttttgattagttggaattttgtgtttattattatgtaaggttaattatttttttaaagattgggtttttgtgtgttttttaggttagttttgatattttggttttaatttttttgtttatgtttaatgttggtattatagtagtgagttattatgtttggttatgatattgttgaggttttagggttagttatatttaaggggtaatttttgtagtgtagtggggaggaaagttaagtagttataggtttttggtgttgtgaatttaattgttgaatatagtaagaatttattatgtaataatttttaattagtggattgtaaataagtttagtttattggttttggtttaatttttgagaaaggtgagaatttgaatttttgagtagaaatatgtagtttattagtataataaagtgattataataaagattaatttattttttgttatttaatgtgggtagagttttattgttgagaattttttagtttttgagtgtttttttatttataattgaagttgataagatggtattaaaagtgagatttttagtaagtaaatttaaaggtttgaaggattagtgattaatgggaagtgttaataagttatatttgaggttatttaatttgagattttgatttaattggtttaaggatattattattttgtgggttagatttgaaaaataataattgttttaaggaaggaatttgttggtatttaaataaaaaatttttttttttgatttttatatgttgttatttagataaatttttggtttatatggttggatttttttttgttagttaaattttgtgttattttgtgaaaaagaattgagattaataaaatggtagtgagaatatagggaaagataaaaattatagtaagtttatttagttatgttgtagttatttttataaggatagggaaggattttgttttttatttattatttaaagtgtgagtataaattttaaaaatatatgatattataggaataatgaagatgtttttatttgtaaa

SEQ ID NO: 19 - DNA, 2501 bp, Artificial Sequence
  chemically treated genomic DNA (Homo sapiens)
ttgttgtatagaatattttattatttaggtattatgttgagtatttaatagtttttttttttgtttttttttttttttttattttgtattttggagttaattatagtgtttgttgtttttttgtttgtgttataagtttttattatttagtttttatttataagtgagaatatttagtatttggatttttgtttttgtattagtttgttaaggataatagtttttagttttatttatgtttttataaaagatatgatttagttttttttaatggttgtattaaatgaagttttaaagatataatataaatattaattttttttttattataaaaattttttgttgaatttgattatatttaaattaatgagttttgttttatgaaagattttttggataaatttgatagttgatggaataggagaagttgtttgttatgtttaaagttaataagagattaatatttagaataaatggagatttgtaaattaatagaaagtaggtagtaaagttaaagaaaatagtttaaggtatagttattaaaaggaatgtgattatgttttttgtagggatatgggtggagttggaagttgttagttttagtaaatttatataggaatagaaaattagtgagattgtatggttttatttataagtgggagttgaataatgagaatatatggttatatggtggtgattaatatatattggtgtttgttgagtggggtgttggggagggagagtattaggaagaatagttaagggatattgggtttaatatttgggtgatgggatgatttgtatagtaaattattatggtgtatatatttatgtaataaatttgtatattttttatatgtattttagaattttaaataaaagttggatggttaggtgtggtggtttatgtttgtaattttagtattttgggaagttgaggtgtgtagattatttaaggttaggagtttgagattagtttggttaatatggtgaaattttgtttttattaaaaatataaaaattagttagatgtggtatgtatttataattttatttatttgggaggttgaagtagaattgtttgaatttgagaggtggaggttgtagtgagttgttgagattgtgttattgtattttagtttgggttatagtgtgagattatgttataaaataaaataaaataatataaaataaaataaaataaaataaaataaaataaaataaaataaaataaaataaaataaaaaaataaaataaaataaaataaaataaagtaatttttttttttttaagtggtttttattttttttttttgttttgtgaagtgggtgtgtaagttttgggattgtagtggttttagggaatttttttttgtgatgttttggtgtgttagtttgttgtgtatattttgttgtggttttttttttgttgtttgtttattttttaggttttgttggggatttgggaaagagggaaaggtttttttggttagttgtgtggtgattttggggattttagggtgtttttttgtggttgatgtttggggtgtagtggttgttggggttggggttggtgggagtttgtgggattttttagaagagtggttggtgttgtgatttagtattggggtggagtggggtgggattatttttataaggtttggaggttgtgaggtttttgttggagttttgttgttgtagtttttgttattagtgagtatgtgtggtttgtgtttttggggatggggtttagagtttttagtatggggttaatttgtagtattaggtttgggtttttggtagggttttttgtttattttgagatttgggatgggggtttaggggatttaggatgtttttagtgttgttagtggtttttagggggtttggagtgttttggggagggatgggattttgggggtggggagggggggtagattgtgtttattgtgttttggtatttttttttgggttttagtaaatttttttttgtttgttgtagtgttgttttatattgtggtttattttttagtttgaggtaggagtatgtgtttggtagggaagggaggtaggggttggggttgtagtttatagttttttgtttatttggagagatttgaatttttttatttttttgttgtgtggtttttattttgggttttttttttgttttttgttttttttgttatgtttgttttttgttttagtgttgtgtgaaatttttggaggaatttgtttttttgttttttttttgtatttttgatttttttttgggttgttgtgaggtggagttggtttggtttttatattttgtatttttttttttttgtaggttgttgtgtggttttgtgtatgttgttggtagattagggttagagttggaaggaggaggtggtgattgtggagatgtggtaggagggtttatttaaagttttttgtgtaagtgattatgtttgggtaaggggagggggtgttgggttttagggggttgtgattaggatt

SEQ ID NO: 20 - DNA, 2501 bp, Artificial Sequence
  chemically treated genomic DNA (Homo sapiens)
gattttagttatagttttttaaggtttagtattttttttttttgtttgggtatggttatttatgtaggaggttttgagtgagtttttttgttatgtttttatggttattattttttttttttagttttggttttgatttgttagtagtatgtgtagggttgtgtagtggtttgtggggagggagaagtatgagatgtggggattgggttgattttgttttgtagtaatttggggaggggttaggagtgtagggagggaatagggaaataggtttttttgaagattttatataatattggggtggggagtaggtatggtgggagaggtggggaataggaaggaggtttggggtaaaagttatatgatggagggataagggggtttggatttttttgggtgggtgaggggttgtgggttgtagttttagtttttgttttttttttttgttagatatatgtttttattttgaattgggaaatagattatggtgtagggtggtattgtagtgaataaagaaaagtttgttggagtttgggggaggatgttaaggtgtggtgagtgtagtttgttttttttttttgtttttggggttttatttttttttgaggtgttttgggttttttgaaagttgttaatggtattggggatgttttgggttttttaggtttttgttttgggttttgaggtgggtgaggagttttgttgggagtttgggtttgatgttgtgggttggttttatgttgggagttttgagttttatttttggggatgtgggttgtgtgtatttattggtggtgaagattgtggtggtgaaattttagtgaaggttttgtggtttttgagttttataagggtggttttgttttgttttgttttagtgttgagttatggtgttggttgtttttttggagggttttgtggatttttgttggttttagttttggtggttgttgtattttgggtgttggttgtagaggggtgttttggagtttttggagttgttgtgtagttggttggggaagttttttttttttttttaggtttttagtggggtttagggagtaaatagatagtaggaagaggattgtagtgaagtgtgtgtagtgaattggtgtgttgggatattgtggggggaaattttttaagattgttgtgattttggagtttgtatatttgttttatagggtaggggagaggggtggaggttgtttagaggaaaggaaattgttttattttattttattttattttatttttttattttattttattttattttattttattttattttattttattttattttattttgtgttattttattttattttatgatgtagttttatgttgtggtttaggttggagtgtagtggtgtgattttggtggtttattgtaatttttgttttttgggtttaagtaattttgttttagttttttgagtaggtggaattataggtgtgtgttatatttggttgatttttgtatttttagtagagatggggttttattatgttggttgggttggttttgaatttttgattttaggtgatttgtatgttttggttttttaaagtgttgggattataggtgtgagttattatgtttggttgtttaatttttatttgaagttttggggtatatgtagaggatgtgtaggtttgttatataggtgtgtgtgttatgatggtttgttgtatagattattttattatttaggtattaagtttagtattttttagttattttttttggtattttttttttttagtattttgtttaataggtattagtgtgtgttgattgttgttatgtgattatgtgtttttattgtttagtttttatttataagtgagattatgtggttttgttggttttttgtttttgtgtgagtttgttgaggttaatggtttttagttttatttatgtttttgtaaaggatatgattatgttttttttagtggttgtgttttaggttattttttttggttttgttgtttattttttgttgatttgtagatttttatttattttagatattgattttttgttggttttagatatgatagatagttttttttattttattaattgttaagtttgtttaaggagttttttatgaaataaaatttgttaatttaagtgtaattaaatttagtaagggatttttgtggtggggaagaggttggtgtttatgttgtatttttaaaattttatttaatgtagttattaaaaagaattagattatgttttttgtgggaatatggatggagttagaggttattatttttagtaaattaatgtaggaatagaaatttaaatattggatgtttttatttgtaagtgggagttaaatgatgagaatttataatataaataaggaaataatagatattgtggttgattttagggtgtaggatgggaggaaggagaggagtagaaaagagaattattgggtatttggtataatatttgggtgatgaaatattttgtataataa

SEQ ID NO: 21 - DNA, 20 bp, Artificial Sequence
  chemically treated genomic DNA (Homo sapiens)
gggattatttttataaggtt

SEQ ID NO: 22 - DNA, 21 bp, Artificial Sequence
  chemically treated genomic DNA (Homo sapiens)
cccatactaaaaactctaaac

SEQ ID NO: 23 - DNA, 33 bp, Artificial Sequence
  chemically treated genomic DNA (Homo sapiens)
ctaaaccccatccccaaaaacacaaaccacaca

SEQ ID NO: 24 - DNA, 24 bp, Artificial Sequence
  chemically treated genomic DNA (Homo sapiens)
agtttcgtcgtcgtagttttcgtt

SEQ ID NO: 25 - DNA, 25 bp, Artificial Sequence
  chemically treated genomic DNA (Homo sapiens)
ttttttaggaatatttttagtattt

SEQ ID NO: 26 - DNA, 17 bp, Artificial Sequence
  chemically treated genomic DNA (Homo sapiens)
ccaaaacatcaccaaac

SEQ ID NO: 27 - DNA, 33 bp, Artificial Sequence
  chemically treated genomic DNA (Homo sapiens)
caaaccaaccatccaacaccttactcaccacaa

SEQ ID NO: 28 - DNA, 23 bp, Artificial Sequence
  chemically treated genomic DNA (Homo sapiens)
cgtagatgaggtcggagatgcgt

SEQ ID NO: 29 - DNA, 18 bp, Artificial Sequence
  chemically treated genomic DNA (Homo sapiens)
ctaaaacctcaacctaac

SEQ ID NO: 30 - DNA, 24 bp, Artificial Sequence
  chemically treated genomic DNA (Homo sapiens)
gatttagagttgaatgtaaagtaa

SEQ ID NO: 31 - DNA, 31 bp, Artificial Sequence
  chemically treated genomic DNA (Homo sapiens)
cctaacatcttctctcaccccaaacaaaaca

SEQ ID NO: 32 - DNA, 23 bp, Artificial Sequence
  chemically treated genomic DNA (Homo sapiens)
aacgaaacaaataccgtaaacga

SEQ ID NO: 33 - DNA, 20 bp, Artificial Sequence
  chemically treated genomic DNA (Homo sapiens)
aaacccaaacctaaattaaa

SEQ ID NO: 34 - DNA, 17 bp, Artificial Sequence
  chemically treated genomic DNA (Homo sapiens)
ggaagtgtgtggtaaag

SEQ ID NO: 35 - DNA, 28 bp, Artificial Sequence
  chemically treated genomic DNA (Homo sapiens)
taaagtgttggggttttgtttggttgtt

SEQ ID NO: 36 - DNA, 23 bp, Artificial Sequence
  chemically treated genomic DNA (Homo sapiens)
aaacaaacgtccgaaaaaaacga

SEQ ID NO: 37 - DNA, 1700 bp, Homo Sapiens
cccctcaggactagagaatcccgaagagcccgtgtattgttttcttgaaagccccgaagctaagcaattctaaaaatgtttagcaaagcccttttgggaatggtgagatggggggtggggaggatgggacattttcattgccaccgcccgccccccgtctccccggccccctcacatgaagtgactggatttgggagacctagcggaaaccttcaggtcacccagggaactgctcctctctctggtcctgcagcggcgaggcggggtagggggtggggggggtgggccgtgagagcgccgagacgcgcggggcgcggagatgtgcaagtggcgaagcttgaccgagagcaggctggagcagccgcccaactcctggcgcgggatctgctgaggggtcacggtgagtctctgtgggtgcgggtcggagggatgcccaggtgcctcgtcgccccacggcccgggcggaggcgggtccggaacctcccccaagtccggcctcagcctggagcccgcgcgccttgggcggatgcacggactgagaggcgcttggctgggctctcagcccgccactgccacccggccccgtgcgctcattggtcgggatgtgtcggctcagcagcctccaacttctcccgaatcccactggtgagtcccaggagagcgagctgagggagaaaggtccaaagggcgcgagcgcccagggcgctgggagcccgtgggacgggcacggggagggcagagccagccgaggggagcggctgccgggaggtgcgctgagcagggtccgcagctaaggtccagcgcgatccgagcgcccaggaccctgcggccgtggaggaggcgtgccctgggaaggagccgctactgggacctgaagagtgaggggagggagaggggcccagagggtgacctggaggagggctctggaagtcacgtcaggttggctcttcaggttcatttccatagttccctgcggcctctgccttggggagttatgttttgttaccgagatccgcgctaccagattgcaccggggctgatttgggggctgggaatttgccattctgctgtacagacactgatttttttttcttctttttaaaaagcaaggtttgttttcattttggtttctgtcggattttctcatttgtaactcacttttctatttttacttttcttaaccggaagatcttttcattatcccacctcttcatcttgcttttaacctgatttcctattatataattaatgcttaaatctcacaagggttggtcggtgcggggctggtgtgtccttttgtagatgaaagtttaagacctcttaactggtttcagtctatttgtctggtttcaaaataagggtaattgcaaccaaatatccctctttagcttctcgatcaagtcgattcctcccccatatatgattcaaatggcaattttattaaactgaaaactagtacctgacttgcagcgtcagcatttggaaagtgcttgtcggcaaatcgactgtatgaaggcgtgtagcttcatcccctgccatttttgtgggttctagcactttaaaaaattgtctggtaatttcttggacttaaccctgtgtctttggttgtagagcagcactaaccaggggagtaatatggttggcacttcgtgtccagaactccgtatttcaggctgggaaacacctaattaatgtctggg

SEQ ID NO: 38 - DNA, 1700 bp, Homo Sapiens
cagctggtaagtggcagacctggaattggaatctagttcagtttgattccccaacccatgctcttgaccactatactgttttttcaagtccagatctgaaatctcattttctgtgtggctgtgtgtttgggacaggggtaaccaattcctgactactctatatgctgcatagaacctggagaggatttttcaaagtaaatgaatctcgaaagctggattgcagagcaaacgagtgcagtcaattcagccaggggcttgcaagagggagaaagagaaaaagactgtggaatggaaagtttcccaacccaagcctttcccaaggggtagccattctctgttctacagtttagggcttgcatgtgctttttctggagtggaaaaatacataagttataaggaatttaacagacagaaaggcgcacagaggaatttaaagtgtgggctggggggcgaggcggtgggcgggaggcgagcgggcgcaggcggaacaccgttttccaagctaagccgccgcaaataaaaaggcgtaaagggagagaagttggtgctcaacgtgagccaggagcagcgtcccggctcctcccctgctcattttaaaagcacttcttgtattgtttttaaggtgagaaataggaaagaaaacgccggcttgtgcgctcgctgcctgcctctctggctgtctgcttttgcagggctgctgggagtttttaagctctgtgagaatcctgggagttggtgatgtcagactagttgggtcatttgaaggttagcagcccgggtagggttcaccgaaagttcactcgcatatattaggcaattcaatctttcattctgtgtgacagaagtagtaggaagtgagctgttcagaggcaggagggtctattctttgccaaaggggggaccagaattcccccatgcgagctgtttgaggactgggatgccgagaacgcgagcgatccgagcagggtttgtctgggcaccgtcggggtaggatccggaacgcattcggaaggctttttgcaagcatttacttggaaggagaacttgggatctttctgggaaccccccgccccggctggattggccgagcaagcctggaaaatggtaaatgatcatttggatcaattacaggcttttagctggcttgtctgtcataattcatgattcggggctgggaaaaagaccaacagcctacgtgccaaaaaaggggcagagtttgatggagttgggtggacttttctatgccatttgcctccacacctagaggataagcacttttgcagacattcagtgcaagggagatcatgtttgactgtatggatgttctgtcagtgagtcctgggcaaatcctggatttctacactgcgagtccgtcttcctgcatgctccaggagaaagctctcaaagcatgcttcagtggattgacccaaaccgaatggcagcatcggcacactgctcaatgtaggtttatttttttcccttcttctaccaagaaaaaaaaaattgtctctcttgcatgcaataaagacgttggaaataaactgcattggtagcaagacaaaggatttaatgatttaatgctgaaggggtgtgatatgctggcatgcatattgattccctttgctgaaaatttcctgttagatgttttcttcccaaataacccctcccgcccccatgctcctctccttaccaaccttata

SEQ ID NO: 39 - DNA, 1700 bp, Homo Sapiens
tctctcctggacaccggagggaaccccgaatataccgctttactgcccacgggattgggaagcacacggaggctttgctggtcaacccctgctttgtcactgttaataagttattattagttctcataactaccggggggctggcgccgatcgcgggtggcgcggggcgggatccgctcctgggacggtggagggcggtggcgctgcagtcgcgccggcttctgagccccagggagcggagggcacggagccgggcgagtcctgcggtgtcagaatcctgcgccgccctgacgctggggctcagggttcattccgggggcgcgcgcgtggctgcggaggcgggaaggtccctctgcctgtcgccgcggagccccgcgtccctgcaccccgagctctgggggtccgccttgggctgccccccttccccggcctcgccgcttacctggctcggtctctgccagcgtctcggcctcctccggcgcggcgcggggcccggtgctcagctggtcgttctgcgccccggcgcggctgcgtcctccctctggctctggctctggctccggctccgggtcgcgctgtctctggctgctcgtgcggccgccttgccgctgcgcgccgtcccgcaggatgtcctggatgaggaaggacgtgagcggcttggacggggtcggcggcgcggccccctccgctttcgcctccccgggccgcggctccggaaccctgagcatcccggcggccccgccttggcctgaatgcacccgcccggcccgcaccgctttcactttccgcctcccggctcgcttatagcccaggggagcgggccgcgccgccctcccattggccgcgggacgggccgcgccgcggattggccgccctgagccccaggtgaccccgccgatctgcgagccctgagcactgccccgctccgctcctccctaggggattccttccccagttccctctccttccccaattccctctccttccccaatttcctctccttctccagctctcctccctccgccctgagccgttttcccaggccaggagcgcaccccaaagcggggctcctctgccccaacccacccgcaacagagagaagctcctggggatgggacgatcaagacaaacagtccagttagctcttcaggaccgtcagagccaattacaggggatgagcacgcagtcactgcagccgcgcggtaccgtgagagttctgggtgctctctctctctgggagagatggtgcaaactcagatctgcaatatctacgacctcaaagaggcatgacaagtaggtgcagccagaccccaggctgaaagaaggaaaacacctttgccaaagccttttcccgttgcagaagtggttttctttggtggttcctttatccgcaataacctgttaactttccttcctccccagacacatgacataccatccttttgaaaagtttccagacggatggaaggcgttttaggccaggacaaatgcaaaaactgcatttaaaaaagtagttcaaaagaacatcagcatactctttttgcagattaccaactgttaacatgtaacccattggctaaacaatgggttagccagattggtccaaccggtactgtgactggactggacggtaccgtgacaacagacgaaagtggaacagagatgatggaagtattttctttccttgtcttttctcttct

SEQ ID NO: 40 - DNA, 1700 bp, Homo Sapiens
ctggcctcccaaagtcctgagattacaggtgtgagccaccgcgccggcccccaaatatgttatttataaactgtccctggcccgtccctcagatcgcaggcccaaccacgcccccagaaaaatctctcaccagccctgctggccttcgggcagtttctacaatgcaccaactcgttcctctcccatgttcccaaggagctttctctgccccacgatctgagactagaactgcctctttctcattgttgatgtctaggcccaaatgacatttcttcatacgtaaaacgggcttcaagtctcactcatggctgtgacgatctgaaagcaaatgcgtgtatatgacaacgtgtgagcgatgagaagcttgtcatctgtaaaactccatgcgcatgttaattatcatggccgtgataattatgtacattatagaaacattaaccaagcgctgggctcgtttcaggaccttcagtattgctgggaggcttggtatccgacaggtgttccaggagggcaggaaaggagagatatgtttggtcccaagtctcgcgagggctctggggagcgctgggtcttcaaggacgttttggatttgaatacttggtgagagcgtcgggtaggggaacagcaggagcaaagcccggaaggtggctatgaggataagaagggaggacacctggcaggctgctaaaaccaggcccacctgccgggcaggtggaggcgagcaaccacctaagctctctgggacagcgggagagggggcaggggagtcgccgcaccattcttcctgtggccgctagggggtccccgctccaggtgagcgccaagctctagccccaggtcgctcccttatgcttcaagcaagctttgcagcgcagccaattgccaggggctgtcccgcctactccccgtcctcgcagccacttgcctgcgcgaaatgcctgccaacttctgactggcaggcagtctggcaaatcaaatcgcgacctttgaaagcaaaacactgcagcatcttggcagctctgaattgggaagggatgaaggaggctgtgcctccgggttgcacgaagagtccgagtcatttctcagaaggttttgataggtgggccttagaggagacgccgccggtgagtagtgattaacaccgggaggaaggggaattgaatttaaccttcgtttttttctggaaaaagcgaagtcacctaacgtcccctagtgtacatacccttccttcttactgtcaccagcctcgccaacctgggtcccgttgccttggaatgttctttccagttttgcatcgaggccaagaggagcgggggcatgggctaccttactaaaggtgatgccaggctctaccaaaccaggaagtgacatggagttaactttgccagaatttctcctcttcgtgccgagcggctcgggcttcctggcggcagcagatggtggagttagcaggtgggatgaggggaggcgttcttggtctaagcccgcttctggaacagaggtgctgtctcctcgagttgtaagtttccagctcagtgggacgggacggaagaatgtaaccttctcgtgagccaaagccgaggaacgggaagcttggcagggaactggcgctcacctccagaagccagatcgtcgggtggtgggaaagagcgtgttttattgatttgttcagaaagaggcaaattcgaatacagacgcta

SEQ ID NO: 41 - DNA, 1700 bp, Homo Sapiens
catgaattagccatattatacagttgttttattaaaaaccaaaactttgtagagtacattaacagagactgtatttaagagaaaggcagtcatcccttagtcatttatacaattatttattaagcacttatgtgctagttgatacttctgggtgctggaataaaataaggaaaaaatcaagatctctaccttcacgaagctcactaaagtgtggtaaatgacagagcaactaccttcaatagtctctagtttgtattctgcaaccaactgaatacagcaaatactaaaagctacttgaggtagggatccatcctatttttacctgttggatcccattccgtgcctagcacaccagcttgtaagcaatcgacatggattaaggggtaaaaacaggcccttaaatagctaaaattttgacaagtaatcacttgtctgatcatcaaatatgtcgttcgaaatcctcatttttttcttcaaagcgcagtaaacaagtatttgcgcacctattacgtgcaaaacagttttcaacacaatgctttgtgtgtaaaaggaacggcgggaatggattcaagttgcgcatcgtgataaatggcgtgaatatcctgagcgggtggacagcccttcctccgtgagttttttctttttcttttctagtcaccaagacagcacgcgtgtgcagaagctgcagcggtgggacgcagtgctaagtctgggggcgcacacaccagctcgcgcgtcgcgagctcgcctgggcagctcggcgggcgcggcgaaacagctcccacgtccgccttccaccgactcctcggtatcagagcggacgaggcgtccccccagaggaaagactgggcgccgcagacgccccttggagaagtccctggccactcgccccacctcggggtcctcttcctcttgggcttctcctgaccctttgggtgggcgctccagacaggcccgtcggacgcgatttggaaacgggtcgtgcccggcctagcccgcatctactcgcccggccacagcccagccacagcttagggactttgaggcgcccgcgcccctttgtcgcggtatctctagcccaagcaggccggacgccgaggcctcgcggtatcgagcctccgtccgccccgcctcctcgccctccactccgccccgccctcgctctctcacccctcccccgctcccgcctcgcttccccgacaatctcctcgcgctcccggcccggcacgcgcgctgcgcccggccgccgctgccgccgccgccgccgccgccgctgccgccgagcgttcctccgctgcgcctggcttccagcttcgggccggaaccggaagtttggggggcggagcccggcggaggccaggagaccgaaaacgcggccgagcccggagcccggagctggagccagagcctggaccagaacttggccgccgcctgcaccgccgccgccgctgccgcccgccgccccttccccgcgccgcagccgcctcgccgccaccgccgcgagctcggccgccagtggtcctcggactttaggtgtctgggttgaaggtcggtccggacatcggacccagtcagttcccagactgtccgggcggcagcggacgccgctgccgccgccgcctcttcgtcgcctcagcctggcgttttgttccgagagacgggagaggcgagcggagctgacagtgattttgacagtgatt

SEQ ID NO: 42 - DNA, 1700 bp, Homo Sapiens
acctccgcggccatccccagccaggccagcacagcgctccgtgcccgctgaaccgtcccaggcagtcctctgatgacccaaaccaccaccacctttgggcccagagcaggacaaatgaaatttcaacaggggcctgccctgtgaccgaaaattactcagcttcacagcccctgaatgcaaggagaggtggtccccccagaaccaacatgccccccaaggtgggtacgcccacggttctgggaaatgcatacctcaggtaattcacaggtgcgatcccccagtggaggcggggcagcctgaacttctggccctgcaggagtgggaagaaatcgcttttgtctttccgacaactgagccttccacctggggctgggagggcggacagcaggaccggctcttccactccccaccgcggccccccgacctggtcagtagctctggcgttcagacaaaaggtcccctgagctgggggtggggggcaggtcgagagtaggccggtaaatcagacttcgcagctccttaagtcaccagtcctgcatttgggtgcagagattccgttttaaaaattcgaagcatcgctttggttcgctctttgttcgcgggcgcgggttctcgctcatccatccatccatccatccaaccatccatccatccattcattctttcttgacgcactcgctcgctccccggtcgctccgggcgcattaccgcttcttcttctgcttgagggacttcctgcgtttcaggatcatctcatagagcttgtccatgccctcggtgaggccctcgccgatgatggcgcacgccggctggacgtgataggtggtggccgggataagctcgtgcagcgccagctgcttctcaatctctgccaccggcagcgacttgggcaggtcctgcttgttggcgatgaccagcagcggcgtgccctggttctcggcgaacttggtcaccttgtgcagctccgtcttggcctcctccagccggtccacgtccaccgagtccaccacgtagatgatgccgtccgtgcagcggctgtaggacttccacagcggccgcagcttctcctggccgcccacgtcccagaagtggcagctgatgcccttggccgtgccgttgctcagcttgatcttctcggtgttgaagccgatggtgggcaccgtgttcacgaactcgttgaacttgagccggtagagcaccgtggtcttgccggccgagtccaagcccaacatgacgatatgcagggactggaaggccgagatgttagaggagatgttgcccatggctcctggctgcggcgccagccactgcggctgcggcgggagggcgccgccccccgagcagtcacgggcccgacgcggccgggcgcacctgggccccgcccgccgccgcccgcaccggccccggggctcggcgtccccgcgggcgcacggcttggcgcgctcagacgccgcagtccccgcgccctcgccggccgggctcgcgatcggcgggcatcgcgtctctacgcgcggctttgtctcccgcgaagccgcctcagatgttcggcgccctccccaggcctgggctcgctctgtggtttcctcgggccgcgcccccgcgctccggcagctgcccgggctctccgctctcccggcgcccggcgctcgcctctggcctgggacggcgctgctcctgctcctgctcccgctcccgctccc

SEQ ID NO: 43 - DNA, 1700 bp, Homo Sapiens
agcaaactgattttgcctgtttagtctcactagccagggtggtatggaagtgggtggggagagggctggtgcgtggcacagataactgggaagcgcgcgcaccacacacacacacacacacacacacacacacacacacacacacggccccagttaacatttatgcagtacttttggggggtctcttacacacacctgaatagcatactctcaataaaggataggcacacccctgtttgtcctcccccaatttccagactagcattttaccctgggtctagaattggtggtgataagccgacttagatggagtatttgagaaaaggggaggtgaccctttgcctgaatgggggagggggcgctctccactcaccgcctgagtccctggaacgaggagggccaggacatcctggatttcttcaggccgggccggtggccggtctccaccgcgtaggaggtgcggtccatggcgcgacagtacaggtagcgctcggtgtccgagtagccgcgatgccggacgcggccggtggccccgctcgaggcgtagcggccgcgggcagccccgggcggcggcgggggcggcggcagggggggcggcggcggcggctgtagcggacactggggctggggctggggcgagggtggcggcggcgggggcaggtggtgatggggatgcaggaggcggaactggggctgccggagcgggtactggtggtgctgctcgtgcctccagagatgcttgggggctttgagcgtggccccgccggcgctgtcgctgccctctccgctgcccgcccgggccggcgcgctgctgccctctgcctccatcctggctcgcggctccgcgacctgctgcccagcccgggttcaccgcgctggcccgagcgccttcctgatgctgctgctgctgctgccgccgccgccgcttctgcagcccagcagaggctggtgcttagcttctcacttgaaggcgccagagcctcagggctaccgagagggggccctgccagcgggaggggtcacagaacgcggcagggctccttccaccgaggctatggcagagacacgctcccgggctccccaagtcaccaagactagggtgcgcggtgcccggtttcgccagaggttggtccttcttcctccctttcttcttcccttcctttgggcgccggcggcgagagcagggattgtcagcaactcgggctgcagaagcagcagcaacggcggcggcggcagtggtagccgcctccagggcaccggaaccctgatcccggagctgctgaaaagttggacagctccaggctggcacgcccgagctgtgactttcaccaaaaccactccagccagtcaggagtcagacctgtcctcttgatccttcccctcgccgtgcccgcgcctccctcctccacactgctccccactgccccggagccttcctcttcacctccgcttgcctcagtgctcttagagaaatggagtttggcgattcctggctggttctcagagcttgttgacgatgagccccatgctggtgcttccaaagataaggaatgtgaccaggcaaagttgcctggaatattgtctatgcgcgtcgctgaaaaattggatctgggtgaagatccaatagtaagattaattcagtctctttccctgacataggataaaacattgtgtggctctttcccacagcagggagaatg

SEQ ID NO: 44 - DNA, 1700 bp, Homo Sapiens
ggacagggaagcttagctaagaaccaggagtttgacatacttaatctctcatttcatcctactgatgactttgtgaggtatatctttttaatcctagatctacaaaagagaaagctgaggcttggagaggttcttcattttgttcaaagtcatacaacttttacgtggtgtagccaggatgggaaccagagcctggctctctgcacttccaacaccactgctgaatccgggtttgtctgttttccctctcttcttttaactattgtcacacagattccatcatagaacccatcacgtggagggccactatttctctttcttttgtatagtatgtttgcttcccctaccccacagagtaaggtgctcagcaaatgtggctgtctggaggcaacacccccttctcaccgccacacttggggttggggtgcgaggcaaggcagggctggcgaggtggatgttgcagctgtcaccactgctaggtgttgggaaacgactctcctgcctgtgcattgacccgaaggcattttccatttcctaggcccctcccgcttcccgtccctcacctctagcccctcggcttcagctcaggcccctcggggagcatcccttgccgtgagactgacagcctttgggggcgcagggtcctgttctctgcgctctagcccatctgtgcgcagagcctcgttcccaggcgcctggaacccggcgggcattgacgtcaagcgccggcggagcgctgcctacagacggttgacccgggccctcctccacacccccttccttcttcgcctcctccctctttcctgcacgggggctcgggctcactataaaaggtgggagcgcgtggtgccccagcaacgacgagtttcagaacgatggagagctcccgcgtgaggctgctgcccctcctgggcgccgccctgctgctgatgctacctctgttgggtacccgtgcccaggaggacgccgagctccagccccgagccctggacatctactctgccgtggatgatgcctcccacgagaaggagctggtcggtattcccctcgctctcgacccccttgagctgtcgccttgtctcttctcttgcacgcctccctcctccccccacccccactcctattcccagagtcagggcgcggggagctgagcgcaacgcccaggcacccactgccatccgaagagcgtctcgagctcacgggctcctggcagtctgttgagcgaatccctcatcccggcccctctgagcaacagggaccccagcggctcagagacccgcggtcagtacctgggacagcgtccgctaagtttccacccctcgaccattccctgtgtccgcggagtcccaccgcagagtgcgtgtgggtccggggctccttataactagggctggaagtgcgcacctgggctgggctcgcagccaaggcggcaacttcaggctccgaagcggtgtgttgcagatcgaagcgctgcaagaagtcttgaagaagctcaagagtaaacgtgttcccatctatgagaagaagtatggccaagtccccatggtaaggtttgtggtcactcccttcccgtgtttttccaagagaaagtacaccgccttgaatcgtacacacagtcttctccgtaggatgtggctaaataacttaggtaatgggcttgcaggattctgtgggctccttcttccttcccgggtgaggaa

SEQ ID NO: 45 - DNA, 1700 bp, Homo Sapiens
gtgtttgcacttcatgttactttttgagtttataaacataaaaacagaatttacttctgttacagacctagttactgggaattcattacttgccatggactacctttgctaagaaaagtctgaatgagaagatggcaggacgtctgaaaaaaaaagttataattaataaaatctgcggagaattgtaaattctgccttcattgttgatccaaacctttcttctaagacctcccctttctagacatctatctacctattttcactttactttccagcatcaaggaggtctgagtggataaggcctaagcactgatgtggctttggtaatgaacctgaagttaaaaactggttagcttatgctaagctgccatttgaaaggtctcctccagcatctgaattaagggaaacaatgttttgaagcagtcacaaaacttttctgatgcattagatctgaggtaggacctgataatttgtacttctaaattatgcaggtgatgctgatgctgctggtccggagaccactttggagagttttactttaaaggaaatgaaactgccctaaaatgctaatactaaatagccacacaaagccggaaaaaagcaaagaatttcaaaccgaagatgagaggtcctgaaaaataacagcggtcatcctactcccggtgcggctatgcggccatgaaaaggagtcttggggccggtcgtgcccctccacgcaggctccctcatagggtcatcccatcaaccctcgccaaaaacgccggccccctcatagggccgtccggccacgcccctccgtgccctaaccacagagccacacgttacgccgccgccccgttacgcccatccggctcccgcatagggcgtcacgtacgtttaacgtcggcccggcgcgcgcgtccgggaggttgcccgtaggccgctaactggtggttcccagtccccgccgactcggcctctttcttctgcggccaccagatcgcgggatatcggagacttcgaacaatccgctggagtcctttacagtttaaaaacagaggaccggccgggcgcggtggctcacgcctgtaatcccagcactttgggaggccgaggcgggcggatcacgagttcaggagatagagaccatcctggctaacaccgtgagaccccgtccctatcaaaagtacaaaaaaattagccgggtgtggtggcacgcgcctgtagtcccagctactcaggaggctgaggcagaagaatcgctagaaccagggaggcggaggttgcagtgaccgagatcgcgccgctgcactccagcctaggcgacagagcgagactgcgtctcaaaacaaaaaaaacggaggatcgaaaattttgaagactgagagcaaggcaacaatcttcccttgacctttttgctcctctcacaagggtttttcagacggctgaaaccaacgaaaagagtgaggaagcaggcagaaaatttcagctttagtctctcagcatcctcctgctttcttctcggggagctggcacttcccccttctctagtagctgtaaatacgcctcccaactccaaggagacctttcttgcatctcccttactgttctcacgcctgtagggctcttcgtgtgaggattcagtgtatgtatgtatgtatatgaatgtgtgcgaataattatttcctaattgagtttttttttaaaaaaatcatcttccg

SEQ ID NO: 46 - DNA, 1700 bp, Homo Sapiens
gtacacgtgaaaacaatactattttatatcacgactcatgccatccgtatgtgtaatcaaagccttctaagtcttgatatgtccttacaaagacgaaggatagagtgaatgtcaaaatttgccaaacaggagttactgaaagtctgaaggggaaagcctagttaggttttatcttagaagtgatttttaaaacgaaacacgcagataagctcgagctttctggagccgattcctaaggccttgcaggtggtaagggggtcgagaattggctgaggttcctcggaagtccgcgccgattcggagggccacctctgactttctgatctggggtgcaacccccagtccctgtcccggtcgacaaaggtgtggagtcaggttccaggcaagctgggggtgggggtgcgcacaagggaggggcaggggactgggaccccctcttcttcggggtgcttcgggccgtttctccagaggctggtggtgtccccgacccaagcagctcagccccgctcgcgaggtccgaagtggcgggaacgggtcctccgcggcggtcccaaggcgagggaggctgtaaaacctggcagagaatccggcgcgccgggtcggggatgtctcgggtgccgggggcaggggccgcaggctcgcaggtagaagcttccccacgcgagcgggggcctcggccgcggaaggggcgcccccggggccgccggcagcggcgagcgggcgggagggagctttccgggcccctcccgcgcctcgggctggcggggcgatggcgacgacggtttcccggctcccccgccccggacgccgatggcggtgcgttccggccctcccccaacagcttctcccccgcggtcgccggagccggtgaggccgagagggcggggagccgcgcagggaaggcgagagggcggaggttgcgtggtgcaggcggcggcggcgggaggaggagggccgggcgggcaggggaggaggaggaggcgggcgccgtgtcgcacgcagctccaggcggggcagccccggtagctgagggacgcagctagaccttggcgggacggggctttcgccggggcccaggcccagggaccaggcggaggcgtcgcgggagcctttggggcaccacagagatgcgggtgagtgtcggcagccgccggggccgagaggaggtggcggcggcggcggcggcgcaggcccggccccgagccgcggtgtcgcagtgactcgggccgcgacggccgaaaccaggcgcctggcgaggcctgagccgccgaaacgcggccgggcagaatggcggccgggccgggggactcgtgggcggaggacgccgaaggtggcagaggagcgagcggggccgggccctgagtcaccccggacgggccggggcggccaggcctggactacgcactgggaaaaagcacgattcatcgcgcctggaggcggcggcctggtggcgccggcccctttccccgccgcgaccgggctgggttctcggagggtgggccgacgtgtggcccggggcggctcgggcgtccgcgcctcgcctggtgaggcgacctccgcgggacgccgggacccgtgcccttgccccctcgcgcccacgcttcttcccggcgctgctttccggccgacgcgtgttcccctgtgagctctgggctcaagcgcaggagcagccgcggagccgggctctctcgctttccggt

SEQ ID NO: 47 - DNA, 1700 bp, Homo Sapiens
gctagagattgaaatcctctggaaccaaccactggacgctctggattggggcggaacgcgcttccactgtctcgaaagcagcaccttctcggatttgagcctttgcggcgcgcctgggctgagatctgagaaatgcgccctgggccacaggcagcagcggaggcggggaaggaacaactttccctccgccctctaaccacaagggcggggtggggcggcctcgctggggtcgggctcctccactttcccagctgggatcccctgccggagctggcgtgggggccggggagggggaccagaggggcttccttctctccccaagccccgccagatgttggaaaggcaaacaataggggaaacgcggagaaacaaaaggccattcgccacgatctcagtcggctgcgtagagagatctctgtggggagggttaggggagagggtcaggaaagagcgcgaggggggggatcccgagagatttagagtctctggtacttgccaggcaccaggggtcggcgctttaccctcccagcaggggtctgaaggtgggaaggggcaatcaaagaaaacttgaagggttttcaccttcttccccagttcagggaaagggaggggctgggattccagatgtttgacagttgttggccccaaagttaagcgcgatttgtacggcctttacacgctccccacctccgtgaggcatcgtcagccccgggggcgacctggctgacggtgcggacttcccctacgctgccccaagccgagactcctccctcaggatcaggacgcccaagcgttgtcccccagcccaggcgcacttttgtaaaagtttgtcttcccgccacccacagcccaacacgcaccctgcaggcgtccgcggctggggatgggctcggagtcttgcaccccgaaagaaggacgatcttattgatttatatcttttcaaatcaaggacaaatcaagatcccggggtggaaagtgggcgacccttgttcctgccgggaagtgcacacgaggagccggccagaaacccaggcgccggcaggggtctgaagaggactccagccccggaagcccgtggccggcgggcagaagaccggtccgccgcctcccgcaccttcttgcacccgcgcccaggtgcgccccaaacctcgccgcccgcccgggcgagatcgggagcgagcagccaccgggctgcgccgcctcccctagcttggagcgggttcttactcaccgtggggttttacctccagcccagggcccggtcggctcccttcgccgggtcagctgcagccagcgcgaaagaaaatggtgagctcggggcaggtggggcgtcgcttgccaatccacgcaagggactctgccgccggtgcgcgaggtcccggcgagccaaccggcggacgggcgagcgcctggctccagctgcagccgccgtcgctccggagcagccccctcatgcacccagcgcgccgcgcagccggccggggctcgactgtctcgcggggacgccgagcggggacccctggcggccgcctgcggtatggcagcaccgtggagagaaaagtcgtctttgggatcatcttccctccggaaagagatgggacatgaccagtctaagggggcttccagccttgcagaaaagacactgggccagggcagtgaagaaggtgtcagatcagaaaggatgatctctgggtccacaa

SEQ ID NO: 48 - DNA, 1700 bp, Homo Sapiens
ctaggatctctaaccactcaccacttgtattcctggggtggacgtgagctcgcgctaaactaaggacaccagccgagcattcagcgcggttccctacgccctccaggaattttggaaagctatccaagctcggcgctatctttaaaaggaatgagttgtggctcctaactagcttgccgaggagagcgtagagtgacacgggacgggggcccagggcactgaggccaagagttcccgagtcacgccagtgctcgcccctttggcctttgcctaaaatcctgcgatgcctcgctgacaaagcacccagtgccagagcctgcggcccagcgaagctgctccctggaaacacacacacacacacacacacacacacacacacacacacacacacacacaaaagggggtggggagagggaaaggtttaggtttaggagaggaagccagccagccagccctgcagatgcaaccgttgcaatttgggtctcttctgtcttctgacgtaatgccagtttcctgctttcgcgctgcagtgtgcagttcgctgctgggaagtcagatcaatcgattgaacgccaagatcatcactgccactatcttgcgatttattttccacgaatacactttaattggcccgagccccgcccccaaattccacgacttttggcttttcaggaagcattgttctcccctccccccaggaaggccgcctctacgcacagccagcatcaggaaacggcgccgctcggggctccgatggagagccggggtggggtcggctcaaaccttctgccccccgctcttttgagattcctgatccctggctttgtcttaagcaaaagtactgtagaagctgcagcgtttcctccccctttccagaaaccgagcgaggacgtgggggaaaagccaccctggaacgtgtaaggacgctcccccacgcccaacacgccacagggccgggatgcgcggctgccgccccgccacctcacgcgcgcagcgctaactgcagctccgcagccaccgccgtcgccgccgccctgccgagcccggggagactcccaagggctctttaaatacgagctataaatactacaggcgctcggcgcgtgcgtcacgacacatccggctcccgattgggcggcgtaacctcgcttatttgcataggccgattgcacaaccgggcggcgacctcagcccggtgctcgtgccacgccttccccacttctgcgggcacgcggggccgcgggcgggacagcggggtggcgattgcggcgtccagagtcccttgaactcgggaacctttcaagtcctgagtcaggcggcggtcctccgggacgccagtgcctctcagaaggctcagttttggccagccaagggcttaccgtttaaactgggtcttttttatttctacagaaaaggatagttttcagagtgacaccccagcaaacgctctgggtggctcgcagaggctgagtttaaaccacgtgagacctggccgcggcgagagtcctgtccaatgacttgcgacttggggtgttcaaaaagggcttttcgcatgatctctaataagacctcgttatcttgaaaacgaaacagggagggttcgaaaacgcaagatgtgtaatgccttgtggtttaaacctagtttttaaaaaatgcaccaatgagccaatatgaaacagcgacatggagactttccccca

SEQ ID NO: 49 - DNA, 1700 bp, Homo Sapiens
cattccaagtcaccaagttccagacaggcacagtagcacacacgtgtgaacagaatccatcagtacagtctggggcccaggcctttgcacaccaacaacttaaggttcttggtaatggcccagcgtgcacccatgaggccactgctcttttacacacgcactcagaacacccacagctttaaaacggcaatcttactacaaaaatgacactttgcatttgcacagcactttgttttttgtgtactttacacatttattacctaatgactgtgttttttgaaatctccaataactctgggtggtggtaagaataggcatccttaagccaccttcccagatgaggaaacttatctgtgttcccctccttcttctcaacagccggctgatggaacccaagccctccacaccagcagtggggccagctgcacagagcggccagaagggagaggccccggggccacgcccttctctctactctcagttcctgactcattgtcacatcatggcgagtaacaggacaaccctccccacagaatcatgggggacactaccttcctgaggccaatgtgttcaaccaagcgggaaactctccgggtagagtgaaatccgaagttgctatgctacaagataacctagagcaaggggaataactgacgttttaatgtcttcttttatggggggctgaggggtgcctactcacactcccccctctcgtcttcccccgctacttccctcctgctggtcggttcaactccacaagcgcgcgtttgtttcgagtacagtaacttagagcagatggggggcactttctagagggcagtggaaagcccacttgctactgctgcgccggggacctgcggctccctgggcggccacgccctgcctcggcggcgcctggcgcgtctggaatgcggggcctccctccgcccctctccccacgccgcctcgccccgctcccgctagggcttcttagagaagcccgaccccccaccacctctgccctgcaccctcccggcccgagggccggtggcccctcacccctgccccaaacaccttgtccctgagccccctccccaaagactctccagtgccggccgtcccttcagccccctgcccccgacggcctccggggccagttgccggggcctccctgccaggccgtacccttcttcccagcgtctccacaccccgctagctggcggcccatcgcgggcgggcagggcgcgccccgcttacctgggccgtgcgccgagagctgagagcgtccccaaatgctgagcagagccggctggcctgggtggggcttttatacccgccacatcctccctaggcgaggcgaggcccgccccgccccgccccgcctcccccttacacggcccgctcccaccccaagtcaggggtgactcaccccggcccgccggcccgcagggaccgcgccctggaacgctcccgagccgcgccacgcgggagcggccccagcgcccctggccctgccctccgagaaagcccggccgaggctgccccgaggcccacagggaccccgtttcttcctcctttcctcattttacttcttaagtgccacgtcgagctctcagcctcggcttgggttaataaattaacaagactttagtagtagactctgggcttgattaagtgtttttttttttttttttttttttttggcgttaatgcaatggagg

SEQ ID NO: 50 - DNA, 1700 bp, Homo Sapiens
ggctccctggtagctcaagtgtcagtccagacattgagacctcggcaaagagcttccctttaccctctcttcccactggcctttgtgggcggtgtggaatgtgctgtaggccttgcgagagcggcatttcttttttttttttctctctttttttttaacctattgtagcctctgagagggcagcatttttgggcgcacctgcacccaagcctctgacagtgccctgggttcctccagcaacgcgcatctagggatgctatgccctgtgggccctatgcccggggcacggagctaggggaggcggtcaccctacttctctcacgtccctgtccaccttgcgcctcagcggagccttaggtaaacaagccaggctcccagtccccaaggacggccttcaccccagaccccaggaggaggcgtcttgggccatctcgggaaggctagcttgctcgggaggggcagatcacgaaaacggttctttaggcagtgtcacttcctctccactgccagccacccaatttaggcggctccatccctcccgctttcatggatatgcagcaggatgcagagagcacagagctgtgattggcacgagctcacaacgggctttcccgctcaatctactcaacttccagcaagggacaggccctgcccattggggttcgctgaggcctcaaacgcgcatgctgtcaagggagcagctgcgggggcccgctagtggggacaaaggcgcagccaccggggcgaaagctccgccccgcagagggtgggcctgcggcgaccgcgagctcagagtccggacttgtcgcgcaatctgtctccttcggttccaccccaacctagctggactctcggtccggtccctgccctctggagactcagccattaaagttgaggtggggggatgaggatgtgggcggcgtccaggtaactctgaggggccctgatgccttccctagagccagccatcaggagtaacacgatgcgcccccacatcaccgaacccagaacctcagtgcttcagcaccccttcctcgacatgagctccgcccaatgaggagaagggtcttctgcattggccggtggtggtcccctatccaattagagaggctaacggccttctctcgtctggtttagaaggaatcgccgggggaatacaggccaaactacaattcccgagaggtagctgggctctgcgggggggggggggggggcgcctgcgtgttcacgtgacttccttccttctcgcggagaccgagttacgcgataggcctgtgagggggcggggcccggcgcctgttcgccgtgttagtggccaatcggcagccaggcagggtgggcgcgcgtagggggcggggccgggcgcgcggcagggcgcgagagcgcacccgcggcggcggtggcggcgactgtgggggggcggcggggaacattggctaagccgacagtggaggcttaggcaccggtggcgggcggctgcggttcctggtgctgctcggcgcgcggccagctttcggaacggaacgctcggcgtcgcgggccccgcccggaaagtttgccgtggagtcgcgacctcttggcccgcgcggcccggcatgaagcggcgttgaggagctgctgccgccgcttgccgctgccgccgccgccgcctgaggaggagctgcagcaccctgggccacgccgatgactactgcaaactg

SEQ ID NO: 51 - DNA, 1700 bp, Homo Sapiens
gccctggacattccctgtcttcctgacccaaggtctagtttggctccctctagggctccctggtagctcaagtgtcagtccagacattgagacctcggcaaagagcttccctttaccctctcttcccactggcctttgtgggcggtgtggaatgtgctgtaggccttgcgagagcggcatttcttttttttttttctctctttttttttaacctattgtagcctctgagagggcagcatttttgggcgcacctgcacccaagcctctgacagtgccctgggttcctccagcaacgcgcatctagggatgctatgccctgtgggccctatgcccggggcacggagctaggggaggcggtcaccctacttctctcacgtccctgtccaccttgcgcctcagcggagccttaggtaaacaagccaggctcccagtccccaaggacggccttcaccccagaccccaggaggaggcgtcttgggccatctcgggaaggctagcttgctcgggaggggcagatcacgaaaacggttctttaggcagtgtcacttcctctccactgccagccacccaatttaggcggctccatccctcccgctttcatggatatgcagcaggatgcagagagcacagagctgtgattggcacgagctcacaacgggctttcccgctcaatctactcaacttccagcaagggacaggccctgcccattggggttcgctgaggcctcaaacgcgcatgctgtcaagggagcagctgcgggggcccgctagtggggacaaaggcgcagccaccggggcgaaagctccgccccgcagagggtgggcctgcggcgaccgcgagctcagagtccggacttgtcgcgcaatctgtctccttcggttccaccccaacctagctggactctcggtccggtccctgccctctggagactcagccattaaagttgaggtggggggatgaggatgtgggcggcgtccaggtaactctgaggggccctgatgccttccctagagccagccatcaggagtaacacgatgcgcccccacatcaccgaacccagaacctcagtgcttcagcaccccttcctcgacatgagctccgcccaatgaggagaagggtcttctgcattggccggtggtggtcccctatccaattagagaggctaacggccttctctcgtctggtttagaaggaatcgccgggggaatacaggccaaactacaattcccgagaggtagctgggctctgcgggggggggggggggggcgcctgcgtgttcacgtgacttccttccttctcgcggagaccgagttacgcgataggcctgtgagggggcggggcccggcgcctgttcgccgtgttagtggccaatcggcagccaggcagggtgggcgcgcgtagggggcggggccgggcgcgcggcagggcgcgagagcgcacccgcggcggcggtggcggcgactgtgggggggcggcggggaacattggctaagccgacagtggaggcttaggcaccggtggcgggcggctgcggttcctggtgctgctcggcgcgcggccagctttcggaacggaacgctcggcgtcgcgggccccgcccggaaagtttgccgtggagtcgcgacctcttggcccgcgcggcccggcatgaagcggcgttgaggagctgctgccgccgcttgccgctgccgccgccgcc

SEQ ID NO: 52 - DNA, 1700 bp, Homo Sapiens
cgagggtctcggctcccctctctgtgtctgcctctcgcccagcgccgggagaagcagcaacaagttttgcatttcagcaatcaatttcagccattacatttgcaccaatcagcgccgcccaagttccgggctcggggcggggctcgctcttaaggtggtccggggtcctggctgccgaagcccccgccacgaacccgcactaccccagctctggggtcccctgcgcccgcctctcctcggggtccggcgtcagtgcgctgggggcgcgccccggcctccgggcgcatcgagctggagagggtgcaggcggaggagaatggcgggagcttggctttgttcacccccttcccattcacactgttcccaactccccacccccaactggagcgagacccaccgggtgggcagggggcggggaggtgggcggtgagcggtacggggcgctcagacaatgaggtcgccaggcaaagaccctagactcgccacggaaagtcgctaagtgtctactgcacttcggctccggggtgaacactccgttctccgctagcggcgcccaagttagccggccctgggattttcttggagggtaaagcagacggggaacctcgaagttagggttctcctcgagagacctgggcgcttgctcactcctccgtcactagggcgttcacaattgttccccttccccactgcctggggttcccggttctcaccggccggcgtgggctccaaggagtcacagtcactcagccactttatgggggagcacatcggttgagcccagtaaccggagcgacttgcgaggcggctgggaaccccgagaagacacgcacgaagagggcgcacccgccgggccgcacaccttgccctgggccacccggtccaagacccagaccctctgcgggtgcgaagttggcctccttaagagatcaattaaactgaagaggccgaggaatggaattgggctgtgatactaaaatcaataggagtaatttaatatctgccctgctctttatgaaatatttatcccaataatcttagttaaaatgtttagatcgtcctgatccccacagaggctgaaatctatcttgcaattagaagttggaagttagaggtgatggaggatagaggtgtttattctgtgcgcctgtgtgcgtgtgctcgcgcctgcgtgcgtgtgtttgtgcatgcgtgtgtgtgtgtgtgtgtgttcctccctaaagtgtgtgtagggaaggaggggggagaaggcctttaattttccttgccataaaatagcacgaggcgattttgctagcagattcgcattcttaggagaagggcgaaaaaagaactttctttgaagtgcccctgatatcgctattaaatctaaataaaattaagcgtggtgtatgaaaatgcctttccccccccacaaaagaaagtgtttatcgcccgagtctgtctagcgtttgctaaattacgcatgaaatctgaaatgaaataaacgttataataaaaccaacccctgagccctttttatttaaaaaccttagattaagcactccaccgtcgcgggcaggagttaaaaagttaccatgtcgtacgcgcagattcattgctcccctgagttgcggctaggaaggcagtgctaagtatgccagaaccgacatgccgggggtggggggaaatgggctcaactttggggtagcggcagaatt

SEQ ID NO: 53 - DNA, 1700 bp, Homo Sapiens
ctttacggggatcttcgccaatgcctggctgagaggcggtctcagggggttgagaactctattcttagaggtctggtatctgggagactgagaaagcctttcttaagtctccctgctgggccacgctgtattcacagatcatcttcaactccttcttcttccccacctactccttcgccctcacctccttggcttatctcttgctagaaaatgcaccaagctccaatctccgttccttttagcttcctccctgagaagacactgtgacctcgctgtccttgttcgtgttaccatggagaggtaggaggataaatatcgcttccagttgtcagcttttttgttttctcctttccctcttagacagcccccagccccaatcccagtacccgtttcaccaccctcccctggagaattagcataaccggagtaaataggtaatcaagcagtgtgcttgcaagccttaattttgggacagataaggaagtgaaattgattggaattatacctgccgctgctggaggctgagggaggcggccgccggaggcgaggccagctctgtcggcccagggccccgccccaccccctccttcctccctcctccttctcagccccacccttctccacctcctcccactttgcactctcctctagcggccgcctgtcctttttaaggaactccaccctaggtggagtcctcacgatagtcacacctctcctttgggcctagcagggccagagaaagacggaatcactctacccactcaggttgcccagagagcactttaggttctgactcgcgtggggcgcgaggtaccccgtggggcagctaggtgcctccaagaaaccccgccccagaggacccgcacgagttgctgccatttttcgctgaagcccctgccctggggctggtgttcttctctatgattctaccaatcgtcggcatttttttcctcccttctttctttttaggaaggcgctggggtgtgcggcggcaacggcggcggtgaagggatcctcttgtggtatctcctcgtgggttcctgtcttgaaggatttcttctccgcctcaactttatgttcaagtagcgctttgggggttgggttggttttcatagcccccacccccggccctctccttttaaagggtgtgtggagttcagaggatgggtggagagcgggcttggccggaactgaattggtagaggtaaaaggggaacggaggaccttaagctctggccacttcggcccagcgaacccactttatttttgtccaaggaaaagctgcaggggggaggattctttttaggaagtctactctgccggcccctgatttttgggttggatgcttgagagctttgtttcccttgttccgagagtggcccagctgggtctgaatcgactccctcccctttcgaccccctcctcattttccagcccggagaaataggggagtgggggcccaagaacgagaagacgagaacgcgtcgccctgcgctatgtcagaatggggcgggtgtgaggggaacagctctcttgcgatcagctcaggagtatgagcctcccggaggacggcatgagttctggacacttcaggagtcctcagctagtgacatggtcggtaagtgactctgggactaatacccacccactcccacctctttcgtctagattctatccccctcccagtccccctgctcccc

SEQ ID NO: 54 - DNA, 1700 bp, Homo Sapiens
ctagacagcagaagcccacatcccttggaggtggaagctggaggggaagggtctgaggcagagataaaagcaagagagggattccccaggctctcctggggacgggaggatggtgtctgcccagctctgcgcctgcaatttccacgccatacatatcacagccatgtctgggtcacgcctgcccagagcccaccgggcatgcacatatgtgacatgtccgaagctgagggcaagccacccagggcagagtatgcccccaggctctgcgcctgtcccagggcagttgcaatgcagcaagcgtgttactgttacggaggcaagaaagccagcgccttcccacgcacgcacacaggctaggtggcctcttctgcacaagatgggccaccaacaaactcacctatgctacatacctgctccccaccgcccccacctcactcgtaaagtatggggaaaagaaagcaaaagccctttccgacctggtcttcttctgcgcctcttcttggagccgaagagtttgacccgggaaaagacatttaacttgtttttgtcgcagctggtcttgcctttgctggggctgctgacacacttgcaggcgttggatttctcgcgctcgcgggcttgcctcttctgacggatgagcgagctggcgatagccgccgccatggccacgacgcccaccaccaccgcttcttttgctgcccctctctgggttcgcctcctcctccttctcctccgctgcttgtccgctgtctcgcgacttcgccgctttggtctccttagcctgcgtttgcccgggcttctccgcactcgggcttcagccaaggagggggctcagcatgccgtccgagctcctccggcggcggtccggctcccgcgcgggctgctggctgcctgggtcggagtcgacgccacagccccgggcccgggatcgcgggcgagactggcacgcggatgctgaactgcgcgactgcgtgtggtcggcccctgcgcccggcggacaccgtgcatgtccagctgctccggtccgaatttcgccggaccccctcgccctgttgcccttctgatgggggccagaggagggaggcgggcggagggagtgagcgcgggcgggagagaggccgggagctcgggcggccggacggaggagggacgaggcagcgcgcgggggagcgcgccctcgccctggcccctccgagtcttcgactagtccttgcaacttcttggcgtgataatcgggaaaagttggcccgtgccaggtttccgacagggatcaaacaggtaatggcctcgcatcttcgctgttgctgctgctctgggcgcggcacagtcgcagcacaggaattcagccgccgcaggcaccctccggaacagcgcgacagcctccttgcagccccctcccgcgacggcccctccctcccggggctgagcccgtaggctcggagccgcaggccgcccctcccacccccacccccacccccacccccccaagtccccggccgcccgttccggctaacacctgtaggggcttccgcactgccccagtcgtccaaatccttctcccctccttttcccgaaaggagcttcgggaggaaacagtgatggaggaggaactgagatgaggtagttgggctgacggagcgggggtttgctcggcattgggggatggagtggtgggggtgtttcctcagtgtttgagctga

SEQ ID NO: 55 - DNA, 1700 bp, Homo Sapiens
ttcttcccctcctcctcctcctcctccttctccttctccttcttcttcttttgttcagcgaggagtccagatcagagaggtggcccagatagttcaccaaatggcaagaccagactagcctctaaaccaagacaatttaccctgtctttctttctttttgaaatatacatcctttgctcttagagatctcaaaatcttctgatgacccatacatttatttgaatttttgtttcctgataaaagtcatcatgcgcaccttggaaaaagtgaagaaaagttaaaagaaaatatccacccagagacaacattttagtggaccttcttgtctttatttctgtatatatgagctcatagggtatatatgtttgtatatgtttaaatttacatcataagcatgcccagttcatctttagaattttttaaacaaatataatttaaaataggaatctaatatttcatgttattgatagaccgtagtagactgtgtttgtatttttagataatatctttttttatcactgtaaataagactttattttgtgccttcaattttcatccttgtcaatattttctgttattttctcaggatacatatgtagaagaaacatcaatcagtcaaatattttaatgctcataagacatgccattccagggcttcctagaaaaacagtattatcgtattttgttcccggaggtgcaggcgagtgcctggtctacgggtgtttacaatcatcaccgggcacccacaagagaccctgaacttagaatatttccgcggtaggagttccgcctcgtccttccaggggatactgacgactcgctgggttctcgccttccaagtgctggaggggggaagtagggccacccggggtagaaacctaacgccagaaaccttccagcgccttatcagttggtggaattttcgtggcagacagtcgcgctctacctaagtcgtagaagaaaagacaccttgtgggggacttggcgtcggaagccttgagttcaaatcgtagccctaggtccccagccttgcccttcctggcttgctgcgaggatgaaatgagatttttgtacgtgaaaatgctttctaacccctctcgaggggtggaaacgtctccattcgaccagagtccgggtctgcggagcaatttacccgccgctttccaagagcaggctccccagcgtagcgagtccttgttatggaaagggtcgtttcggctcaggatgcgcgctcccggcgtagacctggggataggggtccctgtcgcgctcgccccacccctgcagggagcaaggccgtccttctgcagcaccgcgccacgcggcgggaatttgcagtgtcctgctttctggtttcagcttcttcgctcgcttgggtctctctcggtcacctctcggaggggcctggagtccctcctccctcctcctacttcagcgccagaagcattgaaagccgcccgcccgccgagagtggaaccggtacctcgaatctcgctcattccgggtcaattggaggcgcattagcggctgccttgcagtgcaacagaggctcgagtgacaggccacctgcctaagcgctggcgcccaggaaagccggggaggaaactaggaagggaggcgacagcagaaaagccagagagtccagagactcctaggcaggcctccgtgcagccccaacagcgtggtgccgggaggtgtgt

SEQ ID NO: 56 - DNA, 1700 bp, Homo Sapiens
ttaccaaaaaagcggagggggctttgtctaacacctgtatacacctccctccgtagcatcccgctcattctagcttcttagggaaaccatagacaacgacgtttcagagagtgccagaggtgaagccgacccgaaaccacattacagaaaggcttcctgtttcaaacaggtccaataaagccaccaaattaagtctacgattataaagaactgcgaatcagaaaataattccatataggtgcagtcttctattacttttttccccactttcggaaatttcaaagaaaaattgctacattcttaaatatgaggccattatttcaaaggctgtaacaacctggaacgcaaattcgttaaattatgatacaataaggccaacctcgaggatcctcggtggactactagcgcacttccttacaattagaaagtgaaaaactgggtcagccctagagtcacgcaataatgctgcgtcacgaaactgcgcattccaagagaaacttttctcaggcccagaattaagtcggagggatccgccgcgcagctcaatccctaggaagcatgaaagaatgtattgtaaagagtaaaatcacgcgcaatccatcccaatataaccgcagttgttcgtgggccttcttgcagagaaaggctgcccaaccttgacggggccatgggcgctgcgggagggcaaaagaccacagagatctgcgaagccaaagtaaacaacggggttggggcggcgagaatgatcaatagcgatgctccgaaaggactccgcgataggaatcgagcgggaaggattccctccatcctaacagtccatgggttaagaggccgaagcttccctaaataccacactcccgcgagagaagggactgagaacaacttctcaaactattctctctcgaaatcgctcccttcctcgaaaattccatctctgagactcggatgaggtccccaccccctccacccttgtcccgtgaccgtcgcccgctcagcctcccagccgagtccgcggggcctggggcgcccaccccgcccacccaagggaccgcgcaggggtgaactcccccgcgccccacctgcgccttccagacctcgggcgccccggcgttgcctcgagagctccctgcgcggccgccgcggcacggaccagctcccactcccttacactgggcgccgctgcgctcgccgggggccggtcccgaggttcccaaggcctcgcgcgcgcgcttgccgtggcaaccaagacgttccacgacgcgcgctctcgaacgcttcgcgtcacgcggccgcgcggccccgcccgtcggcctcgctcccgccacagagcccgcagcacgccgccgccgcagcctaggtcacgtgagtacccacgcgcgcgtcttgccagcggattcatcaccggcctgctcagactaggttctgcccactctgaccttctaaatggtacgtgggaggacgtccgtccccttcggacccaagagtcaccgtaacactctagaaggggagaaaaggagcgagggcggcaggcgacagagaacctcgcgagtcagcggccccgcgcagacccccccaggcacggtcccctgcggccacgtcggctgctcggcgcctgcgcaatctctttctctccagcgaaaccgaggcctccggagagcctagtagagagtgtgggcagtgagcgcttgtagccgctagag

SEQ ID NO: 57 - DNA, 1700 bp, Homo Sapiens
aatctaggtctggtaggttacttgcacacactgggtggaggtggaaggggcaaagtattcctgaggtgatggccctgatcctacctcacatctccttggggcctggttggacctggctgagcgtgtttgagcagtggctgatttttggaaaacaggaggactcttctccaggaggctgtgactaccaaggttgcaaatgctccacctgaacttgggtgcatgagggatctctctctctcacacacacacacccccccaccgcgtaagataaatttctgtttgtgagctgcctacattaaatgctttgctcctgaccttctgtgattaaaatctgttccgtttgcagggttcggtgcgcttggatttagtgtattttgttggtacagtaaaaaagagaactgcttctctataggtgacccccgccccaaagtcccccctcccactaacttctctttgagaagagagtaaaaccgaattcgagagaaaagaggcagtaactattcgggaggaggcagactggagtgggcaccagtcccggtagtaagagtcttaacgtcttcctcgctttcctctcgcagagtagtatggaagcatcagctcctccccctgggcctccaaaagttacctttcctttgggtgtgattctcgaacccgaagcgtttgagtcctaagaaactagaacaagttagagccaaagaggcctggcgggcgggggtgcgggtggagtccgggccgggtggcgtggaaggccgagcacagctgcgagcggcgagtcccgggagaggaggagcgggagaaagcggggcgggcctgcggtcggggttcggccgggggagggggtccccgtttccctctcctcatcctcgtctcctctggctctggtgacgttgcggtttcctcccccctccagcggagcctcagcgcccgccgcgtccctccctccgcccctttcgcccacacctacccgcccccgccggcccggctctcagtagcgtcgcccgaggctgcagcagcgcatcccggggcatggcgcggcgggggcgcggagggctcggttcggagggggccgggagcccgggcgccctggagtgaggaggaccgggagctggctctggaggctgcggaggcgacgccggagagaacgaagcctcggctgggagcggtaagtggaggggcgcgcaagggacgagctggggcgcgcggcgcaactttcttcgggagtttcgggcgggtagccttcggggctgtcccggcgcgggcgggagacggagagccacgtccccggctgagagcggagcgccgcgggttgcgcgcccgggcccggctcggctggactccgagagagcgcctgacgcgtgaagcctgagccacgggcttccccggcggcggagcagcctcgaggggcagcttttcgatcctccccacccccttgaacgccgagcaccgagccgggaacacgatgagaacctggagatcgcattccgtgcgtcggatctggccggcccagccgcgatttcctcgacggtccttggggcctcggcttcactctgcggggttgggtggcacaccctcgcgtcctcctttccctccgtaccccagccatttgggggcaggggaggtggcggcggcttgtgtgcagatcctggcgctggcaaccctactttgcggctgcagaagccgaggaagagctccccaagcct

SEQ ID NO: 58 - DNA, 1700 bp, Homo Sapiens
aacagaattcagaactctggggcagttctttggagttggagctcctagggcaaagataacgctaatgcgttgtttaaaagggttcaatttttaaaatacttttattggtaacacccattctgttcatcagaaatgcagccatgcatttttgtgcccggtaaagattttctgttctgtgtaaatctacaaactaaaataattagtttagattcttgacgtgaccctgcagtggaagtatttgcaccagtgaatgctgaaaagcaaataaaaatgaaagctttgcggaggccgtgggccggacgggcgcggggcgcctcctgccatcttagcgtctgccgggatccttcccgcgccttcgctgcccgaaccagctcggtcttgggacgtttcccccaaatcctcacaagtcaaatcgaactcaggtccgagttcgtcactgcaaaggaacccggggtgcggggtgggagggataggaggagagatgggctgggtgccaaatggtagaacaaatggttgatttaattaaagttgtcaatctaatttcccatccctctcacaccagccctttttttttttctttttcttttttcctcttaaacttgctgaatcccactatctccgcgttggtaagaaaacctgaataccttgggaattgtgcttaaattgcacttgcaactcccctggaagttcctgaaacttcccgagaatgaggcggggcggctcaaggggaaagggcagggaggagagaccctcgggaagctgatgacgcgtccggggcaagtcagcctgtttccgagacgcgcgcctcacggtcgtagcagcgctgcgcccaccctgcccctttagtgacccagccagcctttgctgggggtttctgataacgctcatcgataagctctccttgctcccaaaaggcgtgcttctctttctctccaggtacaaagtaggggtgggaaggggagagctacacaatgttgtaaaaagaagcgtggctttatttggaagacggttgaatttcttttgcttcctcgacccagttagtaacgacaagaaagatactgaatatcgccctccgaaaatcgggcgcatttgaactcggtgaaaagtgtccctgtgaggaagcctaatgtttacttttgtgcaaggataactggaaattaaagcgtctagtaatgcagagatttcattaagcgagcgctcccctcccccccgcgccaggaatgaaataagccagttagacagaaaacacttttgggagaagactaattacttctgggtaggccgaattgactggaaattataccaagcaaaggcagtacattccattccgtcctggcgatgttgattctatattattagaacattgttcccccagtgtgggggtgctttgtacttcccaaagtcagggtagactggttacctgggaaaatgcagtgttagtgctttgctcctactttcttcactgtttaaacatgttccacatttttctaatgccttctgagggttctcaacccagccactgagttcctcacccttggcgactgtgataccaacaatgctgtcaccctccttgggaaacatggttgcaaagatgctccgcaggaggcagataaaaggctgtgctttcagatactgaaggttgctttgttacttagatgtttccaattcctgcatagagaaggagggaaaacacggggtgc

SEQ ID NO: 59 - DNA, 1700 bp, Homo Sapiens; identical to SEQ ID NO: 58

SEQ ID NO: 60 - DNA, 1700 bp, Homo Sapiens
actggatgagtggggttctctgccccagctggacttccacaggaggatggaggaaagcatcctcttgagcaagtctggagagtgaaggggtggggttgcaaggttggagaaggaggtgtttccctcataaaaatgactgggcctttcttgcaatggctggcacaaagtcggaagggtctggggacgggaggggcctgaagtgatttctgaggagttgggtttttcctgtaaggccctgggagggaaggcaaggtactttgcgcaatgacacatttcagttgttggatgtgaagattctccttcgaagacagaataaagaaaggccccttcattaatgcgggtttgaaactggtctgaaatggtgtgcctgcagggattcgggaggaaatgcgacgcccacccccagacttgggaaaggcgggggcaataattctctaagagaactggagcccgaaagaggaatgaaaagcccccgcgccgcgcacatacactctctctcactggaaagttaggagcgaggaaagaactctgaagtgatttgggaacgatctgaaagcgcgcaagttggaagtcgggttgaagccagcatagcggagcaggggtcaggcctctgcgtggccggagtgggagagcgcttctgagaacaagatggggcaggaccgaaggggcgtgaggtcgacgctccctagggatgattcggagggtgaaggaggtttgggggtggttccgggctctcggaggtgactgagggagtccctgagccaatgcccgggggtgggaggcgctaccggtactcactgagctgatctccagcgccagggcgcactgcagcgccttcaccttcggcatccgtgcggggaggggcggcggggagtgaccccggccacgggcccgagtgaggcggggagacctggggctgggcggggacggggaggagacgaggtggcggcggcggtggcagcagtggcccccaggccggggcccgcggtccagcctgggttccgccggagaagcagctgagcgcggggcgcagactcgttgtcatggcagccaggaggggcggggccggaagagaggccgcgcggggcactccagggctacccgcgctgggacgagcgctggaggcggagcttgacgaacgccgagcgaggcggagtcgggcagggggcggggcttccgaaggggcggggtgaggcaaggcagctcttctgtgacgtcaaactgcgcccaaagccggacgaaagggagctgctgtttgtataatgggacggtgctctgttcagggagttggagttcctgtaaggccgtcggagggaacccctgagttacaacgtggtgatggcaccttgggctagtggcgacccactgtggcctcagttttcccaattgtaaaagaataataattattgttagtcctcctatttaaatttcacggcacggtttacaaagccagcataaggatactgggaattagggaagagacttgcccaaatcaaagtcggaaccaggactggaacccaggagctctcaagttaaaccccagttgcctccctgaggagaggggcttgcatcagatggtccatactcctagtctcctattctgtgattccctttgttagcgcgttcccttttcccttcaaattttcctgtctaattcacccatgtatgcgtcttaatgcatattgcttaagtggaactgt

SEQ ID NO: 61 - DNA, 1700 bp, Homo Sapiens
tcaggagtccagatccgggcaggaaataaagttctaaagcggctttgaagtgctttctccccagacccctccccagcgcgggtcattcatctcagcctagggaagagaccaccttgcctgaacgtagccaaacgcatcgcacgtgggagccaagtgggagatgaaataatccaatgcgtccccctcaccaccaccacccccaccaaattagttaccgtcggcttaaactcatcgggaatgtccgtgtattttaccgatcagggaaataaaagggtgtatataaattagtggaagcgggtgggaaggaacaagggatgcagactgtcgtgagtaaaacgcctcgcggcttggtgagaggttgtttcttttttaagccactcatttcagactgaaatatcaagccagttcaggtcatttcctgagtagcactacaagttaaaacaagatccagaaaatctactaatcttcaattgcgctgcctccagactgggagcacagggggatagggtgtagggctgtggggaggtcgaggggagggggatttgtggaaaagcaaacgcccgagaaaccctagctcccgacctcagctgtttcttacctcactcagctcggccgaggagtcgtttccatatttcatggcaactccggaattcatgatgcacttttcagagctgggctctcagtcccccaggtctcggagcttaagagttggacttcacaggctcaatatccactgcttggtcccttctgccacattttagtctagacagccattttcacccaggacagttgcgaagcgcaaacggaggatgcaggcactcttaaaaagagcagccgtggctcgcgtttcacggacgagccattgctgcaggaggctcggtggcgcggcgcgctcgcagctgcggcagcggcggagcccgcgcggggaccctcccgcgcgcacccagcgccccgttccggggcagtgccaccctgggcgccgcctcgccactcctacggtgcctcgccgccccccattaccaggatggagggcacttcccagcacgagccgacaccctagagggcaccggcgagctcccgaaaaaactagggcggtggggcgaggaagatccgcgcctcggattccagagaaggaaagaaagctagagggagccggagcggagctgcacttcacgccgaagtttccccttcgaatcgtctggagttgtaagtggtttctgattgaacacagctgtctagctgctggccacggggtgcgcggaggaggcggggctagagggagaaaagggtgggggcgagtagcctggaggccgagcagaggagaccgtgctgggagagcagagcaggcatccttccagttctctgcaatagtaattaaggacccgagtccaggagggggagtagaaacaggaaaagccacctgaataatcgtgcaggaaattaaaaaaaaaaaaagaaaagaaaaagaaaaaaaacttccctcataggagccgctttccatgatttaataaggtcaagacaaacctaaacactgtacatcagacagcacggtgcacttctcacggaacattaagattttacaagggtgcagggcagtcattttccctaatcagtctaaagtcaacgctacgggtgtaaattcaatgtcaaactccctctgtctgtcactggggcagagtacattttctgcatgtccataa

SEQ ID NO: 62 - DNA, 1700 bp, Homo Sapiens
gctggcaagtgaaaagcccacagtccacagcatcctcactgtccagccccacttccactgttgagctcaggaatttgtttccctgcctccttttggaagcgatcctgtgtgtgcaggtaggggcagagggagtaaagccaaacgtaactatagcatctcagatttgcagactctcaaaccagcctcacacctcagtgaggtgacaatggctaacattttcatctcagttttacagaagcccagccacagcaggtgacctgcgcaaagctacacagcctctggctccaaggcagggctctctcccctgcctgtgccctctcgccaccgcagcccctgttctccagctgggggaccacccagaggaccacctggaccctgctgcctccgttgggccatttgcttggcggagtagttggggggtggggggcctgcaacctaggaccccacctggcgctgccccagcccaccgagcagtgagccagctctcccctcatcggtggcagggggagctgtcgggatctcagggctgccctgaggggccgcgcagatctgggggatgtaaaggcccaaacggactcggaatggccggtgcggccgggctctgggaaatgaagggctcgcggtgtctcgcactttttgtgggggcgggagtaattaccaggtggccgcggagccagccgagggggcggctgctaacccgggcggcgaccgcaggcggagacgccccgcccgggacagggaccgggccgcgccggcgctcgcggacagacacccgcggtcccggccgctgccgcgtctcccgcccccgccacgccccggcggcctcgcttggggcgcgcccccgcctcgcacgcgcacgcgcctgcacccgcactggcccgcccggttcccccgcgctcgcggggccgctccgggggaggtgccgagacccggccttggcccgcgcgcggtttccggggtccggcgcgccggggacccacaggcgaggcgagagaaaggggttggctcgcggggacacctacctgcgcggcgcggggctgtggcgccggccaccattaccccgcgcccctcctcttgccagcgcgcacgcagatggcggggtggcctggggaggtcttcgggtcccttcctgggaacgcagggccaagttgtgctccgattccacgccccccccacccacgtcgggcacacgcagccctgagtgagggtccatgccctgcctttcacgctgaccacagcccctaggcttggcgcctgccgggaagggccgcaatggctcagggacccctgcccccactaccatcctgccctggggggacaggctctagaacactgcgtggggctcagagtgggcctagctgtaaaaggacaggggtccttctccagccacgcccactcccgggtgccctggctgtcagcagcctctcaccacaaccgcagggtgagaagcccagagaacagtccaggagtagcccttctgtcctttaccacgtccgtgtgcccagcacgggcaaatcagtagccacgcaataaatgaagtgcggtgttgcagctctgggcctctctgcacctgccctgacctcagcctgtccagcccagcctggggatctgcacttaggtttgcagcctcctgggtccctgcccggtgctcctcagagccggagcctgtctgccgcagggatggcttagaggacctccta

SEQ ID NO: 63 - DNA, 1700 bp, Homo Sapiens
gcaagactccatctcaaaaaaaaaaaaaaaaaaaagcgacttgcccagtaatagctctaaaatatggaagagctgggattggacttagactatctggttctggaactgggctggcaagtgaaaagcccacagtccacagcatcctcactgtccagccccacttccactgttgagctcaggaatttgtttccctgcctccttttggaagcgatcctgtgtgtgcaggtaggggcagagggagtaaagccaaacgtaactatagcatctcagatttgcagactctcaaaccagcctcacacctcagtgaggtgacaatggctaacattttcatctcagttttacagaagcccagccacagcaggtgacctgcgcaaagctacacagcctctggctccaaggcagggctctctcccctgcctgtgccctctcgccaccgcagcccctgttctccagctgggggaccacccagaggaccacctggaccctgctgcctccgttgggccatttgcttggcggagtagttggggggtggggggcctgcaacctaggaccccacctggcgctgccccagcccaccgagcagtgagccagctctcccctcatcggtggcagggggagctgtcgggatctcagggctgccctgaggggccgcgcagatctgggggatgtaaaggcccaaacggactcggaatggccggtgcggccgggctctgggaaatgaagggctcgcggtgtctcgcactttttgtgggggcgggagtaattaccaggtggccgcggagccagccgagggggcggctgctaacccgggcggcgaccgcaggcggagacgccccgcccgggacagggaccgggccgcgccggcgctcgcggacagacacccgcggtcccggccgctgccgcgtctcccgcccccgccacgccccggcggcctcgcttggggcgcgcccccgcctcgcacgcgcacgcgcctgcacccgcactggcccgcccggttcccccgcgctcgcggggccgctccgggggaggtgccgagacccggccttggcccgcgcgcggtttccggggtccggcgcgccggggacccacaggcgaggcgagagaaaggggttggctcgcggggacacctacctgcgcggcgcggggctgtggcgccggccaccattaccccgcgcccctcctcttgccagcgcgcacgcagatggcggggtggcctggggaggtcttcgggtcccttcctgggaacgcagggccaagttgtgctccgattccacgccccccccacccacgtcgggcacacgcagccctgagtgagggtccatgccctgcctttcacgctgaccacagcccctaggcttggcgcctgccgggaagggccgcaatggctcagggacccctgcccccactaccatcctgccctggggggacaggctctagaacactgcgtggggctcagagtgggcctagctgtaaaaggacaggggtccttctccagccacgcccactcccgggtgccctggctgtcagcagcctctcaccacaaccgcagggtgagaagcccagagaacagtccaggagtagcccttctgtcctttaccacgtccgtgtgcccagcacgggcaaatcagtagccacgcaataaatgaagtgcggtgttgcagctctgggcctctctgcacctgccctgacctcagcctgtc

SEQ ID NO: 64 - DNA, 1700 bp, Homo Sapiens
aggtgggcaggtcttctggagcagcaggcggctctcccagttcttatggctggagctaggatcctcctctgcctataactccctcaattctcccaacccaacaccccctaccttccttctttcctcctcccttccttctccccttcctcctttccttcccccctctttctttccttccccctctttcttccttccccctcctgccttccttccccctccagtcttcccttcccaccctgttgtgggcgggctgcagtgctgatcccaccgggacgtttggacaggcattgtctgtgacaacggtggcattggaaggccctggcagctgaacgcccagcctcccaccctccatgtcctcacactccccatgcacaaactctgcaggaagcaggccagggaacgcagttcactgtcttcccccgaccagcagccccagcctggccacatcgcagccgcggcagaggacacggcttattcccacgagccaaccggggcttattcccaccctcggcggtcccagcgggctcagcaagcgagagcaggcttccggagttacaggggcttccctgcctccgcagcgagggaagcaggaggcggcaggccctgaggcccgtgggctgaaggctcaggcctcacgtggagccacccgcggaggacccaggctaggccaacctgcccgtggggaacccgcccgaccctggcgagttaggggtctggaaggccgggtggagcttggtcaacagctcgggctcctcaccctgggccccgcttttcccggaggcctcgggggcacccagcgggtaggcctccctggcccggccggtcctggagcaggtgaggaaggccaggcaggccccgcggaggcgggccaggtcccggtgggtggtctcgctgacgaagcagtagagcacggggtcggcgacgcagttgaagctggtgagcaggagggagaagtggtaggcgttgaaaacgcccttggcgaagtcgcagctggcctcccagacgctgcgcaccagcagcaacacgtggtagggcaggaagcaggccaggaagatgaccacggtgctgagcaccagccgctggatctggtccttgcggctcttctgggtgccgtggctccggcgcacggcgcgcaggatgccctggtaggacgccagcagcaggcagatggggaagaggaagcccaccaggaagcggtagtagttgatggcgcgctgccatgcctggatggggtagtgctcaaagcacacgcggtgctggttctcgtcctcgatgacctcctcgtgcatcaggaagtagatgctggtcagcagctccttggcccagatgaccacgctgacgccgacggccgccttcagggtccggaactggtggaagcggaagggatgggccacagccaggtagcggtccacggagatgcagcagaggaagcccacgctgatgtagatgttctcgtacaggaggatgccgcacacctggcaggacaggtcgccgtgagaccagttgtcgtgctgcagcacgtactgcagccagaagggcagcgagcagatgtagaagaggtcggccaccgtcaggttgcacaggtacacgcccagctcgttccgggccttgatctgcaggtagccgaagtagagggacaggcagttggccgggaagcccaccaccagcacggtaacatagaccaccggggccagcgtctg

SEQ ID NO: 65 - DNA, 1700 bp, Homo Sapiens; identical to SEQ ID NO: 64

SEQ ID NO: 66 - DNA, 1700 bp, Artificial Sequence
  chemically treated genomic DNA (Homo sapiens)
ttttttaggattagagaatttcgaagagttcgtgtattgtttttttgaaagtttcgaagttaagtaattttaaaaatgtttagtaaagtttttttgggaatggtgagatggggggtggggaggatgggatatttttattgttatcgttcgtttttcgttttttcggtttttttatatgaagtgattggatttgggagatttagcggaaatttttaggttatttagggaattgtttttttttttggttttgtagcggcgaggcggggtagggggtggggggggtgggtcgtgagagcgtcgagacgcgcggggcgcggagatgtgtaagtggcgaagtttgatcgagagtaggttggagtagtcgtttaatttttggcgcgggatttgttgaggggttacggtgagtttttgtgggtgcgggtcggagggatgtttaggtgtttcgtcgttttacggttcgggcggaggcgggttcggaattttttttaagttcggttttagtttggagttcgcgcgttttgggcggatgtacggattgagaggcgtttggttgggtttttagttcgttattgttattcggtttcgtgcgtttattggtcgggatgtgtcggtttagtagtttttaatttttttcgaattttattggtgagttttaggagagcgagttgagggagaaaggtttaaagggcgcgagcgtttagggcgttgggagttcgtgggacgggtacggggagggtagagttagtcgaggggagcggttgtcgggaggtgcgttgagtagggttcgtagttaaggtttagcgcgattcgagcgtttaggattttgcggtcgtggaggaggcgtgttttgggaaggagtcgttattgggatttgaagagtgaggggagggagaggggtttagagggtgatttggaggagggttttggaagttacgttaggttggttttttaggtttatttttatagttttttgcggtttttgttttggggagttatgttttgttatcgagattcgcgttattagattgtatcggggttgatttgggggttgggaatttgttattttgttgtatagatattgattttttttttttttttttaaaaagtaaggtttgtttttattttggtttttgtcggatttttttatttgtaatttatttttttatttttattttttttaatcggaagatttttttattattttatttttttattttgtttttaatttgattttttattatataattaatgtttaaattttataagggttggtcggtgcggggttggtgtgtttttttgtagatgaaagtttaagattttttaattggttttagtttatttgtttggttttaaaataagggtaattgtaattaaatatttttttttagtttttcgattaagtcgattttttttttatatatgatttaaatggtaattttattaaattgaaaattagtatttgatttgtagcgttagtatttggaaagtgtttgtcggtaaatcgattgtatgaaggcgtgtagttttattttttgttatttttgtgggttttagtattttaaaaaattgtttggtaattttttggatttaattttgtgtttttggttgtagagtagtattaattaggggagtaatatggttggtatttcgtgtttagaatttcgtattttaggttgggaaatatttaattaatgtttggg

SEQ ID NO: 67 - DNA, 1700 bp, Artificial Sequence
  chemically treated genomic DNA (Homo sapiens)
tttagatattaattaggtgttttttagtttgaaatacggagttttggatacgaagtgttaattatattatttttttggttagtgttgttttataattaaagatatagggttaagtttaagaaattattagataattttttaaagtgttagaatttataaaaatggtaggggatgaagttatacgtttttatatagtcgatttgtcgataagtattttttaaatgttgacgttgtaagttaggtattagtttttagtttaataaaattgttatttgaattatatatgggggaggaatcgatttgatcgagaagttaaagagggatatttggttgtaattatttttattttgaaattagataaatagattgaaattagttaagaggttttaaatttttatttataaaaggatatattagtttcgtatcgattaatttttgtgagatttaagtattaattatataataggaaattaggttaaaagtaagatgaagaggtgggataatgaaaagatttttcggttaagaaaagtaaaaatagaaaagtgagttataaatgagaaaattcgatagaaattaaaatgaaaataaattttgttttttaaaaagaagaaaaaaaaattagtgtttgtatagtagaatggtaaatttttagtttttaaattagtttcggtgtaatttggtagcgcggatttcggtaataaaatataattttttaaggtagaggtcgtagggaattatggaaatgaatttgaagagttaatttgacgtgatttttagagtttttttttaggttattttttgggtttttttttttttttttattttttaggttttagtagcggtttttttttagggtacgttttttttacggtcgtagggttttgggcgttcggatcgcgttggattttagttgcggattttgtttagcgtatttttcggtagtcgtttttttcggttggttttgtttttttcgtgttcgttttacgggtttttagcgttttgggcgttcgcgttttttggattttttttttttagttcgtttttttgggatttattagtgggattcgggagaagttggaggttgttgagtcgatatatttcgattaatgagcgtacggggtcgggtggtagtggcgggttgagagtttagttaagcgttttttagttcgtgtattcgtttaaggcgcgcgggttttaggttgaggtcggatttgggggaggtttcggattcgttttcgttcgggtcgtggggcgacgaggtatttgggtattttttcgattcgtatttatagagatttatcgtgattttttagtagatttcgcgttaggagttgggcggttgttttagtttgttttcggttaagtttcgttatttgtatattttcgcgtttcgcgcgtttcggcgtttttacggtttattttttttattttttatttcgtttcgtcgttgtaggattagagagaggagtagttttttgggtgatttgaaggttttcgttaggttttttaaatttagttattttatgtgagggggtcggggagacggggggcgggcggtggtaatgaaaatgttttattttttttattttttattttattatttttaaaagggttttgttaaatatttttagaattgtttagtttcggggtttttaagaaaataatatacgggtttttcgggattttttagttttgagggg

SEQ ID NO: 68 - DNA, 1700 bp, Artificial Sequence
  chemically treated genomic DNA (Homo sapiens)
tagttggtaagtggtagatttggaattggaatttagtttagtttgattttttaatttatgtttttgattattatattgtttttttaagtttagatttgaaattttattttttgtgtggttgtgtgtttgggataggggtaattaatttttgattattttatatgttgtatagaatttggagaggattttttaaagtaaatgaatttcgaaagttggattgtagagtaaacgagtgtagttaatttagttaggggtttgtaagagggagaaagagaaaaagattgtggaatggaaagttttttaatttaagttttttttaaggggtagttattttttgttttatagtttagggtttgtatgtgttttttttggagtggaaaaatatataagttataaggaatttaatagatagaaaggcgtatagaggaatttaaagtgtgggttggggggcgaggcggtgggcgggaggcgagcgggcgtaggcggaatatcgttttttaagttaagtcgtcgtaaataaaaaggcgtaaagggagagaagttggtgtttaacgtgagttaggagtagcgtttcggttttttttttgtttattttaaaagtattttttgtattgtttttaaggtgagaaataggaaagaaaacgtcggtttgtgcgttcgttgtttgtttttttggttgtttgtttttgtagggttgttgggagtttttaagttttgtgagaattttgggagttggtgatgttagattagttgggttatttgaaggttagtagttcgggtagggtttatcgaaagtttattcgtatatattaggtaatttaattttttattttgtgtgatagaagtagtaggaagtgagttgtttagaggtaggagggtttattttttgttaaaggggggattagaatttttttatgcgagttgtttgaggattgggatgtcgagaacgcgagcgattcgagtagggtttgtttgggtatcgtcggggtaggattcggaacgtattcggaaggttttttgtaagtatttatttggaaggagaatttgggatttttttgggaatttttcgtttcggttggattggtcgagtaagtttggaaaatggtaaatgattatttggattaattataggtttttagttggtttgtttgttataatttatgattcggggttgggaaaaagattaatagtttacgtgttaaaaaaggggtagagtttgatggagttgggtggatttttttatgttatttgtttttatatttagaggataagtatttttgtagatatttagtgtaagggagattatgtttgattgtatggatgttttgttagtgagttttgggtaaattttggatttttatattgcgagttcgtttttttgtatgttttaggagaaagtttttaaagtatgttttagtggattgatttaaatcgaatggtagtatcggtatattgtttaatgtaggtttatttttttttttttttttattaagaaaaaaaaaattgttttttttgtatgtaataaagacgttggaaataaattgtattggtagtaagataaaggatttaatgatttaatgttgaaggggtgtgatatgttggtatgtatattgattttttttgttgaaaattttttgttagatgttttttttttaaataatttttttcgtttttatgtttttttttttattaattttata

SEQ ID NO: 69 - DNA, 1700 bp, Artificial Sequence
  chemically treated genomic DNA (Homo sapiens)
tataaggttggtaaggagaggagtatgggggcgggaggggttatttgggaagaaaatatttaataggaaatttttagtaaagggaattaatatgtatgttagtatattatatttttttagtattaaattattaaattttttgttttgttattaatgtagtttatttttaacgtttttattgtatgtaagagagataatttttttttttttggtagaagaagggaaaaaaataaatttatattgagtagtgtgtcgatgttgttattcggtttgggttaatttattgaagtatgttttgagagtttttttttggagtatgtaggaagacggattcgtagtgtagaaatttaggatttgtttaggatttattgatagaatatttatatagttaaatatgattttttttgtattgaatgtttgtaaaagtgtttattttttaggtgtggaggtaaatggtatagaaaagtttatttaattttattaaattttgttttttttttggtacgtaggttgttggttttttttttagtttcgaattatgaattatgatagataagttagttaaaagtttgtaattgatttaaatgattatttattattttttaggtttgttcggttaatttagtcggggcggggggtttttagaaagattttaagttttttttttaagtaaatgtttgtaaaaagtttttcgaatgcgtttcggattttatttcgacggtgtttagataaattttgttcggatcgttcgcgttttcggtattttagtttttaaatagttcgtatgggggaattttggttttttttttggtaaagaatagatttttttgtttttgaatagtttattttttattatttttgttatatagaatgaaagattgaattgtttaatatatgcgagtgaattttcggtgaattttattcgggttgttaatttttaaatgatttaattagtttgatattattaatttttaggatttttatagagtttaaaaatttttagtagttttgtaaaagtagatagttagagaggtaggtagcgagcgtataagtcggcgtttttttttttattttttattttaaaaataatataagaagtgtttttaaaatgagtaggggaggagtcgggacgttgtttttggtttacgttgagtattaatttttttttttttacgtttttttatttgcggcggtttagtttggaaaacggtgtttcgtttgcgttcgttcgtttttcgtttatcgtttcgttttttagtttatattttaaatttttttgtgcgtttttttgtttgttaaattttttataatttatgtatttttttattttagaaaaagtatatgtaagttttaaattgtagaatagagaatggttattttttgggaaaggtttgggttgggaaattttttattttatagttttttttttttttttttttttgtaagtttttggttgaattgattgtattcgtttgttttgtaatttagttttcgagatttatttattttgaaaaattttttttaggttttatgtagtatatagagtagttaggaattggttatttttgttttaaatatatagttatatagaaaatgagattttagatttggatttgaaaaaatagtatagtggttaagagtatgggttggggaattaaattgaattagattttaattttaggtttgttatttattagttg

SEQ ID NO: 70 - DNA, 1700 bp, Artificial Sequence
  chemically treated genomic DNA (Homo sapiens)
ttttttttggatatcggagggaatttcgaatatatcgttttattgtttacgggattgggaagtatacggaggttttgttggttaatttttgttttgttattgttaataagttattattagtttttataattatcggggggttggcgtcgatcgcgggtggcgcggggcgggattcgtttttgggacggtggagggcggtggcgttgtagtcgcgtcggtttttgagttttagggagcggagggtacggagtcgggcgagttttgcggtgttagaattttgcgtcgttttgacgttggggtttagggtttatttcgggggcgcgcgcgtggttgcggaggcgggaaggtttttttgtttgtcgtcgcggagtttcgcgtttttgtatttcgagttttgggggttcgttttgggttgtttttttttttcggtttcgtcgtttatttggttcggtttttgttagcgtttcggtttttttcggcgcggcgcggggttcggtgtttagttggtcgttttgcgtttcggcgcggttgcgttttttttttggttttggttttggtttcggtttcgggtcgcgttgtttttggttgttcgtgcggtcgttttgtcgttgcgcgtcgtttcgtaggatgttttggatgaggaaggacgtgagcggtttggacggggtcggcggcgcggtttttttcgttttcgttttttcgggtcgcggtttcggaattttgagtatttcggcggtttcgttttggtttgaatgtattcgttcggttcgtatcgtttttatttttcgtttttcggttcgtttatagtttaggggagcgggtcgcgtcgtttttttattggtcgcgggacgggtcgcgtcgcggattggtcgttttgagttttaggtgatttcgtcgatttgcgagttttgagtattgtttcgtttcgtttttttttaggggattttttttttagttttttttttttttttaattttttttttttttttaattttttttttttttttagttttttttttttcgttttgagtcgtttttttaggttaggagcgtattttaaagcggggtttttttgttttaatttattcgtaatagagagaagtttttggggatgggacgattaagataaatagtttagttagttttttaggatcgttagagttaattataggggatgagtacgtagttattgtagtcgcgcggtatcgtgagagttttgggtgttttttttttttgggagagatggtgtaaatttagatttgtaatatttacgattttaaagaggtatgataagtaggtgtagttagattttaggttgaaagaaggaaaatatttttgttaaagttttttttcgttgtagaagtggttttttttggtggtttttttattcgtaataatttgttaatttttttttttttttagatatatgatatattatttttttgaaaagtttttagacggatggaaggcgttttaggttaggataaatgtaaaaattgtatttaaaaaagtagtttaaaagaatattagtatattttttttgtagattattaattgttaatatgtaatttattggttaaataatgggttagttagattggtttaatcggtattgtgattggattggacggtatcgtgataatagacgaaagtggaatagagatgatggaagtattttttttttttgttttttttttttt

SEQ ID NO: 71 - DNA, 1700 bp, Artificial Sequence
  chemically treated genomic DNA (Homo sapiens)
agaagagaaaagataaggaaagaaaatatttttattatttttgttttattttcgtttgttgttacggtatcgtttagtttagttatagtatcggttggattaatttggttaatttattgtttagttaatgggttatatgttaatagttggtaatttgtaaaaagagtatgttgatgtttttttgaattatttttttaaatgtagtttttgtatttgttttggtttaaaacgttttttattcgtttggaaattttttaaaaggatggtatgttatgtgtttggggaggaaggaaagttaataggttattgcggataaaggaattattaaagaaaattatttttgtaacgggaaaaggttttggtaaaggtgtttttttttttttagtttggggtttggttgtatttatttgttatgtttttttgaggtcgtagatattgtagatttgagtttgtattattttttttagagagagagagtatttagaatttttacggtatcgcgcggttgtagtgattgcgtgtttattttttgtaattggttttgacggttttgaagagttaattggattgtttgttttgatcgttttatttttaggagtttttttttgttgcgggtgggttggggtagaggagtttcgttttggggtgcgtttttggtttgggaaaacggtttagggcggagggaggagagttggagaaggagaggaaattggggaaggagagggaattggggaaggagagggaattggggaaggaattttttagggaggagcggagcggggtagtgtttagggttcgtagatcggcggggttatttggggtttagggcggttaattcgcggcgcggttcgtttcgcggttaatgggagggcggcgcggttcgtttttttgggttataagcgagtcgggaggcggaaagtgaaagcggtgcgggtcgggcgggtgtatttaggttaaggcggggtcgtcgggatgtttagggtttcggagtcgcggttcggggaggcgaaagcggagggggtcgcgtcgtcgatttcgtttaagtcgtttacgtttttttttatttaggatattttgcgggacggcgcgtagcggtaaggcggtcgtacgagtagttagagatagcgcgattcggagtcggagttagagttagagttagagggaggacgtagtcgcgtcggggcgtagaacgattagttgagtatcgggtttcgcgtcgcgtcggaggaggtcgagacgttggtagagatcgagttaggtaagcggcgaggtcggggaaggggggtagtttaaggcggatttttagagttcggggtgtagggacgcggggtttcgcggcgataggtagagggattttttcgttttcgtagttacgcgcgcgttttcggaatgaattttgagttttagcgttagggcggcgtaggattttgatatcgtaggattcgttcggtttcgtgtttttcgttttttggggtttagaagtcggcgcgattgtagcgttatcgttttttatcgttttaggagcggatttcgtttcgcgttattcgcgatcggcgttagtttttcggtagttatgagaattaataataatttattaatagtgataaagtaggggttgattagtaaagttttcgtgtgttttttaatttcgtgggtagtaaagcggtatattcggggtttttttcggtgtttaggagaga

SEQ ID NO: 72 - DNA, 1700 bp, Artificial Sequence
  chemically treated genomic DNA (Homo sapiens)
ttggttttttaaagttttgagattataggtgtgagttatcgcgtcggtttttaaatatgttatttataaattgtttttggttcgttttttagatcgtaggtttaattacgtttttagaaaaattttttattagttttgttggttttcgggtagtttttataatgtattaattcgttttttttttatgtttttaaggagttttttttgttttacgatttgagattagaattgttttttttttattgttgatgtttaggtttaaatgatatttttttatacgtaaaacgggttttaagttttatttatggttgtgacgatttgaaagtaaatgcgtgtatatgataacgtgtgagcgatgagaagtttgttatttgtaaaattttatgcgtatgttaattattatggtcgtgataattatgtatattatagaaatattaattaagcgttgggttcgttttaggatttttagtattgttgggaggtttggtattcgataggtgttttaggagggtaggaaaggagagatatgtttggttttaagtttcgcgagggttttggggagcgttgggtttttaaggacgttttggatttgaatatttggtgagagcgtcgggtaggggaatagtaggagtaaagttcggaaggtggttatgaggataagaagggaggatatttggtaggttgttaaaattaggtttatttgtcgggtaggtggaggcgagtaattatttaagttttttgggatagcgggagagggggtaggggagtcgtcgtattattttttttgtggtcgttagggggttttcgttttaggtgagcgttaagttttagttttaggtcgtttttttatgttttaagtaagttttgtagcgtagttaattgttaggggttgtttcgtttatttttcgttttcgtagttatttgtttgcgcgaaatgtttgttaatttttgattggtaggtagtttggtaaattaaatcgcgatttttgaaagtaaaatattgtagtattttggtagttttgaattgggaagggatgaaggaggttgtgttttcgggttgtacgaagagttcgagttattttttagaaggttttgataggtgggttttagaggagacgtcgtcggtgagtagtgattaatatcgggaggaaggggaattgaatttaattttcgtttttttttggaaaaagcgaagttatttaacgttttttagtgtatatattttttttttttattgttattagtttcgttaatttgggtttcgttgttttggaatgttttttttagttttgtatcgaggttaagaggagcgggggtatgggttattttattaaaggtgatgttaggttttattaaattaggaagtgatatggagttaattttgttagaatttttttttttcgtgtcgagcggttcgggttttttggcggtagtagatggtggagttagtaggtgggatgaggggaggcgtttttggtttaagttcgtttttggaatagaggtgttgttttttcgagttgtaagtttttagtttagtgggacgggacggaagaatgtaattttttcgtgagttaaagtcgaggaacgggaagtttggtagggaattggcgtttatttttagaagttagatcgtcgggtggtgggaaagagcgtgttttattgatttgtttagaaagaggtaaattcgaatatagacgtta

SEQ ID NO: 73 - DNA, 1700 bp, Artificial Sequence
  chemically treated genomic DNA (Homo sapiens)
tagcgtttgtattcgaatttgtttttttttgaataaattaataaaatacgtttttttttattattcgacgatttggtttttggaggtgagcgttagttttttgttaagtttttcgtttttcggttttggtttacgagaaggttatattttttcgtttcgttttattgagttggaaatttataattcgaggagatagtatttttgttttagaagcgggtttagattaagaacgtttttttttattttatttgttaattttattatttgttgtcgttaggaagttcgagtcgttcggtacgaagaggagaaattttggtaaagttaattttatgttattttttggtttggtagagtttggtattatttttagtaaggtagtttatgttttcgttttttttggtttcgatgtaaaattggaaagaatattttaaggtaacgggatttaggttggcgaggttggtgatagtaagaaggaagggtatgtatattaggggacgttaggtgatttcgttttttttagaaaaaaacgaaggttaaatttaatttttttttttttcggtgttaattattatttatcggcggcgttttttttaaggtttatttattaaaattttttgagaaatgattcggatttttcgtgtaattcggaggtatagttttttttatttttttttaatttagagttgttaagatgttgtagtgttttgtttttaaaggtcgcgatttgatttgttagattgtttgttagttagaagttggtaggtatttcgcgtaggtaagtggttgcgaggacggggagtaggcgggatagtttttggtaattggttgcgttgtaaagtttgtttgaagtataagggagcgatttggggttagagtttggcgtttatttggagcggggattttttagcggttataggaagaatggtgcggcgatttttttgttttttttttcgttgttttagagagtttaggtggttgttcgtttttatttgttcggtaggtgggtttggttttagtagtttgttaggtgttttttttttttatttttatagttatttttcgggttttgtttttgttgtttttttattcgacgtttttattaagtatttaaatttaaaacgtttttgaagatttagcgttttttagagttttcgcgagatttgggattaaatatatttttttttttttgtttttttggaatatttgtcggatattaagttttttagtaatattgaaggttttgaaacgagtttagcgtttggttaatgtttttataatgtatataattattacggttatgataattaatatgcgtatggagttttatagatgataagttttttatcgtttatacgttgttatatatacgtatttgtttttagatcgttatagttatgagtgagatttgaagttcgttttacgtatgaagaaatgttatttgggtttagatattaataatgagaaagaggtagttttagttttagatcgtggggtagagaaagttttttgggaatatgggagaggaacgagttggtgtattgtagaaattgttcgaaggttagtagggttggtgagagatttttttgggggcgtggttgggtttgcgatttgagggacgggttagggatagtttataaataatatatttgggggtcggcgcggtggtttatatttgtaattttaggattttgggaggttag

SEQ ID NO: 74 - DNA, 1700 bp, Artificial Sequence
  chemically treated genomic DNA (Homo sapiens)
tatgaattagttatattatatagttgttttattaaaaattaaaattttgtagagtatattaatagagattgtatttaagagaaaggtagttattttttagttatttatataattatttattaagtatttatgtgttagttgatatttttgggtgttggaataaaataaggaaaaaattaagatttttatttttacgaagtttattaaagtgtggtaaatgatagagtaattatttttaatagtttttagtttgtattttgtaattaattgaatatagtaaatattaaaagttatttgaggtagggatttattttatttttatttgttggattttatttcgtgtttagtatattagtttgtaagtaatcgatatggattaaggggtaaaaataggtttttaaatagttaaaattttgataagtaattatttgtttgattattaaatatgtcgttcgaaatttttatttttttttttaaagcgtagtaaataagtatttgcgtatttattacgtgtaaaatagtttttaatataatgttttgtgtgtaaaaggaacggcgggaatggatttaagttgcgtatcgtgataaatggcgtgaatattttgagcgggtggatagtttttttttcgtgagtttttttttttttttttttagttattaagatagtacgcgtgtgtagaagttgtagcggtgggacgtagtgttaagtttgggggcgtatatattagttcgcgcgtcgcgagttcgtttgggtagttcggcgggcgcggcgaaatagtttttacgttcgttttttatcgatttttcggtattagagcggacgaggcgtttttttagaggaaagattgggcgtcgtagacgttttttggagaagtttttggttattcgttttatttcggggtttttttttttttgggttttttttgattttttgggtgggcgttttagataggttcgtcggacgcgatttggaaacgggtcgtgttcggtttagttcgtatttattcgttcggttatagtttagttatagtttagggattttgaggcgttcgcgtttttttgtcgcggtatttttagtttaagtaggtcggacgtcgaggtttcgcggtatcgagttttcgttcgtttcgttttttcgttttttatttcgtttcgttttcgtttttttatttttttttcgttttcgtttcgttttttcgataattttttcgcgttttcggttcggtacgcgcgttgcgttcggtcgtcgttgtcgtcgtcgtcgtcgtcgtcgttgtcgtcgagcgttttttcgttgcgtttggtttttagtttcgggtcggaatcggaagtttggggggcggagttcggcggaggttaggagatcgaaaacgcggtcgagttcggagttcggagttggagttagagtttggattagaatttggtcgtcgtttgtatcgtcgtcgtcgttgtcgttcgtcgtttttttttcgcgtcgtagtcgtttcgtcgttatcgtcgcgagttcggtcgttagtggttttcggattttaggtgtttgggttgaaggtcggttcggatatcggatttagttagtttttagattgttcgggcggtagcggacgtcgttgtcgtcgtcgttttttcgtcgttttagtttggcgttttgtttcgagagacgggagaggcgagcggagttgatagtgattttgatagtgatt

SEQ ID NO: 75 - DNA, 1700 bp, Artificial Sequence
  chemically treated genomic DNA (Homo sapiens)
aattattgttaaaattattgttagtttcgttcgtttttttcgtttttcggaataaaacgttaggttgaggcgacgaagaggcggcggcggtagcggcgttcgttgtcgttcggatagtttgggaattgattgggttcgatgttcggatcgatttttaatttagatatttaaagttcgaggattattggcggtcgagttcgcggcggtggcggcgaggcggttgcggcgcggggaaggggcggcgggcggtagcggcggcggcggtgtaggcggcggttaagttttggtttaggttttggttttagtttcgggtttcgggttcggtcgcgttttcggttttttggttttcgtcgggtttcgttttttaaattttcggtttcggttcgaagttggaagttaggcgtagcggaggaacgttcggcggtagcggcggcggcggcggcggcggtagcggcggtcgggcgtagcgcgcgtgtcgggtcgggagcgcgaggagattgtcggggaagcgaggcgggagcgggggaggggtgagagagcgagggcggggcggagtggagggcgaggaggcggggcggacggaggttcgatatcgcgaggtttcggcgttcggtttgtttgggttagagatatcgcgataaaggggcgcgggcgttttaaagtttttaagttgtggttgggttgtggtcgggcgagtagatgcgggttaggtcgggtacgattcgtttttaaatcgcgttcgacgggtttgtttggagcgtttatttaaagggttaggagaagtttaagaggaagaggatttcgaggtggggcgagtggttagggatttttttaaggggcgtttgcggcgtttagttttttttttggggggacgtttcgttcgttttgatatcgaggagtcggtggaaggcggacgtgggagttgtttcgtcgcgttcgtcgagttgtttaggcgagttcgcgacgcgcgagttggtgtgtgcgtttttagatttagtattgcgttttatcgttgtagtttttgtatacgcgtgttgttttggtgattagaaaagaaaaagaaaaaatttacggaggaagggttgtttattcgtttaggatatttacgttatttattacgatgcgtaatttgaatttattttcgtcgttttttttatatataaagtattgtgttgaaaattgttttgtacgtaataggtgcgtaaatatttgtttattgcgttttgaagaaaaaaatgaggatttcgaacgatatatttgatgattagataagtgattatttgttaaaattttagttatttaagggtttgtttttattttttaatttatgtcgattgtttataagttggtgtgttaggtacggaatgggatttaataggtaaaaataggatggatttttattttaagtagtttttagtatttgttgtatttagttggttgtagaatataaattagagattattgaaggtagttgttttgttatttattatattttagtgagtttcgtgaaggtagagattttgattttttttttattttattttagtatttagaagtattaattagtatataagtgtttaataaataattgtataaatgattaagggatgattgttttttttttaaatatagtttttgttaatgtattttataaagttttggtttttaataaaataattgtataatatggttaatttatg

SEQ ID NO: 76 - DNA, 1700 bp, Artificial Sequence
  chemically treated genomic DNA (Homo sapiens)
attttcgcggttatttttagttaggttagtatagcgtttcgtgttcgttgaatcgttttaggtagttttttgatgatttaaattattattatttttgggtttagagtaggataaatgaaattttaataggggtttgttttgtgatcgaaaattatttagttttatagtttttgaatgtaaggagaggtggtttttttagaattaatatgttttttaaggtgggtacgtttacggttttgggaaatgtatattttaggtaatttataggtgcgattttttagtggaggcggggtagtttgaatttttggttttgtaggagtgggaagaaatcgtttttgttttttcgataattgagttttttatttggggttgggagggcggatagtaggatcggtttttttattttttatcgcggtttttcgatttggttagtagttttggcgtttagataaaaggttttttgagttgggggtggggggtaggtcgagagtaggtcggtaaattagatttcgtagttttttaagttattagttttgtatttgggtgtagagatttcgttttaaaaattcgaagtatcgttttggttcgttttttgttcgcgggcgcgggttttcgtttatttatttatttatttatttaattatttatttatttatttatttttttttgacgtattcgttcgtttttcggtcgtttcgggcgtattatcgtttttttttttgtttgagggattttttgcgttttaggattattttatagagtttgtttatgttttcggtgaggttttcgtcgatgatggcgtacgtcggttggacgtgataggtggtggtcgggataagttcgtgtagcgttagttgttttttaatttttgttatcggtagcgatttgggtaggttttgtttgttggcgatgattagtagcggcgtgttttggttttcggcgaatttggttattttgtgtagtttcgttttggttttttttagtcggtttacgtttatcgagtttattacgtagatgatgtcgttcgtgtagcggttgtaggatttttatagcggtcgtagttttttttggtcgtttacgttttagaagtggtagttgatgtttttggtcgtgtcgttgtttagtttgattttttcggtgttgaagtcgatggtgggtatcgtgtttacgaattcgttgaatttgagtcggtagagtatcgtggttttgtcggtcgagtttaagtttaatatgacgatatgtagggattggaaggtcgagatgttagaggagatgttgtttatggtttttggttgcggcgttagttattgcggttgcggcgggagggcgtcgtttttcgagtagttacgggttcgacgcggtcgggcgtatttgggtttcgttcgtcgtcgttcgtatcggtttcggggttcggcgttttcgcgggcgtacggtttggcgcgtttagacgtcgtagttttcgcgttttcgtcggtcgggttcgcgatcggcgggtatcgcgtttttacgcgcggttttgtttttcgcgaagtcgttttagatgttcggcgttttttttaggtttgggttcgttttgtggttttttcgggtcgcgttttcgcgtttcggtagttgttcgggtttttcgttttttcggcgttcggcgttcgtttttggtttgggacggcgttgtttttgtttttgttttcgttttcgttttt

SEQ ID NO: 77 - DNA, 1700 bp, Artificial Sequence
  chemically treated genomic DNA (Homo sapiens)
gggagcgggagcgggagtaggagtaggagtagcgtcgttttaggttagaggcgagcgtcgggcgtcgggagagcggagagttcgggtagttgtcggagcgcgggggcgcggttcgaggaaattatagagcgagtttaggtttggggagggcgtcgaatatttgaggcggtttcgcgggagataaagtcgcgcgtagagacgcgatgttcgtcgatcgcgagttcggtcggcgagggcgcggggattgcggcgtttgagcgcgttaagtcgtgcgttcgcggggacgtcgagtttcggggtcggtgcgggcggcggcgggcggggtttaggtgcgttcggtcgcgtcgggttcgtgattgttcggggggcggcgttttttcgtcgtagtcgtagtggttggcgtcgtagttaggagttatgggtaatattttttttaatatttcggttttttagtttttgtatatcgttatgttgggtttggattcggtcggtaagattacggtgttttatcggtttaagtttaacgagttcgtgaatacggtgtttattatcggttttaatatcgagaagattaagttgagtaacggtacggttaagggtattagttgttatttttgggacgtgggcggttaggagaagttgcggtcgttgtggaagttttatagtcgttgtacggacggtattatttacgtggtggattcggtggacgtggatcggttggaggaggttaagacggagttgtataaggtgattaagttcgtcgagaattagggtacgtcgttgttggttatcgttaataagtaggatttgtttaagtcgttgtcggtggtagagattgagaagtagttggcgttgtacgagtttatttcggttattatttattacgtttagtcggcgtgcgttattatcggcgagggttttatcgagggtatggataagttttatgagatgattttgaaacgtaggaagttttttaagtagaagaagaagcggtaatgcgttcggagcgatcggggagcgagcgagtgcgttaagaaagaatgaatggatggatggatggttggatggatggatggatggatgagcgagaattcgcgttcgcgaataaagagcgaattaaagcgatgtttcgaatttttaaaacggaatttttgtatttaaatgtaggattggtgatttaaggagttgcgaagtttgatttatcggtttattttcgatttgttttttatttttagtttaggggattttttgtttgaacgttagagttattgattaggtcggggggtcgcggtggggagtggaagagtcggttttgttgttcgtttttttagttttaggtggaaggtttagttgtcggaaagataaaagcgatttttttttatttttgtagggttagaagtttaggttgtttcgtttttattgggggatcgtatttgtgaattatttgaggtatgtattttttagaatcgtgggcgtatttattttggggggtatgttggttttggggggattatttttttttgtatttaggggttgtgaagttgagtaattttcggttatagggtaggtttttgttgaaattttatttgttttgttttgggtttaaaggtggtggtggtttgggttattagaggattgtttgggacggtttagcgggtacggagcgttgtgttggtttggttggggatggtcgcggaggt

SEQ ID NO: 78 - DNA, 1700 bp, Artificial Sequence
  chemically treated genomic DNA (Homo sapiens)
agtaaattgattttgtttgtttagttttattagttagggtggtatggaagtgggtggggagagggttggtgcgtggtatagataattgggaagcgcgcgtattatatatatatatatatatatatatatatatatatatatatacggttttagttaatatttatgtagtatttttggggggttttttatatatatttgaatagtatatttttaataaaggataggtatatttttgtttgtttttttttaatttttagattagtattttattttgggtttagaattggtggtgataagtcgatttagatggagtatttgagaaaaggggaggtgattttttgtttgaatgggggagggggcgttttttatttatcgtttgagtttttggaacgaggagggttaggatattttggatttttttaggtcgggtcggtggtcggtttttatcgcgtaggaggtgcggtttatggcgcgatagtataggtagcgttcggtgttcgagtagtcgcgatgtcggacgcggtcggtggtttcgttcgaggcgtagcggtcgcgggtagtttcgggcggcggcgggggcggcggtagggggggcggcggcggcggttgtagcggatattggggttggggttggggcgagggtggcggcggcgggggtaggtggtgatggggatgtaggaggcggaattggggttgtcggagcgggtattggtggtgttgttcgtgtttttagagatgtttgggggttttgagcgtggtttcgtcggcgttgtcgttgtttttttcgttgttcgttcgggtcggcgcgttgttgttttttgtttttattttggttcgcggtttcgcgatttgttgtttagttcgggtttatcgcgttggttcgagcgtttttttgatgttgttgttgttgttgtcgtcgtcgtcgtttttgtagtttagtagaggttggtgtttagttttttatttgaaggcgttagagttttagggttatcgagagggggttttgttagcgggaggggttatagaacgcggtagggttttttttatcgaggttatggtagagatacgttttcgggttttttaagttattaagattagggtgcgcggtgttcggtttcgttagaggttggtttttttttttttttttttttttttttttttttgggcgtcggcggcgagagtagggattgttagtaattcgggttgtagaagtagtagtaacggcggcggcggtagtggtagtcgtttttagggtatcggaattttgatttcggagttgttgaaaagttggatagttttaggttggtacgttcgagttgtgatttttattaaaattattttagttagttaggagttagatttgtttttttgattttttttttcgtcgtgttcgcgttttttttttttatattgttttttattgtttcggagtttttttttttattttcgtttgttttagtgtttttagagaaatggagtttggcgatttttggttggtttttagagtttgttgacgatgagttttatgttggtgtttttaaagataaggaatgtgattaggtaaagttgtttggaatattgtttatgcgcgtcgttgaaaaattggatttgggtgaagatttaatagtaagattaatttagtttttttttttgatataggataaaatattgtgtggtttttttttatagtagggagaatg

SEQ ID NO: 79 - DNA, 1700 bp, Artificial Sequence
  chemically treated genomic DNA (Homo sapiens)
tattttttttgttgtgggaaagagttatataatgttttattttatgttagggaaagagattgaattaattttattattggatttttatttagatttaattttttagcgacgcgtatagataatattttaggtaattttgtttggttatattttttatttttggaagtattagtatggggtttatcgttaataagttttgagaattagttaggaatcgttaaattttatttttttaagagtattgaggtaagcggaggtgaagaggaaggtttcggggtagtggggagtagtgtggaggagggaggcgcgggtacggcgaggggaaggattaagaggataggtttgatttttgattggttggagtggttttggtgaaagttatagttcgggcgtgttagtttggagttgtttaattttttagtagtttcgggattagggtttcggtgttttggaggcggttattattgtcgtcgtcgtcgttgttgttgtttttgtagttcgagttgttgataatttttgttttcgtcgtcggcgtttaaaggaagggaagaagaaagggaggaagaaggattaatttttggcgaaatcgggtatcgcgtattttagttttggtgatttggggagttcgggagcgtgtttttgttatagtttcggtggaaggagttttgtcgcgttttgtgatttttttcgttggtagggttttttttcggtagttttgaggttttggcgtttttaagtgagaagttaagtattagtttttgttgggttgtagaagcggcggcggcggtagtagtagtagtagtattaggaaggcgttcgggttagcgcggtgaattcgggttgggtagtaggtcgcggagtcgcgagttaggatggaggtagagggtagtagcgcgtcggttcgggcgggtagcggagagggtagcgatagcgtcggcggggttacgtttaaagtttttaagtatttttggaggtacgagtagtattattagtattcgtttcggtagttttagtttcgttttttgtatttttattattatttgttttcgtcgtcgttattttcgttttagttttagttttagtgttcgttatagtcgtcgtcgtcgttttttttgtcgtcgttttcgtcgtcgttcggggttgttcgcggtcgttacgtttcgagcggggttatcggtcgcgttcggtatcgcggttattcggatatcgagcgttatttgtattgtcgcgttatggatcgtattttttacgcggtggagatcggttatcggttcggtttgaagaaatttaggatgttttggtttttttcgttttagggatttaggcggtgagtggagagcgtttttttttttatttaggtaaagggttattttttttttttttaaatattttatttaagtcggtttattattattaattttagatttagggtaaaatgttagtttggaaattgggggaggataaataggggtgtgtttattttttattgagagtatgttatttaggtgtgtgtaagagattttttaaaagtattgtataaatgttaattggggtcgtgtgtgtgtgtgtgtgtgtgtgtgtgtgtgtgtgtgtgtgtggtgcgcgcgttttttagttatttgtgttacgtattagttttttttttatttatttttatattattttggttagtgagattaaataggtaaaattagtttgtt

SEQ ID NO: 80 - DNA, 1700 bp, Artificial Sequence
  chemically treated genomic DNA (Homo sapiens)
ggatagggaagtttagttaagaattaggagtttgatatatttaattttttattttattttattgatgattttgtgaggtatatttttttaattttagatttataaaagagaaagttgaggtttggagaggttttttattttgtttaaagttatataatttttacgtggtgtagttaggatgggaattagagtttggttttttgtatttttaatattattgttgaattcgggtttgtttgttttttttttttttttttaattattgttatatagattttattatagaatttattacgtggagggttattattttttttttttttgtatagtatgtttgttttttttattttatagagtaaggtgtttagtaaatgtggttgtttggaggtaatattttttttttatcgttatatttggggttggggtgcgaggtaaggtagggttggcgaggtggatgttgtagttgttattattgttaggtgttgggaaacgatttttttgtttgtgtattgattcgaaggtattttttattttttaggtttttttcgtttttcgttttttatttttagtttttcggttttagtttaggtttttcggggagtattttttgtcgtgagattgatagtttttgggggcgtagggttttgttttttgcgttttagtttatttgtgcgtagagtttcgtttttaggcgtttggaattcggcgggtattgacgttaagcgtcggcggagcgttgtttatagacggttgattcgggtttttttttatattttttttttttttcgtttttttttttttttttgtacgggggttcgggtttattataaaaggtgggagcgcgtggtgttttagtaacgacgagttttagaacgatggagagttttcgcgtgaggttgttgttttttttgggcgtcgttttgttgttgatgttatttttgttgggtattcgtgtttaggaggacgtcgagttttagtttcgagttttggatatttattttgtcgtggatgatgttttttacgagaaggagttggtcggtatttttttcgttttcgatttttttgagttgtcgttttgtttttttttttgtacgttttttttttttttttatttttatttttatttttagagttagggcgcggggagttgagcgtaacgtttaggtatttattgttattcgaagagcgtttcgagtttacgggtttttggtagtttgttgagcgaattttttatttcggtttttttgagtaatagggattttagcggtttagagattcgcggttagtatttgggatagcgttcgttaagtttttatttttcgattattttttgtgttcgcggagttttatcgtagagtgcgtgtgggttcggggttttttataattagggttggaagtgcgtatttgggttgggttcgtagttaaggcggtaattttaggtttcgaagcggtgtgttgtagatcgaagcgttgtaagaagttttgaagaagtttaagagtaaacgtgtttttatttatgagaagaagtatggttaagtttttatggtaaggtttgtggttatttttttttcgtgtttttttaagagaaagtatatcgttttgaatcgtatatatagtttttttcgtaggatgtggttaaataatttaggtaatgggtttgtaggattttgtgggtttttttttttttttcgggtgaggaa

SEQ ID NO: 81 - DNA, 1700 bp, Artificial Sequence
  chemically treated genomic DNA (Homo sapiens)
ttttttattcgggaaggaagaaggagtttatagaattttgtaagtttattatttaagttatttagttatattttacggagaagattgtgtgtacgatttaaggcggtgtatttttttttggaaaaatacgggaagggagtgattataaattttattatggggatttggttatatttttttttatagatgggaatacgtttatttttgagtttttttaagattttttgtagcgtttcgatttgtaatatatcgtttcggagtttgaagttgtcgttttggttgcgagtttagtttaggtgcgtatttttagttttagttataaggagtttcggatttatacgtattttgcggtgggatttcgcggatatagggaatggtcgaggggtggaaatttagcggacgttgttttaggtattgatcgcgggtttttgagtcgttggggtttttgttgtttagaggggtcgggatgagggattcgtttaatagattgttaggagttcgtgagttcgagacgtttttcggatggtagtgggtgtttgggcgttgcgtttagtttttcgcgttttgattttgggaataggagtgggggtggggggaggagggaggcgtgtaagagaagagataaggcgatagtttaagggggtcgagagcgaggggaatatcgattagttttttttcgtgggaggtattatttacggtagagtagatgtttagggttcggggttggagttcggcgtttttttgggtacgggtatttaatagaggtagtattagtagtagggcggcgtttaggaggggtagtagttttacgcgggagttttttatcgttttgaaattcgtcgttgttggggtattacgcgtttttattttttatagtgagttcgagttttcgtgtaggaaagagggaggaggcgaagaaggaagggggtgtggaggagggttcgggttaatcgtttgtaggtagcgtttcgtcggcgtttgacgttaatgttcgtcgggttttaggcgtttgggaacgaggttttgcgtatagatgggttagagcgtagagaataggattttgcgtttttaaaggttgttagttttacggtaagggatgtttttcgaggggtttgagttgaagtcgaggggttagaggtgagggacgggaagcgggaggggtttaggaaatggaaaatgttttcgggttaatgtataggtaggagagtcgttttttaatatttagtagtggtgatagttgtaatatttatttcgttagttttgttttgtttcgtattttaattttaagtgtggcggtgagaagggggtgttgtttttagatagttatatttgttgagtattttattttgtggggtaggggaagtaaatatattatataaaagaaagagaaatagtggttttttacgtgatgggttttatgatggaatttgtgtgataatagttaaaagaagagagggaaaatagataaattcggatttagtagtggtgttggaagtgtagagagttaggttttggtttttattttggttatattacgtaaaagttgtatgattttgaataaaatgaagaatttttttaagttttagtttttttttttgtagatttaggattaaaaagatatattttataaagttattagtaggatgaaatgagagattaagtatgttaaatttttggtttttagttaagtttttttgttt

SEQ ID NO: 82 - DNA, 1700 bp, Artificial Sequence
  chemically treated genomic DNA (Homo sapiens)
gtgtttgtattttatgttattttttgagtttataaatataaaaatagaatttatttttgttatagatttagttattgggaatttattatttgttatggattatttttgttaagaaaagtttgaatgagaagatggtaggacgtttgaaaaaaaaagttataattaataaaatttgcggagaattgtaaattttgtttttattgttgatttaaattttttttttaagattttttttttttagatatttatttatttatttttattttattttttagtattaaggaggtttgagtggataaggtttaagtattgatgtggttttggtaatgaatttgaagttaaaaattggttagtttatgttaagttgttatttgaaaggttttttttagtatttgaattaagggaaataatgttttgaagtagttataaaatttttttgatgtattagatttgaggtaggatttgataatttgtatttttaaattatgtaggtgatgttgatgttgttggttcggagattattttggagagttttattttaaaggaaatgaaattgttttaaaatgttaatattaaatagttatataaagtcggaaaaaagtaaagaattttaaatcgaagatgagaggttttgaaaaataatagcggttattttattttcggtgcggttatgcggttatgaaaaggagttttggggtcggtcgtgtttttttacgtaggtttttttatagggttattttattaattttcgttaaaaacgtcggtttttttatagggtcgttcggttacgttttttcgtgttttaattatagagttatacgttacgtcgtcgtttcgttacgtttattcggttttcgtatagggcgttacgtacgtttaacgtcggttcggcgcgcgcgttcgggaggttgttcgtaggtcgttaattggtggtttttagttttcgtcgattcggttttttttttttgcggttattagatcgcgggatatcggagatttcgaataattcgttggagttttttatagtttaaaaatagaggatcggtcgggcgcggtggtttacgtttgtaattttagtattttgggaggtcgaggcgggcggattacgagtttaggagatagagattattttggttaatatcgtgagatttcgtttttattaaaagtataaaaaaattagtcgggtgtggtggtacgcgtttgtagttttagttatttaggaggttgaggtagaagaatcgttagaattagggaggcggaggttgtagtgatcgagatcgcgtcgttgtattttagtttaggcgatagagcgagattgcgttttaaaataaaaaaaacggaggatcgaaaattttgaagattgagagtaaggtaataatttttttttgatttttttgttttttttataagggttttttagacggttgaaattaacgaaaagagtgaggaagtaggtagaaaattttagttttagttttttagtatttttttgtttttttttcggggagttggtattttttttttttttagtagttgtaaatacgttttttaattttaaggagattttttttgtattttttttattgtttttacgtttgtagggtttttcgtgtgaggatttagtgtatgtatgtatgtatatgaatgtgtgcgaataattattttttaattgagtttttttttaaaaaaattatttttcg

SEQ ID NO: 83 - DNA, 1700 bp, Artificial Sequence
  chemically treated genomic DNA (Homo sapiens)
cggaagatgatttttttaaaaaaaaatttaattaggaaataattattcgtatatatttatatatatatatatatatattgaatttttatacgaagagttttataggcgtgagaatagtaagggagatgtaagaaaggtttttttggagttgggaggcgtatttatagttattagagaagggggaagtgttagtttttcgagaagaaagtaggaggatgttgagagattaaagttgaaattttttgtttgtttttttatttttttcgttggttttagtcgtttgaaaaatttttgtgagaggagtaaaaaggttaagggaagattgttgttttgtttttagtttttaaaattttcgatttttcgttttttttgttttgagacgtagtttcgttttgtcgtttaggttggagtgtagcggcgcgatttcggttattgtaattttcgtttttttggttttagcgatttttttgttttagttttttgagtagttgggattataggcgcgtgttattatattcggttaatttttttgtatttttgatagggacggggttttacggtgttagttaggatggtttttattttttgaattcgtgattcgttcgtttcggttttttaaagtgttgggattataggcgtgagttatcgcgttcggtcggttttttgtttttaaattgtaaaggattttagcggattgttcgaagttttcgatatttcgcgatttggtggtcgtagaagaaagaggtcgagtcggcggggattgggaattattagttagcggtttacgggtaatttttcggacgcgcgcgtcgggtcgacgttaaacgtacgtgacgttttatgcgggagtcggatgggcgtaacggggcggcggcgtaacgtgtggttttgtggttagggtacggaggggcgtggtcggacggttttatgagggggtcggcgtttttggcgagggttgatgggatgattttatgagggagtttgcgtggaggggtacgatcggttttaagatttttttttatggtcgtatagtcgtatcgggagtaggatgatcgttgttattttttaggattttttattttcggtttgaaattttttgttttttttcggttttgtgtggttatttagtattagtattttagggtagttttattttttttaaagtaaaattttttaaagtggttttcggattagtagtattagtattatttgtataatttagaagtataaattattaggttttattttagatttaatgtattagaaaagttttgtgattgttttaaaatattgttttttttaatttagatgttggaggagattttttaaatggtagtttagtataagttaattagtttttaattttaggtttattattaaagttatattagtgtttaggttttatttatttagatttttttgatgttggaaagtaaagtgaaaataggtagatagatgtttagaaaggggaggttttagaagaaaggtttggattaataatgaaggtagaatttataatttttcgtagattttattaattataatttttttttttagacgttttgttatttttttatttagattttttttagtaaaggtagtttatggtaagtaatgaatttttagtaattaggtttgtaatagaagtaaattttgtttttatgtttataaatttaaaaagtaatatgaagtgtaaatat

SEQ ID NO: 84 - DNA, 1700 bp, Artificial Sequence
  chemically treated genomic DNA (Homo sapiens)
gtatacgtgaaaataatattattttatattacgatttatgttattcgtatgtgtaattaaagttttttaagttttgatatgtttttataaagacgaaggatagagtgaatgttaaaatttgttaaataggagttattgaaagtttgaaggggaaagtttagttaggttttattttagaagtgatttttaaaacgaaatacgtagataagttcgagttttttggagtcgatttttaaggttttgtaggtggtaagggggtcgagaattggttgaggtttttcggaagttcgcgtcgattcggagggttatttttgattttttgatttggggtgtaatttttagtttttgtttcggtcgataaaggtgtggagttaggttttaggtaagttgggggtgggggtgcgtataagggaggggtaggggattgggattttttttttttcggggtgtttcgggtcgtttttttagaggttggtggtgttttcgatttaagtagtttagtttcgttcgcgaggttcgaagtggcgggaacgggtttttcgcggcggttttaaggcgagggaggttgtaaaatttggtagagaattcggcgcgtcgggtcggggatgtttcgggtgtcgggggtaggggtcgtaggttcgtaggtagaagtttttttacgcgagcgggggtttcggtcgcggaaggggcgttttcggggtcgtcggtagcggcgagcgggcgggagggagtttttcgggtttttttcgcgtttcgggttggcggggcgatggcgacgacggtttttcggttttttcgtttcggacgtcgatggcggtgcgtttcggtttttttttaatagtttttttttcgcggtcgtcggagtcggtgaggtcgagagggcggggagtcgcgtagggaaggcgagagggcggaggttgcgtggtgtaggcggcggcggcgggaggaggagggtcgggcgggtaggggaggaggaggaggcgggcgtcgtgtcgtacgtagttttaggcggggtagtttcggtagttgagggacgtagttagattttggcgggacggggttttcgtcggggtttaggtttagggattaggcggaggcgtcgcgggagtttttggggtattatagagatgcgggtgagtgtcggtagtcgtcggggtcgagaggaggtggcggcggcggcggcggcgtaggttcggtttcgagtcgcggtgtcgtagtgattcgggtcgcgacggtcgaaattaggcgtttggcgaggtttgagtcgtcgaaacgcggtcgggtagaatggcggtcgggtcgggggattcgtgggcggaggacgtcgaaggtggtagaggagcgagcggggtcgggttttgagttatttcggacgggtcggggcggttaggtttggattacgtattgggaaaaagtacgatttatcgcgtttggaggcggcggtttggtggcgtcggttttttttttcgtcgcgatcgggttgggttttcggagggtgggtcgacgtgtggttcggggcggttcgggcgttcgcgtttcgtttggtgaggcgattttcgcgggacgtcgggattcgtgtttttgttttttcgcgtttacgttttttttcggcgttgtttttcggtcgacgcgtgtttttttgtgagttttgggtttaagcgtaggagtagtcgcggagtcgggttttttcgtttttcggt

SEQ ID NO: 85 - DNA, 1700 bp, Artificial Sequence
  chemically treated genomic DNA (Homo sapiens)
atcggaaagcgagagagttcggtttcgcggttgtttttgcgtttgagtttagagtttataggggaatacgcgtcggtcggaaagtagcgtcgggaagaagcgtgggcgcgagggggtaagggtacgggtttcggcgtttcgcggaggtcgttttattaggcgaggcgcggacgttcgagtcgtttcgggttatacgtcggtttatttttcgagaatttagttcggtcgcggcggggaaaggggtcggcgttattaggtcgtcgtttttaggcgcgatgaatcgtgtttttttttagtgcgtagtttaggtttggtcgtttcggttcgttcggggtgatttagggttcggtttcgttcgtttttttgttattttcggcgtttttcgtttacgagtttttcggttcggtcgttattttgttcggtcgcgtttcggcggtttaggtttcgttaggcgtttggtttcggtcgtcgcggttcgagttattgcgatatcgcggttcggggtcgggtttgcgtcgtcgtcgtcgtcgttattttttttcggtttcggcggttgtcgatatttattcgtatttttgtggtgttttaaaggttttcgcgacgttttcgtttggtttttgggtttgggtttcggcgaaagtttcgtttcgttaaggtttagttgcgttttttagttatcggggttgtttcgtttggagttgcgtgcgatacggcgttcgtttttttttttttttttgttcgttcggttttttttttttcgtcgtcgtcgtttgtattacgtaattttcgttttttcgttttttttgcgcggtttttcgttttttcggttttatcggtttcggcgatcgcgggggagaagttgttgggggagggtcggaacgtatcgttatcggcgttcggggcgggggagtcgggaaatcgtcgtcgttatcgtttcgttagttcgaggcgcgggaggggttcggaaagttttttttcgttcgttcgtcgttgtcggcggtttcgggggcgtttttttcgcggtcgaggttttcgttcgcgtggggaagtttttatttgcgagtttgcggtttttgttttcggtattcgagatattttcgattcggcgcgtcggattttttgttaggttttatagtttttttcgttttgggatcgtcgcggaggattcgttttcgttatttcggatttcgcgagcggggttgagttgtttgggtcggggatattattagtttttggagaaacggttcgaagtatttcgaagaagagggggttttagttttttgttttttttttgtgcgtatttttatttttagtttgtttggaatttgattttatatttttgtcgatcgggatagggattgggggttgtattttagattagaaagttagaggtggtttttcgaatcggcgcggattttcgaggaattttagttaattttcgatttttttattatttgtaaggttttaggaatcggttttagaaagttcgagtttatttgcgtgtttcgttttaaaaattatttttaagataaaatttaattaggtttttttttttagatttttagtaatttttgtttggtaaattttgatatttattttatttttcgtttttgtaaggatatattaagatttagaaggttttgattatatatacggatggtatgagtcgtgatataaaatagtattgtttttacgtgtat

SEQ ID NO: 86 - DNA, 1700 bp, Artificial Sequence
  chemically treated genomic DNA (Homo sapiens)
gttagagattgaaattttttggaattaattattggacgttttggattggggcggaacgcgtttttattgtttcgaaagtagtattttttcggatttgagtttttgcggcgcgtttgggttgagatttgagaaatgcgttttgggttataggtagtagcggaggcggggaaggaataatttttttttcgttttttaattataagggcggggtggggcggtttcgttggggtcgggtttttttatttttttagttgggattttttgtcggagttggcgtgggggtcggggagggggattagaggggtttttttttttttttaagtttcgttagatgttggaaaggtaaataataggggaaacgcggagaaataaaaggttattcgttacgattttagtcggttgcgtagagagatttttgtggggagggttaggggagagggttaggaaagagcgcgaggggggggatttcgagagatttagagtttttggtatttgttaggtattaggggtcggcgttttatttttttagtaggggtttgaaggtgggaaggggtaattaaagaaaatttgaagggtttttatttttttttttagtttagggaaagggaggggttgggattttagatgtttgatagttgttggttttaaagttaagcgcgatttgtacggtttttatacgttttttattttcgtgaggtatcgttagtttcgggggcgatttggttgacggtgcggattttttttacgttgttttaagtcgagattttttttttaggattaggacgtttaagcgttgttttttagtttaggcgtatttttgtaaaagtttgttttttcgttatttatagtttaatacgtattttgtaggcgttcgcggttggggatgggttcggagttttgtatttcgaaagaaggacgattttattgatttatatttttttaaattaaggataaattaagatttcggggtggaaagtgggcgatttttgtttttgtcgggaagtgtatacgaggagtcggttagaaatttaggcgtcggtaggggtttgaagaggattttagtttcggaagttcgtggtcggcgggtagaagatcggttcgtcgtttttcgtatttttttgtattcgcgtttaggtgcgttttaaatttcgtcgttcgttcgggcgagatcgggagcgagtagttatcgggttgcgtcgttttttttagtttggagcgggtttttatttatcgtggggttttatttttagtttagggttcggtcggtttttttcgtcgggttagttgtagttagcgcgaaagaaaatggtgagttcggggtaggtggggcgtcgtttgttaatttacgtaagggattttgtcgtcggtgcgcgaggtttcggcgagttaatcggcggacgggcgagcgtttggttttagttgtagtcgtcgtcgtttcggagtagtttttttatgtatttagcgcgtcgcgtagtcggtcggggttcgattgtttcgcggggacgtcgagcggggatttttggcggtcgtttgcggtatggtagtatcgtggagagaaaagtcgtttttgggattatttttttttcggaaagagatgggatatgattagtttaagggggtttttagttttgtagaaaagatattgggttagggtagtgaagaaggtgttagattagaaaggatgatttttgggtttataa

SEQ ID NO: 87 - DNA, 1700 bp, Artificial Sequence
  chemically treated genomic DNA (Homo sapiens)
ttgtggatttagagattattttttttgatttgatattttttttattgttttggtttagtgttttttttgtaaggttggaagtttttttagattggttatgttttattttttttcggagggaagatgattttaaagacgatttttttttttacggtgttgttatatcgtaggcggtcgttaggggttttcgttcggcgttttcgcgagatagtcgagtttcggtcggttgcgcggcgcgttgggtgtatgagggggttgtttcggagcgacggcggttgtagttggagttaggcgttcgttcgttcgtcggttggttcgtcgggatttcgcgtatcggcggtagagttttttgcgtggattggtaagcgacgttttatttgtttcgagtttattattttttttcgcgttggttgtagttgattcggcgaagggagtcgatcgggttttgggttggaggtaaaattttacggtgagtaagaattcgttttaagttaggggaggcggcgtagttcggtggttgttcgttttcgatttcgttcgggcgggcggcgaggtttggggcgtatttgggcgcgggtgtaagaaggtgcgggaggcggcggatcggttttttgttcgtcggttacgggttttcggggttggagttttttttagatttttgtcggcgtttgggtttttggtcggtttttcgtgtgtatttttcggtaggaataagggtcgtttattttttatttcgggattttgatttgtttttgatttgaaaagatataaattaataagatcgtttttttttcggggtgtaagatttcgagtttatttttagtcgcggacgtttgtagggtgcgtgttgggttgtgggtggcgggaagataaatttttataaaagtgcgtttgggttgggggataacgtttgggcgttttgattttgagggaggagtttcggtttggggtagcgtaggggaagttcgtatcgttagttaggtcgttttcggggttgacgatgttttacggaggtggggagcgtgtaaaggtcgtataaatcgcgtttaattttggggttaataattgttaaatatttggaattttagtttttttttttttttgaattggggaagaaggtgaaaattttttaagttttttttgattgtttttttttatttttagatttttgttgggagggtaaagcgtcgatttttggtgtttggtaagtattagagattttaaatttttcgggattttttttttcgcgtttttttttgatttttttttttaattttttttatagagatttttttacgtagtcgattgagatcgtggcgaatggttttttgttttttcgcgttttttttattgtttgtttttttaatatttggcggggtttggggagagaaggaagtttttttggttttttttttcggtttttacgttagtttcggtaggggattttagttgggaaagtggaggagttcgattttagcgaggtcgttttatttcgtttttgtggttagagggcggagggaaagttgtttttttttcgttttcgttgttgtttgtggtttagggcgtattttttagattttagtttaggcgcgtcgtaaaggtttaaattcgagaaggtgttgttttcgagatagtggaagcgcgtttcgttttaatttagagcgtttagtggttggttttagaggattttaatttttagt

SEQ ID NO: 88 - DNA, 1700 bp, Artificial Sequence
  chemically treated genomic DNA (Homo sapiens)
ttaggatttttaattatttattatttgtatttttggggtggacgtgagttcgcgttaaattaaggatattagtcgagtatttagcgcggttttttacgttttttaggaattttggaaagttatttaagttcggcgttatttttaaaaggaatgagttgtggtttttaattagtttgtcgaggagagcgtagagtgatacgggacgggggtttagggtattgaggttaagagttttcgagttacgttagtgttcgtttttttggtttttgtttaaaattttgcgatgtttcgttgataaagtatttagtgttagagtttgcggtttagcgaagttgttttttggaaatatatatatatatatatatatatatatatatatatatatatatatatataaaagggggtggggagagggaaaggtttaggtttaggagaggaagttagttagttagttttgtagatgtaatcgttgtaatttgggttttttttgttttttgacgtaatgttagttttttgttttcgcgttgtagtgtgtagttcgttgttgggaagttagattaatcgattgaacgttaagattattattgttattattttgcgatttattttttacgaatatattttaattggttcgagtttcgtttttaaattttacgatttttggttttttaggaagtattgtttttttttttttttaggaaggtcgtttttacgtatagttagtattaggaaacggcgtcgttcggggtttcgatggagagtcggggtggggtcggtttaaattttttgtttttcgtttttttgagatttttgatttttggttttgttttaagtaaaagtattgtagaagttgtagcgttttttttttttttttagaaatcgagcgaggacgtgggggaaaagttattttggaacgtgtaaggacgtttttttacgtttaatacgttatagggtcgggatgcgcggttgtcgtttcgttattttacgcgcgtagcgttaattgtagtttcgtagttatcgtcgtcgtcgtcgttttgtcgagttcggggagatttttaagggttttttaaatacgagttataaatattataggcgttcggcgcgtgcgttacgatatattcggttttcgattgggcggcgtaatttcgtttatttgtataggtcgattgtataatcgggcggcgattttagttcggtgttcgtgttacgttttttttatttttgcgggtacgcggggtcgcgggcgggatagcggggtggcgattgcggcgtttagagttttttgaattcgggaattttttaagttttgagttaggcggcggtttttcgggacgttagtgttttttagaaggtttagttttggttagttaagggtttatcgtttaaattgggttttttttatttttatagaaaaggatagtttttagagtgatattttagtaaacgttttgggtggttcgtagaggttgagtttaaattacgtgagatttggtcgcggcgagagttttgtttaatgatttgcgatttggggtgtttaaaaagggtttttcgtatgatttttaataagatttcgttattttgaaaacgaaatagggagggttcgaaaacgtaagatgtgtaatgttttgtggtttaaatttagtttttaaaaaatgtattaatgagttaatatgaaatagcgatatggagattttttttta

SEQ ID NO: 89 - DNA, 1700 bp, Artificial Sequence
  chemically treated genomic DNA (Homo sapiens)
tgggggaaagtttttatgtcgttgttttatattggtttattggtgtattttttaaaaattaggtttaaattataaggtattatatattttgcgttttcgaattttttttgtttcgtttttaagataacgaggttttattagagattatgcgaaaagttttttttgaatattttaagtcgtaagttattggataggattttcgtcgcggttaggttttacgtggtttaaatttagtttttgcgagttatttagagcgtttgttggggtgttattttgaaaattatttttttttgtagaaataaaaaagatttagtttaaacggtaagtttttggttggttaaaattgagttttttgagaggtattggcgtttcggaggatcgtcgtttgatttaggatttgaaaggttttcgagtttaagggattttggacgtcgtaatcgttatttcgttgtttcgttcgcggtttcgcgtgttcgtagaagtggggaaggcgtggtacgagtatcgggttgaggtcgtcgttcggttgtgtaatcggtttatgtaaataagcgaggttacgtcgtttaatcgggagtcggatgtgtcgtgacgtacgcgtcgagcgtttgtagtatttatagttcgtatttaaagagtttttgggagttttttcgggttcggtagggcggcggcgacggcggtggttgcggagttgtagttagcgttgcgcgcgtgaggtggcggggcggtagtcgcgtatttcggttttgtggcgtgttgggcgtgggggagcgtttttatacgttttagggtggttttttttttacgttttcgttcggtttttggaaagggggaggaaacgttgtagtttttatagtatttttgtttaagataaagttagggattaggaattttaaaagagcggggggtagaaggtttgagtcgattttatttcggttttttatcggagtttcgagcggcgtcgttttttgatgttggttgtgcgtagaggcggtttttttggggggaggggagaataatgttttttgaaaagttaaaagtcgtggaatttgggggcggggttcgggttaattaaagtgtattcgtggaaaataaatcgtaagatagtggtagtgatgattttggcgtttaatcgattgatttgattttttagtagcgaattgtatattgtagcgcgaaagtaggaaattggtattacgttagaagatagaagagatttaaattgtaacggttgtatttgtagggttggttggttggtttttttttttaaatttaaatttttttttttttttatttttttttgtgtgtgtgtgtgtgtgtgtgtgtgtgtgtgtgtgtgtgtgtgtgtgtgtttttagggagtagtttcgttgggtcgtaggttttggtattgggtgttttgttagcgaggtatcgtaggattttaggtaaaggttaaaggggcgagtattggcgtgattcgggaatttttggttttagtgttttgggttttcgtttcgtgttattttacgtttttttcggtaagttagttaggagttataatttattttttttaaagatagcgtcgagtttggatagttttttaaaatttttggagggcgtagggaatcgcgttgaatgttcggttggtgtttttagtttagcgcgagtttacgtttattttaggaatataagtggtgagtggttagagattttag

SEQ ID NO: 90 - DNA, 1700 bp, Artificial Sequence
  chemically treated genomic DNA (Homo sapiens)
tattttaagttattaagttttagataggtatagtagtatatacgtgtgaatagaatttattagtatagtttggggtttaggtttttgtatattaataatttaaggtttttggtaatggtttagcgtgtatttatgaggttattgtttttttatatacgtatttagaatatttatagttttaaaacggtaattttattataaaaatgatattttgtatttgtatagtattttgttttttgtgtattttatatatttattatttaatgattgtgttttttgaaatttttaataattttgggtggtggtaagaataggtatttttaagttatttttttagatgaggaaatttatttgtgtttttttttttttttttaatagtcggttgatggaatttaagttttttatattagtagtggggttagttgtatagagcggttagaagggagaggtttcggggttacgtttttttttttatttttagtttttgatttattgttatattatggcgagtaataggataattttttttatagaattatgggggatattatttttttgaggttaatgtgtttaattaagcgggaaatttttcgggtagagtgaaattcgaagttgttatgttataagataatttagagtaaggggaataattgacgttttaatgtttttttttatggggggttgaggggtgtttatttatatttttttttttcgttttttttcgttattttttttttgttggtcggtttaattttataagcgcgcgtttgtttcgagtatagtaatttagagtagatggggggtattttttagagggtagtggaaagtttatttgttattgttgcgtcggggatttgcggttttttgggcggttacgttttgtttcggcggcgtttggcgcgtttggaatgcggggttttttttcgtttttttttttacgtcgtttcgtttcgttttcgttagggttttttagagaagttcgattttttattatttttgttttgtattttttcggttcgagggtcggtggttttttatttttgttttaaatattttgtttttgagttttttttttaaagattttttagtgtcggtcgtttttttagttttttgttttcgacggttttcggggttagttgtcggggtttttttgttaggtcgtatttttttttttagcgtttttatatttcgttagttggcggtttatcgcgggcgggtagggcgcgtttcgtttatttgggtcgtgcgtcgagagttgagagcgtttttaaatgttgagtagagtcggttggtttgggtggggtttttatattcgttatattttttttaggcgaggcgaggttcgtttcgtttcgtttcgttttttttttatacggttcgtttttattttaagttaggggtgatttatttcggttcgtcggttcgtagggatcgcgttttggaacgttttcgagtcgcgttacgcgggagcggttttagcgtttttggttttgtttttcgagaaagttcggtcgaggttgtttcgaggtttatagggatttcgttttttttttttttttttattttattttttaagtgttacgtcgagtttttagtttcggtttgggttaataaattaataagattttagtagtagattttgggtttgattaagtgtttttttttttttttttttttttttggcgttaatgtaatggagg

SEQ ID NO: 91 - DNA, 1700 bp, Artificial Sequence
  chemically treated genomic DNA (Homo sapiens)
tttttattgtattaacgttaaaaaaaaaaaaaaaaaaaaaaaaatatttaattaagtttagagtttattattaaagttttgttaatttattaatttaagtcgaggttgagagttcgacgtggtatttaagaagtaaaatgaggaaaggaggaagaaacggggtttttgtgggtttcggggtagtttcggtcgggttttttcggagggtagggttaggggcgttggggtcgttttcgcgtggcgcggttcgggagcgttttagggcgcggtttttgcgggtcggcgggtcggggtgagttatttttgatttggggtgggagcgggtcgtgtaagggggaggcggggcggggcggggcgggtttcgtttcgtttagggaggatgtggcgggtataaaagttttatttaggttagtcggttttgtttagtatttggggacgtttttagttttcggcgtacggtttaggtaagcggggcgcgttttgttcgttcgcgatgggtcgttagttagcggggtgtggagacgttgggaagaagggtacggtttggtagggaggtttcggtaattggtttcggaggtcgtcgggggtagggggttgaagggacggtcggtattggagagtttttggggagggggtttagggataaggtgtttggggtaggggtgaggggttatcggttttcgggtcgggagggtgtagggtagaggtggtggggggtcgggtttttttaagaagttttagcgggagcggggcgaggcggcgtggggagaggggcggagggaggtttcgtattttagacgcgttaggcgtcgtcgaggtagggcgtggtcgtttagggagtcgtaggttttcggcgtagtagtagtaagtgggttttttattgttttttagaaagtgttttttatttgttttaagttattgtattcgaaataaacgcgcgtttgtggagttgaatcgattagtaggagggaagtagcgggggaagacgagaggggggagtgtgagtaggtattttttagttttttataaaagaagatattaaaacgttagttattttttttgttttaggttattttgtagtatagtaatttcggattttattttattcggagagtttttcgtttggttgaatatattggttttaggaaggtagtgttttttatgattttgtggggagggttgttttgttattcgttatgatgtgataatgagttaggaattgagagtagagagaagggcgtggtttcggggttttttttttttggtcgttttgtgtagttggttttattgttggtgtggagggtttgggttttattagtcggttgttgagaagaaggaggggaatatagataagtttttttatttgggaaggtggtttaaggatgtttatttttattattatttagagttattggagattttaaaaaatatagttattaggtaataaatgtgtaaagtatataaaaaataaagtgttgtgtaaatgtaaagtgttatttttgtagtaagattgtcgttttaaagttgtgggtgttttgagtgcgtgtgtaaaagagtagtggttttatgggtgtacgttgggttattattaagaattttaagttgttggtgtgtaaaggtttgggttttagattgtattgatggattttgtttatacgtgtgtgttattgtgtttgtttggaatttggtgatttggaatg

SEQ ID NO: 92 - DNA, 1700 bp, Artificial Sequence
  chemically treated genomic DNA (Homo sapiens)
ggttttttggtagtttaagtgttagtttagatattgagatttcggtaaagagtttttttttattttttttttttattggtttttgtgggcggtgtggaatgtgttgtaggttttgcgagagcggtattttttttttttttttttttttttttttttaatttattgtagtttttgagagggtagtatttttgggcgtatttgtatttaagtttttgatagtgttttgggtttttttagtaacgcgtatttagggatgttatgttttgtgggttttatgttcggggtacggagttaggggaggcggttattttattttttttacgtttttgtttattttgcgttttagcggagttttaggtaaataagttaggtttttagtttttaaggacggtttttattttagattttaggaggaggcgttttgggttatttcgggaaggttagtttgttcgggaggggtagattacgaaaacggttttttaggtagtgttattttttttttattgttagttatttaatttaggcggttttatttttttcgtttttatggatatgtagtaggatgtagagagtatagagttgtgattggtacgagtttataacgggttttttcgtttaatttatttaatttttagtaagggataggttttgtttattggggttcgttgaggttttaaacgcgtatgttgttaagggagtagttgcgggggttcgttagtggggataaaggcgtagttatcggggcgaaagtttcgtttcgtagagggtgggtttgcggcgatcgcgagtttagagttcggatttgtcgcgtaatttgtttttttcggttttattttaatttagttggattttcggttcggtttttgttttttggagatttagttattaaagttgaggtggggggatgaggatgtgggcggcgtttaggtaattttgaggggttttgatgttttttttagagttagttattaggagtaatacgatgcgtttttatattatcgaatttagaattttagtgttttagtatttttttttcgatatgagtttcgtttaatgaggagaagggttttttgtattggtcggtggtggttttttatttaattagagaggttaacggttttttttcgtttggtttagaaggaatcgtcgggggaatataggttaaattataattttcgagaggtagttgggttttgcgggggggggggggggggcgtttgcgtgtttacgtgatttttttttttttcgcggagatcgagttacgcgataggtttgtgagggggcggggttcggcgtttgttcgtcgtgttagtggttaatcggtagttaggtagggtgggcgcgcgtagggggcggggtcgggcgcgcggtagggcgcgagagcgtattcgcggcggcggtggcggcgattgtgggggggcggcggggaatattggttaagtcgatagtggaggtttaggtatcggtggcgggcggttgcggtttttggtgttgttcggcgcgcggttagttttcggaacggaacgttcggcgtcgcgggtttcgttcggaaagtttgtcgtggagtcgcgattttttggttcgcgcggttcggtatgaagcggcgttgaggagttgttgtcgtcgtttgtcgttgtcgtcgtcgtcgtttgaggaggagttgtagtattttgggttacgtcgatgattattgtaaattg

SEQ ID NO: 93 - DNA, 1700 bp, Artificial Sequence
  chemically treated genomic DNA (Homo sapiens)
tagtttgtagtagttatcggcgtggtttagggtgttgtagtttttttttaggcggcggcggcggtagcggtaagcggcggtagtagttttttaacgtcgttttatgtcgggtcgcgcgggttaagaggtcgcgattttacggtaaatttttcgggcggggttcgcgacgtcgagcgtttcgtttcgaaagttggtcgcgcgtcgagtagtattaggaatcgtagtcgttcgttatcggtgtttaagtttttattgtcggtttagttaatgtttttcgtcgtttttttatagtcgtcgttatcgtcgtcgcgggtgcgttttcgcgttttgtcgcgcgttcggtttcgttttttacgcgcgtttattttgtttggttgtcgattggttattaatacggcgaataggcgtcgggtttcgtttttttataggtttatcgcgtaattcggttttcgcgagaaggaaggaagttacgtgaatacgtaggcgttttttttttttttttcgtagagtttagttatttttcgggaattgtagtttggtttgtattttttcggcgattttttttaaattagacgagagaaggtcgttagtttttttaattggataggggattattatcggttaatgtagaagatttttttttttattgggcggagtttatgtcgaggaaggggtgttgaagtattgaggttttgggttcggtgatgtgggggcgtatcgtgttatttttgatggttggttttagggaaggtattagggttttttagagttatttggacgtcgtttatatttttatttttttattttaattttaatggttgagtttttagagggtagggatcggatcgagagtttagttaggttggggtggaatcgaaggagatagattgcgcgataagttcggattttgagttcgcggtcgtcgtaggtttattttttgcggggcggagttttcgtttcggtggttgcgtttttgtttttattagcgggttttcgtagttgtttttttgatagtatgcgcgtttgaggttttagcgaattttaatgggtagggtttgttttttgttggaagttgagtagattgagcgggaaagttcgttgtgagttcgtgttaattatagttttgtgttttttgtattttgttgtatatttatgaaagcgggagggatggagtcgtttaaattgggtggttggtagtggagaggaagtgatattgtttaaagaatcgttttcgtgatttgtttttttcgagtaagttagttttttcgagatggtttaagacgtttttttttggggtttggggtgaaggtcgtttttggggattgggagtttggtttgtttatttaaggtttcgttgaggcgtaaggtggatagggacgtgagagaagtagggtgatcgttttttttagtttcgtgtttcgggtatagggtttatagggtatagtatttttagatgcgcgttgttggaggaatttagggtattgttagaggtttgggtgtaggtgcgtttaaaaatgttgtttttttagaggttataataggttaaaaaaaaagagagaaaaaaaaaaaagaaatgtcgttttcgtaaggtttatagtatattttatatcgtttataaaggttagtgggaagagagggtaaagggaagttttttgtcgaggttttaatgtttggattgatatttgagttattagggagtt

SEQ ID NO: 94 - DNA, 1700 bp, Artificial Sequence
  chemically treated genomic DNA (Homo sapiens)
gttttggatattttttgtttttttgatttaaggtttagtttggttttttttagggttttttggtagtttaagtgttagtttagatattgagatttcggtaaagagtttttttttattttttttttttattggtttttgtgggcggtgtggaatgtgttgtaggttttgcgagagcggtattttttttttttttttttttttttttttttaatttattgtagtttttgagagggtagtatttttgggcgtatttgtatttaagtttttgatagtgttttgggtttttttagtaacgcgtatttagggatgttatgttttgtgggttttatgttcggggtacggagttaggggaggcggttattttattttttttacgtttttgtttattttgcgttttagcggagttttaggtaaataagttaggtttttagtttttaaggacggtttttattttagattttaggaggaggcgttttgggttatttcgggaaggttagtttgttcgggaggggtagattacgaaaacggttttttaggtagtgttattttttttttattgttagttatttaatttaggcggttttatttttttcgtttttatggatatgtagtaggatgtagagagtatagagttgtgattggtacgagtttataacgggttttttcgtttaatttatttaatttttagtaagggataggttttgtttattggggttcgttgaggttttaaacgcgtatgttgttaagggagtagttgcgggggttcgttagtggggataaaggcgtagttatcggggcgaaagtttcgtttcgtagagggtgggtttgcggcgatcgcgagtttagagttcggatttgtcgcgtaatttgtttttttcggttttattttaatttagttggattttcggttcggtttttgttttttggagatttagttattaaagttgaggtggggggatgaggatgtgggcggcgtttaggtaattttgaggggttttgatgttttttttagagttagttattaggagtaatacgatgcgtttttatattatcgaatttagaattttagtgttttagtatttttttttcgatatgagtttcgtttaatgaggagaagggttttttgtattggtcggtggtggttttttatttaattagagaggttaacggttttttttcgtttggtttagaaggaatcgtcgggggaatataggttaaattataattttcgagaggtagttgggttttgcgggggggggggggggggcgtttgcgtgtttacgtgatttttttttttttcgcggagatcgagttacgcgataggtttgtgagggggcggggttcggcgtttgttcgtcgtgttagtggttaatcggtagttaggtagggtgggcgcgcgtagggggcggggtcgggcgcgcggtagggcgcgagagcgtattcgcggcggcggtggcggcgattgtgggggggcggcggggaatattggttaagtcgatagtggaggtttaggtatcggtggcgggcggttgcggtttttggtgttgttcggcgcgcggttagttttcggaacggaacgttcggcgtcgcgggtttcgttcggaaagtttgtcgtggagtcgcgattttttggttcgcgcggttcggtatgaagcggcgttgaggagttgttgtcgtcgtttgtcgttgtcgtcgtcgtt

SEQ ID NO: 95 - DNA, 1700 bp, Artificial Sequence
  chemically treated genomic DNA (Homo sapiens)
ggcggcggcggtagcggtaagcggcggtagtagttttttaacgtcgttttatgtcgggtcgcgcgggttaagaggtcgcgattttacggtaaatttttcgggcggggttcgcgacgtcgagcgtttcgtttcgaaagttggtcgcgcgtcgagtagtattaggaatcgtagtcgttcgttatcggtgtttaagtttttattgtcggtttagttaatgtttttcgtcgtttttttatagtcgtcgttatcgtcgtcgcgggtgcgttttcgcgttttgtcgcgcgttcggtttcgttttttacgcgcgtttattttgtttggttgtcgattggttattaatacggcgaataggcgtcgggtttcgtttttttataggtttatcgcgtaattcggttttcgcgagaaggaaggaagttacgtgaatacgtaggcgttttttttttttttttcgtagagtttagttatttttcgggaattgtagtttggtttgtattttttcggcgattttttttaaattagacgagagaaggtcgttagtttttttaattggataggggattattatcggttaatgtagaagatttttttttttattgggcggagtttatgtcgaggaaggggtgttgaagtattgaggttttgggttcggtgatgtgggggcgtatcgtgttatttttgatggttggttttagggaaggtattagggttttttagagttatttggacgtcgtttatatttttatttttttattttaattttaatggttgagtttttagagggtagggatcggatcgagagtttagttaggttggggtggaatcgaaggagatagattgcgcgataagttcggattttgagttcgcggtcgtcgtaggtttattttttgcggggcggagttttcgtttcggtggttgcgtttttgtttttattagcgggttttcgtagttgtttttttgatagtatgcgcgtttgaggttttagcgaattttaatgggtagggtttgttttttgttggaagttgagtagattgagcgggaaagttcgttgtgagttcgtgttaattatagttttgtgttttttgtattttgttgtatatttatgaaagcgggagggatggagtcgtttaaattgggtggttggtagtggagaggaagtgatattgtttaaagaatcgttttcgtgatttgtttttttcgagtaagttagttttttcgagatggtttaagacgtttttttttggggtttggggtgaaggtcgtttttggggattgggagtttggtttgtttatttaaggtttcgttgaggcgtaaggtggatagggacgtgagagaagtagggtgatcgttttttttagtttcgtgtttcgggtatagggtttatagggtatagtatttttagatgcgcgttgttggaggaatttagggtattgttagaggtttgggtgtaggtgcgtttaaaaatgttgtttttttagaggttataataggttaaaaaaaaagagagaaaaaaaaaaaagaaatgtcgttttcgtaaggtttatagtatattttatatcgtttataaaggttagtgggaagagagggtaaagggaagttttttgtcgaggttttaatgtttggattgatatttgagttattagggagttttagagggagttaaattagattttgggttaggaagatagggaatgtttagggt

SEQ ID NO: 96 - DNA, 1700 bp, Artificial Sequence
  chemically treated genomic DNA (Homo sapiens)
cgagggtttcggtttttttttttgtgtttgtttttcgtttagcgtcgggagaagtagtaataagttttgtattttagtaattaattttagttattatatttgtattaattagcgtcgtttaagtttcgggttcggggcggggttcgtttttaaggtggttcggggttttggttgtcgaagttttcgttacgaattcgtattattttagttttggggttttttgcgttcgttttttttcggggttcggcgttagtgcgttgggggcgcgtttcggttttcgggcgtatcgagttggagagggtgtaggcggaggagaatggcgggagtttggttttgtttattttttttttatttatattgtttttaattttttatttttaattggagcgagatttatcgggtgggtagggggcggggaggtgggcggtgagcggtacggggcgtttagataatgaggtcgttaggtaaagattttagattcgttacggaaagtcgttaagtgtttattgtatttcggtttcggggtgaatatttcgtttttcgttagcggcgtttaagttagtcggttttgggatttttttggagggtaaagtagacggggaatttcgaagttagggtttttttcgagagatttgggcgtttgtttattttttcgttattagggcgtttataattgttttttttttttattgtttggggttttcggtttttatcggtcggcgtgggttttaaggagttatagttatttagttattttatgggggagtatatcggttgagtttagtaatcggagcgatttgcgaggcggttgggaatttcgagaagatacgtacgaagagggcgtattcgtcgggtcgtatattttgttttgggttattcggtttaagatttagattttttgcgggtgcgaagttggtttttttaagagattaattaaattgaagaggtcgaggaatggaattgggttgtgatattaaaattaataggagtaatttaatatttgttttgttttttatgaaatatttattttaataattttagttaaaatgtttagatcgttttgatttttatagaggttgaaatttattttgtaattagaagttggaagttagaggtgatggaggatagaggtgtttattttgtgcgtttgtgtgcgtgtgttcgcgtttgcgtgcgtgtgtttgtgtatgcgtgtgtgtgtgtgtgtgtgtttttttttaaagtgtgtgtagggaaggaggggggagaaggtttttaattttttttgttataaaatagtacgaggcgattttgttagtagattcgtatttttaggagaagggcgaaaaaagaattttttttgaagtgtttttgatatcgttattaaatttaaataaaattaagcgtggtgtatgaaaatgtttttttttttttataaaagaaagtgtttatcgttcgagtttgtttagcgtttgttaaattacgtatgaaatttgaaatgaaataaacgttataataaaattaatttttgagttttttttatttaaaaattttagattaagtattttatcgtcgcgggtaggagttaaaaagttattatgtcgtacgcgtagatttattgtttttttgagttgcggttaggaaggtagtgttaagtatgttagaatcgatatgtcgggggtggggggaaatgggtttaattttggggtagcggtagaatt

SEQ ID NO: 97 - DNA, 1700 bp, Artificial Sequence
  chemically treated genomic DNA (Homo sapiens)
aattttgtcgttattttaaagttgagtttatttttttttattttcggtatgtcggttttggtatatttagtattgtttttttagtcgtaatttaggggagtaatgaatttgcgcgtacgatatggtaattttttaatttttgttcgcgacggtggagtgtttaatttaaggtttttaaataaaaagggtttaggggttggttttattataacgtttattttattttagattttatgcgtaatttagtaaacgttagatagattcgggcgataaatatttttttttgtggggggggaaaggtatttttatatattacgtttaattttatttagatttaatagcgatattaggggtattttaaagaaagtttttttttcgttttttttttaagaatgcgaatttgttagtaaaatcgtttcgtgttattttatggtaaggaaaattaaaggtttttttttttttttttttttatatatattttagggaggaatatatatatatatatatacgtatgtataaatatacgtacgtaggcgcgagtatacgtatataggcgtatagaataaatatttttattttttattatttttaatttttaatttttaattgtaagatagattttagtttttgtggggattaggacgatttaaatattttaattaagattattgggataaatattttataaagagtagggtagatattaaattatttttattgattttagtattatagtttaattttatttttcggtttttttagtttaattgattttttaaggaggttaatttcgtattcgtagagggtttgggttttggatcgggtggtttagggtaaggtgtgcggttcggcgggtgcgtttttttcgtgcgtgttttttcggggtttttagtcgtttcgtaagtcgtttcggttattgggtttaatcgatgtgtttttttataaagtggttgagtgattgtgattttttggagtttacgtcggtcggtgagaatcgggaattttaggtagtggggaaggggaataattgtgaacgttttagtgacggaggagtgagtaagcgtttaggtttttcgaggagaattttaatttcgaggtttttcgtttgttttattttttaagaaaattttagggtcggttaatttgggcgtcgttagcggagaacggagtgtttatttcggagtcgaagtgtagtagatatttagcgatttttcgtggcgagtttagggtttttgtttggcgattttattgtttgagcgtttcgtatcgtttatcgtttattttttcgttttttgtttattcggtgggtttcgttttagttgggggtggggagttgggaatagtgtgaatgggaagggggtgaataaagttaagttttcgttattttttttcgtttgtattttttttagttcgatgcgttcggaggtcggggcgcgtttttagcgtattgacgtcggatttcgaggagaggcgggcgtaggggattttagagttggggtagtgcgggttcgtggcgggggtttcggtagttaggatttcggattattttaagagcgagtttcgtttcgagttcggaatttgggcggcgttgattggtgtaaatgtaatggttgaaattgattgttgaaatgtaaaatttgttgttgtttttttcggcgttgggcgagaggtagatatagagaggggagtcgagattttcg

SEQ ID NO: 98 - DNA, 1700 bp, Artificial Sequence
  chemically treated genomic DNA (Homo sapiens)
ttttacggggattttcgttaatgtttggttgagaggcggttttagggggttgagaattttatttttagaggtttggtatttgggagattgagaaagttttttttaagtttttttgttgggttacgttgtatttatagattatttttaattttttttttttttttatttattttttcgtttttatttttttggtttattttttgttagaaaatgtattaagttttaattttcgttttttttagttttttttttgagaagatattgtgatttcgttgtttttgttcgtgttattatggagaggtaggaggataaatatcgtttttagttgttagtttttttgttttttttttttttttttagatagtttttagttttaattttagtattcgttttattatttttttttggagaattagtataatcggagtaaataggtaattaagtagtgtgtttgtaagttttaattttgggatagataaggaagtgaaattgattggaattatatttgtcgttgttggaggttgagggaggcggtcgtcggaggcgaggttagttttgtcggtttagggtttcgttttatttttttttttttttttttttttttttagttttatttttttttatttttttttattttgtatttttttttagcggtcgtttgttttttttaaggaattttattttaggtggagtttttacgatagttatattttttttttgggtttagtagggttagagaaagacggaattattttatttatttaggttgtttagagagtattttaggttttgattcgcgtggggcgcgaggtatttcgtggggtagttaggtgtttttaagaaatttcgttttagaggattcgtacgagttgttgttatttttcgttgaagtttttgttttggggttggtgtttttttttatgattttattaatcgtcggtatttttttttttttttttttttttttaggaaggcgttggggtgtgcggcggtaacggcggcggtgaagggatttttttgtggtattttttcgtgggtttttgttttgaaggatttttttttcgttttaattttatgtttaagtagcgttttgggggttgggttggtttttatagtttttattttcggttttttttttttaaagggtgtgtggagtttagaggatgggtggagagcgggtttggtcggaattgaattggtagaggtaaaaggggaacggaggattttaagttttggttatttcggtttagcgaatttattttatttttgtttaaggaaaagttgtaggggggaggattttttttaggaagtttattttgtcggtttttgatttttgggttggatgtttgagagttttgttttttttgtttcgagagtggtttagttgggtttgaatcgatttttttttttttcgattttttttttattttttagttcggagaaataggggagtgggggtttaagaacgagaagacgagaacgcgtcgttttgcgttatgttagaatggggcgggtgtgaggggaatagtttttttgcgattagtttaggagtatgagtttttcggaggacggtatgagttttggatattttaggagtttttagttagtgatatggtcggtaagtgattttgggattaatatttatttatttttatttttttcgtttagattttattttttttttagtttttttgtttttt

SEQ ID NO: 99 - DNA, 1700 bp, Artificial Sequence
  chemically treated genomic DNA (Homo sapiens)
ggggagtagggggattgggagggggatagaatttagacgaaagaggtgggagtgggtgggtattagttttagagttatttatcgattatgttattagttgaggatttttgaagtgtttagaatttatgtcgtttttcgggaggtttatatttttgagttgatcgtaagagagttgttttttttatattcgttttattttgatatagcgtagggcgacgcgttttcgttttttcgtttttgggtttttatttttttattttttcgggttggaaaatgaggagggggtcgaaaggggagggagtcgatttagatttagttgggttattttcggaataagggaaataaagtttttaagtatttaatttaaaaattaggggtcggtagagtagattttttaaaaagaatttttttttttgtagtttttttttggataaaaataaagtgggttcgttgggtcgaagtggttagagtttaaggtttttcgttttttttttatttttattaatttagtttcggttaagttcgttttttatttattttttgaattttatatattttttaaaaggagagggtcgggggtgggggttatgaaaattaatttaatttttaaagcgttatttgaatataaagttgaggcggagaagaaattttttaagataggaatttacgaggagatattataagaggatttttttatcgtcgtcgttgtcgtcgtatattttagcgtttttttaaaaagaaagaagggaggaaaaaaatgtcgacgattggtagaattatagagaagaatattagttttagggtaggggttttagcgaaaaatggtagtaattcgtgcgggttttttggggcggggttttttggaggtatttagttgttttacggggtatttcgcgttttacgcgagttagaatttaaagtgttttttgggtaatttgagtgggtagagtgatttcgtttttttttggttttgttaggtttaaaggagaggtgtgattatcgtgaggattttatttagggtggagttttttaaaaaggataggcggtcgttagaggagagtgtaaagtgggaggaggtggagaagggtggggttgagaaggaggagggaggaaggagggggtggggcggggttttgggtcgatagagttggtttcgttttcggcggtcgttttttttagtttttagtagcggtaggtataattttaattaattttatttttttatttgttttaaaattaaggtttgtaagtatattgtttgattatttatttatttcggttatgttaattttttaggggagggtggtgaaacgggtattgggattggggttgggggttgtttaagagggaaaggagaaaataaaaaagttgataattggaagcgatatttatttttttatttttttatggtaatacgaataaggatagcgaggttatagtgtttttttagggaggaagttaaaaggaacggagattggagtttggtgtattttttagtaagagataagttaaggaggtgagggcgaaggagtaggtggggaagaagaaggagttgaagatgatttgtgaatatagcgtggtttagtagggagatttaagaaaggtttttttagttttttagatattagatttttaagaatagagtttttaattttttgagatcgttttttagttaggtattggcgaagattttcgtaaag

SEQ ID NO: 100 - DNA, 1700 bp, Artificial Sequence
  chemically treated genomic DNA (Homo sapiens)
ttagatagtagaagtttatattttttggaggtggaagttggaggggaagggtttgaggtagagataaaagtaagagagggattttttaggtttttttggggacgggaggatggtgtttgtttagttttgcgtttgtaatttttacgttatatatattatagttatgtttgggttacgtttgtttagagtttatcgggtatgtatatatgtgatatgttcgaagttgagggtaagttatttagggtagagtatgtttttaggttttgcgtttgttttagggtagttgtaatgtagtaagcgtgttattgttacggaggtaagaaagttagcgtttttttacgtacgtatataggttaggtggttttttttgtataagatgggttattaataaatttatttatgttatatatttgttttttatcgtttttattttattcgtaaagtatggggaaaagaaagtaaaagtttttttcgatttggtttttttttgcgtttttttttggagtcgaagagtttgattcgggaaaagatatttaatttgtttttgtcgtagttggttttgtttttgttggggttgttgatatatttgtaggcgttggatttttcgcgttcgcgggtttgttttttttgacggatgagcgagttggcgatagtcgtcgttatggttacgacgtttattattatcgttttttttgttgtttttttttgggttcgtttttttttttttttttttcgttgtttgttcgttgtttcgcgatttcgtcgttttggtttttttagtttgcgtttgttcgggttttttcgtattcgggttttagttaaggagggggtttagtatgtcgttcgagttttttcggcggcggttcggttttcgcgcgggttgttggttgtttgggtcggagtcgacgttatagtttcgggttcgggatcgcgggcgagattggtacgcggatgttgaattgcgcgattgcgtgtggtcggtttttgcgttcggcggatatcgtgtatgtttagttgtttcggttcgaatttcgtcggattttttcgttttgttgtttttttgatgggggttagaggagggaggcgggcggagggagtgagcgcgggcgggagagaggtcgggagttcgggcggtcggacggaggagggacgaggtagcgcgcgggggagcgcgttttcgttttggttttttcgagttttcgattagtttttgtaattttttggcgtgataatcgggaaaagttggttcgtgttaggttttcgatagggattaaataggtaatggtttcgtattttcgttgttgttgttgttttgggcgcggtatagtcgtagtataggaatttagtcgtcgtaggtatttttcggaatagcgcgatagtttttttgtagttttttttcgcgacggtttttttttttcggggttgagttcgtaggttcggagtcgtaggtcgttttttttatttttatttttatttttatttttttaagttttcggtcgttcgtttcggttaatatttgtaggggttttcgtattgttttagtcgtttaaattttttttttttttttttttcgaaaggagtttcgggaggaaatagtgatggaggaggaattgagatgaggtagttgggttgacggagcgggggtttgttcggtattgggggatggagtggtgggggtgtttttttagtgtttgagttga

SEQ ID NO: 101 - DNA, 1700 bp, Artificial Sequence
  chemically treated genomic DNA (Homo sapiens)
ttagtttaaatattgaggaaatatttttattattttattttttaatgtcgagtaaattttcgtttcgttagtttaattattttattttagttttttttttattattgttttttttcgaagtttttttcgggaaaaggaggggagaaggatttggacgattggggtagtgcggaagtttttataggtgttagtcggaacgggcggtcggggatttgggggggtgggggtgggggtgggggtgggaggggcggtttgcggtttcgagtttacgggtttagtttcgggagggaggggtcgtcgcgggagggggttgtaaggaggttgtcgcgttgtttcggagggtgtttgcggcggttgaatttttgtgttgcgattgtgtcgcgtttagagtagtagtaatagcgaagatgcgaggttattatttgtttgatttttgtcggaaatttggtacgggttaatttttttcgattattacgttaagaagttgtaaggattagtcgaagattcggaggggttagggcgagggcgcgttttttcgcgcgttgtttcgtttttttttcgttcggtcgttcgagttttcggtttttttttcgttcgcgtttatttttttcgttcgttttttttttttggtttttattagaagggtaatagggcgagggggttcggcgaaattcggatcggagtagttggatatgtacggtgttcgtcgggcgtaggggtcgattatacgtagtcgcgtagtttagtattcgcgtgttagtttcgttcgcgatttcgggttcggggttgtggcgtcgatttcgatttaggtagttagtagttcgcgcgggagtcggatcgtcgtcggaggagttcggacggtatgttgagttttttttttggttgaagttcgagtgcggagaagttcgggtaaacgtaggttaaggagattaaagcggcgaagtcgcgagatagcggataagtagcggaggagaaggaggaggaggcgaatttagagaggggtagtaaaagaagcggtggtggtgggcgtcgtggttatggcggcggttatcgttagttcgtttattcgttagaagaggtaagttcgcgagcgcgagaaatttaacgtttgtaagtgtgttagtagttttagtaaaggtaagattagttgcgataaaaataagttaaatgttttttttcgggttaaatttttcggttttaagaagaggcgtagaagaagattaggtcggaaagggtttttgtttttttttttttatattttacgagtgaggtgggggcggtggggagtaggtatgtagtataggtgagtttgttggtggtttattttgtgtagaagaggttatttagtttgtgtgcgtgcgtgggaaggcgttggtttttttgttttcgtaatagtaatacgtttgttgtattgtaattgttttgggataggcgtagagtttgggggtatattttgttttgggtggtttgtttttagtttcggatatgttatatatgtgtatgttcggtgggttttgggtaggcgtgatttagatatggttgtgatatgtatggcgtggaaattgtaggcgtagagttgggtagatattattttttcgtttttaggagagtttggggaattttttttttgtttttatttttgttttagattttttttttttagtttttatttttaagggatgtgggtttttgttgtttag

SEQ ID NO: 102 - DNA, 1700 bp, Artificial Sequence
  chemically treated genomic DNA (Homo sapiens)
ttttttttttttttttttttttttttttttttttttttttttttttttttttgtttagcgaggagtttagattagagaggtggtttagatagtttattaaatggtaagattagattagtttttaaattaagataatttattttgtttttttttttttttgaaatatatattttttgtttttagagattttaaaattttttgatgatttatatatttatttgaatttttgttttttgataaaagttattatgcgtattttggaaaaagtgaagaaaagttaaaagaaaatatttatttagagataatattttagtggatttttttgtttttatttttgtatatatgagtttatagggtatatatgtttgtatatgtttaaatttatattataagtatgtttagtttatttttagaattttttaaataaatataatttaaaataggaatttaatattttatgttattgatagatcgtagtagattgtgtttgtatttttagataatatttttttttattattgtaaataagattttattttgtgtttttaatttttatttttgttaatattttttgttatttttttaggatatatatgtagaagaaatattaattagttaaatattttaatgtttataagatatgttattttagggttttttagaaaaatagtattatcgtattttgttttcggaggtgtaggcgagtgtttggtttacgggtgtttataattattatcgggtatttataagagattttgaatttagaatattttcgcggtaggagtttcgtttcgtttttttaggggatattgacgattcgttgggttttcgttttttaagtgttggaggggggaagtagggttattcggggtagaaatttaacgttagaaattttttagcgttttattagttggtggaattttcgtggtagatagtcgcgttttatttaagtcgtagaagaaaagatattttgtgggggatttggcgtcggaagttttgagtttaaatcgtagttttaggtttttagttttgttttttttggtttgttgcgaggatgaaatgagatttttgtacgtgaaaatgttttttaatttttttcgaggggtggaaacgtttttattcgattagagttcgggtttgcggagtaatttattcgtcgttttttaagagtaggttttttagcgtagcgagtttttgttatggaaagggtcgtttcggtttaggatgcgcgttttcggcgtagatttggggataggggtttttgtcgcgttcgttttatttttgtagggagtaaggtcgtttttttgtagtatcgcgttacgcggcgggaatttgtagtgttttgttttttggttttagttttttcgttcgtttgggtttttttcggttatttttcggaggggtttggagttttttttttttttttttattttagcgttagaagtattgaaagtcgttcgttcgtcgagagtggaatcggtatttcgaatttcgtttatttcgggttaattggaggcgtattagcggttgttttgtagtgtaatagaggttcgagtgataggttatttgtttaagcgttggcgtttaggaaagtcggggaggaaattaggaagggaggcgatagtagaaaagttagagagtttagagatttttaggtaggttttcgtgtagttttaatagcgtggtgtcgggaggtgtgt

SEQ ID NO: 103 - DNA, 1700 bp, Artificial Sequence
  chemically treated genomic DNA (Homo sapiens)
atatatttttcggtattacgttgttggggttgtacggaggtttgtttaggagtttttggattttttggtttttttgttgtcgtttttttttttagtttttttttcggtttttttgggcgttagcgtttaggtaggtggtttgttattcgagtttttgttgtattgtaaggtagtcgttaatgcgtttttaattgattcggaatgagcgagattcgaggtatcggttttattttcggcgggcgggcggtttttaatgtttttggcgttgaagtaggaggagggaggagggattttaggttttttcgagaggtgatcgagagagatttaagcgagcgaagaagttgaaattagaaagtaggatattgtaaattttcgtcgcgtggcgcggtgttgtagaaggacggttttgttttttgtaggggtggggcgagcgcgatagggatttttatttttaggtttacgtcgggagcgcgtattttgagtcgaaacgattttttttataataaggattcgttacgttggggagtttgtttttggaaagcggcgggtaaattgtttcgtagattcggattttggtcgaatggagacgtttttatttttcgagaggggttagaaagtatttttacgtataaaaattttattttattttcgtagtaagttaggaagggtaaggttggggatttagggttacgatttgaatttaaggttttcgacgttaagttttttataaggtgtttttttttttacgatttaggtagagcgcgattgtttgttacgaaaattttattaattgataaggcgttggaaggtttttggcgttaggtttttatttcgggtggttttattttttttttttagtatttggaaggcgagaatttagcgagtcgttagtattttttggaaggacgaggcggaatttttatcgcggaaatattttaagtttagggttttttgtgggtgttcggtgatgattgtaaatattcgtagattaggtattcgtttgtattttcgggaataaaatacgataatattgtttttttaggaagttttggaatggtatgttttatgagtattaaaatatttgattgattgatgttttttttatatatgtattttgagaaaataatagaaaatattgataaggatgaaaattgaaggtataaaataaagttttatttatagtgataaaaaaagatattatttaaaaatataaatatagtttattacggtttattaataatatgaaatattagatttttattttaaattatatttgtttaaaaaattttaaagatgaattgggtatgtttatgatgtaaatttaaatatatataaatatatatattttatgagtttatatatatagaaataaagataagaaggtttattaaaatgttgtttttgggtggatatttttttttaatttttttttattttttttaaggtgcgtatgatgatttttattaggaaataaaaatttaaataaatgtatgggttattagaagattttgagatttttaagagtaaaggatgtatattttaaaaagaaagaaagatagggtaaattgttttggtttagaggttagtttggttttgttatttggtgaattatttgggttatttttttgatttggatttttcgttgaataaaagaagaagaaggagaaggagaaggaggaggaggaggaggaggggaagaa

SEQ ID NO: 104 - DNA, 1700 bp, Artificial Sequence
  chemically treated genomic DNA (Homo sapiens)
ttattaaaaaagcggagggggttttgtttaatatttgtatatattttttttcgtagtatttcgtttattttagttttttagggaaattatagataacgacgttttagagagtgttagaggtgaagtcgattcgaaattatattatagaaaggttttttgttttaaataggtttaataaagttattaaattaagtttacgattataaagaattgcgaattagaaaataattttatataggtgtagttttttattatttttttttttattttcggaaattttaaagaaaaattgttatatttttaaatatgaggttattattttaaaggttgtaataatttggaacgtaaattcgttaaattatgatataataaggttaatttcgaggattttcggtggattattagcgtatttttttataattagaaagtgaaaaattgggttagttttagagttacgtaataatgttgcgttacgaaattgcgtattttaagagaaattttttttaggtttagaattaagtcggagggattcgtcgcgtagtttaatttttaggaagtatgaaagaatgtattgtaaagagtaaaattacgcgtaatttattttaatataatcgtagttgttcgtgggtttttttgtagagaaaggttgtttaattttgacggggttatgggcgttgcgggagggtaaaagattatagagatttgcgaagttaaagtaaataacggggttggggcggcgagaatgattaatagcgatgtttcgaaaggatttcgcgataggaatcgagcgggaaggattttttttattttaatagtttatgggttaagaggtcgaagtttttttaaatattatattttcgcgagagaagggattgagaataattttttaaattattttttttcgaaatcgttttttttttcgaaaattttatttttgagattcggatgaggtttttattttttttatttttgtttcgtgatcgtcgttcgtttagttttttagtcgagttcgcggggtttggggcgtttatttcgtttatttaagggatcgcgtaggggtgaattttttcgcgttttatttgcgttttttagatttcgggcgtttcggcgttgtttcgagagttttttgcgcggtcgtcgcggtacggattagtttttatttttttatattgggcgtcgttgcgttcgtcgggggtcggtttcgaggtttttaaggtttcgcgcgcgcgtttgtcgtggtaattaagacgttttacgacgcgcgttttcgaacgtttcgcgttacgcggtcgcgcggtttcgttcgtcggtttcgttttcgttatagagttcgtagtacgtcgtcgtcgtagtttaggttacgtgagtatttacgcgcgcgttttgttagcggatttattatcggtttgtttagattaggttttgtttattttgattttttaaatggtacgtgggaggacgttcgtttttttcggatttaagagttatcgtaatattttagaaggggagaaaaggagcgagggcggtaggcgatagagaatttcgcgagttagcggtttcgcgtagatttttttaggtacggttttttgcggttacgtcggttgttcggcgtttgcgtaatttttttttttttagcgaaatcgaggttttcggagagtttagtagagagtgtgggtagtgagcgtttgtagtcgttagag

SEQ ID NO: 105 - DNA, 1700 bp, Artificial Sequence
  chemically treated genomic DNA (Homo sapiens)
ttttagcggttataagcgtttattgtttatattttttattaggtttttcggaggtttcggtttcgttggagagaaagagattgcgtaggcgtcgagtagtcgacgtggtcgtaggggatcgtgtttgggggggtttgcgcggggtcgttgattcgcgaggttttttgtcgtttgtcgttttcgttttttttttttttttttagagtgttacggtgatttttgggttcgaaggggacggacgtttttttacgtattatttagaaggttagagtgggtagaatttagtttgagtaggtcggtgatgaattcgttggtaagacgcgcgcgtgggtatttacgtgatttaggttgcggcggcggcgtgttgcgggttttgtggcgggagcgaggtcgacgggcggggtcgcgcggtcgcgtgacgcgaagcgttcgagagcgcgcgtcgtggaacgttttggttgttacggtaagcgcgcgcgcgaggttttgggaatttcgggatcggttttcggcgagcgtagcggcgtttagtgtaagggagtgggagttggttcgtgtcgcggcggtcgcgtagggagttttcgaggtaacgtcggggcgttcgaggtttggaaggcgtaggtggggcgcgggggagtttatttttgcgcggttttttgggtgggcggggtgggcgttttaggtttcgcggattcggttgggaggttgagcgggcgacggttacgggataagggtggagggggtggggattttattcgagttttagagatggaattttcgaggaagggagcgatttcgagagagaatagtttgagaagttgtttttagttttttttttcgcgggagtgtggtatttagggaagtttcggttttttaatttatggattgttaggatggagggaatttttttcgttcgatttttatcgcggagttttttcggagtatcgttattgattattttcgtcgttttaatttcgttgtttattttggtttcgtagatttttgtggttttttgttttttcgtagcgtttatggtttcgttaaggttgggtagtttttttttgtaagaaggtttacgaataattgcggttatattgggatggattgcgcgtgattttattttttataatatatttttttatgttttttagggattgagttgcgcggcggattttttcgatttaattttgggtttgagaaaagttttttttggaatgcgtagtttcgtgacgtagtattattgcgtgattttagggttgatttagttttttattttttaattgtaaggaagtgcgttagtagtttatcgaggattttcgaggttggttttattgtattataatttaacgaatttgcgttttaggttgttatagtttttgaaataatggttttatatttaagaatgtagtaatttttttttgaaattttcgaaagtggggaaaaaagtaatagaagattgtatttatatggaattattttttgattcgtagttttttataatcgtagatttaatttggtggttttattggatttgtttgaaataggaagtttttttgtaatgtggtttcgggtcggttttatttttggtattttttgaaacgtcgttgtttatggtttttttaagaagttagaatgagcgggatgttacggagggaggtgtatataggtgttagataaagtttttttcgtttttttggtaa

SEQ ID NO: 106 - DNA, 1700 bp, Artificial Sequence
  chemically treated genomic DNA (Homo sapiens)
aatttaggtttggtaggttatttgtatatattgggtggaggtggaaggggtaaagtatttttgaggtgatggttttgattttattttatatttttttggggtttggttggatttggttgagcgtgtttgagtagtggttgatttttggaaaataggaggatttttttttaggaggttgtgattattaaggttgtaaatgttttatttgaatttgggtgtatgagggattttttttttttatatatatatatttttttatcgcgtaagataaatttttgtttgtgagttgtttatattaaatgttttgtttttgattttttgtgattaaaatttgtttcgtttgtagggttcggtgcgtttggatttagtgtattttgttggtatagtaaaaaagagaattgtttttttataggtgattttcgttttaaagtttttttttttattaatttttttttgagaagagagtaaaatcgaattcgagagaaaagaggtagtaattattcgggaggaggtagattggagtgggtattagtttcggtagtaagagttttaacgtttttttcgtttttttttcgtagagtagtatggaagtattagtttttttttttgggtttttaaaagttattttttttttgggtgtgattttcgaattcgaagcgtttgagttttaagaaattagaataagttagagttaaagaggtttggcgggcgggggtgcgggtggagttcgggtcgggtggcgtggaaggtcgagtatagttgcgagcggcgagtttcgggagaggaggagcgggagaaagcggggcgggtttgcggtcggggttcggtcgggggagggggttttcgtttttttttttttattttcgttttttttggttttggtgacgttgcggtttttttttttttttagcggagttttagcgttcgtcgcgttttttttttcgtttttttcgtttatatttattcgttttcgtcggttcggtttttagtagcgtcgttcgaggttgtagtagcgtatttcggggtatggcgcggcgggggcgcggagggttcggttcggagggggtcgggagttcgggcgttttggagtgaggaggatcgggagttggttttggaggttgcggaggcgacgtcggagagaacgaagtttcggttgggagcggtaagtggaggggcgcgtaagggacgagttggggcgcgcggcgtaatttttttcgggagtttcgggcgggtagttttcggggttgtttcggcgcgggcgggagacggagagttacgttttcggttgagagcggagcgtcgcgggttgcgcgttcgggttcggttcggttggatttcgagagagcgtttgacgcgtgaagtttgagttacgggttttttcggcggcggagtagtttcgaggggtagtttttcgattttttttatttttttgaacgtcgagtatcgagtcgggaatacgatgagaatttggagatcgtatttcgtgcgtcggatttggtcggtttagtcgcgattttttcgacggtttttggggtttcggttttattttgcggggttgggtggtatattttcgcgttttttttttttttcgtattttagttatttgggggtaggggaggtggcggcggtttgtgtgtagattttggcgttggtaattttattttgcggttgtagaagtcgaggaagagttttttaagttt

SEQ ID NO: 107 - DNA, 1700 bp, Artificial Sequence
  chemically treated genomic DNA (Homo sapiens)
aggtttggggagttttttttcggtttttgtagtcgtaaagtagggttgttagcgttaggatttgtatataagtcgtcgttattttttttgtttttaaatggttggggtacggagggaaaggaggacgcgagggtgtgttatttaatttcgtagagtgaagtcgaggttttaaggatcgtcgaggaaatcgcggttgggtcggttagattcgacgtacggaatgcgatttttaggtttttatcgtgttttcggttcggtgttcggcgtttaagggggtggggaggatcgaaaagttgtttttcgaggttgtttcgtcgtcggggaagttcgtggtttaggttttacgcgttaggcgttttttcggagtttagtcgagtcgggttcgggcgcgtaattcgcggcgtttcgtttttagtcggggacgtggtttttcgtttttcgttcgcgtcgggatagtttcgaaggttattcgttcgaaattttcgaagaaagttgcgtcgcgcgttttagttcgttttttgcgcgtttttttatttatcgtttttagtcgaggtttcgtttttttcggcgtcgttttcgtagtttttagagttagttttcggttttttttattttagggcgttcgggttttcggtttttttcgaatcgagtttttcgcgttttcgtcgcgttatgtttcgggatgcgttgttgtagtttcgggcgacgttattgagagtcgggtcggcgggggcgggtaggtgtgggcgaaaggggcggagggagggacgcggcgggcgttgaggtttcgttggaggggggaggaaatcgtaacgttattagagttagaggagacgaggatgaggagagggaaacggggattttttttttcggtcgaatttcgatcgtaggttcgtttcgttttttttcgttttttttttttcgggattcgtcgttcgtagttgtgttcggttttttacgttattcggttcggattttattcgtattttcgttcgttaggtttttttggttttaatttgttttagttttttaggatttaaacgtttcgggttcgagaattatatttaaaggaaaggtaatttttggaggtttagggggaggagttgatgtttttatattattttgcgagaggaaagcgaggaagacgttaagatttttattatcgggattggtgtttattttagtttgttttttttcgaatagttattgttttttttttttcgaattcggttttattttttttttaaagagaagttagtgggaggggggattttggggcgggggttatttatagagaagtagtttttttttttattgtattaataaaatatattaaatttaagcgtatcgaattttgtaaacggaatagattttaattatagaaggttaggagtaaagtatttaatgtaggtagtttataaatagaaatttattttacgcggtgggggggtgtgtgtgtgtgagagagagagattttttatgtatttaagtttaggtggagtatttgtaattttggtagttatagttttttggagaagagtttttttgttttttaaaaattagttattgtttaaatacgtttagttaggtttaattaggttttaaggagatgtgaggtaggattagggttattattttaggaatattttgttttttttatttttatttagtgtgtgtaagtaatttattagatttagatt

SEQ ID NO: 108 - DNA, 1700 bp, Artificial Sequence
  chemically treated genomic DNA (Homo sapiens)
aatagaatttagaattttggggtagttttttggagttggagtttttagggtaaagataacgttaatgcgttgtttaaaagggtttaatttttaaaatatttttattggtaatatttattttgtttattagaaatgtagttatgtatttttgtgttcggtaaagattttttgttttgtgtaaatttataaattaaaataattagtttagatttttgacgtgattttgtagtggaagtatttgtattagtgaatgttgaaaagtaaataaaaatgaaagttttgcggaggtcgtgggtcggacgggcgcggggcgttttttgttattttagcgtttgtcgggatttttttcgcgttttcgttgttcgaattagttcggttttgggacgttttttttaaatttttataagttaaatcgaatttaggttcgagttcgttattgtaaaggaattcggggtgcggggtgggagggataggaggagagatgggttgggtgttaaatggtagaataaatggttgatttaattaaagttgttaatttaattttttattttttttatattagtttttttttttttttttttttttttttttttttaaatttgttgaattttattattttcgcgttggtaagaaaatttgaatattttgggaattgtgtttaaattgtatttgtaatttttttggaagtttttgaaatttttcgagaatgaggcggggcggtttaaggggaaagggtagggaggagagattttcgggaagttgatgacgcgttcggggtaagttagtttgttttcgagacgcgcgttttacggtcgtagtagcgttgcgtttattttgtttttttagtgatttagttagtttttgttgggggtttttgataacgtttatcgataagttttttttgtttttaaaaggcgtgtttttttttttttttaggtataaagtaggggtgggaaggggagagttatataatgttgtaaaaagaagcgtggttttatttggaagacggttgaattttttttgttttttcgatttagttagtaacgataagaaagatattgaatatcgtttttcgaaaatcgggcgtatttgaattcggtgaaaagtgtttttgtgaggaagtttaatgtttatttttgtgtaaggataattggaaattaaagcgtttagtaatgtagagattttattaagcgagcgtttttttttttttcgcgttaggaatgaaataagttagttagatagaaaatatttttgggagaagattaattatttttgggtaggtcgaattgattggaaattatattaagtaaaggtagtatattttatttcgttttggcgatgttgattttatattattagaatattgtttttttagtgtgggggtgttttgtattttttaaagttagggtagattggttatttgggaaaatgtagtgttagtgttttgtttttattttttttattgtttaaatatgttttatatttttttaatgttttttgagggtttttaatttagttattgagttttttatttttggcgattgtgatattaataatgttgttattttttttgggaaatatggttgtaaagatgtttcgtaggaggtagataaaaggttgtgtttttagatattgaaggttgttttgttatttagatgtttttaatttttgtatagagaaggagggaaaatacggggtgt

SEQ ID NO: 109 - DNA, 1700 bp, Artificial Sequence
  chemically treated genomic DNA (Homo sapiens)
gtatttcgtgttttttttttttttttatgtaggaattggaaatatttaagtaataaagtaatttttagtatttgaaagtatagttttttatttgttttttgcggagtatttttgtaattatgttttttaaggagggtgatagtattgttggtattatagtcgttaagggtgaggaatttagtggttgggttgagaatttttagaaggtattagaaaaatgtggaatatgtttaaatagtgaagaaagtaggagtaaagtattaatattgtatttttttaggtaattagtttattttgattttgggaagtataaagtatttttatattgggggaataatgttttaataatatagaattaatatcgttaggacggaatggaatgtattgtttttgtttggtataatttttagttaattcggtttatttagaagtaattagtttttttttaaaagtgttttttgtttaattggtttattttatttttggcgcgggggggaggggagcgttcgtttaatgaaatttttgtattattagacgttttaatttttagttatttttgtataaaagtaaatattaggtttttttatagggatattttttatcgagtttaaatgcgttcgattttcggagggcgatatttagtattttttttgtcgttattaattgggtcgaggaagtaaaagaaatttaatcgttttttaaataaagttacgtttttttttataatattgtgtagtttttttttttttatttttattttgtatttggagagaaagagaagtacgttttttgggagtaaggagagtttatcgatgagcgttattagaaatttttagtaaaggttggttgggttattaaaggggtagggtgggcgtagcgttgttacgatcgtgaggcgcgcgtttcggaaataggttgatttgtttcggacgcgttattagtttttcgagggttttttttttttgttttttttttttgagtcgtttcgttttattttcgggaagttttaggaatttttaggggagttgtaagtgtaatttaagtataatttttaaggtatttaggtttttttattaacgcggagatagtgggatttagtaagtttaagaggaaaaaagaaaaagaaaaaaaaaaagggttggtgtgagagggatgggaaattagattgataattttaattaaattaattatttgttttattatttggtatttagtttatttttttttttattttttttatttcgtatttcgggtttttttgtagtgacgaattcggatttgagttcgatttgatttgtgaggatttgggggaaacgttttaagatcgagttggttcgggtagcgaaggcgcgggaaggatttcggtagacgttaagatggtaggaggcgtttcgcgttcgttcggtttacggttttcgtaaagtttttatttttatttgttttttagtatttattggtgtaaatatttttattgtagggttacgttaagaatttaaattaattattttagtttgtagatttatatagaatagaaaatttttatcgggtataaaaatgtatggttgtatttttgatgaatagaatgggtgttattaataaaagtattttaaaaattgaatttttttaaataacgtattagcgttatttttgttttaggagttttaattttaaagaattgttttagagttttgaattttgtt

SEQ ID NO: 110 - DNA, 1700 bp, Artificial Sequence; identical to SEQ ID NO: 108
  chemically treated genomic DNA (Homo sapiens)

SEQ ID NO: 111 - DNA, 1700 bp, Artificial Sequence; identical to SEQ ID NO: 109
  chemically treated genomic DNA (Homo sapiens)

SEQ ID NO: 112 - DNA, 1700 bp, Artificial Sequence
  chemically treated genomic DNA (Homo sapiens)
attggatgagtggggttttttgttttagttggatttttataggaggatggaggaaagtatttttttgagtaagtttggagagtgaaggggtggggttgtaaggttggagaaggaggtgttttttttataaaaatgattgggttttttttgtaatggttggtataaagtcggaagggtttggggacgggaggggtttgaagtgatttttgaggagttgggttttttttgtaaggttttgggagggaaggtaaggtattttgcgtaatgatatattttagttgttggatgtgaagattttttttcgaagatagaataaagaaaggtttttttattaatgcgggtttgaaattggtttgaaatggtgtgtttgtagggattcgggaggaaatgcgacgtttatttttagatttgggaaaggcgggggtaataattttttaagagaattggagttcgaaagaggaatgaaaagttttcgcgtcgcgtatatatatttttttttattggaaagttaggagcgaggaaagaattttgaagtgatttgggaacgatttgaaagcgcgtaagttggaagtcgggttgaagttagtatagcggagtaggggttaggtttttgcgtggtcggagtgggagagcgtttttgagaataagatggggtaggatcgaaggggcgtgaggtcgacgttttttagggatgattcggagggtgaaggaggtttgggggtggtttcgggttttcggaggtgattgagggagtttttgagttaatgttcgggggtgggaggcgttatcggtatttattgagttgatttttagcgttagggcgtattgtagcgtttttattttcggtattcgtgcggggaggggcggcggggagtgatttcggttacgggttcgagtgaggcggggagatttggggttgggcggggacggggaggagacgaggtggcggcggcggtggtagtagtggtttttaggtcggggttcgcggtttagtttgggtttcgtcggagaagtagttgagcgcggggcgtagattcgttgttatggtagttaggaggggcggggtcggaagagaggtcgcgcggggtattttagggttattcgcgttgggacgagcgttggaggcggagtttgacgaacgtcgagcgaggcggagtcgggtagggggcggggttttcgaaggggcggggtgaggtaaggtagtttttttgtgacgttaaattgcgtttaaagtcggacgaaagggagttgttgtttgtataatgggacggtgttttgtttagggagttggagtttttgtaaggtcgtcggagggaatttttgagttataacgtggtgatggtattttgggttagtggcgatttattgtggttttagtttttttaattgtaaaagaataataattattgttagtttttttatttaaattttacggtacggtttataaagttagtataaggatattgggaattagggaagagatttgtttaaattaaagtcggaattaggattggaatttaggagtttttaagttaaattttagttgtttttttgaggagaggggtttgtattagatggtttatatttttagttttttattttgtgattttttttgttagcgcgtttttttttttttttaaatttttttgtttaatttatttatgtatgcgttttaatgtatattgtttaagtggaattgt

SEQ ID NO: 113 - DNA, 1700 bp, Artificial Sequence
  chemically treated genomic DNA (Homo sapiens)
atagttttatttaagtaatatgtattaagacgtatatatgggtgaattagataggaaaatttgaagggaaaagggaacgcgttaataaagggaattatagaataggagattaggagtatggattatttgatgtaagtttttttttttagggaggtaattggggtttaatttgagagtttttgggttttagttttggtttcgattttgatttgggtaagttttttttttaatttttagtatttttatgttggttttgtaaatcgtgtcgtgaaatttaaataggaggattaataataattattatttttttataattgggaaaattgaggttatagtgggtcgttattagtttaaggtgttattattacgttgtaatttaggggtttttttcgacggttttataggaattttaattttttgaatagagtatcgttttattatataaatagtagttttttttcgttcggttttgggcgtagtttgacgttatagaagagttgttttgttttatttcgttttttcggaagtttcgttttttgttcgatttcgtttcgttcggcgttcgttaagtttcgtttttagcgttcgttttagcgcgggtagttttggagtgtttcgcgcggtttttttttcggtttcgttttttttggttgttatgataacgagtttgcgtttcgcgtttagttgttttttcggcggaatttaggttggatcgcgggtttcggtttgggggttattgttgttatcgtcgtcgttatttcgtttttttttcgttttcgtttagttttaggttttttcgttttattcgggttcgtggtcggggttatttttcgtcgttttttttcgtacggatgtcgaaggtgaaggcgttgtagtgcgttttggcgttggagattagtttagtgagtatcggtagcgttttttattttcgggtattggtttagggatttttttagttattttcgagagttcggaattatttttaaattttttttatttttcgaattatttttagggagcgtcgattttacgttttttcggttttgttttattttgtttttagaagcgtttttttatttcggttacgtagaggtttgatttttgtttcgttatgttggttttaattcgatttttaatttgcgcgtttttagatcgtttttaaattattttagagtttttttttcgtttttaattttttagtgagagagagtgtatgtgcgcggcgcgggggttttttatttttttttcgggttttagtttttttagagaattattgttttcgttttttttaagtttgggggtgggcgtcgtattttttttcgaatttttgtaggtatattattttagattagttttaaattcgtattaatgaaggggtttttttttattttgttttcgaaggagaatttttatatttaataattgaaatgtgttattgcgtaaagtattttgtttttttttttagggttttataggaaaaatttaattttttagaaattattttaggtttttttcgtttttagattttttcgattttgtgttagttattgtaagaaaggtttagttatttttatgagggaaatatttttttttttaattttgtaattttatttttttattttttagatttgtttaagaggatgtttttttttatttttttgtggaagtttagttggggtagagaattttatttatttagt

SEQ ID NO: 114 - DNA, 1700 bp, Artificial Sequence
  chemically treated genomic DNA (Homo sapiens)
ttaggagtttagattcgggtaggaaataaagttttaaagcggttttgaagtgttttttttttagatttttttttagcgcgggttatttattttagtttagggaagagattattttgtttgaacgtagttaaacgtatcgtacgtgggagttaagtgggagatgaaataatttaatgcgttttttttattattattatttttattaaattagttatcgtcggtttaaatttatcgggaatgttcgtgtattttatcgattagggaaataaaagggtgtatataaattagtggaagcgggtgggaaggaataagggatgtagattgtcgtgagtaaaacgtttcgcggtttggtgagaggttgttttttttttaagttatttattttagattgaaatattaagttagtttaggttattttttgagtagtattataagttaaaataagatttagaaaatttattaatttttaattgcgttgtttttagattgggagtatagggggatagggtgtagggttgtggggaggtcgaggggagggggatttgtggaaaagtaaacgttcgagaaattttagttttcgattttagttgttttttattttatttagttcggtcgaggagtcgtttttatattttatggtaatttcggaatttatgatgtattttttagagttgggtttttagttttttaggtttcggagtttaagagttggattttataggtttaatatttattgtttggttttttttgttatattttagtttagatagttatttttatttaggatagttgcgaagcgtaaacggaggatgtaggtatttttaaaaagagtagtcgtggttcgcgttttacggacgagttattgttgtaggaggttcggtggcgcggcgcgttcgtagttgcggtagcggcggagttcgcgcggggattttttcgcgcgtatttagcgtttcgtttcggggtagtgttattttgggcgtcgtttcgttatttttacggtgtttcgtcgttttttattattaggatggagggtattttttagtacgagtcgatattttagagggtatcggcgagttttcgaaaaaattagggcggtggggcgaggaagattcgcgtttcggattttagagaaggaaagaaagttagagggagtcggagcggagttgtattttacgtcgaagtttttttttcgaatcgtttggagttgtaagtggtttttgattgaatatagttgtttagttgttggttacggggtgcgcggaggaggcggggttagagggagaaaagggtgggggcgagtagtttggaggtcgagtagaggagatcgtgttgggagagtagagtaggtatttttttagttttttgtaatagtaattaaggattcgagtttaggagggggagtagaaataggaaaagttatttgaataatcgtgtaggaaattaaaaaaaaaaaaagaaaagaaaaagaaaaaaaattttttttataggagtcgttttttatgatttaataaggttaagataaatttaaatattgtatattagatagtacggtgtattttttacggaatattaagattttataagggtgtagggtagttattttttttaattagtttaaagttaacgttacgggtgtaaatttaatgttaaattttttttgtttgttattggggtagagtatattttttgtatgtttataa

SEQ ID NO: 115 - DNA, 1700 bp, Artificial Sequence
  chemically treated genomic DNA (Homo sapiens)
ttatggatatgtagaaaatgtattttgttttagtgatagatagagggagtttgatattgaatttatattcgtagcgttgattttagattgattagggaaaatgattgttttgtatttttgtaaaattttaatgtttcgtgagaagtgtatcgtgttgtttgatgtatagtgtttaggtttgttttgattttattaaattatggaaagcggtttttatgagggaagtttttttttttttttttttttttttttttttttaattttttgtacgattatttaggtggttttttttgtttttatttttttttttggattcgggtttttaattattattgtagagaattggaaggatgtttgttttgtttttttagtacggttttttttgttcggtttttaggttattcgtttttatttttttttttttttagtttcgtttttttcgcgtatttcgtggttagtagttagatagttgtgtttaattagaaattatttataattttagacgattcgaaggggaaatttcggcgtgaagtgtagtttcgtttcggttttttttagttttttttttttttttggaattcgaggcgcggatttttttcgttttatcgttttagttttttcgggagttcgtcggtgttttttagggtgtcggttcgtgttgggaagtgttttttattttggtaatggggggcggcgaggtatcgtaggagtggcgaggcggcgtttagggtggtattgtttcggaacggggcgttgggtgcgcgcgggagggttttcgcgcgggtttcgtcgttgtcgtagttgcgagcgcgtcgcgttatcgagttttttgtagtaatggttcgttcgtgaaacgcgagttacggttgttttttttaagagtgtttgtatttttcgtttgcgtttcgtaattgttttgggtgaaaatggttgtttagattaaaatgtggtagaagggattaagtagtggatattgagtttgtgaagtttaatttttaagtttcgagatttgggggattgagagtttagttttgaaaagtgtattatgaatttcggagttgttatgaaatatggaaacgatttttcggtcgagttgagtgaggtaagaaatagttgaggtcgggagttagggtttttcgggcgtttgtttttttataaattttttttttttcgatttttttatagttttatattttatttttttgtgtttttagtttggaggtagcgtaattgaagattagtagattttttggattttgttttaatttgtagtgttatttaggaaatgatttgaattggtttgatattttagtttgaaatgagtggtttaaaaaagaaataattttttattaagtcgcgaggcgttttatttacgatagtttgtattttttgtttttttttattcgtttttattaatttatatatatttttttatttttttgatcggtaaaatatacggatattttcgatgagtttaagtcgacggtaattaatttggtgggggtggtggtggtgagggggacgtattggattattttattttttatttggtttttacgtgcgatgcgtttggttacgtttaggtaaggtggttttttttttaggttgagatgaatgattcgcgttggggaggggtttggggagaaagtattttaaagtcgttttagaattttattttttgttcggatttggatttttga

SEQ ID NO: 116 - DNA, 1700 bp, Artificial Sequence
  chemically treated genomic DNA (Homo sapiens)
gttggtaagtgaaaagtttatagtttatagtatttttattgtttagttttatttttattgttgagtttaggaatttgtttttttgtttttttttggaagcgattttgtgtgtgtaggtaggggtagagggagtaaagttaaacgtaattatagtattttagatttgtagatttttaaattagttttatattttagtgaggtgataatggttaatatttttattttagttttatagaagtttagttatagtaggtgatttgcgtaaagttatatagtttttggttttaaggtagggtttttttttttgtttgtgttttttcgttatcgtagtttttgttttttagttgggggattatttagaggattatttggattttgttgttttcgttgggttatttgtttggcggagtagttggggggtggggggtttgtaatttaggattttatttggcgttgttttagtttatcgagtagtgagttagttttttttttatcggtggtagggggagttgtcgggattttagggttgttttgaggggtcgcgtagatttgggggatgtaaaggtttaaacggattcggaatggtcggtgcggtcgggttttgggaaatgaagggttcgcggtgtttcgtattttttgtgggggcgggagtaattattaggtggtcgcggagttagtcgagggggcggttgttaattcgggcggcgatcgtaggcggagacgtttcgttcgggatagggatcgggtcgcgtcggcgttcgcggatagatattcgcggtttcggtcgttgtcgcgtttttcgttttcgttacgtttcggcggtttcgtttggggcgcgttttcgtttcgtacgcgtacgcgtttgtattcgtattggttcgttcggttttttcgcgttcgcggggtcgtttcgggggaggtgtcgagattcggttttggttcgcgcgcggttttcggggttcggcgcgtcggggatttataggcgaggcgagagaaaggggttggttcgcggggatatttatttgcgcggcgcggggttgtggcgtcggttattattatttcgcgtttttttttttgttagcgcgtacgtagatggcggggtggtttggggaggttttcgggtttttttttgggaacgtagggttaagttgtgtttcgattttacgttttttttatttacgtcgggtatacgtagttttgagtgagggtttatgttttgttttttacgttgattatagtttttaggtttggcgtttgtcgggaagggtcgtaatggtttagggatttttgtttttattattattttgttttggggggataggttttagaatattgcgtggggtttagagtgggtttagttgtaaaaggataggggtttttttttagttacgtttattttcgggtgttttggttgttagtagttttttattataatcgtagggtgagaagtttagagaatagtttaggagtagtttttttgttttttattacgttcgtgtgtttagtacgggtaaattagtagttacgtaataaatgaagtgcggtgttgtagttttgggtttttttgtatttgttttgattttagtttgtttagtttagtttggggatttgtatttaggtttgtagttttttgggtttttgttcggtgttttttagagtcggagtttgtttgtcgtagggatggtttagaggatttttta

SEQ ID NO: 117 - DNA, 1700 bp, Artificial Sequence
  chemically treated genomic DNA (Homo sapiens)
taggaggttttttaagttatttttgcggtagataggtttcggttttgaggagtatcgggtagggatttaggaggttgtaaatttaagtgtagatttttaggttgggttggataggttgaggttagggtaggtgtagagaggtttagagttgtaatatcgtattttatttattgcgtggttattgatttgttcgtgttgggtatacggacgtggtaaaggatagaagggttatttttggattgttttttgggttttttattttgcggttgtggtgagaggttgttgatagttagggtattcgggagtgggcgtggttggagaaggatttttgtttttttatagttaggtttattttgagttttacgtagtgttttagagtttgtttttttagggtaggatggtagtgggggtaggggtttttgagttattgcggtttttttcggtaggcgttaagtttaggggttgtggttagcgtgaaaggtagggtatggatttttatttagggttgcgtgtgttcgacgtgggtggggggggcgtggaatcggagtataatttggttttgcgtttttaggaagggattcgaagatttttttaggttatttcgttatttgcgtgcgcgttggtaagaggaggggcgcggggtaatggtggtcggcgttatagtttcgcgtcgcgtaggtaggtgttttcgcgagttaatttttttttttcgtttcgtttgtgggttttcggcgcgtcggatttcggaaatcgcgcgcgggttaaggtcgggtttcggtatttttttcggagcggtttcgcgagcgcgggggaatcgggcgggttagtgcgggtgtaggcgcgtgcgcgtgcgaggcgggggcgcgttttaagcgaggtcgtcggggcgtggcgggggcgggagacgcggtagcggtcgggatcgcgggtgtttgttcgcgagcgtcggcgcggttcggtttttgtttcgggcggggcgttttcgtttgcggtcgtcgttcgggttagtagtcgttttttcggttggtttcgcggttatttggtaattattttcgtttttataaaaagtgcgagatatcgcgagttttttattttttagagttcggtcgtatcggttatttcgagttcgtttgggtttttatattttttagatttgcgcggttttttagggtagttttgagatttcgatagttttttttgttatcgatgaggggagagttggtttattgttcggtgggttggggtagcgttaggtggggttttaggttgtaggttttttattttttaattatttcgttaagtaaatggtttaacggaggtagtagggtttaggtggttttttgggtggttttttagttggagaataggggttgcggtggcgagagggtataggtaggggagagagttttgttttggagttagaggttgtgtagttttgcgtaggttatttgttgtggttgggtttttgtaaaattgagatgaaaatgttagttattgttattttattgaggtgtgaggttggtttgagagtttgtaaatttgagatgttatagttacgtttggttttattttttttgtttttatttgtatatataggatcgtttttaaaaggaggtagggaaataaatttttgagtttaatagtggaagtggggttggatagtgaggatgttgtggattgtgggttttttatttgttagt

SEQ ID NO: 118 - DNA, 1700 bp, Artificial Sequence
  chemically treated genomic DNA (Homo sapiens)
gtaagattttattttaaaaaaaaaaaaaaaaaaaagcgatttgtttagtaatagttttaaaatatggaagagttgggattggatttagattatttggttttggaattgggttggtaagtgaaaagtttatagtttatagtatttttattgtttagttttatttttattgttgagtttaggaatttgtttttttgtttttttttggaagcgattttgtgtgtgtaggtaggggtagagggagtaaagttaaacgtaattatagtattttagatttgtagatttttaaattagttttatattttagtgaggtgataatggttaatatttttattttagttttatagaagtttagttatagtaggtgatttgcgtaaagttatatagtttttggttttaaggtagggtttttttttttgtttgtgttttttcgttatcgtagtttttgttttttagttgggggattatttagaggattatttggattttgttgttttcgttgggttatttgtttggcggagtagttggggggtggggggtttgtaatttaggattttatttggcgttgttttagtttatcgagtagtgagttagttttttttttatcggtggtagggggagttgtcgggattttagggttgttttgaggggtcgcgtagatttgggggatgtaaaggtttaaacggattcggaatggtcggtgcggtcgggttttgggaaatgaagggttcgcggtgtttcgtattttttgtgggggcgggagtaattattaggtggtcgcggagttagtcgagggggcggttgttaattcgggcggcgatcgtaggcggagacgtttcgttcgggatagggatcgggtcgcgtcggcgttcgcggatagatattcgcggtttcggtcgttgtcgcgtttttcgttttcgttacgtttcggcggtttcgtttggggcgcgttttcgtttcgtacgcgtacgcgtttgtattcgtattggttcgttcggttttttcgcgttcgcggggtcgtttcgggggaggtgtcgagattcggttttggttcgcgcgcggttttcggggttcggcgcgtcggggatttataggcgaggcgagagaaaggggttggttcgcggggatatttatttgcgcggcgcggggttgtggcgtcggttattattatttcgcgtttttttttttgttagcgcgtacgtagatggcggggtggtttggggaggttttcgggtttttttttgggaacgtagggttaagttgtgtttcgattttacgttttttttatttacgtcgggtatacgtagttttgagtgagggtttatgttttgttttttacgttgattatagtttttaggtttggcgtttgtcgggaagggtcgtaatggtttagggatttttgtttttattattattttgttttggggggataggttttagaatattgcgtggggtttagagtgggtttagttgtaaaaggataggggtttttttttagttacgtttattttcgggtgttttggttgttagtagttttttattataatcgtagggtgagaagtttagagaatagtttaggagtagtttttttgttttttattacgttcgtgtgtttagtacgggtaaattagtagttacgtaataaatgaagtgcggtgttgtagttttgggtttttttgtatttgttttgattttagtttgtt

SEQ ID NO: 119 - DNA, 1700 bp, Artificial Sequence
  chemically treated genomic DNA (Homo sapiens)
gataggttgaggttagggtaggtgtagagaggtttagagttgtaatatcgtattttatttattgcgtggttattgatttgttcgtgttgggtatacggacgtggtaaaggatagaagggttatttttggattgttttttgggttttttattttgcggttgtggtgagaggttgttgatagttagggtattcgggagtgggcgtggttggagaaggatttttgtttttttatagttaggtttattttgagttttacgtagtgttttagagtttgtttttttagggtaggatggtagtgggggtaggggtttttgagttattgcggtttttttcggtaggcgttaagtttaggggttgtggttagcgtgaaaggtagggtatggatttttatttagggttgcgtgtgttcgacgtgggtggggggggcgtggaatcggagtataatttggttttgcgtttttaggaagggattcgaagatttttttaggttatttcgttatttgcgtgcgcgttggtaagaggaggggcgcggggtaatggtggtcggcgttatagtttcgcgtcgcgtaggtaggtgttttcgcgagttaatttttttttttcgtttcgtttgtgggttttcggcgcgtcggatttcggaaatcgcgcgcgggttaaggtcgggtttcggtatttttttcggagcggtttcgcgagcgcgggggaatcgggcgggttagtgcgggtgtaggcgcgtgcgcgtgcgaggcgggggcgcgttttaagcgaggtcgtcggggcgtggcgggggcgggagacgcggtagcggtcgggatcgcgggtgtttgttcgcgagcgtcggcgcggttcggtttttgtttcgggcggggcgttttcgtttgcggtcgtcgttcgggttagtagtcgttttttcggttggtttcgcggttatttggtaattattttcgtttttataaaaagtgcgagatatcgcgagttttttattttttagagttcggtcgtatcggttatttcgagttcgtttgggtttttatattttttagatttgcgcggttttttagggtagttttgagatttcgatagttttttttgttatcgatgaggggagagttggtttattgttcggtgggttggggtagcgttaggtggggttttaggttgtaggttttttattttttaattatttcgttaagtaaatggtttaacggaggtagtagggtttaggtggttttttgggtggttttttagttggagaataggggttgcggtggcgagagggtataggtaggggagagagttttgttttggagttagaggttgtgtagttttgcgtaggttatttgttgtggttgggtttttgtaaaattgagatgaaaatgttagttattgttattttattgaggtgtgaggttggtttgagagtttgtaaatttgagatgttatagttacgtttggttttattttttttgtttttatttgtatatataggatcgtttttaaaaggaggtagggaaataaatttttgagtttaatagtggaagtggggttggatagtgaggatgttgtggattgtgggttttttatttgttagtttagttttagaattagatagtttaagtttaattttagtttttttatattttagagttattattgggtaagtcgtttttttttttttttttttttgagatggagttttgt

SEQ ID NO: 120 - DNA, 1700 bp, Artificial Sequence
  chemically treated genomic DNA (Homo sapiens)
aggtgggtaggttttttggagtagtaggcggtttttttagtttttatggttggagttaggatttttttttgtttataatttttttaatttttttaatttaatattttttattttttttttttttttttttttttttttttttttttttttttttttttttttttttttttttttttttttttttttttttttttttttttttgttttttttttttttttagttttttttttttattttgttgtgggcgggttgtagtgttgattttatcgggacgtttggataggtattgtttgtgataacggtggtattggaaggttttggtagttgaacgtttagttttttattttttatgtttttatattttttatgtataaattttgtaggaagtaggttagggaacgtagtttattgttttttttcgattagtagttttagtttggttatatcgtagtcgcggtagaggatacggtttatttttacgagttaatcggggtttatttttattttcggcggttttagcgggtttagtaagcgagagtaggttttcggagttataggggtttttttgttttcgtagcgagggaagtaggaggcggtaggttttgaggttcgtgggttgaaggtttaggttttacgtggagttattcgcggaggatttaggttaggttaatttgttcgtggggaattcgttcgattttggcgagttaggggtttggaaggtcgggtggagtttggttaatagttcgggttttttattttgggtttcgtttttttcggaggtttcgggggtatttagcgggtaggtttttttggttcggtcggttttggagtaggtgaggaaggttaggtaggtttcgcggaggcgggttaggtttcggtgggtggtttcgttgacgaagtagtagagtacggggtcggcgacgtagttgaagttggtgagtaggagggagaagtggtaggcgttgaaaacgtttttggcgaagtcgtagttggttttttagacgttgcgtattagtagtaatacgtggtagggtaggaagtaggttaggaagatgattacggtgttgagtattagtcgttggatttggtttttgcggtttttttgggtgtcgtggtttcggcgtacggcgcgtaggatgttttggtaggacgttagtagtaggtagatggggaagaggaagtttattaggaagcggtagtagttgatggcgcgttgttatgtttggatggggtagtgtttaaagtatacgcggtgttggttttcgttttcgatgattttttcgtgtattaggaagtagatgttggttagtagttttttggtttagatgattacgttgacgtcgacggtcgtttttagggttcggaattggtggaagcggaagggatgggttatagttaggtagcggtttacggagatgtagtagaggaagtttacgttgatgtagatgttttcgtataggaggatgtcgtatatttggtaggataggtcgtcgtgagattagttgtcgtgttgtagtacgtattgtagttagaagggtagcgagtagatgtagaagaggtcggttatcgttaggttgtataggtatacgtttagttcgtttcgggttttgatttgtaggtagtcgaagtagagggataggtagttggtcgggaagtttattattagtacggtaatatagattatcggggttagcgtttg

SEQ ID NO: 121 - DNA, 1700 bp, Artificial Sequence
  chemically treated genomic DNA (Homo sapiens)
tagacgttggtttcggtggtttatgttatcgtgttggtggtgggtttttcggttaattgtttgtttttttatttcggttatttgtagattaaggttcggaacgagttgggcgtgtatttgtgtaatttgacggtggtcgattttttttatatttgttcgttgtttttttggttgtagtacgtgttgtagtacgataattggttttacggcgatttgttttgttaggtgtgcggtatttttttgtacgagaatatttatattagcgtgggttttttttgttgtattttcgtggatcgttatttggttgtggtttatttttttcgtttttattagtttcggattttgaaggcggtcgtcggcgttagcgtggttatttgggttaaggagttgttgattagtatttattttttgatgtacgaggaggttatcgaggacgagaattagtatcgcgtgtgttttgagtattattttatttaggtatggtagcgcgttattaattattatcgttttttggtgggtttttttttttttatttgtttgttgttggcgttttattagggtattttgcgcgtcgtgcgtcggagttacggtatttagaagagtcgtaaggattagatttagcggttggtgtttagtatcgtggttatttttttggtttgttttttgttttattacgtgttgttgttggtgcgtagcgtttgggaggttagttgcgatttcgttaagggcgtttttaacgtttattattttttttttttgtttattagttttaattgcgtcgtcgatttcgtgttttattgtttcgttagcgagattatttatcgggatttggttcgttttcgcggggtttgtttggttttttttatttgttttaggatcggtcgggttagggaggtttattcgttgggtgttttcgaggttttcgggaaaagcggggtttagggtgaggagttcgagttgttgattaagttttattcggttttttagatttttaattcgttagggtcgggcgggttttttacgggtaggttggtttagtttgggtttttcgcgggtggttttacgtgaggtttgagtttttagtttacgggttttagggtttgtcgttttttgtttttttcgttgcggaggtagggaagtttttgtaatttcggaagtttgttttcgtttgttgagttcgttgggatcgtcgagggtgggaataagtttcggttggttcgtgggaataagtcgtgttttttgtcgcggttgcgatgtggttaggttggggttgttggtcgggggaagatagtgaattgcgttttttggtttgttttttgtagagtttgtgtatggggagtgtgaggatatggagggtgggaggttgggcgtttagttgttagggttttttaatgttatcgttgttatagataatgtttgtttaaacgtttcggtgggattagtattgtagttcgtttataatagggtgggaagggaagattggagggggaaggaaggtaggagggggaaggaagaaagagggggaaggaaagaaagaggggggaaggaaaggaggaaggggagaaggaagggaggaggaaagaaggaaggtagggggtgttgggttgggagaattgagggagttataggtagaggaggattttagttttagttataagaattgggagagtcgtttgttgttttagaagatttgtttattt

SEQ ID NO: 122 - DNA, 1700 bp, Artificial Sequence; identical to SEQ ID NO: 120
  chemically treated genomic DNA (Homo sapiens)

SEQ ID NO: 123 - DNA, 1700 bp, Artificial Sequence; identical to SEQ ID NO: 121
  chemically treated genomic DNA (Homo sapiens)

SEQ ID NO: 124 - DNA, 1700 bp, Artificial Sequence
  chemically treated genomic DNA (Homo sapiens)
ttttttaggattagagaattttgaagagtttgtgtattgtttttttgaaagttttgaagttaagtaattttaaaaatgtttagtaaagtttttttgggaatggtgagatggggggtggggaggatgggatatttttattgttattgtttgttttttgtttttttggtttttttatatgaagtgattggatttgggagatttagtggaaatttttaggttatttagggaattgtttttttttttggttttgtagtggtgaggtggggtagggggtggggggggtgggttgtgagagtgttgagatgtgtggggtgtggagatgtgtaagtggtgaagtttgattgagagtaggttggagtagttgtttaatttttggtgtgggatttgttgaggggttatggtgagtttttgtgggtgtgggttggagggatgtttaggtgttttgttgttttatggtttgggtggaggtgggtttggaattttttttaagtttggttttagtttggagtttgtgtgttttgggtggatgtatggattgagaggtgtttggttgggtttttagtttgttattgttatttggttttgtgtgtttattggttgggatgtgttggtttagtagtttttaattttttttgaattttattggtgagttttaggagagtgagttgagggagaaaggtttaaagggtgtgagtgtttagggtgttgggagtttgtgggatgggtatggggagggtagagttagttgaggggagtggttgttgggaggtgtgttgagtagggtttgtagttaaggtttagtgtgatttgagtgtttaggattttgtggttgtggaggaggtgtgttttgggaaggagttgttattgggatttgaagagtgaggggagggagaggggtttagagggtgatttggaggagggttttggaagttatgttaggttggttttttaggtttatttttatagttttttgtggtttttgttttggggagttatgttttgttattgagatttgtgttattagattgtattggggttgatttgggggttgggaatttgttattttgttgtatagatattgattttttttttttttttttaaaaagtaaggtttgtttttattttggtttttgttggatttttttatttgtaatttatttttttatttttattttttttaattggaagatttttttattattttatttttttattttgtttttaatttgattttttattatataattaatgtttaaattttataagggttggttggtgtggggttggtgtgtttttttgtagatgaaagtttaagattttttaattggttttagtttatttgtttggttttaaaataagggtaattgtaattaaatatttttttttagttttttgattaagttgattttttttttatatatgatttaaatggtaattttattaaattgaaaattagtatttgatttgtagtgttagtatttggaaagtgtttgttggtaaattgattgtatgaaggtgtgtagttttattttttgttatttttgtgggttttagtattttaaaaaattgtttggtaattttttggatttaattttgtgtttttggttgtagagtagtattaattaggggagtaatatggttggtattttgtgtttagaattttgtattttaggttgggaaatatttaattaatgtttggg

SEQ ID NO: 125 - DNA, 1700 bp, Artificial Sequence
  chemically treated genomic DNA (Homo sapiens)
tttagatattaattaggtgttttttagtttgaaatatggagttttggatatgaagtgttaattatattatttttttggttagtgttgttttataattaaagatatagggttaagtttaagaaattattagataattttttaaagtgttagaatttataaaaatggtaggggatgaagttatatgtttttatatagttgatttgttgataagtattttttaaatgttgatgttgtaagttaggtattagtttttagtttaataaaattgttatttgaattatatatgggggaggaattgatttgattgagaagttaaagagggatatttggttgtaattatttttattttgaaattagataaatagattgaaattagttaagaggttttaaatttttatttataaaaggatatattagttttgtattgattaatttttgtgagatttaagtattaattatataataggaaattaggttaaaagtaagatgaagaggtgggataatgaaaagattttttggttaagaaaagtaaaaatagaaaagtgagttataaatgagaaaatttgatagaaattaaaatgaaaataaattttgttttttaaaaagaagaaaaaaaaattagtgtttgtatagtagaatggtaaatttttagtttttaaattagttttggtgtaatttggtagtgtggattttggtaataaaatataattttttaaggtagaggttgtagggaattatggaaatgaatttgaagagttaatttgatgtgatttttagagtttttttttaggttattttttgggtttttttttttttttttattttttaggttttagtagtggtttttttttagggtatgttttttttatggttgtagggttttgggtgtttggattgtgttggattttagttgtggattttgtttagtgtattttttggtagttgttttttttggttggttttgttttttttgtgtttgttttatgggtttttagtgttttgggtgtttgtgttttttggattttttttttttagtttgtttttttgggatttattagtgggatttgggagaagttggaggttgttgagttgatatattttgattaatgagtgtatggggttgggtggtagtggtgggttgagagtttagttaagtgttttttagtttgtgtatttgtttaaggtgtgtgggttttaggttgaggttggatttgggggaggttttggatttgtttttgtttgggttgtggggtgatgaggtatttgggtatttttttgatttgtatttatagagatttattgtgattttttagtagattttgtgttaggagttgggtggttgttttagtttgtttttggttaagttttgttatttgtatatttttgtgttttgtgtgttttggtgtttttatggtttattttttttattttttattttgttttgttgttgtaggattagagagaggagtagttttttgggtgatttgaaggtttttgttaggttttttaaatttagttattttatgtgagggggttggggagatggggggtgggtggtggtaatgaaaatgttttattttttttattttttattttattatttttaaaagggttttgttaaatatttttagaattgtttagttttggggtttttaagaaaataatatatgggttttttgggattttttagttttgagggg

SEQ ID NO: 126 - DNA, 1700 bp, Artificial Sequence
  chemically treated genomic DNA (Homo sapiens)
tagttggtaagtggtagatttggaattggaatttagtttagtttgattttttaatttatgtttttgattattatattgtttttttaagtttagatttgaaattttattttttgtgtggttgtgtgtttgggataggggtaattaatttttgattattttatatgttgtatagaatttggagaggattttttaaagtaaatgaattttgaaagttggattgtagagtaaatgagtgtagttaatttagttaggggtttgtaagagggagaaagagaaaaagattgtggaatggaaagttttttaatttaagttttttttaaggggtagttattttttgttttatagtttagggtttgtatgtgttttttttggagtggaaaaatatataagttataaggaatttaatagatagaaaggtgtatagaggaatttaaagtgtgggttggggggtgaggtggtgggtgggaggtgagtgggtgtaggtggaatattgttttttaagttaagttgttgtaaataaaaaggtgtaaagggagagaagttggtgtttaatgtgagttaggagtagtgttttggttttttttttgtttattttaaaagtattttttgtattgtttttaaggtgagaaataggaaagaaaatgttggtttgtgtgtttgttgtttgtttttttggttgtttgtttttgtagggttgttgggagtttttaagttttgtgagaattttgggagttggtgatgttagattagttgggttatttgaaggttagtagtttgggtagggtttattgaaagtttatttgtatatattaggtaatttaattttttattttgtgtgatagaagtagtaggaagtgagttgtttagaggtaggagggtttattttttgttaaaggggggattagaatttttttatgtgagttgtttgaggattgggatgttgagaatgtgagtgatttgagtagggtttgtttgggtattgttggggtaggatttggaatgtatttggaaggttttttgtaagtatttatttggaaggagaatttgggatttttttgggaattttttgttttggttggattggttgagtaagtttggaaaatggtaaatgattatttggattaattataggtttttagttggtttgtttgttataatttatgatttggggttgggaaaaagattaatagtttatgtgttaaaaaaggggtagagtttgatggagttgggtggatttttttatgttatttgtttttatatttagaggataagtatttttgtagatatttagtgtaagggagattatgtttgattgtatggatgttttgttagtgagttttgggtaaattttggatttttatattgtgagtttgtttttttgtatgttttaggagaaagtttttaaagtatgttttagtggattgatttaaattgaatggtagtattggtatattgtttaatgtaggtttatttttttttttttttttattaagaaaaaaaaaattgttttttttgtatgtaataaagatgttggaaataaattgtattggtagtaagataaaggatttaatgatttaatgttgaaggggtgtgatatgttggtatgtatattgattttttttgttgaaaattttttgttagatgttttttttttaaataattttttttgtttttatgtttttttttttattaattttata

SEQ ID NO: 127 - DNA, 1700 bp, Artificial Sequence
  chemically treated genomic DNA (Homo sapiens)
tataaggttggtaaggagaggagtatgggggtgggaggggttatttgggaagaaaatatttaataggaaatttttagtaaagggaattaatatgtatgttagtatattatatttttttagtattaaattattaaattttttgttttgttattaatgtagtttatttttaatgtttttattgtatgtaagagagataatttttttttttttggtagaagaagggaaaaaaataaatttatattgagtagtgtgttgatgttgttatttggtttgggttaatttattgaagtatgttttgagagtttttttttggagtatgtaggaagatggatttgtagtgtagaaatttaggatttgtttaggatttattgatagaatatttatatagttaaatatgattttttttgtattgaatgtttgtaaaagtgtttattttttaggtgtggaggtaaatggtatagaaaagtttatttaattttattaaattttgttttttttttggtatgtaggttgttggttttttttttagttttgaattatgaattatgatagataagttagttaaaagtttgtaattgatttaaatgattatttattattttttaggtttgtttggttaatttagttggggtggggggtttttagaaagattttaagttttttttttaagtaaatgtttgtaaaaagttttttgaatgtgttttggattttattttgatggtgtttagataaattttgtttggattgtttgtgtttttggtattttagtttttaaatagtttgtatgggggaattttggttttttttttggtaaagaatagatttttttgtttttgaatagtttattttttattatttttgttatatagaatgaaagattgaattgtttaatatatgtgagtgaatttttggtgaattttatttgggttgttaatttttaaatgatttaattagtttgatattattaatttttaggatttttatagagtttaaaaatttttagtagttttgtaaaagtagatagttagagaggtaggtagtgagtgtataagttggtgtttttttttttattttttattttaaaaataatataagaagtgtttttaaaatgagtaggggaggagttgggatgttgtttttggtttatgttgagtattaatttttttttttttatgtttttttatttgtggtggtttagtttggaaaatggtgttttgtttgtgtttgtttgttttttgtttattgttttgttttttagtttatattttaaatttttttgtgtgtttttttgtttgttaaattttttataatttatgtatttttttattttagaaaaagtatatgtaagttttaaattgtagaatagagaatggttattttttgggaaaggtttgggttgggaaattttttattttatagttttttttttttttttttttttgtaagtttttggttgaattgattgtatttgtttgttttgtaatttagtttttgagatttatttattttgaaaaattttttttaggttttatgtagtatatagagtagttaggaattggttatttttgttttaaatatatagttatatagaaaatgagattttagatttggatttgaaaaaatagtatagtggttaagagtatgggttggggaattaaattgaattagattttaattttaggtttgttatttattagttg

SEQ ID NO: 128 - DNA, 1700 bp, Artificial Sequence
  chemically treated genomic DNA (Homo sapiens)
ttttttttggatattggagggaattttgaatatattgttttattgtttatgggattgggaagtatatggaggttttgttggttaatttttgttttgttattgttaataagttattattagtttttataattattggggggttggtgttgattgtgggtggtgtggggtgggatttgtttttgggatggtggagggtggtggtgttgtagttgtgttggtttttgagttttagggagtggagggtatggagttgggtgagttttgtggtgttagaattttgtgttgttttgatgttggggtttagggtttattttgggggtgtgtgtgtggttgtggaggtgggaaggtttttttgtttgttgttgtggagttttgtgtttttgtattttgagttttgggggtttgttttgggttgttttttttttttggttttgttgtttatttggtttggtttttgttagtgttttggttttttttggtgtggtgtggggtttggtgtttagttggttgttttgtgttttggtgtggttgtgttttttttttggttttggttttggttttggttttgggttgtgttgtttttggttgtttgtgtggttgttttgttgttgtgtgttgttttgtaggatgttttggatgaggaaggatgtgagtggtttggatggggttggtggtgtggttttttttgtttttgtttttttgggttgtggttttggaattttgagtattttggtggttttgttttggtttgaatgtatttgtttggtttgtattgtttttattttttgttttttggtttgtttatagtttaggggagtgggttgtgttgtttttttattggttgtgggatgggttgtgttgtggattggttgttttgagttttaggtgattttgttgatttgtgagttttgagtattgttttgttttgtttttttttaggggattttttttttagttttttttttttttttaattttttttttttttttaattttttttttttttttagtttttttttttttgttttgagttgtttttttaggttaggagtgtattttaaagtggggtttttttgttttaatttatttgtaatagagagaagtttttggggatgggatgattaagataaatagtttagttagttttttaggattgttagagttaattataggggatgagtatgtagttattgtagttgtgtggtattgtgagagttttgggtgttttttttttttgggagagatggtgtaaatttagatttgtaatatttatgattttaaagaggtatgataagtaggtgtagttagattttaggttgaaagaaggaaaatatttttgttaaagtttttttttgttgtagaagtggttttttttggtggtttttttatttgtaataatttgttaatttttttttttttttagatatatgatatattatttttttgaaaagtttttagatggatggaaggtgttttaggttaggataaatgtaaaaattgtatttaaaaaagtagtttaaaagaatattagtatattttttttgtagattattaattgttaatatgtaatttattggttaaataatgggttagttagattggtttaattggtattgtgattggattggatggtattgtgataatagatgaaagtggaatagagatgatggaagtattttttttttttgttttttttttttt

SEQ ID NO: 129 - DNA, 1700 bp, Artificial Sequence
  chemically treated genomic DNA (Homo sapiens)
agaagagaaaagataaggaaagaaaatatttttattatttttgttttatttttgtttgttgttatggtattgtttagtttagttatagtattggttggattaatttggttaatttattgtttagttaatgggttatatgttaatagttggtaatttgtaaaaagagtatgttgatgtttttttgaattatttttttaaatgtagtttttgtatttgttttggtttaaaatgttttttatttgtttggaaattttttaaaaggatggtatgttatgtgtttggggaggaaggaaagttaataggttattgtggataaaggaattattaaagaaaattatttttgtaatgggaaaaggttttggtaaaggtgtttttttttttttagtttggggtttggttgtatttatttgttatgtttttttgaggttgtagatattgtagatttgagtttgtattattttttttagagagagagagtatttagaatttttatggtattgtgtggttgtagtgattgtgtgtttattttttgtaattggttttgatggttttgaagagttaattggattgtttgttttgattgttttatttttaggagtttttttttgttgtgggtgggttggggtagaggagttttgttttggggtgtgtttttggtttgggaaaatggtttagggtggagggaggagagttggagaaggagaggaaattggggaaggagagggaattggggaaggagagggaattggggaaggaattttttagggaggagtggagtggggtagtgtttagggtttgtagattggtggggttatttggggtttagggtggttaatttgtggtgtggtttgttttgtggttaatgggagggtggtgtggtttgtttttttgggttataagtgagttgggaggtggaaagtgaaagtggtgtgggttgggtgggtgtatttaggttaaggtggggttgttgggatgtttagggttttggagttgtggtttggggaggtgaaagtggagggggttgtgttgttgattttgtttaagttgtttatgtttttttttatttaggatattttgtgggatggtgtgtagtggtaaggtggttgtatgagtagttagagatagtgtgatttggagttggagttagagttagagttagagggaggatgtagttgtgttggggtgtagaatgattagttgagtattgggttttgtgttgtgttggaggaggttgagatgttggtagagattgagttaggtaagtggtgaggttggggaaggggggtagtttaaggtggatttttagagtttggggtgtagggatgtggggttttgtggtgataggtagagggatttttttgtttttgtagttatgtgtgtgtttttggaatgaattttgagttttagtgttagggtggtgtaggattttgatattgtaggatttgtttggttttgtgttttttgttttttggggtttagaagttggtgtgattgtagtgttattgttttttattgttttaggagtggattttgttttgtgttatttgtgattggtgttagttttttggtagttatgagaattaataataatttattaatagtgataaagtaggggttgattagtaaagtttttgtgtgttttttaattttgtgggtagtaaagtggtatatttggggttttttttggtgtttaggagaga

SEQ ID NO: 130 - DNA, 1700 bp, Artificial Sequence
  chemically treated genomic DNA (Homo sapiens)
ttggttttttaaagttttgagattataggtgtgagttattgtgttggtttttaaatatgttatttataaattgtttttggtttgttttttagattgtaggtttaattatgtttttagaaaaattttttattagttttgttggtttttgggtagtttttataatgtattaatttgttttttttttatgtttttaaggagttttttttgttttatgatttgagattagaattgttttttttttattgttgatgtttaggtttaaatgatatttttttatatgtaaaatgggttttaagttttatttatggttgtgatgatttgaaagtaaatgtgtgtatatgataatgtgtgagtgatgagaagtttgttatttgtaaaattttatgtgtatgttaattattatggttgtgataattatgtatattatagaaatattaattaagtgttgggtttgttttaggatttttagtattgttgggaggtttggtatttgataggtgttttaggagggtaggaaaggagagatatgtttggttttaagttttgtgagggttttggggagtgttgggtttttaaggatgttttggatttgaatatttggtgagagtgttgggtaggggaatagtaggagtaaagtttggaaggtggttatgaggataagaagggaggatatttggtaggttgttaaaattaggtttatttgttgggtaggtggaggtgagtaattatttaagttttttgggatagtgggagagggggtaggggagttgttgtattattttttttgtggttgttagggggtttttgttttaggtgagtgttaagttttagttttaggttgtttttttatgttttaagtaagttttgtagtgtagttaattgttaggggttgttttgtttattttttgtttttgtagttatttgtttgtgtgaaatgtttgttaatttttgattggtaggtagtttggtaaattaaattgtgatttttgaaagtaaaatattgtagtattttggtagttttgaattgggaagggatgaaggaggttgtgtttttgggttgtatgaagagtttgagttattttttagaaggttttgataggtgggttttagaggagatgttgttggtgagtagtgattaatattgggaggaaggggaattgaatttaatttttgtttttttttggaaaaagtgaagttatttaatgttttttagtgtatatattttttttttttattgttattagttttgttaatttgggttttgttgttttggaatgttttttttagttttgtattgaggttaagaggagtgggggtatgggttattttattaaaggtgatgttaggttttattaaattaggaagtgatatggagttaattttgttagaattttttttttttgtgttgagtggtttgggttttttggtggtagtagatggtggagttagtaggtgggatgaggggaggtgtttttggtttaagtttgtttttggaatagaggtgttgtttttttgagttgtaagtttttagtttagtgggatgggatggaagaatgtaatttttttgtgagttaaagttgaggaatgggaagtttggtagggaattggtgtttatttttagaagttagattgttgggtggtgggaaagagtgtgttttattgatttgtttagaaagaggtaaatttgaatatagatgtta

SEQ ID NO: 131 - DNA, 1700 bp, Artificial Sequence
  chemically treated genomic DNA (Homo sapiens)
tagtgtttgtatttgaatttgtttttttttgaataaattaataaaatatgtttttttttattatttgatgatttggtttttggaggtgagtgttagttttttgttaagttttttgttttttggttttggtttatgagaaggttatatttttttgttttgttttattgagttggaaatttataatttgaggagatagtatttttgttttagaagtgggtttagattaagaatgtttttttttattttatttgttaattttattatttgttgttgttaggaagtttgagttgtttggtatgaagaggagaaattttggtaaagttaattttatgttattttttggtttggtagagtttggtattatttttagtaaggtagtttatgtttttgttttttttggttttgatgtaaaattggaaagaatattttaaggtaatgggatttaggttggtgaggttggtgatagtaagaaggaagggtatgtatattaggggatgttaggtgattttgttttttttagaaaaaaatgaaggttaaatttaattttttttttttttggtgttaattattatttattggtggtgttttttttaaggtttatttattaaaattttttgagaaatgatttggattttttgtgtaatttggaggtatagttttttttatttttttttaatttagagttgttaagatgttgtagtgttttgtttttaaaggttgtgatttgatttgttagattgtttgttagttagaagttggtaggtattttgtgtaggtaagtggttgtgaggatggggagtaggtgggatagtttttggtaattggttgtgttgtaaagtttgtttgaagtataagggagtgatttggggttagagtttggtgtttatttggagtggggattttttagtggttataggaagaatggtgtggtgatttttttgtttttttttttgttgttttagagagtttaggtggttgtttgtttttatttgtttggtaggtgggtttggttttagtagtttgttaggtgttttttttttttatttttatagttattttttgggttttgtttttgttgtttttttatttgatgtttttattaagtatttaaatttaaaatgtttttgaagatttagtgttttttagagtttttgtgagatttgggattaaatatatttttttttttttgtttttttggaatatttgttggatattaagttttttagtaatattgaaggttttgaaatgagtttagtgtttggttaatgtttttataatgtatataattattatggttatgataattaatatgtgtatggagttttatagatgataagttttttattgtttatatgttgttatatatatgtatttgtttttagattgttatagttatgagtgagatttgaagtttgttttatgtatgaagaaatgttatttgggtttagatattaataatgagaaagaggtagttttagttttagattgtggggtagagaaagttttttgggaatatgggagaggaatgagttggtgtattgtagaaattgtttgaaggttagtagggttggtgagagatttttttgggggtgtggttgggtttgtgatttgagggatgggttagggatagtttataaataatatatttgggggttggtgtggtggtttatatttgtaattttaggattttgggaggttag

SEQ ID NO: 132 - DNA, 1700 bp, Artificial Sequence
  chemically treated genomic DNA (Homo sapiens)
tatgaattagttatattatatagttgttttattaaaaattaaaattttgtagagtatattaatagagattgtatttaagagaaaggtagttattttttagttatttatataattatttattaagtatttatgtgttagttgatatttttgggtgttggaataaaataaggaaaaaattaagatttttatttttatgaagtttattaaagtgtggtaaatgatagagtaattatttttaatagtttttagtttgtattttgtaattaattgaatatagtaaatattaaaagttatttgaggtagggatttattttatttttatttgttggattttattttgtgtttagtatattagtttgtaagtaattgatatggattaaggggtaaaaataggtttttaaatagttaaaattttgataagtaattatttgtttgattattaaatatgttgtttgaaatttttatttttttttttaaagtgtagtaaataagtatttgtgtatttattatgtgtaaaatagtttttaatataatgttttgtgtgtaaaaggaatggtgggaatggatttaagttgtgtattgtgataaatggtgtgaatattttgagtgggtggatagttttttttttgtgagtttttttttttttttttttagttattaagatagtatgtgtgtgtagaagttgtagtggtgggatgtagtgttaagtttgggggtgtatatattagtttgtgtgttgtgagtttgtttgggtagtttggtgggtgtggtgaaatagtttttatgtttgttttttattgattttttggtattagagtggatgaggtgtttttttagaggaaagattgggtgttgtagatgttttttggagaagtttttggttatttgttttattttggggtttttttttttttgggttttttttgattttttgggtgggtgttttagataggtttgttggatgtgatttggaaatgggttgtgtttggtttagtttgtatttatttgtttggttatagtttagttatagtttagggattttgaggtgtttgtgtttttttgttgtggtatttttagtttaagtaggttggatgttgaggttttgtggtattgagtttttgtttgttttgtttttttgttttttattttgttttgtttttgtttttttattttttttttgtttttgttttgtttttttgataatttttttgtgtttttggtttggtatgtgtgttgtgtttggttgttgttgttgttgttgttgttgttgttgttgttgttgagtgtttttttgttgtgtttggtttttagttttgggttggaattggaagtttggggggtggagtttggtggaggttaggagattgaaaatgtggttgagtttggagtttggagttggagttagagtttggattagaatttggttgttgtttgtattgttgttgttgttgttgtttgttgttttttttttgtgttgtagttgttttgttgttattgttgtgagtttggttgttagtggtttttggattttaggtgtttgggttgaaggttggtttggatattggatttagttagtttttagattgtttgggtggtagtggatgttgttgttgttgttgtttttttgttgttttagtttggtgttttgttttgagagatgggagaggtgagtggagttgatagtgattttgatagtgatt

SEQ ID NO: 133 - DNA, 1700 bp, Artificial Sequence
  chemically treated genomic DNA (Homo sapiens)
aattattgttaaaattattgttagttttgtttgttttttttgttttttggaataaaatgttaggttgaggtgatgaagaggtggtggtggtagtggtgtttgttgttgtttggatagtttgggaattgattgggtttgatgtttggattgatttttaatttagatatttaaagtttgaggattattggtggttgagtttgtggtggtggtggtgaggtggttgtggtgtggggaaggggtggtgggtggtagtggtggtggtggtgtaggtggtggttaagttttggtttaggttttggttttagttttgggttttgggtttggttgtgtttttggttttttggtttttgttgggttttgttttttaaatttttggttttggtttgaagttggaagttaggtgtagtggaggaatgtttggtggtagtggtggtggtggtggtggtggtagtggtggttgggtgtagtgtgtgtgttgggttgggagtgtgaggagattgttggggaagtgaggtgggagtgggggaggggtgagagagtgagggtggggtggagtggagggtgaggaggtggggtggatggaggtttgatattgtgaggttttggtgtttggtttgtttgggttagagatattgtgataaaggggtgtgggtgttttaaagtttttaagttgtggttgggttgtggttgggtgagtagatgtgggttaggttgggtatgatttgtttttaaattgtgtttgatgggtttgtttggagtgtttatttaaagggttaggagaagtttaagaggaagaggattttgaggtggggtgagtggttagggatttttttaaggggtgtttgtggtgtttagttttttttttggggggatgttttgtttgttttgatattgaggagttggtggaaggtggatgtgggagttgttttgttgtgtttgttgagttgtttaggtgagtttgtgatgtgtgagttggtgtgtgtgtttttagatttagtattgtgttttattgttgtagtttttgtatatgtgtgttgttttggtgattagaaaagaaaaagaaaaaatttatggaggaagggttgtttatttgtttaggatatttatgttatttattatgatgtgtaatttgaatttatttttgttgttttttttatatataaagtattgtgttgaaaattgttttgtatgtaataggtgtgtaaatatttgtttattgtgttttgaagaaaaaaatgaggattttgaatgatatatttgatgattagataagtgattatttgttaaaattttagttatttaagggtttgtttttattttttaatttatgttgattgtttataagttggtgtgttaggtatggaatgggatttaataggtaaaaataggatggatttttattttaagtagtttttagtatttgttgtatttagttggttgtagaatataaattagagattattgaaggtagttgttttgttatttattatattttagtgagttttgtgaaggtagagattttgattttttttttattttattttagtatttagaagtattaattagtatataagtgtttaataaataattgtataaatgattaagggatgattgttttttttttaaatatagtttttgttaatgtattttataaagttttggtttttaataaaataattgtataatatggttaatttatg

SEQ ID NO: 134 - DNA, 1700 bp, Artificial Sequence
  chemically treated genomic DNA (Homo sapiens)
atttttgtggttatttttagttaggttagtatagtgttttgtgtttgttgaattgttttaggtagttttttgatgatttaaattattattatttttgggtttagagtaggataaatgaaattttaataggggtttgttttgtgattgaaaattatttagttttatagtttttgaatgtaaggagaggtggtttttttagaattaatatgttttttaaggtgggtatgtttatggttttgggaaatgtatattttaggtaatttataggtgtgattttttagtggaggtggggtagtttgaatttttggttttgtaggagtgggaagaaattgtttttgtttttttgataattgagttttttatttggggttgggagggtggatagtaggattggtttttttattttttattgtggttttttgatttggttagtagttttggtgtttagataaaaggttttttgagttgggggtggggggtaggttgagagtaggttggtaaattagattttgtagttttttaagttattagttttgtatttgggtgtagagattttgttttaaaaatttgaagtattgttttggtttgttttttgtttgtgggtgtgggtttttgtttatttatttatttatttatttaattatttatttatttatttatttttttttgatgtatttgtttgttttttggttgttttgggtgtattattgtttttttttttgtttgagggattttttgtgttttaggattattttatagagtttgtttatgtttttggtgaggtttttgttgatgatggtgtatgttggttggatgtgataggtggtggttgggataagtttgtgtagtgttagttgttttttaatttttgttattggtagtgatttgggtaggttttgtttgttggtgatgattagtagtggtgtgttttggtttttggtgaatttggttattttgtgtagttttgttttggttttttttagttggtttatgtttattgagtttattatgtagatgatgttgtttgtgtagtggttgtaggatttttatagtggttgtagttttttttggttgtttatgttttagaagtggtagttgatgtttttggttgtgttgttgtttagtttgatttttttggtgttgaagttgatggtgggtattgtgtttatgaatttgttgaatttgagttggtagagtattgtggttttgttggttgagtttaagtttaatatgatgatatgtagggattggaaggttgagatgttagaggagatgttgtttatggtttttggttgtggtgttagttattgtggttgtggtgggagggtgttgttttttgagtagttatgggtttgatgtggttgggtgtatttgggttttgtttgttgttgtttgtattggttttggggtttggtgtttttgtgggtgtatggtttggtgtgtttagatgttgtagtttttgtgtttttgttggttgggtttgtgattggtgggtattgtgtttttatgtgtggttttgttttttgtgaagttgttttagatgtttggtgttttttttaggtttgggtttgttttgtggtttttttgggttgtgtttttgtgttttggtagttgtttgggttttttgtttttttggtgtttggtgtttgtttttggtttgggatggtgttgtttttgtttttgtttttgtttttgttttt

SEQ ID NO: 135 - DNA, 1700 bp, Artificial Sequence
  chemically treated genomic DNA (Homo sapiens)
gggagtgggagtgggagtaggagtaggagtagtgttgttttaggttagaggtgagtgttgggtgttgggagagtggagagtttgggtagttgttggagtgtgggggtgtggtttgaggaaattatagagtgagtttaggtttggggagggtgttgaatatttgaggtggttttgtgggagataaagttgtgtgtagagatgtgatgtttgttgattgtgagtttggttggtgagggtgtggggattgtggtgtttgagtgtgttaagttgtgtgtttgtggggatgttgagttttggggttggtgtgggtggtggtgggtggggtttaggtgtgtttggttgtgttgggtttgtgattgtttggggggtggtgtttttttgttgtagttgtagtggttggtgttgtagttaggagttatgggtaatattttttttaatattttggttttttagtttttgtatattgttatgttgggtttggatttggttggtaagattatggtgttttattggtttaagtttaatgagtttgtgaatatggtgtttattattggttttaatattgagaagattaagttgagtaatggtatggttaagggtattagttgttatttttgggatgtgggtggttaggagaagttgtggttgttgtggaagttttatagttgttgtatggatggtattatttatgtggtggatttggtggatgtggattggttggaggaggttaagatggagttgtataaggtgattaagtttgttgagaattagggtatgttgttgttggttattgttaataagtaggatttgtttaagttgttgttggtggtagagattgagaagtagttggtgttgtatgagtttattttggttattatttattatgtttagttggtgtgtgttattattggtgagggttttattgagggtatggataagttttatgagatgattttgaaatgtaggaagttttttaagtagaagaagaagtggtaatgtgtttggagtgattggggagtgagtgagtgtgttaagaaagaatgaatggatggatggatggttggatggatggatggatggatgagtgagaatttgtgtttgtgaataaagagtgaattaaagtgatgttttgaatttttaaaatggaatttttgtatttaaatgtaggattggtgatttaaggagttgtgaagtttgatttattggtttatttttgatttgttttttatttttagtttaggggattttttgtttgaatgttagagttattgattaggttggggggttgtggtggggagtggaagagttggttttgttgtttgtttttttagttttaggtggaaggtttagttgttggaaagataaaagtgatttttttttatttttgtagggttagaagtttaggttgttttgtttttattgggggattgtatttgtgaattatttgaggtatgtattttttagaattgtgggtgtatttattttggggggtatgttggttttggggggattatttttttttgtatttaggggttgtgaagttgagtaatttttggttatagggtaggtttttgttgaaattttatttgttttgttttgggtttaaaggtggtggtggtttgggttattagaggattgtttgggatggtttagtgggtatggagtgttgtgttggtttggttggggatggttgtggaggt

SEQ ID NO: 136 - DNA, 1700 bp, Artificial Sequence
  chemically treated genomic DNA (Homo sapiens)
agtaaattgattttgtttgtttagttttattagttagggtggtatggaagtgggtggggagagggttggtgtgtggtatagataattgggaagtgtgtgtattatatatatatatatatatatatatatatatatatatatatatggttttagttaatatttatgtagtatttttggggggttttttatatatatttgaatagtatatttttaataaaggataggtatatttttgtttgtttttttttaatttttagattagtattttattttgggtttagaattggtggtgataagttgatttagatggagtatttgagaaaaggggaggtgattttttgtttgaatgggggagggggtgttttttatttattgtttgagtttttggaatgaggagggttaggatattttggatttttttaggttgggttggtggttggtttttattgtgtaggaggtgtggtttatggtgtgatagtataggtagtgtttggtgtttgagtagttgtgatgttggatgtggttggtggttttgtttgaggtgtagtggttgtgggtagttttgggtggtggtgggggtggtggtagggggggtggtggtggtggttgtagtggatattggggttggggttggggtgagggtggtggtggtgggggtaggtggtgatggggatgtaggaggtggaattggggttgttggagtgggtattggtggtgttgtttgtgtttttagagatgtttgggggttttgagtgtggttttgttggtgttgttgttgttttttttgttgtttgtttgggttggtgtgttgttgttttttgtttttattttggtttgtggttttgtgatttgttgtttagtttgggtttattgtgttggtttgagtgtttttttgatgttgttgttgttgttgttgttgttgttgtttttgtagtttagtagaggttggtgtttagttttttatttgaaggtgttagagttttagggttattgagagggggttttgttagtgggaggggttatagaatgtggtagggttttttttattgaggttatggtagagatatgtttttgggttttttaagttattaagattagggtgtgtggtgtttggttttgttagaggttggtttttttttttttttttttttttttttttttttgggtgttggtggtgagagtagggattgttagtaatttgggttgtagaagtagtagtaatggtggtggtggtagtggtagttgtttttagggtattggaattttgattttggagttgttgaaaagttggatagttttaggttggtatgtttgagttgtgatttttattaaaattattttagttagttaggagttagatttgtttttttgatttttttttttgttgtgtttgtgttttttttttttatattgttttttattgttttggagtttttttttttatttttgtttgttttagtgtttttagagaaatggagtttggtgatttttggttggtttttagagtttgttgatgatgagttttatgttggtgtttttaaagataaggaatgtgattaggtaaagttgtttggaatattgtttatgtgtgttgttgaaaaattggatttgggtgaagatttaatagtaagattaatttagtttttttttttgatataggataaaatattgtgtggtttttttttatagtagggagaatg

SEQ ID NO: 137 - DNA, 1700 bp, Artificial Sequence
  chemically treated genomic DNA (Homo sapiens)
tattttttttgttgtgggaaagagttatataatgttttattttatgttagggaaagagattgaattaattttattattggatttttatttagatttaattttttagtgatgtgtatagataatattttaggtaattttgtttggttatattttttatttttggaagtattagtatggggtttattgttaataagttttgagaattagttaggaattgttaaattttatttttttaagagtattgaggtaagtggaggtgaagaggaaggttttggggtagtggggagtagtgtggaggagggaggtgtgggtatggtgaggggaaggattaagaggataggtttgatttttgattggttggagtggttttggtgaaagttatagtttgggtgtgttagtttggagttgtttaattttttagtagttttgggattagggttttggtgttttggaggtggttattattgttgttgttgttgttgttgttgtttttgtagtttgagttgttgataatttttgtttttgttgttggtgtttaaaggaagggaagaagaaagggaggaagaaggattaatttttggtgaaattgggtattgtgtattttagttttggtgatttggggagtttgggagtgtgtttttgttatagttttggtggaaggagttttgttgtgttttgtgattttttttgttggtagggtttttttttggtagttttgaggttttggtgtttttaagtgagaagttaagtattagtttttgttgggttgtagaagtggtggtggtggtagtagtagtagtagtattaggaaggtgtttgggttagtgtggtgaatttgggttgggtagtaggttgtggagttgtgagttaggatggaggtagagggtagtagtgtgttggtttgggtgggtagtggagagggtagtgatagtgttggtggggttatgtttaaagtttttaagtatttttggaggtatgagtagtattattagtatttgttttggtagttttagttttgttttttgtatttttattattatttgtttttgttgttgttatttttgttttagttttagttttagtgtttgttatagttgttgttgttgttttttttgttgttgtttttgttgttgtttggggttgtttgtggttgttatgttttgagtggggttattggttgtgtttggtattgtggttatttggatattgagtgttatttgtattgttgtgttatggattgtattttttatgtggtggagattggttattggtttggtttgaagaaatttaggatgttttggttttttttgttttagggatttaggtggtgagtggagagtgtttttttttttatttaggtaaagggttattttttttttttttaaatattttatttaagttggtttattattattaattttagatttagggtaaaatgttagtttggaaattgggggaggataaataggggtgtgtttattttttattgagagtatgttatttaggtgtgtgtaagagattttttaaaagtattgtataaatgttaattggggttgtgtgtgtgtgtgtgtgtgtgtgtgtgtgtgtgtgtgtgtgtggtgtgtgtgttttttagttatttgtgttatgtattagttttttttttatttatttttatattattttggttagtgagattaaataggtaaaattagtttgtt

SEQ ID NO: 138 - DNA, 1700 bp, Artificial Sequence
  chemically treated genomic DNA (Homo sapiens)
ggatagggaagtttagttaagaattaggagtttgatatatttaattttttattttattttattgatgattttgtgaggtatatttttttaattttagatttataaaagagaaagttgaggtttggagaggttttttattttgtttaaagttatataatttttatgtggtgtagttaggatgggaattagagtttggttttttgtatttttaatattattgttgaatttgggtttgtttgttttttttttttttttttaattattgttatatagattttattatagaatttattatgtggagggttattattttttttttttttgtatagtatgtttgttttttttattttatagagtaaggtgtttagtaaatgtggttgtttggaggtaatattttttttttattgttatatttggggttggggtgtgaggtaaggtagggttggtgaggtggatgttgtagttgttattattgttaggtgttgggaaatgatttttttgtttgtgtattgatttgaaggtattttttattttttaggttttttttgttttttgttttttatttttagttttttggttttagtttaggttttttggggagtattttttgttgtgagattgatagtttttgggggtgtagggttttgttttttgtgttttagtttatttgtgtgtagagttttgtttttaggtgtttggaatttggtgggtattgatgttaagtgttggtggagtgttgtttatagatggttgatttgggtttttttttatatttttttttttttttgtttttttttttttttttgtatgggggtttgggtttattataaaaggtgggagtgtgtggtgttttagtaatgatgagttttagaatgatggagagtttttgtgtgaggttgttgttttttttgggtgttgttttgttgttgatgttatttttgttgggtatttgtgtttaggaggatgttgagttttagttttgagttttggatatttattttgttgtggatgatgttttttatgagaaggagttggttggtattttttttgtttttgatttttttgagttgttgttttgtttttttttttgtatgttttttttttttttttatttttatttttatttttagagttagggtgtggggagttgagtgtaatgtttaggtatttattgttatttgaagagtgttttgagtttatgggtttttggtagtttgttgagtgaattttttattttggtttttttgagtaatagggattttagtggtttagagatttgtggttagtatttgggatagtgtttgttaagtttttattttttgattattttttgtgtttgtggagttttattgtagagtgtgtgtgggtttggggttttttataattagggttggaagtgtgtatttgggttgggtttgtagttaaggtggtaattttaggttttgaagtggtgtgttgtagattgaagtgttgtaagaagttttgaagaagtttaagagtaaatgtgtttttatttatgagaagaagtatggttaagtttttatggtaaggtttgtggttattttttttttgtgtttttttaagagaaagtatattgttttgaattgtatatatagttttttttgtaggatgtggttaaataatttaggtaatgggtttgtaggattttgtgggttttttttttttttttgggtgaggaa

SEQ ID NO: 139 - DNA, 1700 bp, Artificial Sequence
  chemically treated genomic DNA (Homo sapiens)
ttttttatttgggaaggaagaaggagtttatagaattttgtaagtttattatttaagttatttagttatattttatggagaagattgtgtgtatgatttaaggtggtgtatttttttttggaaaaatatgggaagggagtgattataaattttattatggggatttggttatatttttttttatagatgggaatatgtttatttttgagtttttttaagattttttgtagtgttttgatttgtaatatattgttttggagtttgaagttgttgttttggttgtgagtttagtttaggtgtgtatttttagttttagttataaggagttttggatttatatgtattttgtggtgggattttgtggatatagggaatggttgaggggtggaaatttagtggatgttgttttaggtattgattgtgggtttttgagttgttggggtttttgttgtttagaggggttgggatgagggatttgtttaatagattgttaggagtttgtgagtttgagatgttttttggatggtagtgggtgtttgggtgttgtgtttagttttttgtgttttgattttgggaataggagtgggggtggggggaggagggaggtgtgtaagagaagagataaggtgatagtttaagggggttgagagtgaggggaatattgattagtttttttttgtgggaggtattatttatggtagagtagatgtttagggtttggggttggagtttggtgtttttttgggtatgggtatttaatagaggtagtattagtagtagggtggtgtttaggaggggtagtagttttatgtgggagttttttattgttttgaaatttgttgttgttggggtattatgtgtttttattttttatagtgagtttgagtttttgtgtaggaaagagggaggaggtgaagaaggaagggggtgtggaggagggtttgggttaattgtttgtaggtagtgttttgttggtgtttgatgttaatgtttgttgggttttaggtgtttgggaatgaggttttgtgtatagatgggttagagtgtagagaataggattttgtgtttttaaaggttgttagttttatggtaagggatgttttttgaggggtttgagttgaagttgaggggttagaggtgagggatgggaagtgggaggggtttaggaaatggaaaatgtttttgggttaatgtataggtaggagagttgttttttaatatttagtagtggtgatagttgtaatatttattttgttagttttgttttgttttgtattttaattttaagtgtggtggtgagaagggggtgttgtttttagatagttatatttgttgagtattttattttgtggggtaggggaagtaaatatattatataaaagaaagagaaatagtggttttttatgtgatgggttttatgatggaatttgtgtgataatagttaaaagaagagagggaaaatagataaatttggatttagtagtggtgttggaagtgtagagagttaggttttggtttttattttggttatattatgtaaaagttgtatgattttgaataaaatgaagaatttttttaagttttagtttttttttttgtagatttaggattaaaaagatatattttataaagttattagtaggatgaaatgagagattaagtatgttaaatttttggtttttagttaagtttttttgttt

SEQ ID NO: 140 - DNA, 1700 bp, Artificial Sequence
  chemically treated genomic DNA (Homo sapiens)
gtgtttgtattttatgttattttttgagtttataaatataaaaatagaatttatttttgttatagatttagttattgggaatttattatttgttatggattatttttgttaagaaaagtttgaatgagaagatggtaggatgtttgaaaaaaaaagttataattaataaaatttgtggagaattgtaaattttgtttttattgttgatttaaattttttttttaagattttttttttttagatatttatttatttatttttattttattttttagtattaaggaggtttgagtggataaggtttaagtattgatgtggttttggtaatgaatttgaagttaaaaattggttagtttatgttaagttgttatttgaaaggttttttttagtatttgaattaagggaaataatgttttgaagtagttataaaatttttttgatgtattagatttgaggtaggatttgataatttgtatttttaaattatgtaggtgatgttgatgttgttggtttggagattattttggagagttttattttaaaggaaatgaaattgttttaaaatgttaatattaaatagttatataaagttggaaaaaagtaaagaattttaaattgaagatgagaggttttgaaaaataatagtggttattttatttttggtgtggttatgtggttatgaaaaggagttttggggttggttgtgtttttttatgtaggtttttttatagggttattttattaatttttgttaaaaatgttggtttttttatagggttgtttggttatgtttttttgtgttttaattatagagttatatgttatgttgttgttttgttatgtttatttggtttttgtatagggtgttatgtatgtttaatgttggtttggtgtgtgtgtttgggaggttgtttgtaggttgttaattggtggtttttagtttttgttgatttggttttttttttttgtggttattagattgtgggatattggagattttgaataatttgttggagttttttatagtttaaaaatagaggattggttgggtgtggtggtttatgtttgtaattttagtattttgggaggttgaggtgggtggattatgagtttaggagatagagattattttggttaatattgtgagattttgtttttattaaaagtataaaaaaattagttgggtgtggtggtatgtgtttgtagttttagttatttaggaggttgaggtagaagaattgttagaattagggaggtggaggttgtagtgattgagattgtgttgttgtattttagtttaggtgatagagtgagattgtgttttaaaataaaaaaaatggaggattgaaaattttgaagattgagagtaaggtaataatttttttttgatttttttgttttttttataagggttttttagatggttgaaattaatgaaaagagtgaggaagtaggtagaaaattttagttttagttttttagtatttttttgttttttttttggggagttggtattttttttttttttagtagttgtaaatatgttttttaattttaaggagattttttttgtattttttttattgtttttatgtttgtagggttttttgtgtgaggatttagtgtatgtatgtatgtatatgaatgtgtgtgaataattattttttaattgagtttttttttaaaaaaattattttttg

SEQ ID NO: 141 - DNA, 1700 bp, Artificial Sequence
  chemically treated genomic DNA (Homo sapiens)
tggaagatgatttttttaaaaaaaaatttaattaggaaataattatttgtatatatttatatatatatatatatatattgaatttttatatgaagagttttataggtgtgagaatagtaagggagatgtaagaaaggtttttttggagttgggaggtgtatttatagttattagagaagggggaagtgttagttttttgagaagaaagtaggaggatgttgagagattaaagttgaaattttttgtttgtttttttattttttttgttggttttagttgtttgaaaaatttttgtgagaggagtaaaaaggttaagggaagattgttgttttgtttttagtttttaaaatttttgattttttgttttttttgttttgagatgtagttttgttttgttgtttaggttggagtgtagtggtgtgattttggttattgtaatttttgtttttttggttttagtgatttttttgttttagttttttgagtagttgggattataggtgtgtgttattatatttggttaatttttttgtatttttgatagggatggggttttatggtgttagttaggatggtttttattttttgaatttgtgatttgtttgttttggttttttaaagtgttgggattataggtgtgagttattgtgtttggttggttttttgtttttaaattgtaaaggattttagtggattgtttgaagtttttgatattttgtgatttggtggttgtagaagaaagaggttgagttggtggggattgggaattattagttagtggtttatgggtaattttttggatgtgtgtgttgggttgatgttaaatgtatgtgatgttttatgtgggagttggatgggtgtaatggggtggtggtgtaatgtgtggttttgtggttagggtatggaggggtgtggttggatggttttatgagggggttggtgtttttggtgagggttgatgggatgattttatgagggagtttgtgtggaggggtatgattggttttaagatttttttttatggttgtatagttgtattgggagtaggatgattgttgttattttttaggattttttatttttggtttgaaattttttgtttttttttggttttgtgtggttatttagtattagtattttagggtagttttattttttttaaagtaaaattttttaaagtggtttttggattagtagtattagtattatttgtataatttagaagtataaattattaggttttattttagatttaatgtattagaaaagttttgtgattgttttaaaatattgttttttttaatttagatgttggaggagattttttaaatggtagtttagtataagttaattagtttttaattttaggtttattattaaagttatattagtgtttaggttttatttatttagatttttttgatgttggaaagtaaagtgaaaataggtagatagatgtttagaaaggggaggttttagaagaaaggtttggattaataatgaaggtagaatttataattttttgtagattttattaattataatttttttttttagatgttttgttatttttttatttagattttttttagtaaaggtagtttatggtaagtaatgaatttttagtaattaggtttgtaatagaagtaaattttgtttttatgtttataaatttaaaaagtaatatgaagtgtaaatat

SEQ ID NO: 142 - DNA, 1700 bp, Artificial Sequence
  chemically treated genomic DNA (Homo sapiens)
gtatatgtgaaaataatattattttatattatgatttatgttatttgtatgtgtaattaaagttttttaagttttgatatgtttttataaagatgaaggatagagtgaatgttaaaatttgttaaataggagttattgaaagtttgaaggggaaagtttagttaggttttattttagaagtgatttttaaaatgaaatatgtagataagtttgagttttttggagttgatttttaaggttttgtaggtggtaagggggttgagaattggttgaggttttttggaagtttgtgttgatttggagggttatttttgattttttgatttggggtgtaatttttagtttttgttttggttgataaaggtgtggagttaggttttaggtaagttgggggtgggggtgtgtataagggaggggtaggggattgggatttttttttttttggggtgttttgggttgtttttttagaggttggtggtgtttttgatttaagtagtttagttttgtttgtgaggtttgaagtggtgggaatgggttttttgtggtggttttaaggtgagggaggttgtaaaatttggtagagaatttggtgtgttgggttggggatgttttgggtgttgggggtaggggttgtaggtttgtaggtagaagtttttttatgtgagtgggggttttggttgtggaaggggtgtttttggggttgttggtagtggtgagtgggtgggagggagttttttgggttttttttgtgttttgggttggtggggtgatggtgatgatggttttttggtttttttgttttggatgttgatggtggtgtgttttggtttttttttaatagttttttttttgtggttgttggagttggtgaggttgagagggtggggagttgtgtagggaaggtgagagggtggaggttgtgtggtgtaggtggtggtggtgggaggaggagggttgggtgggtaggggaggaggaggaggtgggtgttgtgttgtatgtagttttaggtggggtagttttggtagttgagggatgtagttagattttggtgggatggggtttttgttggggtttaggtttagggattaggtggaggtgttgtgggagtttttggggtattatagagatgtgggtgagtgttggtagttgttggggttgagaggaggtggtggtggtggtggtggtgtaggtttggttttgagttgtggtgttgtagtgatttgggttgtgatggttgaaattaggtgtttggtgaggtttgagttgttgaaatgtggttgggtagaatggtggttgggttgggggatttgtgggtggaggatgttgaaggtggtagaggagtgagtggggttgggttttgagttattttggatgggttggggtggttaggtttggattatgtattgggaaaaagtatgatttattgtgtttggaggtggtggtttggtggtgttggtttttttttttgttgtgattgggttgggtttttggagggtgggttgatgtgtggtttggggtggtttgggtgtttgtgttttgtttggtgaggtgatttttgtgggatgttgggatttgtgtttttgtttttttgtgtttatgtttttttttggtgttgttttttggttgatgtgtgtttttttgtgagttttgggtttaagtgtaggagtagttgtggagttgggtttttttgttttttggt

SEQ ID NO: 143 - DNA, 1700 bp, Artificial Sequence
  chemically treated genomic DNA (Homo sapiens)
attggaaagtgagagagtttggttttgtggttgtttttgtgtttgagtttagagtttataggggaatatgtgttggttggaaagtagtgttgggaagaagtgtgggtgtgagggggtaagggtatgggttttggtgttttgtggaggttgttttattaggtgaggtgtggatgtttgagttgttttgggttatatgttggtttattttttgagaatttagtttggttgtggtggggaaaggggttggtgttattaggttgttgtttttaggtgtgatgaattgtgtttttttttagtgtgtagtttaggtttggttgttttggtttgtttggggtgatttagggtttggttttgtttgtttttttgttatttttggtgttttttgtttatgagttttttggtttggttgttattttgtttggttgtgttttggtggtttaggttttgttaggtgtttggttttggttgttgtggtttgagttattgtgatattgtggtttggggttgggtttgtgttgttgttgttgttgttatttttttttggttttggtggttgttgatatttatttgtatttttgtggtgttttaaaggtttttgtgatgtttttgtttggtttttgggtttgggttttggtgaaagttttgttttgttaaggtttagttgtgttttttagttattggggttgttttgtttggagttgtgtgtgatatggtgtttgtttttttttttttttttgtttgtttggtttttttttttttgttgttgttgtttgtattatgtaatttttgtttttttgttttttttgtgtggttttttgtttttttggttttattggttttggtgattgtgggggagaagttgttgggggagggttggaatgtattgttattggtgtttggggtgggggagttgggaaattgttgttgttattgttttgttagtttgaggtgtgggaggggtttggaaagtttttttttgtttgtttgttgttgttggtggttttgggggtgttttttttgtggttgaggtttttgtttgtgtggggaagtttttatttgtgagtttgtggtttttgtttttggtatttgagatatttttgatttggtgtgttggattttttgttaggttttatagttttttttgttttgggattgttgtggaggatttgtttttgttattttggattttgtgagtggggttgagttgtttgggttggggatattattagtttttggagaaatggtttgaagtattttgaagaagagggggttttagttttttgttttttttttgtgtgtatttttatttttagtttgtttggaatttgattttatatttttgttgattgggatagggattgggggttgtattttagattagaaagttagaggtggttttttgaattggtgtggatttttgaggaattttagttaatttttgatttttttattatttgtaaggttttaggaattggttttagaaagtttgagtttatttgtgtgttttgttttaaaaattatttttaagataaaatttaattaggtttttttttttagatttttagtaatttttgtttggtaaattttgatatttattttattttttgtttttgtaaggatatattaagatttagaaggttttgattatatatatggatggtatgagttgtgatataaaatagtattgtttttatgtgtat

SEQ ID NO: 144 - DNA, 1700 bp, Artificial Sequence
  chemically treated genomic DNA (Homo sapiens)
gttagagattgaaattttttggaattaattattggatgttttggattggggtggaatgtgtttttattgttttgaaagtagtatttttttggatttgagtttttgtggtgtgtttgggttgagatttgagaaatgtgttttgggttataggtagtagtggaggtggggaaggaataattttttttttgttttttaattataagggtggggtggggtggttttgttggggttgggtttttttatttttttagttgggattttttgttggagttggtgtgggggttggggagggggattagaggggtttttttttttttttaagttttgttagatgttggaaaggtaaataataggggaaatgtggagaaataaaaggttatttgttatgattttagttggttgtgtagagagatttttgtggggagggttaggggagagggttaggaaagagtgtgaggggggggattttgagagatttagagtttttggtatttgttaggtattaggggttggtgttttatttttttagtaggggtttgaaggtgggaaggggtaattaaagaaaatttgaagggtttttatttttttttttagtttagggaaagggaggggttgggattttagatgtttgatagttgttggttttaaagttaagtgtgatttgtatggtttttatatgttttttatttttgtgaggtattgttagttttgggggtgatttggttgatggtgtggattttttttatgttgttttaagttgagattttttttttaggattaggatgtttaagtgttgttttttagtttaggtgtatttttgtaaaagtttgtttttttgttatttatagtttaatatgtattttgtaggtgtttgtggttggggatgggtttggagttttgtattttgaaagaaggatgattttattgatttatatttttttaaattaaggataaattaagattttggggtggaaagtgggtgatttttgtttttgttgggaagtgtatatgaggagttggttagaaatttaggtgttggtaggggtttgaagaggattttagttttggaagtttgtggttggtgggtagaagattggtttgttgttttttgtatttttttgtatttgtgtttaggtgtgttttaaattttgttgtttgtttgggtgagattgggagtgagtagttattgggttgtgttgttttttttagtttggagtgggtttttatttattgtggggttttatttttagtttagggtttggttggttttttttgttgggttagttgtagttagtgtgaaagaaaatggtgagtttggggtaggtggggtgttgtttgttaatttatgtaagggattttgttgttggtgtgtgaggttttggtgagttaattggtggatgggtgagtgtttggttttagttgtagttgttgttgttttggagtagtttttttatgtatttagtgtgttgtgtagttggttggggtttgattgttttgtggggatgttgagtggggatttttggtggttgtttgtggtatggtagtattgtggagagaaaagttgtttttgggattattttttttttggaaagagatgggatatgattagtttaagggggtttttagttttgtagaaaagatattgggttagggtagtgaagaaggtgttagattagaaaggatgatttttgggtttataa

SEQ ID NO: 145 - DNA, 1700 bp, Artificial Sequence
  chemically treated genomic DNA (Homo sapiens)
ttgtggatttagagattattttttttgatttgatattttttttattgttttggtttagtgttttttttgtaaggttggaagtttttttagattggttatgttttatttttttttggagggaagatgattttaaagatgatttttttttttatggtgttgttatattgtaggtggttgttaggggtttttgtttggtgtttttgtgagatagttgagttttggttggttgtgtggtgtgttgggtgtatgagggggttgttttggagtgatggtggttgtagttggagttaggtgtttgtttgtttgttggttggtttgttgggattttgtgtattggtggtagagttttttgtgtggattggtaagtgatgttttatttgttttgagtttattatttttttttgtgttggttgtagttgatttggtgaagggagttgattgggttttgggttggaggtaaaattttatggtgagtaagaatttgttttaagttaggggaggtggtgtagtttggtggttgtttgtttttgattttgtttgggtgggtggtgaggtttggggtgtatttgggtgtgggtgtaagaaggtgtgggaggtggtggattggttttttgtttgttggttatgggtttttggggttggagttttttttagatttttgttggtgtttgggtttttggttggttttttgtgtgtattttttggtaggaataagggttgtttattttttattttgggattttgatttgtttttgatttgaaaagatataaattaataagattgttttttttttggggtgtaagattttgagtttatttttagttgtggatgtttgtagggtgtgtgttgggttgtgggtggtgggaagataaatttttataaaagtgtgtttgggttgggggataatgtttgggtgttttgattttgagggaggagttttggtttggggtagtgtaggggaagtttgtattgttagttaggttgtttttggggttgatgatgttttatggaggtggggagtgtgtaaaggttgtataaattgtgtttaattttggggttaataattgttaaatatttggaattttagtttttttttttttttgaattggggaagaaggtgaaaattttttaagttttttttgattgtttttttttatttttagatttttgttgggagggtaaagtgttgatttttggtgtttggtaagtattagagattttaaattttttgggatttttttttttgtgtttttttttgatttttttttttaattttttttatagagatttttttatgtagttgattgagattgtggtgaatggttttttgtttttttgtgttttttttattgtttgtttttttaatatttggtggggtttggggagagaaggaagtttttttggtttttttttttggtttttatgttagttttggtaggggattttagttgggaaagtggaggagtttgattttagtgaggttgttttattttgtttttgtggttagagggtggagggaaagttgttttttttttgtttttgttgttgtttgtggtttagggtgtattttttagattttagtttaggtgtgttgtaaaggtttaaatttgagaaggtgttgtttttgagatagtggaagtgtgttttgttttaatttagagtgtttagtggttggttttagaggattttaatttttagt

SEQ ID NO: 146 - DNA, 1700 bp, Artificial Sequence
  chemically treated genomic DNA (Homo sapiens)
ttaggatttttaattatttattatttgtatttttggggtggatgtgagtttgtgttaaattaaggatattagttgagtatttagtgtggttttttatgttttttaggaattttggaaagttatttaagtttggtgttatttttaaaaggaatgagttgtggtttttaattagtttgttgaggagagtgtagagtgatatgggatgggggtttagggtattgaggttaagagtttttgagttatgttagtgtttgtttttttggtttttgtttaaaattttgtgatgttttgttgataaagtatttagtgttagagtttgtggtttagtgaagttgttttttggaaatatatatatatatatatatatatatatatatatatatatatatatatataaaagggggtggggagagggaaaggtttaggtttaggagaggaagttagttagttagttttgtagatgtaattgttgtaatttgggttttttttgttttttgatgtaatgttagttttttgtttttgtgttgtagtgtgtagtttgttgttgggaagttagattaattgattgaatgttaagattattattgttattattttgtgatttattttttatgaatatattttaattggtttgagttttgtttttaaattttatgatttttggttttttaggaagtattgtttttttttttttttaggaaggttgtttttatgtatagttagtattaggaaatggtgttgtttggggttttgatggagagttggggtggggttggtttaaattttttgttttttgtttttttgagatttttgatttttggttttgttttaagtaaaagtattgtagaagttgtagtgttttttttttttttttagaaattgagtgaggatgtgggggaaaagttattttggaatgtgtaaggatgtttttttatgtttaatatgttatagggttgggatgtgtggttgttgttttgttattttatgtgtgtagtgttaattgtagttttgtagttattgttgttgttgttgttttgttgagtttggggagatttttaagggttttttaaatatgagttataaatattataggtgtttggtgtgtgtgttatgatatatttggtttttgattgggtggtgtaattttgtttatttgtataggttgattgtataattgggtggtgattttagtttggtgtttgtgttatgttttttttatttttgtgggtatgtggggttgtgggtgggatagtggggtggtgattgtggtgtttagagttttttgaatttgggaattttttaagttttgagttaggtggtggttttttgggatgttagtgttttttagaaggtttagttttggttagttaagggtttattgtttaaattgggttttttttatttttatagaaaaggatagtttttagagtgatattttagtaaatgttttgggtggtttgtagaggttgagtttaaattatgtgagatttggttgtggtgagagttttgtttaatgatttgtgatttggggtgtttaaaaagggttttttgtatgatttttaataagattttgttattttgaaaatgaaatagggagggtttgaaaatgtaagatgtgtaatgttttgtggtttaaatttagtttttaaaaaatgtattaatgagttaatatgaaatagtgatatggagattttttttta

SEQ ID NO: 147 - DNA, 1700 bp, Artificial Sequence
  chemically treated genomic DNA (Homo sapiens)
tgggggaaagtttttatgttgttgttttatattggtttattggtgtattttttaaaaattaggtttaaattataaggtattatatattttgtgtttttgaattttttttgttttgtttttaagataatgaggttttattagagattatgtgaaaagttttttttgaatattttaagttgtaagttattggataggatttttgttgtggttaggttttatgtggtttaaatttagtttttgtgagttatttagagtgtttgttggggtgttattttgaaaattatttttttttgtagaaataaaaaagatttagtttaaatggtaagtttttggttggttaaaattgagttttttgagaggtattggtgttttggaggattgttgtttgatttaggatttgaaaggtttttgagtttaagggattttggatgttgtaattgttattttgttgttttgtttgtggttttgtgtgtttgtagaagtggggaaggtgtggtatgagtattgggttgaggttgttgtttggttgtgtaattggtttatgtaaataagtgaggttatgttgtttaattgggagttggatgtgttgtgatgtatgtgttgagtgtttgtagtatttatagtttgtatttaaagagtttttgggagtttttttgggtttggtagggtggtggtgatggtggtggttgtggagttgtagttagtgttgtgtgtgtgaggtggtggggtggtagttgtgtattttggttttgtggtgtgttgggtgtgggggagtgtttttatatgttttagggtggttttttttttatgtttttgtttggtttttggaaagggggaggaaatgttgtagtttttatagtatttttgtttaagataaagttagggattaggaattttaaaagagtggggggtagaaggtttgagttgattttattttggttttttattggagttttgagtggtgttgttttttgatgttggttgtgtgtagaggtggtttttttggggggaggggagaataatgttttttgaaaagttaaaagttgtggaatttgggggtggggtttgggttaattaaagtgtatttgtggaaaataaattgtaagatagtggtagtgatgattttggtgtttaattgattgatttgattttttagtagtgaattgtatattgtagtgtgaaagtaggaaattggtattatgttagaagatagaagagatttaaattgtaatggttgtatttgtagggttggttggttggtttttttttttaaatttaaatttttttttttttttatttttttttgtgtgtgtgtgtgtgtgtgtgtgtgtgtgtgtgtgtgtgtgtgtgtgtgtttttagggagtagttttgttgggttgtaggttttggtattgggtgttttgttagtgaggtattgtaggattttaggtaaaggttaaaggggtgagtattggtgtgatttgggaatttttggttttagtgttttgggtttttgttttgtgttattttatgttttttttggtaagttagttaggagttataatttattttttttaaagatagtgttgagtttggatagttttttaaaatttttggagggtgtagggaattgtgttgaatgtttggttggtgtttttagtttagtgtgagtttatgtttattttaggaatataagtggtgagtggttagagattttag

SEQ ID NO: 148 - DNA, 1700 bp, Artificial Sequence
  chemically treated genomic DNA (Homo sapiens)
tattttaagttattaagttttagataggtatagtagtatatatgtgtgaatagaatttattagtatagtttggggtttaggtttttgtatattaataatttaaggtttttggtaatggtttagtgtgtatttatgaggttattgtttttttatatatgtatttagaatatttatagttttaaaatggtaattttattataaaaatgatattttgtatttgtatagtattttgttttttgtgtattttatatatttattatttaatgattgtgttttttgaaatttttaataattttgggtggtggtaagaataggtatttttaagttatttttttagatgaggaaatttatttgtgtttttttttttttttttaatagttggttgatggaatttaagttttttatattagtagtggggttagttgtatagagtggttagaagggagaggttttggggttatgtttttttttttatttttagtttttgatttattgttatattatggtgagtaataggataattttttttatagaattatgggggatattatttttttgaggttaatgtgtttaattaagtgggaaattttttgggtagagtgaaatttgaagttgttatgttataagataatttagagtaaggggaataattgatgttttaatgtttttttttatggggggttgaggggtgtttatttatattttttttttttgtttttttttgttattttttttttgttggttggtttaattttataagtgtgtgtttgttttgagtatagtaatttagagtagatggggggtattttttagagggtagtggaaagtttatttgttattgttgtgttggggatttgtggttttttgggtggttatgttttgttttggtggtgtttggtgtgtttggaatgtggggtttttttttgtttttttttttatgttgttttgttttgtttttgttagggttttttagagaagtttgattttttattatttttgttttgtatttttttggtttgagggttggtggttttttatttttgttttaaatattttgtttttgagttttttttttaaagattttttagtgttggttgtttttttagttttttgtttttgatggtttttggggttagttgttggggtttttttgttaggttgtatttttttttttagtgtttttatattttgttagttggtggtttattgtgggtgggtagggtgtgttttgtttatttgggttgtgtgttgagagttgagagtgtttttaaatgttgagtagagttggttggtttgggtggggtttttatatttgttatattttttttaggtgaggtgaggtttgttttgttttgttttgttttttttttatatggtttgtttttattttaagttaggggtgatttattttggtttgttggtttgtagggattgtgttttggaatgtttttgagttgtgttatgtgggagtggttttagtgtttttggttttgttttttgagaaagtttggttgaggttgttttgaggtttatagggattttgttttttttttttttttttattttattttttaagtgttatgttgagtttttagttttggtttgggttaataaattaataagattttagtagtagattttgggtttgattaagtgtttttttttttttttttttttttttggtgttaatgtaatggagg

SEQ ID NO: 149 - DNA, 1700 bp, Artificial Sequence
  chemically treated genomic DNA (Homo sapiens)
tttttattgtattaatgttaaaaaaaaaaaaaaaaaaaaaaaaatatttaattaagtttagagtttattattaaagttttgttaatttattaatttaagttgaggttgagagtttgatgtggtatttaagaagtaaaatgaggaaaggaggaagaaatggggtttttgtgggttttggggtagttttggttgggtttttttggagggtagggttaggggtgttggggttgtttttgtgtggtgtggtttgggagtgttttagggtgtggtttttgtgggttggtgggttggggtgagttatttttgatttggggtgggagtgggttgtgtaagggggaggtggggtggggtggggtgggttttgttttgtttagggaggatgtggtgggtataaaagttttatttaggttagttggttttgtttagtatttggggatgtttttagtttttggtgtatggtttaggtaagtggggtgtgttttgtttgtttgtgatgggttgttagttagtggggtgtggagatgttgggaagaagggtatggtttggtagggaggttttggtaattggttttggaggttgttgggggtagggggttgaagggatggttggtattggagagtttttggggagggggtttagggataaggtgtttggggtaggggtgaggggttattggtttttgggttgggagggtgtagggtagaggtggtggggggttgggtttttttaagaagttttagtgggagtggggtgaggtggtgtggggagaggggtggagggaggttttgtattttagatgtgttaggtgttgttgaggtagggtgtggttgtttagggagttgtaggtttttggtgtagtagtagtaagtgggttttttattgttttttagaaagtgttttttatttgttttaagttattgtatttgaaataaatgtgtgtttgtggagttgaattgattagtaggagggaagtagtgggggaagatgagaggggggagtgtgagtaggtattttttagttttttataaaagaagatattaaaatgttagttattttttttgttttaggttattttgtagtatagtaattttggattttattttatttggagagttttttgtttggttgaatatattggttttaggaaggtagtgttttttatgattttgtggggagggttgttttgttatttgttatgatgtgataatgagttaggaattgagagtagagagaagggtgtggttttggggttttttttttttggttgttttgtgtagttggttttattgttggtgtggagggtttgggttttattagttggttgttgagaagaaggaggggaatatagataagtttttttatttgggaaggtggtttaaggatgtttatttttattattatttagagttattggagattttaaaaaatatagttattaggtaataaatgtgtaaagtatataaaaaataaagtgttgtgtaaatgtaaagtgttatttttgtagtaagattgttgttttaaagttgtgggtgttttgagtgtgtgtgtaaaagagtagtggttttatgggtgtatgttgggttattattaagaattttaagttgttggtgtgtaaaggtttgggttttagattgtattgatggattttgtttatatgtgtgtgttattgtgtttgtttggaatttggtgatttggaatg

SEQ ID NO: 150 - DNA, 1700 bp, Artificial Sequence
  chemically treated genomic DNA (Homo sapiens)
ggttttttggtagtttaagtgttagtttagatattgagattttggtaaagagtttttttttattttttttttttattggtttttgtgggtggtgtggaatgtgttgtaggttttgtgagagtggtattttttttttttttttttttttttttttttaatttattgtagtttttgagagggtagtatttttgggtgtatttgtatttaagtttttgatagtgttttgggtttttttagtaatgtgtatttagggatgttatgttttgtgggttttatgtttggggtatggagttaggggaggtggttattttattttttttatgtttttgtttattttgtgttttagtggagttttaggtaaataagttaggtttttagtttttaaggatggtttttattttagattttaggaggaggtgttttgggttattttgggaaggttagtttgtttgggaggggtagattatgaaaatggttttttaggtagtgttattttttttttattgttagttatttaatttaggtggttttattttttttgtttttatggatatgtagtaggatgtagagagtatagagttgtgattggtatgagtttataatgggtttttttgtttaatttatttaatttttagtaagggataggttttgtttattggggtttgttgaggttttaaatgtgtatgttgttaagggagtagttgtgggggtttgttagtggggataaaggtgtagttattggggtgaaagttttgttttgtagagggtgggtttgtggtgattgtgagtttagagtttggatttgttgtgtaatttgttttttttggttttattttaatttagttggatttttggtttggtttttgttttttggagatttagttattaaagttgaggtggggggatgaggatgtgggtggtgtttaggtaattttgaggggttttgatgttttttttagagttagttattaggagtaatatgatgtgtttttatattattgaatttagaattttagtgttttagtattttttttttgatatgagttttgtttaatgaggagaagggttttttgtattggttggtggtggttttttatttaattagagaggttaatggtttttttttgtttggtttagaaggaattgttgggggaatataggttaaattataatttttgagaggtagttgggttttgtgggggggggggggggggtgtttgtgtgtttatgtgattttttttttttttgtggagattgagttatgtgataggtttgtgagggggtggggtttggtgtttgtttgttgtgttagtggttaattggtagttaggtagggtgggtgtgtgtagggggtggggttgggtgtgtggtagggtgtgagagtgtatttgtggtggtggtggtggtgattgtgggggggtggtggggaatattggttaagttgatagtggaggtttaggtattggtggtgggtggttgtggtttttggtgttgtttggtgtgtggttagtttttggaatggaatgtttggtgttgtgggttttgtttggaaagtttgttgtggagttgtgattttttggtttgtgtggtttggtatgaagtggtgttgaggagttgttgttgttgtttgttgttgttgttgttgttgtttgaggaggagttgtagtattttgggttatgttgatgattattgtaaattg

SEQ ID NO: 151 - DNA, 1700 bp, Artificial Sequence
  chemically treated genomic DNA (Homo sapiens)
tagtttgtagtagttattggtgtggtttagggtgttgtagtttttttttaggtggtggtggtggtagtggtaagtggtggtagtagttttttaatgttgttttatgttgggttgtgtgggttaagaggttgtgattttatggtaaattttttgggtggggtttgtgatgttgagtgttttgttttgaaagttggttgtgtgttgagtagtattaggaattgtagttgtttgttattggtgtttaagtttttattgttggtttagttaatgttttttgttgtttttttatagttgttgttattgttgttgtgggtgtgtttttgtgttttgttgtgtgtttggttttgttttttatgtgtgtttattttgtttggttgttgattggttattaatatggtgaataggtgttgggttttgtttttttataggtttattgtgtaatttggtttttgtgagaaggaaggaagttatgtgaatatgtaggtgtttttttttttttttttgtagagtttagttattttttgggaattgtagtttggtttgtatttttttggtgattttttttaaattagatgagagaaggttgttagtttttttaattggataggggattattattggttaatgtagaagatttttttttttattgggtggagtttatgttgaggaaggggtgttgaagtattgaggttttgggtttggtgatgtgggggtgtattgtgttatttttgatggttggttttagggaaggtattagggttttttagagttatttggatgttgtttatatttttatttttttattttaattttaatggttgagtttttagagggtagggattggattgagagtttagttaggttggggtggaattgaaggagatagattgtgtgataagtttggattttgagtttgtggttgttgtaggtttattttttgtggggtggagtttttgttttggtggttgtgtttttgtttttattagtgggtttttgtagttgtttttttgatagtatgtgtgtttgaggttttagtgaattttaatgggtagggtttgttttttgttggaagttgagtagattgagtgggaaagtttgttgtgagtttgtgttaattatagttttgtgttttttgtattttgttgtatatttatgaaagtgggagggatggagttgtttaaattgggtggttggtagtggagaggaagtgatattgtttaaagaattgtttttgtgatttgttttttttgagtaagttagtttttttgagatggtttaagatgtttttttttggggtttggggtgaaggttgtttttggggattgggagtttggtttgtttatttaaggttttgttgaggtgtaaggtggatagggatgtgagagaagtagggtgattgttttttttagttttgtgttttgggtatagggtttatagggtatagtatttttagatgtgtgttgttggaggaatttagggtattgttagaggtttgggtgtaggtgtgtttaaaaatgttgtttttttagaggttataataggttaaaaaaaaagagagaaaaaaaaaaaagaaatgttgtttttgtaaggtttatagtatattttatattgtttataaaggttagtgggaagagagggtaaagggaagttttttgttgaggttttaatgtttggattgatatttgagttattagggagtt

SEQ ID NO: 152 - DNA, 1700 bp, Artificial Sequence
  chemically treated genomic DNA (Homo sapiens)
gttttggatattttttgtttttttgatttaaggtttagtttggttttttttagggttttttggtagtttaagtgttagtttagatattgagattttggtaaagagtttttttttattttttttttttattggtttttgtgggtggtgtggaatgtgttgtaggttttgtgagagtggtattttttttttttttttttttttttttttttaatttattgtagtttttgagagggtagtatttttgggtgtatttgtatttaagtttttgatagtgttttgggtttttttagtaatgtgtatttagggatgttatgttttgtgggttttatgtttggggtatggagttaggggaggtggttattttattttttttatgtttttgtttattttgtgttttagtggagttttaggtaaataagttaggtttttagtttttaaggatggtttttattttagattttaggaggaggtgttttgggttattttgggaaggttagtttgtttgggaggggtagattatgaaaatggttttttaggtagtgttattttttttttattgttagttatttaatttaggtggttttattttttttgtttttatggatatgtagtaggatgtagagagtatagagttgtgattggtatgagtttataatgggtttttttgtttaatttatttaatttttagtaagggataggttttgtttattggggtttgttgaggttttaaatgtgtatgttgttaagggagtagttgtgggggtttgttagtggggataaaggtgtagttattggggtgaaagttttgttttgtagagggtgggtttgtggtgattgtgagtttagagtttggatttgttgtgtaatttgttttttttggttttattttaatttagttggatttttggtttggtttttgttttttggagatttagttattaaagttgaggtggggggatgaggatgtgggtggtgtttaggtaattttgaggggttttgatgttttttttagagttagttattaggagtaatatgatgtgtttttatattattgaatttagaattttagtgttttagtattttttttttgatatgagttttgtttaatgaggagaagggttttttgtattggttggtggtggttttttatttaattagagaggttaatggtttttttttgtttggtttagaaggaattgttgggggaatataggttaaattataatttttgagaggtagttgggttttgtgggggggggggggggggtgtttgtgtgtttatgtgattttttttttttttgtggagattgagttatgtgataggtttgtgagggggtggggtttggtgtttgtttgttgtgttagtggttaattggtagttaggtagggtgggtgtgtgtagggggtggggttgggtgtgtggtagggtgtgagagtgtatttgtggtggtggtggtggtgattgtgggggggtggtggggaatattggttaagttgatagtggaggtttaggtattggtggtgggtggttgtggtttttggtgttgtttggtgtgtggttagtttttggaatggaatgtttggtgttgtgggttttgtttggaaagtttgttgtggagttgtgattttttggtttgtgtggtttggtatgaagtggtgttgaggagttgttgttgttgtttgttgttgttgttgttgtt

SEQ ID NO: 153 - DNA, 1700 bp, Artificial Sequence
  chemically treated genomic DNA (Homo sapiens)
ggtggtggtggtagtggtaagtggtggtagtagttttttaatgttgttttatgttgggttgtgtgggttaagaggttgtgattttatggtaaattttttgggtggggtttgtgatgttgagtgttttgttttgaaagttggttgtgtgttgagtagtattaggaattgtagttgtttgttattggtgtttaagtttttattgttggtttagttaatgttttttgttgtttttttatagttgttgttattgttgttgtgggtgtgtttttgtgttttgttgtgtgtttggttttgttttttatgtgtgtttattttgtttggttgttgattggttattaatatggtgaataggtgttgggttttgtttttttataggtttattgtgtaatttggtttttgtgagaaggaaggaagttatgtgaatatgtaggtgtttttttttttttttttgtagagtttagttattttttgggaattgtagtttggtttgtatttttttggtgattttttttaaattagatgagagaaggttgttagtttttttaattggataggggattattattggttaatgtagaagatttttttttttattgggtggagtttatgttgaggaaggggtgttgaagtattgaggttttgggtttggtgatgtgggggtgtattgtgttatttttgatggttggttttagggaaggtattagggttttttagagttatttggatgttgtttatatttttatttttttattttaattttaatggttgagtttttagagggtagggattggattgagagtttagttaggttggggtggaattgaaggagatagattgtgtgataagtttggattttgagtttgtggttgttgtaggtttattttttgtggggtggagtttttgttttggtggttgtgtttttgtttttattagtgggtttttgtagttgtttttttgatagtatgtgtgtttgaggttttagtgaattttaatgggtagggtttgttttttgttggaagttgagtagattgagtgggaaagtttgttgtgagtttgtgttaattatagttttgtgttttttgtattttgttgtatatttatgaaagtgggagggatggagttgtttaaattgggtggttggtagtggagaggaagtgatattgtttaaagaattgtttttgtgatttgttttttttgagtaagttagtttttttgagatggtttaagatgtttttttttggggtttggggtgaaggttgtttttggggattgggagtttggtttgtttatttaaggttttgttgaggtgtaaggtggatagggatgtgagagaagtagggtgattgttttttttagttttgtgttttgggtatagggtttatagggtatagtatttttagatgtgtgttgttggaggaatttagggtattgttagaggtttgggtgtaggtgtgtttaaaaatgttgtttttttagaggttataataggttaaaaaaaaagagagaaaaaaaaaaaagaaatgttgtttttgtaaggtttatagtatattttatattgtttataaaggttagtgggaagagagggtaaagggaagttttttgttgaggttttaatgtttggattgatatttgagttattagggagttttagagggagttaaattagattttgggttaggaagatagggaatgtttagggt

SEQ ID NO: 154 - DNA, 1700 bp, Artificial Sequence
  chemically treated genomic DNA (Homo sapiens)
tgagggttttggtttttttttttgtgtttgttttttgtttagtgttgggagaagtagtaataagttttgtattttagtaattaattttagttattatatttgtattaattagtgttgtttaagttttgggtttggggtggggtttgtttttaaggtggtttggggttttggttgttgaagtttttgttatgaatttgtattattttagttttggggttttttgtgtttgtttttttttggggtttggtgttagtgtgttgggggtgtgttttggtttttgggtgtattgagttggagagggtgtaggtggaggagaatggtgggagtttggttttgtttattttttttttatttatattgtttttaattttttatttttaattggagtgagatttattgggtgggtagggggtggggaggtgggtggtgagtggtatggggtgtttagataatgaggttgttaggtaaagattttagatttgttatggaaagttgttaagtgtttattgtattttggttttggggtgaatattttgttttttgttagtggtgtttaagttagttggttttgggatttttttggagggtaaagtagatggggaattttgaagttagggttttttttgagagatttgggtgtttgtttatttttttgttattagggtgtttataattgttttttttttttattgtttggggtttttggtttttattggttggtgtgggttttaaggagttatagttatttagttattttatgggggagtatattggttgagtttagtaattggagtgatttgtgaggtggttgggaattttgagaagatatgtatgaagagggtgtatttgttgggttgtatattttgttttgggttatttggtttaagatttagattttttgtgggtgtgaagttggtttttttaagagattaattaaattgaagaggttgaggaatggaattgggttgtgatattaaaattaataggagtaatttaatatttgttttgttttttatgaaatatttattttaataattttagttaaaatgtttagattgttttgatttttatagaggttgaaatttattttgtaattagaagttggaagttagaggtgatggaggatagaggtgtttattttgtgtgtttgtgtgtgtgtgtttgtgtttgtgtgtgtgtgtttgtgtatgtgtgtgtgtgtgtgtgtgtgtttttttttaaagtgtgtgtagggaaggaggggggagaaggtttttaattttttttgttataaaatagtatgaggtgattttgttagtagatttgtatttttaggagaagggtgaaaaaagaattttttttgaagtgtttttgatattgttattaaatttaaataaaattaagtgtggtgtatgaaaatgtttttttttttttataaaagaaagtgtttattgtttgagtttgtttagtgtttgttaaattatgtatgaaatttgaaatgaaataaatgttataataaaattaatttttgagttttttttatttaaaaattttagattaagtattttattgttgtgggtaggagttaaaaagttattatgttgtatgtgtagatttattgtttttttgagttgtggttaggaaggtagtgttaagtatgttagaattgatatgttgggggtggggggaaatgggtttaattttggggtagtggtagaatt

SEQ ID NO: 155 - DNA, 1700 bp, Artificial Sequence
  chemically treated genomic DNA (Homo sapiens)
aattttgttgttattttaaagttgagtttatttttttttatttttggtatgttggttttggtatatttagtattgtttttttagttgtaatttaggggagtaatgaatttgtgtgtatgatatggtaattttttaatttttgtttgtgatggtggagtgtttaatttaaggtttttaaataaaaagggtttaggggttggttttattataatgtttattttattttagattttatgtgtaatttagtaaatgttagatagatttgggtgataaatatttttttttgtggggggggaaaggtatttttatatattatgtttaattttatttagatttaatagtgatattaggggtattttaaagaaagttttttttttgttttttttttaagaatgtgaatttgttagtaaaattgttttgtgttattttatggtaaggaaaattaaaggtttttttttttttttttttttatatatattttagggaggaatatatatatatatatatatgtatgtataaatatatgtatgtaggtgtgagtatatgtatataggtgtatagaataaatatttttattttttattatttttaatttttaatttttaattgtaagatagattttagtttttgtggggattaggatgatttaaatattttaattaagattattgggataaatattttataaagagtagggtagatattaaattatttttattgattttagtattatagtttaattttattttttggtttttttagtttaattgattttttaaggaggttaattttgtatttgtagagggtttgggttttggattgggtggtttagggtaaggtgtgtggtttggtgggtgtgttttttttgtgtgtgtttttttggggtttttagttgttttgtaagttgttttggttattgggtttaattgatgtgtttttttataaagtggttgagtgattgtgattttttggagtttatgttggttggtgagaattgggaattttaggtagtggggaaggggaataattgtgaatgttttagtgatggaggagtgagtaagtgtttaggttttttgaggagaattttaattttgaggttttttgtttgttttattttttaagaaaattttagggttggttaatttgggtgttgttagtggagaatggagtgtttattttggagttgaagtgtagtagatatttagtgattttttgtggtgagtttagggtttttgtttggtgattttattgtttgagtgttttgtattgtttattgtttatttttttgttttttgtttatttggtgggttttgttttagttgggggtggggagttgggaatagtgtgaatgggaagggggtgaataaagttaagtttttgttatttttttttgtttgtattttttttagtttgatgtgtttggaggttggggtgtgtttttagtgtattgatgttggattttgaggagaggtgggtgtaggggattttagagttggggtagtgtgggtttgtggtgggggttttggtagttaggattttggattattttaagagtgagttttgttttgagtttggaatttgggtggtgttgattggtgtaaatgtaatggttgaaattgattgttgaaatgtaaaatttgttgttgttttttttggtgttgggtgagaggtagatatagagaggggagttgagatttttg

SEQ ID NO: 156 - DNA, 1700 bp, Artificial Sequence
  chemically treated genomic DNA (Homo sapiens)
ttttatggggatttttgttaatgtttggttgagaggtggttttagggggttgagaattttatttttagaggtttggtatttgggagattgagaaagttttttttaagtttttttgttgggttatgttgtatttatagattatttttaattttttttttttttttatttatttttttgtttttatttttttggtttattttttgttagaaaatgtattaagttttaatttttgttttttttagttttttttttgagaagatattgtgattttgttgtttttgtttgtgttattatggagaggtaggaggataaatattgtttttagttgttagtttttttgttttttttttttttttttagatagtttttagttttaattttagtatttgttttattatttttttttggagaattagtataattggagtaaataggtaattaagtagtgtgtttgtaagttttaattttgggatagataaggaagtgaaattgattggaattatatttgttgttgttggaggttgagggaggtggttgttggaggtgaggttagttttgttggtttagggttttgttttatttttttttttttttttttttttttttagttttatttttttttatttttttttattttgtatttttttttagtggttgtttgttttttttaaggaattttattttaggtggagtttttatgatagttatattttttttttgggtttagtagggttagagaaagatggaattattttatttatttaggttgtttagagagtattttaggttttgatttgtgtggggtgtgaggtattttgtggggtagttaggtgtttttaagaaattttgttttagaggatttgtatgagttgttgttattttttgttgaagtttttgttttggggttggtgtttttttttatgattttattaattgttggtatttttttttttttttttttttttttaggaaggtgttggggtgtgtggtggtaatggtggtggtgaagggatttttttgtggtatttttttgtgggtttttgttttgaaggattttttttttgttttaattttatgtttaagtagtgttttgggggttgggttggtttttatagtttttatttttggttttttttttttaaagggtgtgtggagtttagaggatgggtggagagtgggtttggttggaattgaattggtagaggtaaaaggggaatggaggattttaagttttggttattttggtttagtgaatttattttatttttgtttaaggaaaagttgtaggggggaggattttttttaggaagtttattttgttggtttttgatttttgggttggatgtttgagagttttgttttttttgttttgagagtggtttagttgggtttgaattgattttttttttttttgattttttttttattttttagtttggagaaataggggagtgggggtttaagaatgagaagatgagaatgtgttgttttgtgttatgttagaatggggtgggtgtgaggggaatagtttttttgtgattagtttaggagtatgagttttttggaggatggtatgagttttggatattttaggagtttttagttagtgatatggttggtaagtgattttgggattaatatttatttatttttattttttttgtttagattttattttttttttagtttttttgtttttt

SEQ ID NO: 157 - DNA, 1700 bp, Artificial Sequence
  chemically treated genomic DNA (Homo sapiens)
ggggagtagggggattgggagggggatagaatttagatgaaagaggtgggagtgggtgggtattagttttagagttatttattgattatgttattagttgaggatttttgaagtgtttagaatttatgttgttttttgggaggtttatatttttgagttgattgtaagagagttgttttttttatatttgttttattttgatatagtgtagggtgatgtgtttttgtttttttgtttttgggtttttatttttttatttttttgggttggaaaatgaggagggggttgaaaggggagggagttgatttagatttagttgggttatttttggaataagggaaataaagtttttaagtatttaatttaaaaattaggggttggtagagtagattttttaaaaagaatttttttttttgtagtttttttttggataaaaataaagtgggtttgttgggttgaagtggttagagtttaaggttttttgttttttttttatttttattaatttagttttggttaagtttgttttttatttattttttgaattttatatattttttaaaaggagagggttgggggtgggggttatgaaaattaatttaatttttaaagtgttatttgaatataaagttgaggtggagaagaaattttttaagataggaatttatgaggagatattataagaggatttttttattgttgttgttgttgttgtatattttagtgtttttttaaaaagaaagaagggaggaaaaaaatgttgatgattggtagaattatagagaagaatattagttttagggtaggggttttagtgaaaaatggtagtaatttgtgtgggttttttggggtggggttttttggaggtatttagttgttttatggggtattttgtgttttatgtgagttagaatttaaagtgttttttgggtaatttgagtgggtagagtgattttgtttttttttggttttgttaggtttaaaggagaggtgtgattattgtgaggattttatttagggtggagttttttaaaaaggataggtggttgttagaggagagtgtaaagtgggaggaggtggagaagggtggggttgagaaggaggagggaggaaggagggggtggggtggggttttgggttgatagagttggttttgtttttggtggttgttttttttagtttttagtagtggtaggtataattttaattaattttatttttttatttgttttaaaattaaggtttgtaagtatattgtttgattatttatttattttggttatgttaattttttaggggagggtggtgaaatgggtattgggattggggttgggggttgtttaagagggaaaggagaaaataaaaaagttgataattggaagtgatatttatttttttatttttttatggtaatatgaataaggatagtgaggttatagtgtttttttagggaggaagttaaaaggaatggagattggagtttggtgtattttttagtaagagataagttaaggaggtgagggtgaaggagtaggtggggaagaagaaggagttgaagatgatttgtgaatatagtgtggtttagtagggagatttaagaaaggtttttttagttttttagatattagatttttaagaatagagtttttaattttttgagattgttttttagttaggtattggtgaagatttttgtaaag

SEQ ID NO: 158 - DNA, 1700 bp, Artificial Sequence
  chemically treated genomic DNA (Homo sapiens)
ttagatagtagaagtttatattttttggaggtggaagttggaggggaagggtttgaggtagagataaaagtaagagagggattttttaggtttttttggggatgggaggatggtgtttgtttagttttgtgtttgtaatttttatgttatatatattatagttatgtttgggttatgtttgtttagagtttattgggtatgtatatatgtgatatgtttgaagttgagggtaagttatttagggtagagtatgtttttaggttttgtgtttgttttagggtagttgtaatgtagtaagtgtgttattgttatggaggtaagaaagttagtgtttttttatgtatgtatataggttaggtggttttttttgtataagatgggttattaataaatttatttatgttatatatttgttttttattgtttttattttatttgtaaagtatggggaaaagaaagtaaaagttttttttgatttggtttttttttgtgtttttttttggagttgaagagtttgatttgggaaaagatatttaatttgtttttgttgtagttggttttgtttttgttggggttgttgatatatttgtaggtgttggattttttgtgtttgtgggtttgttttttttgatggatgagtgagttggtgatagttgttgttatggttatgatgtttattattattgttttttttgttgtttttttttgggtttgttttttttttttttttttttgttgtttgtttgttgttttgtgattttgttgttttggtttttttagtttgtgtttgtttgggtttttttgtatttgggttttagttaaggagggggtttagtatgttgtttgagtttttttggtggtggtttggtttttgtgtgggttgttggttgtttgggttggagttgatgttatagttttgggtttgggattgtgggtgagattggtatgtggatgttgaattgtgtgattgtgtgtggttggtttttgtgtttggtggatattgtgtatgtttagttgttttggtttgaattttgttggatttttttgttttgttgtttttttgatgggggttagaggagggaggtgggtggagggagtgagtgtgggtgggagagaggttgggagtttgggtggttggatggaggagggatgaggtagtgtgtgggggagtgtgtttttgttttggtttttttgagtttttgattagtttttgtaattttttggtgtgataattgggaaaagttggtttgtgttaggtttttgatagggattaaataggtaatggttttgtatttttgttgttgttgttgttttgggtgtggtatagttgtagtataggaatttagttgttgtaggtattttttggaatagtgtgatagtttttttgtagtttttttttgtgatggttttttttttttggggttgagtttgtaggtttggagttgtaggttgttttttttatttttatttttatttttatttttttaagtttttggttgtttgttttggttaatatttgtaggggtttttgtattgttttagttgtttaaatttttttttttttttttttttgaaaggagttttgggaggaaatagtgatggaggaggaattgagatgaggtagttgggttgatggagtgggggtttgtttggtattgggggatggagtggtgggggtgtttttttagtgtttgagttga

SEQ ID NO: 159 - DNA, 1700 bp, Artificial Sequence
  chemically treated genomic DNA (Homo sapiens)
ttagtttaaatattgaggaaatatttttattattttattttttaatgttgagtaaatttttgttttgttagtttaattattttattttagttttttttttattattgtttttttttgaagttttttttgggaaaaggaggggagaaggatttggatgattggggtagtgtggaagtttttataggtgttagttggaatgggtggttggggatttgggggggtgggggtgggggtgggggtgggaggggtggtttgtggttttgagtttatgggtttagttttgggagggaggggttgttgtgggagggggttgtaaggaggttgttgtgttgttttggagggtgtttgtggtggttgaatttttgtgttgtgattgtgttgtgtttagagtagtagtaatagtgaagatgtgaggttattatttgtttgatttttgttggaaatttggtatgggttaattttttttgattattatgttaagaagttgtaaggattagttgaagatttggaggggttagggtgagggtgtgtttttttgtgtgttgttttgttttttttttgtttggttgtttgagtttttggttttttttttgtttgtgtttattttttttgtttgttttttttttttggtttttattagaagggtaatagggtgagggggtttggtgaaatttggattggagtagttggatatgtatggtgtttgttgggtgtaggggttgattatatgtagttgtgtagtttagtatttgtgtgttagttttgtttgtgattttgggtttggggttgtggtgttgattttgatttaggtagttagtagtttgtgtgggagttggattgttgttggaggagtttggatggtatgttgagttttttttttggttgaagtttgagtgtggagaagtttgggtaaatgtaggttaaggagattaaagtggtgaagttgtgagatagtggataagtagtggaggagaaggaggaggaggtgaatttagagaggggtagtaaaagaagtggtggtggtgggtgttgtggttatggtggtggttattgttagtttgtttatttgttagaagaggtaagtttgtgagtgtgagaaatttaatgtttgtaagtgtgttagtagttttagtaaaggtaagattagttgtgataaaaataagttaaatgtttttttttgggttaaattttttggttttaagaagaggtgtagaagaagattaggttggaaagggtttttgtttttttttttttatattttatgagtgaggtgggggtggtggggagtaggtatgtagtataggtgagtttgttggtggtttattttgtgtagaagaggttatttagtttgtgtgtgtgtgtgggaaggtgttggtttttttgtttttgtaatagtaatatgtttgttgtattgtaattgttttgggataggtgtagagtttgggggtatattttgttttgggtggtttgtttttagttttggatatgttatatatgtgtatgtttggtgggttttgggtaggtgtgatttagatatggttgtgatatgtatggtgtggaaattgtaggtgtagagttgggtagatattatttttttgtttttaggagagtttggggaattttttttttgtttttatttttgttttagattttttttttttagtttttatttttaagggatgtgggtttttgttgtttag

SEQ ID NO: 160 - DNA, 1700 bp, Artificial Sequence
  chemically treated genomic DNA (Homo sapiens)
ttttttttttttttttttttttttttttttttttttttttttttttttttttgtttagtgaggagtttagattagagaggtggtttagatagtttattaaatggtaagattagattagtttttaaattaagataatttattttgtttttttttttttttgaaatatatattttttgtttttagagattttaaaattttttgatgatttatatatttatttgaatttttgttttttgataaaagttattatgtgtattttggaaaaagtgaagaaaagttaaaagaaaatatttatttagagataatattttagtggatttttttgtttttatttttgtatatatgagtttatagggtatatatgtttgtatatgtttaaatttatattataagtatgtttagtttatttttagaattttttaaataaatataatttaaaataggaatttaatattttatgttattgatagattgtagtagattgtgtttgtatttttagataatatttttttttattattgtaaataagattttattttgtgtttttaatttttatttttgttaatattttttgttatttttttaggatatatatgtagaagaaatattaattagttaaatattttaatgtttataagatatgttattttagggttttttagaaaaatagtattattgtattttgtttttggaggtgtaggtgagtgtttggtttatgggtgtttataattattattgggtatttataagagattttgaatttagaatatttttgtggtaggagttttgttttgtttttttaggggatattgatgatttgttgggtttttgttttttaagtgttggaggggggaagtagggttatttggggtagaaatttaatgttagaaattttttagtgttttattagttggtggaatttttgtggtagatagttgtgttttatttaagttgtagaagaaaagatattttgtgggggatttggtgttggaagttttgagtttaaattgtagttttaggtttttagttttgttttttttggtttgttgtgaggatgaaatgagatttttgtatgtgaaaatgttttttaattttttttgaggggtggaaatgtttttatttgattagagtttgggtttgtggagtaatttatttgttgttttttaagagtaggttttttagtgtagtgagtttttgttatggaaagggttgttttggtttaggatgtgtgtttttggtgtagatttggggataggggtttttgttgtgtttgttttatttttgtagggagtaaggttgtttttttgtagtattgtgttatgtggtgggaatttgtagtgttttgttttttggttttagtttttttgtttgtttgggttttttttggttattttttggaggggtttggagttttttttttttttttttattttagtgttagaagtattgaaagttgtttgtttgttgagagtggaattggtattttgaattttgtttattttgggttaattggaggtgtattagtggttgttttgtagtgtaatagaggtttgagtgataggttatttgtttaagtgttggtgtttaggaaagttggggaggaaattaggaagggaggtgatagtagaaaagttagagagtttagagatttttaggtaggtttttgtgtagttttaatagtgtggtgttgggaggtgtgt

SEQ ID NO: 161 - DNA, 1700 bp, Artificial Sequence
  chemically treated genomic DNA (Homo sapiens)
atatattttttggtattatgttgttggggttgtatggaggtttgtttaggagtttttggattttttggtttttttgttgttgtttttttttttagttttttttttggtttttttgggtgttagtgtttaggtaggtggtttgttatttgagtttttgttgtattgtaaggtagttgttaatgtgtttttaattgatttggaatgagtgagatttgaggtattggttttatttttggtgggtgggtggtttttaatgtttttggtgttgaagtaggaggagggaggagggattttaggtttttttgagaggtgattgagagagatttaagtgagtgaagaagttgaaattagaaagtaggatattgtaaatttttgttgtgtggtgtggtgttgtagaaggatggttttgttttttgtaggggtggggtgagtgtgatagggatttttatttttaggtttatgttgggagtgtgtattttgagttgaaatgattttttttataataaggatttgttatgttggggagtttgtttttggaaagtggtgggtaaattgttttgtagatttggattttggttgaatggagatgtttttattttttgagaggggttagaaagtatttttatgtataaaaattttattttatttttgtagtaagttaggaagggtaaggttggggatttagggttatgatttgaatttaaggtttttgatgttaagttttttataaggtgtttttttttttatgatttaggtagagtgtgattgtttgttatgaaaattttattaattgataaggtgttggaaggtttttggtgttaggtttttattttgggtggttttattttttttttttagtatttggaaggtgagaatttagtgagttgttagtattttttggaaggatgaggtggaatttttattgtggaaatattttaagtttagggttttttgtgggtgtttggtgatgattgtaaatatttgtagattaggtatttgtttgtatttttgggaataaaatatgataatattgtttttttaggaagttttggaatggtatgttttatgagtattaaaatatttgattgattgatgttttttttatatatgtattttgagaaaataatagaaaatattgataaggatgaaaattgaaggtataaaataaagttttatttatagtgataaaaaaagatattatttaaaaatataaatatagtttattatggtttattaataatatgaaatattagatttttattttaaattatatttgtttaaaaaattttaaagatgaattgggtatgtttatgatgtaaatttaaatatatataaatatatatattttatgagtttatatatatagaaataaagataagaaggtttattaaaatgttgtttttgggtggatatttttttttaatttttttttattttttttaaggtgtgtatgatgatttttattaggaaataaaaatttaaataaatgtatgggttattagaagattttgagatttttaagagtaaaggatgtatattttaaaaagaaagaaagatagggtaaattgttttggtttagaggttagtttggttttgttatttggtgaattatttgggttatttttttgatttggattttttgttgaataaaagaagaagaaggagaaggagaaggaggaggaggaggaggaggggaagaa

SEQ ID NO: 162 - DNA, 1700 bp, Artificial Sequence
  chemically treated genomic DNA (Homo sapiens)
ttattaaaaaagtggagggggttttgtttaatatttgtatatatttttttttgtagtattttgtttattttagttttttagggaaattatagataatgatgttttagagagtgttagaggtgaagttgatttgaaattatattatagaaaggttttttgttttaaataggtttaataaagttattaaattaagtttatgattataaagaattgtgaattagaaaataattttatataggtgtagttttttattatttttttttttatttttggaaattttaaagaaaaattgttatatttttaaatatgaggttattattttaaaggttgtaataatttggaatgtaaatttgttaaattatgatataataaggttaattttgaggatttttggtggattattagtgtatttttttataattagaaagtgaaaaattgggttagttttagagttatgtaataatgttgtgttatgaaattgtgtattttaagagaaattttttttaggtttagaattaagttggagggatttgttgtgtagtttaatttttaggaagtatgaaagaatgtattgtaaagagtaaaattatgtgtaatttattttaatataattgtagttgtttgtgggtttttttgtagagaaaggttgtttaattttgatggggttatgggtgttgtgggagggtaaaagattatagagatttgtgaagttaaagtaaataatggggttggggtggtgagaatgattaatagtgatgttttgaaaggattttgtgataggaattgagtgggaaggattttttttattttaatagtttatgggttaagaggttgaagtttttttaaatattatatttttgtgagagaagggattgagaataattttttaaattatttttttttgaaattgtttttttttttgaaaattttatttttgagatttggatgaggtttttattttttttatttttgttttgtgattgttgtttgtttagttttttagttgagtttgtggggtttggggtgtttattttgtttatttaagggattgtgtaggggtgaatttttttgtgttttatttgtgttttttagattttgggtgttttggtgttgttttgagagttttttgtgtggttgttgtggtatggattagtttttatttttttatattgggtgttgttgtgtttgttgggggttggttttgaggtttttaaggttttgtgtgtgtgtttgttgtggtaattaagatgttttatgatgtgtgtttttgaatgttttgtgttatgtggttgtgtggttttgtttgttggttttgtttttgttatagagtttgtagtatgttgttgttgtagtttaggttatgtgagtatttatgtgtgtgttttgttagtggatttattattggtttgtttagattaggttttgtttattttgattttttaaatggtatgtgggaggatgtttgttttttttggatttaagagttattgtaatattttagaaggggagaaaaggagtgagggtggtaggtgatagagaattttgtgagttagtggttttgtgtagatttttttaggtatggttttttgtggttatgttggttgtttggtgtttgtgtaatttttttttttttagtgaaattgaggtttttggagagtttagtagagagtgtgggtagtgagtgtttgtagttgttagag

SEQ ID NO: 163 - DNA, 1700 bp, Artificial Sequence
  chemically treated genomic DNA (Homo sapiens)
ttttagtggttataagtgtttattgtttatattttttattaggttttttggaggttttggttttgttggagagaaagagattgtgtaggtgttgagtagttgatgtggttgtaggggattgtgtttgggggggtttgtgtggggttgttgatttgtgaggttttttgttgtttgttgtttttgttttttttttttttttttagagtgttatggtgatttttgggtttgaaggggatggatgtttttttatgtattatttagaaggttagagtgggtagaatttagtttgagtaggttggtgatgaatttgttggtaagatgtgtgtgtgggtatttatgtgatttaggttgtggtggtggtgtgttgtgggttttgtggtgggagtgaggttgatgggtggggttgtgtggttgtgtgatgtgaagtgtttgagagtgtgtgttgtggaatgttttggttgttatggtaagtgtgtgtgtgaggttttgggaattttgggattggtttttggtgagtgtagtggtgtttagtgtaagggagtgggagttggtttgtgttgtggtggttgtgtagggagtttttgaggtaatgttggggtgtttgaggtttggaaggtgtaggtggggtgtgggggagtttatttttgtgtggttttttgggtgggtggggtgggtgttttaggttttgtggatttggttgggaggttgagtgggtgatggttatgggataagggtggagggggtggggattttatttgagttttagagatggaatttttgaggaagggagtgattttgagagagaatagtttgagaagttgtttttagtttttttttttgtgggagtgtggtatttagggaagttttggttttttaatttatggattgttaggatggagggaattttttttgtttgatttttattgtggagtttttttggagtattgttattgattatttttgttgttttaattttgttgtttattttggttttgtagatttttgtggttttttgtttttttgtagtgtttatggttttgttaaggttgggtagtttttttttgtaagaaggtttatgaataattgtggttatattgggatggattgtgtgtgattttattttttataatatatttttttatgttttttagggattgagttgtgtggtggatttttttgatttaattttgggtttgagaaaagttttttttggaatgtgtagttttgtgatgtagtattattgtgtgattttagggttgatttagttttttattttttaattgtaaggaagtgtgttagtagtttattgaggatttttgaggttggttttattgtattataatttaatgaatttgtgttttaggttgttatagtttttgaaataatggttttatatttaagaatgtagtaatttttttttgaaatttttgaaagtggggaaaaaagtaatagaagattgtatttatatggaattattttttgatttgtagttttttataattgtagatttaatttggtggttttattggatttgtttgaaataggaagtttttttgtaatgtggttttgggttggttttatttttggtattttttgaaatgttgttgtttatggtttttttaagaagttagaatgagtgggatgttatggagggaggtgtatataggtgttagataaagttttttttgtttttttggtaa

SEQ ID NO: 164 - DNA, 1700 bp, Artificial Sequence
  chemically treated genomic DNA (Homo sapiens)
aatttaggtttggtaggttatttgtatatattgggtggaggtggaaggggtaaagtatttttgaggtgatggttttgattttattttatatttttttggggtttggttggatttggttgagtgtgtttgagtagtggttgatttttggaaaataggaggatttttttttaggaggttgtgattattaaggttgtaaatgttttatttgaatttgggtgtatgagggattttttttttttatatatatatatttttttattgtgtaagataaatttttgtttgtgagttgtttatattaaatgttttgtttttgattttttgtgattaaaatttgttttgtttgtagggtttggtgtgtttggatttagtgtattttgttggtatagtaaaaaagagaattgtttttttataggtgatttttgttttaaagtttttttttttattaatttttttttgagaagagagtaaaattgaatttgagagaaaagaggtagtaattatttgggaggaggtagattggagtgggtattagttttggtagtaagagttttaatgttttttttgttttttttttgtagagtagtatggaagtattagtttttttttttgggtttttaaaagttattttttttttgggtgtgatttttgaatttgaagtgtttgagttttaagaaattagaataagttagagttaaagaggtttggtgggtgggggtgtgggtggagtttgggttgggtggtgtggaaggttgagtatagttgtgagtggtgagttttgggagaggaggagtgggagaaagtggggtgggtttgtggttggggtttggttgggggagggggtttttgtttttttttttttatttttgttttttttggttttggtgatgttgtggtttttttttttttttagtggagttttagtgtttgttgtgtttttttttttgttttttttgtttatatttatttgtttttgttggtttggtttttagtagtgttgtttgaggttgtagtagtgtattttggggtatggtgtggtgggggtgtggagggtttggtttggagggggttgggagtttgggtgttttggagtgaggaggattgggagttggttttggaggttgtggaggtgatgttggagagaatgaagttttggttgggagtggtaagtggaggggtgtgtaagggatgagttggggtgtgtggtgtaattttttttgggagttttgggtgggtagtttttggggttgttttggtgtgggtgggagatggagagttatgtttttggttgagagtggagtgttgtgggttgtgtgtttgggtttggtttggttggattttgagagagtgtttgatgtgtgaagtttgagttatgggtttttttggtggtggagtagttttgaggggtagttttttgattttttttatttttttgaatgttgagtattgagttgggaatatgatgagaatttggagattgtattttgtgtgttggatttggttggtttagttgtgatttttttgatggtttttggggttttggttttattttgtggggttgggtggtatatttttgtgtttttttttttttttgtattttagttatttgggggtaggggaggtggtggtggtttgtgtgtagattttggtgttggtaattttattttgtggttgtagaagttgaggaagagttttttaagttt

SEQ ID NO: 165 - DNA, 1700 bp, Artificial Sequence
  chemically treated genomic DNA (Homo sapiens)
aggtttggggagtttttttttggtttttgtagttgtaaagtagggttgttagtgttaggatttgtatataagttgttgttattttttttgtttttaaatggttggggtatggagggaaaggaggatgtgagggtgtgttatttaattttgtagagtgaagttgaggttttaaggattgttgaggaaattgtggttgggttggttagatttgatgtatggaatgtgatttttaggtttttattgtgtttttggtttggtgtttggtgtttaagggggtggggaggattgaaaagttgttttttgaggttgttttgttgttggggaagtttgtggtttaggttttatgtgttaggtgtttttttggagtttagttgagttgggtttgggtgtgtaatttgtggtgttttgtttttagttggggatgtggttttttgttttttgtttgtgttgggatagttttgaaggttatttgtttgaaatttttgaagaaagttgtgttgtgtgttttagtttgttttttgtgtgtttttttatttattgtttttagttgaggttttgttttttttggtgttgtttttgtagtttttagagttagtttttggttttttttattttagggtgtttgggtttttggttttttttgaattgagttttttgtgtttttgttgtgttatgttttgggatgtgttgttgtagttttgggtgatgttattgagagttgggttggtgggggtgggtaggtgtgggtgaaaggggtggagggagggatgtggtgggtgttgaggttttgttggaggggggaggaaattgtaatgttattagagttagaggagatgaggatgaggagagggaaatggggatttttttttttggttgaattttgattgtaggtttgttttgtttttttttgtttttttttttttgggatttgttgtttgtagttgtgtttggttttttatgttatttggtttggattttatttgtatttttgtttgttaggtttttttggttttaatttgttttagttttttaggatttaaatgttttgggtttgagaattatatttaaaggaaaggtaatttttggaggtttagggggaggagttgatgtttttatattattttgtgagaggaaagtgaggaagatgttaagatttttattattgggattggtgtttattttagtttgtttttttttgaatagttattgtttttttttttttgaatttggttttattttttttttaaagagaagttagtgggaggggggattttggggtgggggttatttatagagaagtagtttttttttttattgtattaataaaatatattaaatttaagtgtattgaattttgtaaatggaatagattttaattatagaaggttaggagtaaagtatttaatgtaggtagtttataaatagaaatttattttatgtggtgggggggtgtgtgtgtgtgagagagagagattttttatgtatttaagtttaggtggagtatttgtaattttggtagttatagttttttggagaagagtttttttgttttttaaaaattagttattgtttaaatatgtttagttaggtttaattaggttttaaggagatgtgaggtaggattagggttattattttaggaatattttgttttttttatttttatttagtgtgtgtaagtaatttattagatttagatt

SEQ ID NO: 166 - DNA, 1700 bp, Artificial Sequence
  chemically treated genomic DNA (Homo sapiens)
aatagaatttagaattttggggtagttttttggagttggagtttttagggtaaagataatgttaatgtgttgtttaaaagggtttaatttttaaaatatttttattggtaatatttattttgtttattagaaatgtagttatgtatttttgtgtttggtaaagattttttgttttgtgtaaatttataaattaaaataattagtttagatttttgatgtgattttgtagtggaagtatttgtattagtgaatgttgaaaagtaaataaaaatgaaagttttgtggaggttgtgggttggatgggtgtggggtgttttttgttattttagtgtttgttgggattttttttgtgtttttgttgtttgaattagtttggttttgggatgttttttttaaatttttataagttaaattgaatttaggtttgagtttgttattgtaaaggaatttggggtgtggggtgggagggataggaggagagatgggttgggtgttaaatggtagaataaatggttgatttaattaaagttgttaatttaattttttattttttttatattagtttttttttttttttttttttttttttttttttaaatttgttgaattttattatttttgtgttggtaagaaaatttgaatattttgggaattgtgtttaaattgtatttgtaatttttttggaagtttttgaaattttttgagaatgaggtggggtggtttaaggggaaagggtagggaggagagatttttgggaagttgatgatgtgtttggggtaagttagtttgtttttgagatgtgtgttttatggttgtagtagtgttgtgtttattttgtttttttagtgatttagttagtttttgttgggggtttttgataatgtttattgataagttttttttgtttttaaaaggtgtgtttttttttttttttaggtataaagtaggggtgggaaggggagagttatataatgttgtaaaaagaagtgtggttttatttggaagatggttgaattttttttgtttttttgatttagttagtaatgataagaaagatattgaatattgttttttgaaaattgggtgtatttgaatttggtgaaaagtgtttttgtgaggaagtttaatgtttatttttgtgtaaggataattggaaattaaagtgtttagtaatgtagagattttattaagtgagtgttttttttttttttgtgttaggaatgaaataagttagttagatagaaaatatttttgggagaagattaattatttttgggtaggttgaattgattggaaattatattaagtaaaggtagtatattttattttgttttggtgatgttgattttatattattagaatattgtttttttagtgtgggggtgttttgtattttttaaagttagggtagattggttatttgggaaaatgtagtgttagtgttttgtttttattttttttattgtttaaatatgttttatatttttttaatgttttttgagggtttttaatttagttattgagttttttatttttggtgattgtgatattaataatgttgttattttttttgggaaatatggttgtaaagatgttttgtaggaggtagataaaaggttgtgtttttagatattgaaggttgttttgttatttagatgtttttaatttttgtatagagaaggagggaaaatatggggtgt

SEQ ID NO: 167 - DNA, 1700 bp, Artificial Sequence
  chemically treated genomic DNA (Homo sapiens)
gtattttgtgttttttttttttttttatgtaggaattggaaatatttaagtaataaagtaatttttagtatttgaaagtatagttttttatttgttttttgtggagtatttttgtaattatgttttttaaggagggtgatagtattgttggtattatagttgttaagggtgaggaatttagtggttgggttgagaatttttagaaggtattagaaaaatgtggaatatgtttaaatagtgaagaaagtaggagtaaagtattaatattgtatttttttaggtaattagtttattttgattttgggaagtataaagtatttttatattgggggaataatgttttaataatatagaattaatattgttaggatggaatggaatgtattgtttttgtttggtataatttttagttaatttggtttatttagaagtaattagtttttttttaaaagtgttttttgtttaattggtttattttatttttggtgtgggggggaggggagtgtttgtttaatgaaatttttgtattattagatgttttaatttttagttatttttgtataaaagtaaatattaggtttttttatagggatattttttattgagtttaaatgtgtttgatttttggagggtgatatttagtattttttttgttgttattaattgggttgaggaagtaaaagaaatttaattgttttttaaataaagttatgtttttttttataatattgtgtagtttttttttttttatttttattttgtatttggagagaaagagaagtatgttttttgggagtaaggagagtttattgatgagtgttattagaaatttttagtaaaggttggttgggttattaaaggggtagggtgggtgtagtgttgttatgattgtgaggtgtgtgttttggaaataggttgatttgttttggatgtgttattagttttttgagggttttttttttttgttttttttttttgagttgttttgttttatttttgggaagttttaggaatttttaggggagttgtaagtgtaatttaagtataatttttaaggtatttaggtttttttattaatgtggagatagtgggatttagtaagtttaagaggaaaaaagaaaaagaaaaaaaaaaagggttggtgtgagagggatgggaaattagattgataattttaattaaattaattatttgttttattatttggtatttagtttatttttttttttattttttttattttgtattttgggtttttttgtagtgatgaatttggatttgagtttgatttgatttgtgaggatttgggggaaatgttttaagattgagttggtttgggtagtgaaggtgtgggaaggattttggtagatgttaagatggtaggaggtgttttgtgtttgtttggtttatggtttttgtaaagtttttatttttatttgttttttagtatttattggtgtaaatatttttattgtagggttatgttaagaatttaaattaattattttagtttgtagatttatatagaatagaaaatttttattgggtataaaaatgtatggttgtatttttgatgaatagaatgggtgttattaataaaagtattttaaaaattgaatttttttaaataatgtattagtgttatttttgttttaggagttttaattttaaagaattgttttagagttttgaattttgtt

SEQ ID NO: 168 - DNA, 1700 bp, Artificial Sequence; identical to SEQ ID NO: 166
  chemically treated genomic DNA (Homo sapiens)

SEQ ID NO: 169 - DNA, 1700 bp, Artificial Sequence; identical to SEQ ID NO: 167
  chemically treated genomic DNA (Homo sapiens)

SEQ ID NO: 170 - DNA, 1700 bp, Artificial Sequence
  chemically treated genomic DNA (Homo sapiens)
attggatgagtggggttttttgttttagttggatttttataggaggatggaggaaagtatttttttgagtaagtttggagagtgaaggggtggggttgtaaggttggagaaggaggtgttttttttataaaaatgattgggttttttttgtaatggttggtataaagttggaagggtttggggatgggaggggtttgaagtgatttttgaggagttgggttttttttgtaaggttttgggagggaaggtaaggtattttgtgtaatgatatattttagttgttggatgtgaagatttttttttgaagatagaataaagaaaggtttttttattaatgtgggtttgaaattggtttgaaatggtgtgtttgtagggatttgggaggaaatgtgatgtttatttttagatttgggaaaggtgggggtaataattttttaagagaattggagtttgaaagaggaatgaaaagtttttgtgttgtgtatatatatttttttttattggaaagttaggagtgaggaaagaattttgaagtgatttgggaatgatttgaaagtgtgtaagttggaagttgggttgaagttagtatagtggagtaggggttaggtttttgtgtggttggagtgggagagtgtttttgagaataagatggggtaggattgaaggggtgtgaggttgatgttttttagggatgatttggagggtgaaggaggtttgggggtggttttgggtttttggaggtgattgagggagtttttgagttaatgtttgggggtgggaggtgttattggtatttattgagttgatttttagtgttagggtgtattgtagtgtttttatttttggtatttgtgtggggaggggtggtggggagtgattttggttatgggtttgagtgaggtggggagatttggggttgggtggggatggggaggagatgaggtggtggtggtggtggtagtagtggtttttaggttggggtttgtggtttagtttgggttttgttggagaagtagttgagtgtggggtgtagatttgttgttatggtagttaggaggggtggggttggaagagaggttgtgtggggtattttagggttatttgtgttgggatgagtgttggaggtggagtttgatgaatgttgagtgaggtggagttgggtagggggtggggtttttgaaggggtggggtgaggtaaggtagtttttttgtgatgttaaattgtgtttaaagttggatgaaagggagttgttgtttgtataatgggatggtgttttgtttagggagttggagtttttgtaaggttgttggagggaatttttgagttataatgtggtgatggtattttgggttagtggtgatttattgtggttttagtttttttaattgtaaaagaataataattattgttagtttttttatttaaattttatggtatggtttataaagttagtataaggatattgggaattagggaagagatttgtttaaattaaagttggaattaggattggaatttaggagtttttaagttaaattttagttgtttttttgaggagaggggtttgtattagatggtttatatttttagttttttattttgtgattttttttgttagtgtgtttttttttttttttaaatttttttgtttaatttatttatgtatgtgttttaatgtatattgtttaagtggaattgt

SEQ ID NO: 171 - DNA, 1700 bp, Artificial Sequence
  chemically treated genomic DNA (Homo sapiens)
atagttttatttaagtaatatgtattaagatgtatatatgggtgaattagataggaaaatttgaagggaaaagggaatgtgttaataaagggaattatagaataggagattaggagtatggattatttgatgtaagtttttttttttagggaggtaattggggtttaatttgagagtttttgggttttagttttggttttgattttgatttgggtaagttttttttttaatttttagtatttttatgttggttttgtaaattgtgttgtgaaatttaaataggaggattaataataattattatttttttataattgggaaaattgaggttatagtgggttgttattagtttaaggtgttattattatgttgtaatttaggggttttttttgatggttttataggaattttaattttttgaatagagtattgttttattatataaatagtagtttttttttgtttggttttgggtgtagtttgatgttatagaagagttgttttgttttattttgtttttttggaagttttgttttttgtttgattttgttttgtttggtgtttgttaagttttgtttttagtgtttgttttagtgtgggtagttttggagtgttttgtgtggttttttttttggttttgttttttttggttgttatgataatgagtttgtgttttgtgtttagttgtttttttggtggaatttaggttggattgtgggttttggtttgggggttattgttgttattgttgttgttattttgttttttttttgtttttgtttagttttaggtttttttgttttatttgggtttgtggttggggttattttttgttgtttttttttgtatggatgttgaaggtgaaggtgttgtagtgtgttttggtgttggagattagtttagtgagtattggtagtgttttttatttttgggtattggtttagggatttttttagttatttttgagagtttggaattatttttaaattttttttattttttgaattatttttagggagtgttgattttatgtttttttggttttgttttattttgtttttagaagtgtttttttattttggttatgtagaggtttgatttttgttttgttatgttggttttaatttgatttttaatttgtgtgtttttagattgtttttaaattattttagagttttttttttgtttttaattttttagtgagagagagtgtatgtgtgtggtgtgggggttttttattttttttttgggttttagtttttttagagaattattgtttttgttttttttaagtttgggggtgggtgttgtatttttttttgaatttttgtaggtatattattttagattagttttaaatttgtattaatgaaggggtttttttttattttgtttttgaaggagaatttttatatttaataattgaaatgtgttattgtgtaaagtattttgtttttttttttagggttttataggaaaaatttaattttttagaaattattttaggttttttttgtttttagatttttttgattttgtgttagttattgtaagaaaggtttagttatttttatgagggaaatatttttttttttaattttgtaattttatttttttattttttagatttgtttaagaggatgtttttttttatttttttgtggaagtttagttggggtagagaattttatttatttagt

SEQ ID NO: 172 - DNA, 1700 bp, Artificial Sequence
  chemically treated genomic DNA (Homo sapiens)
ttaggagtttagatttgggtaggaaataaagttttaaagtggttttgaagtgttttttttttagatttttttttagtgtgggttatttattttagtttagggaagagattattttgtttgaatgtagttaaatgtattgtatgtgggagttaagtgggagatgaaataatttaatgtgttttttttattattattatttttattaaattagttattgttggtttaaatttattgggaatgtttgtgtattttattgattagggaaataaaagggtgtatataaattagtggaagtgggtgggaaggaataagggatgtagattgttgtgagtaaaatgttttgtggtttggtgagaggttgttttttttttaagttatttattttagattgaaatattaagttagtttaggttattttttgagtagtattataagttaaaataagatttagaaaatttattaatttttaattgtgttgtttttagattgggagtatagggggatagggtgtagggttgtggggaggttgaggggagggggatttgtggaaaagtaaatgtttgagaaattttagtttttgattttagttgttttttattttatttagtttggttgaggagttgtttttatattttatggtaattttggaatttatgatgtattttttagagttgggtttttagttttttaggttttggagtttaagagttggattttataggtttaatatttattgtttggttttttttgttatattttagtttagatagttatttttatttaggatagttgtgaagtgtaaatggaggatgtaggtatttttaaaaagagtagttgtggtttgtgttttatggatgagttattgttgtaggaggtttggtggtgtggtgtgtttgtagttgtggtagtggtggagtttgtgtggggatttttttgtgtgtatttagtgttttgttttggggtagtgttattttgggtgttgttttgttatttttatggtgttttgttgttttttattattaggatggagggtattttttagtatgagttgatattttagagggtattggtgagtttttgaaaaaattagggtggtggggtgaggaagatttgtgttttggattttagagaaggaaagaaagttagagggagttggagtggagttgtattttatgttgaagttttttttttgaattgtttggagttgtaagtggtttttgattgaatatagttgtttagttgttggttatggggtgtgtggaggaggtggggttagagggagaaaagggtgggggtgagtagtttggaggttgagtagaggagattgtgttgggagagtagagtaggtatttttttagttttttgtaatagtaattaaggatttgagtttaggagggggagtagaaataggaaaagttatttgaataattgtgtaggaaattaaaaaaaaaaaaagaaaagaaaaagaaaaaaaattttttttataggagttgttttttatgatttaataaggttaagataaatttaaatattgtatattagatagtatggtgtattttttatggaatattaagattttataagggtgtagggtagttattttttttaattagtttaaagttaatgttatgggtgtaaatttaatgttaaattttttttgtttgttattggggtagagtatattttttgtatgtttataa

SEQ ID NO: 173 - DNA, 1700 bp, Artificial Sequence
  chemically treated genomic DNA (Homo sapiens)
ttatggatatgtagaaaatgtattttgttttagtgatagatagagggagtttgatattgaatttatatttgtagtgttgattttagattgattagggaaaatgattgttttgtatttttgtaaaattttaatgttttgtgagaagtgtattgtgttgtttgatgtatagtgtttaggtttgttttgattttattaaattatggaaagtggtttttatgagggaagtttttttttttttttttttttttttttttttttaattttttgtatgattatttaggtggttttttttgtttttatttttttttttggatttgggtttttaattattattgtagagaattggaaggatgtttgttttgtttttttagtatggttttttttgtttggtttttaggttatttgtttttatttttttttttttttagttttgttttttttgtgtattttgtggttagtagttagatagttgtgtttaattagaaattatttataattttagatgatttgaaggggaaattttggtgtgaagtgtagttttgttttggttttttttagttttttttttttttttggaatttgaggtgtggattttttttgttttattgttttagtttttttgggagtttgttggtgttttttagggtgttggtttgtgttgggaagtgttttttattttggtaatggggggtggtgaggtattgtaggagtggtgaggtggtgtttagggtggtattgttttggaatggggtgttgggtgtgtgtgggagggtttttgtgtgggttttgttgttgttgtagttgtgagtgtgttgtgttattgagttttttgtagtaatggtttgtttgtgaaatgtgagttatggttgttttttttaagagtgtttgtattttttgtttgtgttttgtaattgttttgggtgaaaatggttgtttagattaaaatgtggtagaagggattaagtagtggatattgagtttgtgaagtttaatttttaagttttgagatttgggggattgagagtttagttttgaaaagtgtattatgaattttggagttgttatgaaatatggaaatgattttttggttgagttgagtgaggtaagaaatagttgaggttgggagttagggttttttgggtgtttgtttttttataaatttttttttttttgatttttttatagttttatattttatttttttgtgtttttagtttggaggtagtgtaattgaagattagtagattttttggattttgttttaatttgtagtgttatttaggaaatgatttgaattggtttgatattttagtttgaaatgagtggtttaaaaaagaaataattttttattaagttgtgaggtgttttatttatgatagtttgtattttttgtttttttttatttgtttttattaatttatatatatttttttatttttttgattggtaaaatatatggatatttttgatgagtttaagttgatggtaattaatttggtgggggtggtggtggtgagggggatgtattggattattttattttttatttggtttttatgtgtgatgtgtttggttatgtttaggtaaggtggttttttttttaggttgagatgaatgatttgtgttggggaggggtttggggagaaagtattttaaagttgttttagaattttattttttgtttggatttggatttttga

SEQ ID NO: 174 - DNA, 1700 bp, Artificial Sequence
  chemically treated genomic DNA (Homo sapiens)
gttggtaagtgaaaagtttatagtttatagtatttttattgtttagttttatttttattgttgagtttaggaatttgtttttttgtttttttttggaagtgattttgtgtgtgtaggtaggggtagagggagtaaagttaaatgtaattatagtattttagatttgtagatttttaaattagttttatattttagtgaggtgataatggttaatatttttattttagttttatagaagtttagttatagtaggtgatttgtgtaaagttatatagtttttggttttaaggtagggtttttttttttgtttgtgtttttttgttattgtagtttttgttttttagttgggggattatttagaggattatttggattttgttgtttttgttgggttatttgtttggtggagtagttggggggtggggggtttgtaatttaggattttatttggtgttgttttagtttattgagtagtgagttagttttttttttattggtggtagggggagttgttgggattttagggttgttttgaggggttgtgtagatttgggggatgtaaaggtttaaatggatttggaatggttggtgtggttgggttttgggaaatgaagggtttgtggtgttttgtattttttgtgggggtgggagtaattattaggtggttgtggagttagttgagggggtggttgttaatttgggtggtgattgtaggtggagatgttttgtttgggatagggattgggttgtgttggtgtttgtggatagatatttgtggttttggttgttgttgtgttttttgtttttgttatgttttggtggttttgtttggggtgtgtttttgttttgtatgtgtatgtgtttgtatttgtattggtttgtttggtttttttgtgtttgtggggttgttttgggggaggtgttgagatttggttttggtttgtgtgtggtttttggggtttggtgtgttggggatttataggtgaggtgagagaaaggggttggtttgtggggatatttatttgtgtggtgtggggttgtggtgttggttattattattttgtgtttttttttttgttagtgtgtatgtagatggtggggtggtttggggaggtttttgggtttttttttgggaatgtagggttaagttgtgttttgattttatgttttttttatttatgttgggtatatgtagttttgagtgagggtttatgttttgttttttatgttgattatagtttttaggtttggtgtttgttgggaagggttgtaatggtttagggatttttgtttttattattattttgttttggggggataggttttagaatattgtgtggggtttagagtgggtttagttgtaaaaggataggggtttttttttagttatgtttatttttgggtgttttggttgttagtagttttttattataattgtagggtgagaagtttagagaatagtttaggagtagtttttttgttttttattatgtttgtgtgtttagtatgggtaaattagtagttatgtaataaatgaagtgtggtgttgtagttttgggtttttttgtatttgttttgattttagtttgtttagtttagtttggggatttgtatttaggtttgtagttttttgggtttttgtttggtgttttttagagttggagtttgtttgttgtagggatggtttagaggatttttta

SEQ ID NO: 175 - DNA, 1700 bp, Artificial Sequence
  chemically treated genomic DNA (Homo sapiens)
taggaggttttttaagttatttttgtggtagataggttttggttttgaggagtattgggtagggatttaggaggttgtaaatttaagtgtagatttttaggttgggttggataggttgaggttagggtaggtgtagagaggtttagagttgtaatattgtattttatttattgtgtggttattgatttgtttgtgttgggtatatggatgtggtaaaggatagaagggttatttttggattgttttttgggttttttattttgtggttgtggtgagaggttgttgatagttagggtatttgggagtgggtgtggttggagaaggatttttgtttttttatagttaggtttattttgagttttatgtagtgttttagagtttgtttttttagggtaggatggtagtgggggtaggggtttttgagttattgtggttttttttggtaggtgttaagtttaggggttgtggttagtgtgaaaggtagggtatggatttttatttagggttgtgtgtgtttgatgtgggtggggggggtgtggaattggagtataatttggttttgtgtttttaggaagggatttgaagatttttttaggttattttgttatttgtgtgtgtgttggtaagaggaggggtgtggggtaatggtggttggtgttatagttttgtgttgtgtaggtaggtgtttttgtgagttaattttttttttttgttttgtttgtgggtttttggtgtgttggattttggaaattgtgtgtgggttaaggttgggttttggtattttttttggagtggttttgtgagtgtgggggaattgggtgggttagtgtgggtgtaggtgtgtgtgtgtgtgaggtgggggtgtgttttaagtgaggttgttggggtgtggtgggggtgggagatgtggtagtggttgggattgtgggtgtttgtttgtgagtgttggtgtggtttggtttttgttttgggtggggtgtttttgtttgtggttgttgtttgggttagtagttgtttttttggttggttttgtggttatttggtaattatttttgtttttataaaaagtgtgagatattgtgagttttttattttttagagtttggttgtattggttattttgagtttgtttgggtttttatattttttagatttgtgtggttttttagggtagttttgagattttgatagttttttttgttattgatgaggggagagttggtttattgtttggtgggttggggtagtgttaggtggggttttaggttgtaggttttttattttttaattattttgttaagtaaatggtttaatggaggtagtagggtttaggtggttttttgggtggttttttagttggagaataggggttgtggtggtgagagggtataggtaggggagagagttttgttttggagttagaggttgtgtagttttgtgtaggttatttgttgtggttgggtttttgtaaaattgagatgaaaatgttagttattgttattttattgaggtgtgaggttggtttgagagtttgtaaatttgagatgttatagttatgtttggttttattttttttgtttttatttgtatatataggattgtttttaaaaggaggtagggaaataaatttttgagtttaatagtggaagtggggttggatagtgaggatgttgtggattgtgggttttttatttgttagt

SEQ ID NO: 176 - DNA, 1700 bp, Artificial Sequence
  chemically treated genomic DNA (Homo sapiens)
gtaagattttattttaaaaaaaaaaaaaaaaaaaagtgatttgtttagtaatagttttaaaatatggaagagttgggattggatttagattatttggttttggaattgggttggtaagtgaaaagtttatagtttatagtatttttattgtttagttttatttttattgttgagtttaggaatttgtttttttgtttttttttggaagtgattttgtgtgtgtaggtaggggtagagggagtaaagttaaatgtaattatagtattttagatttgtagatttttaaattagttttatattttagtgaggtgataatggttaatatttttattttagttttatagaagtttagttatagtaggtgatttgtgtaaagttatatagtttttggttttaaggtagggtttttttttttgtttgtgtttttttgttattgtagtttttgttttttagttgggggattatttagaggattatttggattttgttgtttttgttgggttatttgtttggtggagtagttggggggtggggggtttgtaatttaggattttatttggtgttgttttagtttattgagtagtgagttagttttttttttattggtggtagggggagttgttgggattttagggttgttttgaggggttgtgtagatttgggggatgtaaaggtttaaatggatttggaatggttggtgtggttgggttttgggaaatgaagggtttgtggtgttttgtattttttgtgggggtgggagtaattattaggtggttgtggagttagttgagggggtggttgttaatttgggtggtgattgtaggtggagatgttttgtttgggatagggattgggttgtgttggtgtttgtggatagatatttgtggttttggttgttgttgtgttttttgtttttgttatgttttggtggttttgtttggggtgtgtttttgttttgtatgtgtatgtgtttgtatttgtattggtttgtttggtttttttgtgtttgtggggttgttttgggggaggtgttgagatttggttttggtttgtgtgtggtttttggggtttggtgtgttggggatttataggtgaggtgagagaaaggggttggtttgtggggatatttatttgtgtggtgtggggttgtggtgttggttattattattttgtgtttttttttttgttagtgtgtatgtagatggtggggtggtttggggaggtttttgggtttttttttgggaatgtagggttaagttgtgttttgattttatgttttttttatttatgttgggtatatgtagttttgagtgagggtttatgttttgttttttatgttgattatagtttttaggtttggtgtttgttgggaagggttgtaatggtttagggatttttgtttttattattattttgttttggggggataggttttagaatattgtgtggggtttagagtgggtttagttgtaaaaggataggggtttttttttagttatgtttatttttgggtgttttggttgttagtagttttttattataattgtagggtgagaagtttagagaatagtttaggagtagtttttttgttttttattatgtttgtgtgtttagtatgggtaaattagtagttatgtaataaatgaagtgtggtgttgtagttttgggtttttttgtatttgttttgattttagtttgtt

SEQ ID NO: 177 - DNA, 1700 bp, Artificial Sequence
  chemically treated genomic DNA (Homo sapiens)
gataggttgaggttagggtaggtgtagagaggtttagagttgtaatattgtattttatttattgtgtggttattgatttgtttgtgttgggtatatggatgtggtaaaggatagaagggttatttttggattgttttttgggttttttattttgtggttgtggtgagaggttgttgatagttagggtatttgggagtgggtgtggttggagaaggatttttgtttttttatagttaggtttattttgagttttatgtagtgttttagagtttgtttttttagggtaggatggtagtgggggtaggggtttttgagttattgtggttttttttggtaggtgttaagtttaggggttgtggttagtgtgaaaggtagggtatggatttttatttagggttgtgtgtgtttgatgtgggtggggggggtgtggaattggagtataatttggttttgtgtttttaggaagggatttgaagatttttttaggttattttgttatttgtgtgtgtgttggtaagaggaggggtgtggggtaatggtggttggtgttatagttttgtgttgtgtaggtaggtgtttttgtgagttaattttttttttttgttttgtttgtgggtttttggtgtgttggattttggaaattgtgtgtgggttaaggttgggttttggtattttttttggagtggttttgtgagtgtgggggaattgggtgggttagtgtgggtgtaggtgtgtgtgtgtgtgaggtgggggtgtgttttaagtgaggttgttggggtgtggtgggggtgggagatgtggtagtggttgggattgtgggtgtttgtttgtgagtgttggtgtggtttggtttttgttttgggtggggtgtttttgtttgtggttgttgtttgggttagtagttgtttttttggttggttttgtggttatttggtaattatttttgtttttataaaaagtgtgagatattgtgagttttttattttttagagtttggttgtattggttattttgagtttgtttgggtttttatattttttagatttgtgtggttttttagggtagttttgagattttgatagttttttttgttattgatgaggggagagttggtttattgtttggtgggttggggtagtgttaggtggggttttaggttgtaggttttttattttttaattattttgttaagtaaatggtttaatggaggtagtagggtttaggtggttttttgggtggttttttagttggagaataggggttgtggtggtgagagggtataggtaggggagagagttttgttttggagttagaggttgtgtagttttgtgtaggttatttgttgtggttgggtttttgtaaaattgagatgaaaatgttagttattgttattttattgaggtgtgaggttggtttgagagtttgtaaatttgagatgttatagttatgtttggttttattttttttgtttttatttgtatatataggattgtttttaaaaggaggtagggaaataaatttttgagtttaatagtggaagtggggttggatagtgaggatgttgtggattgtgggttttttatttgttagtttagttttagaattagatagtttaagtttaattttagtttttttatattttagagttattattgggtaagttgtttttttttttttttttttttgagatggagttttgt

SEQ ID NO: 178 - DNA, 1700 bp, Artificial Sequence
  chemically treated genomic DNA (Homo sapiens)
aggtgggtaggttttttggagtagtaggtggtttttttagtttttatggttggagttaggatttttttttgtttataatttttttaatttttttaatttaatattttttattttttttttttttttttttttttttttttttttttttttttttttttttttttttttttttttttttttttttttttttttttttttttttgttttttttttttttttagttttttttttttattttgttgtgggtgggttgtagtgttgattttattgggatgtttggataggtattgtttgtgataatggtggtattggaaggttttggtagttgaatgtttagttttttattttttatgtttttatattttttatgtataaattttgtaggaagtaggttagggaatgtagtttattgtttttttttgattagtagttttagtttggttatattgtagttgtggtagaggatatggtttatttttatgagttaattggggtttatttttatttttggtggttttagtgggtttagtaagtgagagtaggtttttggagttataggggtttttttgtttttgtagtgagggaagtaggaggtggtaggttttgaggtttgtgggttgaaggtttaggttttatgtggagttatttgtggaggatttaggttaggttaatttgtttgtggggaatttgtttgattttggtgagttaggggtttggaaggttgggtggagtttggttaatagtttgggttttttattttgggttttgttttttttggaggttttgggggtatttagtgggtaggtttttttggtttggttggttttggagtaggtgaggaaggttaggtaggttttgtggaggtgggttaggttttggtgggtggttttgttgatgaagtagtagagtatggggttggtgatgtagttgaagttggtgagtaggagggagaagtggtaggtgttgaaaatgtttttggtgaagttgtagttggttttttagatgttgtgtattagtagtaatatgtggtagggtaggaagtaggttaggaagatgattatggtgttgagtattagttgttggatttggtttttgtggtttttttgggtgttgtggttttggtgtatggtgtgtaggatgttttggtaggatgttagtagtaggtagatggggaagaggaagtttattaggaagtggtagtagttgatggtgtgttgttatgtttggatggggtagtgtttaaagtatatgtggtgttggtttttgtttttgatgatttttttgtgtattaggaagtagatgttggttagtagttttttggtttagatgattatgttgatgttgatggttgtttttagggtttggaattggtggaagtggaagggatgggttatagttaggtagtggtttatggagatgtagtagaggaagtttatgttgatgtagatgtttttgtataggaggatgttgtatatttggtaggataggttgttgtgagattagttgttgtgttgtagtatgtattgtagttagaagggtagtgagtagatgtagaagaggttggttattgttaggttgtataggtatatgtttagtttgttttgggttttgatttgtaggtagttgaagtagagggataggtagttggttgggaagtttattattagtatggtaatatagattattggggttagtgtttg

SEQ ID NO: 179 - DNA, 1700 bp, Artificial Sequence
  chemically treated genomic DNA (Homo sapiens)
tagatgttggttttggtggtttatgttattgtgttggtggtgggttttttggttaattgtttgtttttttattttggttatttgtagattaaggtttggaatgagttgggtgtgtatttgtgtaatttgatggtggttgattttttttatatttgtttgttgtttttttggttgtagtatgtgttgtagtatgataattggttttatggtgatttgttttgttaggtgtgtggtatttttttgtatgagaatatttatattagtgtgggttttttttgttgtatttttgtggattgttatttggttgtggtttattttttttgtttttattagttttggattttgaaggtggttgttggtgttagtgtggttatttgggttaaggagttgttgattagtatttattttttgatgtatgaggaggttattgaggatgagaattagtattgtgtgtgttttgagtattattttatttaggtatggtagtgtgttattaattattattgttttttggtgggtttttttttttttatttgtttgttgttggtgttttattagggtattttgtgtgttgtgtgttggagttatggtatttagaagagttgtaaggattagatttagtggttggtgtttagtattgtggttatttttttggtttgttttttgttttattatgtgttgttgttggtgtgtagtgtttgggaggttagttgtgattttgttaagggtgtttttaatgtttattattttttttttttgtttattagttttaattgtgttgttgattttgtgttttattgttttgttagtgagattatttattgggatttggtttgtttttgtggggtttgtttggttttttttatttgttttaggattggttgggttagggaggtttatttgttgggtgtttttgaggtttttgggaaaagtggggtttagggtgaggagtttgagttgttgattaagttttatttggttttttagatttttaatttgttagggttgggtgggttttttatgggtaggttggtttagtttgggttttttgtgggtggttttatgtgaggtttgagtttttagtttatgggttttagggtttgttgttttttgttttttttgttgtggaggtagggaagtttttgtaattttggaagtttgtttttgtttgttgagtttgttgggattgttgagggtgggaataagttttggttggtttgtgggaataagttgtgttttttgttgtggttgtgatgtggttaggttggggttgttggttgggggaagatagtgaattgtgttttttggtttgttttttgtagagtttgtgtatggggagtgtgaggatatggagggtgggaggttgggtgtttagttgttagggttttttaatgttattgttgttatagataatgtttgtttaaatgttttggtgggattagtattgtagtttgtttataatagggtgggaagggaagattggagggggaaggaaggtaggagggggaaggaagaaagagggggaaggaaagaaagaggggggaaggaaaggaggaaggggagaaggaagggaggaggaaagaaggaaggtagggggtgttgggttgggagaattgagggagttataggtagaggaggattttagttttagttataagaattgggagagttgtttgttgttttagaagatttgtttattt

SEQ ID NO: 180 - DNA, 1700 bp, Artificial Sequence; identical to SEQ ID NO: 178
  chemically treated genomic DNA (Homo sapiens)

SEQ ID NO: 181 - DNA, 1700 bp, Artificial Sequence; identical to SEQ ID NO: 179
  chemically treated genomic DNA (Homo sapiens)

SEQ ID NO: 182 - DNA, 22 bp, Artificial Sequence
  chemically treated genomic DNA (Homo sapiens)
aaacgactaccgaaaaatacgc

SEQ ID NO: 183 - DNA, 23 bp, Artificial Sequence
  chemically treated genomic DNA (Homo sapiens)
ggtacgttttttttacggtcgta

SEQ ID NO: 184 - DNA, 22 bp, Artificial Sequence
  chemically treated genomic DNA (Homo sapiens)
aaatccaacgcgatccgaacgc

SEQ ID NO: 185 - DNA, 26 bp, Artificial Sequence
  chemically treated genomic DNA (Homo sapiens)
gggattagaatttttttatgcgagtt

SEQ ID NO: 186 - DNA, 20 bp, Artificial Sequence
  chemically treated genomic DNA (Homo sapiens)
cgaatcctaccccgacgata

SEQ ID NO: 187 - DNA, 23 bp, Artificial Sequence
  chemically treated genomic DNA (Homo sapiens)
cgaatcgctcgcgttctcgacat

SEQ ID NO: 188 - DNA, 22 bp, Artificial Sequence
  chemically treated genomic DNA (Homo sapiens)
gtattcgttcggttcgtatcgt

SEQ ID NO: 189 - DNA, 17 bp, Artificial Sequence
  chemically treated genomic DNA (Homo sapiens)
cgtcccgcgaccaataa

SEQ ID NO: 190 - DNA, 33 bp, Artificial Sequence
  chemically treated genomic DNA (Homo sapiens)
cgctcccctaaactataaacgaaccgaaaaacg

SEQ ID NO: 191 - DNA, 18 bp, Artificial Sequence
  chemically treated genomic DNA (Homo sapiens)
caaacacccgaaaaccga

SEQ ID NO: 192 - DNA, 19 bp, Artificial Sequence
  chemically treated genomic DNA (Homo sapiens)
gggaagaggatacggcgta

SEQ ID NO: 193 - DNA, 23 bp, Artificial Sequence
  chemically treated genomic DNA (Homo sapiens)
ctccgcgaacgtcccatacgaaa

SEQ ID NO: 194 - DNA, 17 bp, Artificial Sequence
  chemically treated genomic DNA (Homo sapiens)
cgacgaacgcgacgaaa

SEQ ID NO: 195 - DNA, 18 bp, Artificial Sequence
  chemically treated genomic DNA (Homo sapiens)
gcgtttgcggcgtttagt

SEQ ID NO: 196 - DNA, 30 bp, Artificial Sequence
  chemically treated genomic DNA (Homo sapiens)
cgactcctcgatatcaaaacgaacgaaacg

SEQ ID NO: 197 - DNA, 19 bp, Artificial Sequence
  chemically treated genomic DNA (Homo sapiens)
tcggtgaggttttcgtcga

SEQ ID NO: 198 - DNA, 21 bp, Artificial Sequence
  chemically treated genomic DNA (Homo sapiens)
cgctacacgaacttatcccga

SEQ ID NO: 199 - DNA, 23 bp, Artificial Sequence
  chemically treated genomic DNA (Homo sapiens)
atcacgtccaaccgacgtacgcc

SEQ ID NO: 200 - DNA, 17 bp, Artificial Sequence
  chemically treated genomic DNA (Homo sapiens)
aaacgtaaccccgccga

SEQ ID NO: 201 - DNA, 19 bp, Artificial Sequence
  chemically treated genomic DNA (Homo sapiens)
gtagtagcgcgtcggttcg

SEQ ID NO: 202 - DNA, 22 bp, Artificial Sequence
  chemically treated genomic DNA (Homo sapiens)
cgctaccctctccgctacccgc

SEQ ID NO: 203 - DNA, 17 bp, Artificial Sequence
  chemically treated genomic DNA (Homo sapiens)
gcgtttggaattcggcg

SEQ ID NO: 204 - DNA, 24 bp, Artificial Sequence
  chemically treated genomic DNA (Homo sapiens)
gaatcaaccgtctataaacaacgc

SEQ ID NO: 205 - DNA, 25 bp, Artificial Sequence
  chemically treated genomic DNA (Homo sapiens)
tccgccgacgcttaacgtcaatacc

SEQ ID NO: 206 - DNA, 23 bp, Artificial Sequence
  chemically treated genomic DNA (Homo sapiens)
attttcgttaaaaacgtcggttt

SEQ ID NO: 207 - DNA, 21 bp, Artificial Sequence
  chemically treated genomic DNA (Homo sapiens)
aacgaaacgacgacgtaacgt

SEQ ID NO: 208 - DNA, 34 bp, Artificial Sequence
  chemically treated genomic DNA (Homo sapiens)
ataattaaaacacgaaaaaacgtaaccgaacgac

SEQ ID NO: 209 - DNA, 22 bp, Artificial Sequence
  chemically treated genomic DNA (Homo sapiens)
tgtcgtacgtagttttaggcgg

SEQ ID NO: 210 - DNA, 17 bp, Artificial Sequence
  chemically treated genomic DNA (Homo sapiens)
cgacgcctccgcctaat

SEQ ID NO: 211 - DNA, 23 bp, Artificial Sequence
  chemically treated genomic DNA (Homo sapiens)
ctaaaccccgacgaaaaccccgt

SEQ ID NO: 212 - DNA, 20 bp, Artificial Sequence
  chemically treated genomic DNA (Homo sapiens)
tattttgtaggcgttcgcgg

SEQ ID NO: 213 - DNA, 20 bp, Artificial Sequence
  chemically treated genomic DNA (Homo sapiens)
ccgacaaaaacaaaaatcgc

SEQ ID NO: 214 - DNA, 31 bp, Artificial Sequence
  chemically treated genomic DNA (Homo sapiens)
atcgtccttctttcgaaatacaaaactccga

SEQ ID NO: 215 - DNA, 17 bp, Artificial Sequence
  chemically treated genomic DNA (Homo sapiens)
gccctaccgaacccgaa

SEQ ID NO: 216 - DNA, 20 bp, Artificial Sequence
  chemically treated genomic DNA (Homo sapiens)
gtcgtttaatcgggagtcgg

SEQ ID NO: 217 - DNA, 24 bp, Artificial Sequence
  chemically treated genomic DNA (Homo sapiens)
acaaacgctcgacgcgtacgtcac

SEQ ID NO: 218 - DNA, 21 bp, Artificial Sequence
  chemically treated genomic DNA (Homo sapiens)
actacgccgaaaacctacgac

SEQ ID NO: 219 - DNA, 23 bp, Artificial Sequence
  chemically treated genomic DNA (Homo sapiens)
gaggtttcgtattttagacgcgt

SEQ ID NO: 220 - DNA, 23 bp, Artificial Sequence
  chemically treated genomic DNA (Homo sapiens)
cgaccacgccctacctcgacgac

SEQ ID NO: 221 - DNA, 21 bp, Artificial Sequence
  chemically treated genomic DNA (Homo sapiens)
gcgaaagtttcgtttcgtaga

SEQ ID NO: 222 - DNA, 21 bp, Artificial Sequence
  chemically treated genomic DNA (Homo sapiens)
acaaaaaccgaaccgaaaatc

SEQ ID NO: 223 - DNA, 24 bp, Artificial Sequence
  chemically treated genomic DNA (Homo sapiens)
tccgaactctaaactcgcgatcgc

SEQ ID NO: 224 - DNA, 23 bp, Artificial Sequence
  chemically treated genomic DNA (Homo sapiens)
ttcgttgaggttttaaacgcgta

SEQ ID NO: 225 - DNA, 23 bp, Artificial Sequence
  chemically treated genomic DNA (Homo sapiens)
aatccgaactctaaactcgcgat

SEQ ID NO: 226 - DNA, 26 bp, Artificial Sequence
  chemically treated genomic DNA (Homo sapiens)
tacgaaacgaaactttcgccccgata

SEQ ID NO: 227 - DNA, 21 bp, Artificial Sequence
  chemically treated genomic DNA (Homo sapiens)
tagtaatcggagcgatttgcg

SEQ ID NO: 228 - DNA, 23 bp, Artificial Sequence
  chemically treated genomic DNA (Homo sapiens)
caaaatatacgacccgacgaata

SEQ ID NO: 229 - DNA, 30 bp, Artificial Sequence
  chemically treated genomic DNA (Homo sapiens)
cgtacgtatcttctcgaaattcccaaccgc

SEQ ID NO: 230 - DNA, 24 bp, Artificial Sequence
  chemically treated genomic DNA (Homo sapiens)
gtattttaggttttgattcgcgtg

SEQ ID NO: 231 - DNA, 25 bp, Artificial Sequence
  chemically treated genomic DNA (Homo sapiens)
actcgtacgaatcctctaaaacgaa

SEQ ID NO: 232 - DNA, 24 bp, Artificial Sequence
  chemically treated genomic DNA (Homo sapiens)
actaccccacgaaatacctcgcgc

SEQ ID NO: 233 - DNA, 23 bp, Artificial Sequence
  chemically treated genomic DNA (Homo sapiens)
gagggggtttagtatgtcgttcg

SEQ ID NO: 234 - DNA, 23 bp, Artificial Sequence
  chemically treated genomic DNA (Homo sapiens)
cgaaactataacgtcgactccga

SEQ ID NO: 235 - DNA, 22 bp, Artificial Sequence
  chemically treated genomic DNA (Homo sapiens)
aaaaccgaaccgccgccgaaaa

SEQ ID NO: 236 - DNA, 24 bp, Artificial Sequence
  chemically treated genomic DNA (Homo sapiens)
acgaatatttacaatcatcaccga

SEQ ID NO: 237 - DNA, 20 bp, Artificial Sequence
  chemically treated genomic DNA (Homo sapiens)
gcgagaatttagcgagtcgt

SEQ ID NO: 238 - DNA, 23 bp, Artificial Sequence
  chemically treated genomic DNA (Homo sapiens)
ccgcgataaaaattccgcctcgt

SEQ ID NO: 239 - DNA, 24 bp, Artificial Sequence
  chemically treated genomic DNA (Homo sapiens)
aaacaacgaaattaaaacgacgaa

SEQ ID NO: 240 - DNA, 22 bp, Artificial Sequence
  chemically treated genomic DNA (Homo sapiens)
gagggaatttttttcgttcgat

SEQ ID NO: 241 - DNA, 29 bp, Artificial Sequence
  chemically treated genomic DNA (Homo sapiens)
acgatactccgaaaaaactccgcgataaa

SEQ ID NO: 242 - DNA, 27 bp, Artificial Sequence
  chemically treated genomic DNA (Homo sapiens)
aaaacctaacgaacgaaaatacgaata

SEQ ID NO: 243 - DNA, 18 bp, Artificial Sequence
  chemically treated genomic DNA (Homo sapiens)
tcgggattcgtcgttcgt

SEQ ID NO: 244 - DNA, 29 bp, Artificial Sequence
  chemically treated genomic DNA (Homo sapiens)
tccgaaccgaataacgtaaaaaaccgaac

SEQ ID NO: 245 - DNA, 19 bp, Artificial Sequence
  chemically treated genomic DNA (Homo sapiens)
tttttcgagaatgaggcgg

SEQ ID NO: 246 - DNA, 23 bp, Artificial Sequence
  chemically treated genomic DNA (Homo sapiens)
aaataaacgcaacgctactacga

SEQ ID NO: 247 - DNA, 23 bp, Artificial Sequence
  chemically treated genomic DNA (Homo sapiens)
cgaacgcgtcatcaacttcccga

SEQ ID NO: 248 - DNA, 19 bp, Artificial Sequence
  chemically treated genomic DNA (Homo sapiens)
aagttgatgacgcgttcgg

SEQ ID NO: 249 - DNA, 23 bp, Artificial Sequence
  chemically treated genomic DNA (Homo sapiens)
aaataaacgcaacgctactacga

SEQ ID NO: 250 - DNA, 29 bp, Artificial Sequence
  chemically treated genomic DNA (Homo sapiens)
acgcgcgtctcgaaaacaaactaacttac

SEQ ID NO: 251 - DNA, 24 bp, Artificial Sequence
  chemically treated genomic DNA (Homo sapiens)
cgaaaataaaaaacgctaccgata

SEQ ID NO: 252 - DNA, 20 bp, Artificial Sequence
  chemically treated genomic DNA (Homo sapiens)
ttcgtacggatgtcgaaggt

SEQ ID NO: 253 - DNA, 23 bp, Artificial Sequence
  chemically treated genomic DNA (Homo sapiens)
acgccaaaacgcactacaacgcc

SEQ ID NO: 254 - DNA, 20 bp, Artificial Sequence
  chemically treated genomic DNA (Homo sapiens)
tagttgcgaagcgtaaacgg

SEQ ID NO: 255 - DNA, 18 bp, Artificial Sequence
  chemically treated genomic DNA (Homo sapiens)
cgccaccgaacctcctac

SEQ ID NO: 256 - DNA, 24 bp, Artificial Sequence
  chemically treated genomic DNA (Homo sapiens)
tcgtccgtaaaacgcgaaccacga

SEQ ID NO: 257 - DNA, 21 bp, Artificial Sequence
  chemically treated genomic DNA (Homo sapiens)
aaaaaactcgcgatatctcgc

SEQ ID NO: 258 - DNA, 21 bp, Artificial Sequence
  chemically treated genomic DNA (Homo sapiens)
cgtcgttcgggttagtagtcg

SEQ ID NO: 259 - DNA, 32 bp, Artificial Sequence
  chemically treated genomic DNA (Homo sapiens)
acgaaaataattaccaaataaccgcgaaacca

SEQ ID NO: 260 - DNA, 18 bp, Artificial Sequence
  chemically treated genomic DNA (Homo sapiens)
taggcggagacgtttcgt

SEQ ID NO: 261 - DNA, 17 bp, Artificial Sequence
  chemically treated genomic DNA (Homo sapiens)
aacgcgacaacgaccga

SEQ ID NO: 262 - DNA, 24 bp, Artificial Sequence
  chemically treated genomic DNA (Homo sapiens)
cgaatatctatccgcgaacgccga

SEQ ID NO: 263 - DNA, 20 bp, Artificial Sequence
  chemically treated genomic DNA (Homo sapiens)
aggtttttttggttcggtcg

SEQ ID NO: 264 - DNA, 25 bp, Artificial Sequence
  chemically treated genomic DNA (Homo sapiens)
ccgtactctactacttcgtcaacga

SEQ ID NO: 265 - DNA, 22 bp, Artificial Sequence
  chemically treated genomic DNA (Homo sapiens)
cgaaacctaacccgcctccgcg

SEQ ID NO: 266 - DNA, 19 bp, Artificial Sequence
  chemically treated genomic DNA (Homo sapiens)
ctccctaacccgaccgatc

SEQ ID NO: 267 - DNA, 24 bp, Artificial Sequence
  chemically treated genomic DNA (Homo sapiens)
cgtgttttattgtttcgttagcga

SEQ ID NO: 268 - DNA, 23 bp, Artificial Sequence
  chemically treated genomic DNA (Homo sapiens)
cgcgaaaacgaaccaaatcccga

SEQ ID NO: 269 - DNA, 20 bp, Artificial Sequence
  chemically treated genomic DNA (Homo sapiens)
tagtgagtacgcgcggttcg